(19) United States
(12) Patent Application Publication
(10) Pub. No.: US 2012/0046305 A1
(43) Pub. Date: Feb. 23, 2012

(54) METHODS AND COMPOSITIONS FOR TREATING RESPIRATORY DISORDERS

(76) Inventors: Magdalene M. Moran, Brookline, MA (US); Colleen McNamara, Brookline, MA (US); Neil Hayward, Grafton, MA (US)

(21) Appl. No.: 13/120,891

(22) PCT Filed: Sep. 24, 2009
(86) PCT No.: PCT/US09/58259

§ 371 Date: Nov. 8, 2011

Related U.S. Application Data
(60) Provisional application No. 61/099,760, filed on Sep. 24, 2008.

(51) Int. Cl. A61K 31/52; A61P 11/08; A61P 11/00

(52) U.S. Cl. 514/263.2; 514/263.1

(57) ABSTRACT

Compounds and compositions for treating disorders related to TRPA1 are described herein.

### CLAIM OF PRIORITY

This application claims priority from U.S. Ser. No. 61/099,760, filed Sep. 24, 2008, which is incorporated herein by reference in its entirety.

### BACKGROUND

A variety of ion channel proteins exist to mediate ion flux across cellular membranes. The proper expression and function of ion channel proteins is essential for the maintenance of cell function, intracellular communication, and the like. Numerous diseases are the result of misregulation of membrane potential or aberrant calcium handling. Given the central importance of ion channels in modulating membrane potential and ion flux in cells, identification of agents that can promote or inhibit particular ion channels are of great interest as research tools and as therapeutic agents.

### SUMMARY OF THE INVENTION

The present invention provides compounds, methods and compositions for treating or preventing respiratory conditions by modulating the activity of the TRPA1 channel. The compounds described herein can modulate the function of TRPA1 by inhibiting a TRPA1-mediated ion flux or by inhibiting the inward current, the outward current, or both currents mediated by TRPA1. The inhibition of a particular current is the ability to inhibit or reduce such current (e.g., inward and/or outward) in an in vitro or an in vivo assay. The following articles are exemplary of the state of the art regarding the structure and function of TRPA1 (Jordt et al. (2004) Nature 427:260-265; Bautista et al., (2005) PNAS: 102(34):12248-12252). The foregoing articles are incorporated by reference in their entirety.

One aspect of the present invention relates to a method for treating or preventing a condition involving activation of TRPA1 or for which reduced TRPA1 activity can reduce the severity by administering a TRPA1 antagonist that inhibits TRPA1-mediated current and/or TRPA1-mediated ion flux. The compounds described herein can be useful for the treatment or prevention of respiratory conditions. Such conditions affect the lung, pleural cavity, bronchial tubes, trachea, upper respiratory tract as well as the nerves and muscles involved in breathing. Respiratory diseases that may be treated with the compounds described herein include obstructive diseases such as chronic obstructive pulmonary disease (COPD), emphysema, chronic bronchitis, asthma (including asthma caused by industrial irritants), cystic fibrosis, bronchiectasis, bronchiolitis, allergic bronchopulmonary aspergillosis, and tuberculosis; restrictive lung disease including asbestosis, radiation fibrosis, hypersensitivity pneumonitis, acute respiratory distress syndrome, infant respiratory distress syndrome (ARDS), idiopathic pulmonary fibrosis, idiopathic pulmonary fibrosis, idiopathic interstial pneumonia sarcoidosis, eosinophilic pneumonialymphangioleiomyomatosis, pulmonary Langerhan's cell histiocytosis, and pulmonary alveolar proteinosis; respiratory tract infections including upper respiratory tract infections (e.g., common cold, sinusitis, tonsillitis, pharyngitis and laryngitis) and lower respiratory tract infections (e.g., pneumonia); respiratory tumors whether malignant (e.g., small cell lung cancer, non-small cell lung cancer, adenocarcinoma, squamous cell carcinoma, large cell undifferentiated carcinoma, carcinoid, mesothelioma, metastatic cancer of the lung, metastatic germ cell cancer, metastatic renal cell carcinoma) or benign (e.g., pulmonary hamartoma, congenital malformations such as pulmonary sequestration and congenital cystic adenomatoid malformation (CCAM)); pleural cavity diseases (e.g., empyema and mesothelioma); and pulmonary vascular diseases (e.g, pulmonary embolism such as thromboembolism, and air embolism (iatrogenic), pulmonary arterial hypertension, pulmonary edema, pulmonary hemorrhage, inflammation and damage to capillaries in the lung resulting in blood leaking into the alveoli. Other conditions that may be treated include disorders that affect breathing mechanics (e.g., obstructive sleep apnea, central sleep apnea, amyotrophic lateral sclerosis, Guillan-Barre syndrome, and myasthenia gravis). In one embodiment, the respiratory condition is not asthma, COPD, or chronic cough. The present compounds can also be useful for treating, reducing, or preventing one or more symptoms associated with respiratory conditions including, for example, shortness of breath or dyspnea, cough (with or without the production of sputum), coughing blood (haemoptysis), chest pain including pleuritic chest pain, noisy breathing, wheezing, and cyanosis.

Described in greater detail below are TRPA1 antagonists that have measured IC₅₀'s for inhibition of TRPA1 of 10 micromolar or less, 5 micromolar or less, 2 micromolar or less, 1 micromolar or less, 500 nanomolar or less, 200 nanomolar or less, 100 nanomolar or less, or 10 nanomolar or less. In certain embodiments, the TRPA1 antagonist inhibit one or both of inward and outward TRPA1-mediated current with an IC₅₀ of 1 micromolar or less, and more preferably with an IC₅₀ of 500 nanomolar or less, 200 nanomolar or less, 100 nanomolar or less, 25 nanomolar or less or 10 nanomolar or less. In certain embodiments, the TRPA1 antagonist inhibits at least 95% of TRPA1-mediated current or TRPA1-mediated ion flux when administered at 5 micromolar or less, and more preferably at 1 micromolar or less.

In certain embodiments, the subject TRPA1 antagonists inhibit TRPA1 with an IC₅₀ at least one order of magnitude lower than its IC₅₀ for inhibition of one or more of TRPV5, TRPV6, NaV 1.2, TRPV1, mitochondrial uniporter and hERG channel activities, and more preferably two or three orders of magnitude lower.

In certain embodiments, the subject TRPA1 antagonists are at least 10, 20, 30, 40, or 50 fold selective for inhibiting TRPA1 activity over that of one or more of TRPV5, TRPV6, NaV 1.2, TRPV1, mitochondrial uniporter, or hERG channel activities. In other words, the antagonist inhibits TRPA1 activity (one or more functions of TRPA1) 10, 20, 30, 40, or 50 times more potently than that of one or more of the other foregoing channels.

In certain embodiments, the subject TRPA1 antagonists inhibit TRPA1 with an IC₅₀ at least one order of magnitude more potent than its Ki for the AMPA receptor. In certain other embodiments, the subject TRPA1 antagonists inhibit TRPA1 with an IC₅₀ at least two orders of magnitude, or three orders of magnitude, or four orders of magnitude more potent than its Ki for the AMPA receptor. In certain embodiments, the subject TRPA1 antagonists do not appreciably bind the AMPA receptor. In other words, the subject antagonists inhibit TRPA1 with a particular IC₅₀ and, when administered at that concentration, the antagonist does not appreciably bind AMPA receptor (e.g., does specifically and appreciably bind the AMPA receptor). In certain embodiments, compounds of the invention inhibit a TRPA1-mediated current with an IC₅₀ that is more potent than its Ki for the AMPA receptor. In such embodiments, the ability of the subject TRPA1 inhibitors to decrease pain would thus be independent of binding to and modulation of the alpha-amino-3-hydroxy-5-methyl-4-isoxazolepropionic acid (AMPA) receptor.

In certain embodiments, the TRPA1 antagonists inhibit TRPA1 with an IC₅₀ at least one order of magnitude lower than its IC₅₀ for inhibition of TRPV1, and more preferably two or three orders of magnitude lower. In certain embodiments, the subject TRPA1 antagonists can be selected for selectivity for TRPA1 versus TRPV1 on the basis of having IC₅₀ for TRPV1 inhibition greater than 10 micromolar.

In certain embodiments, the TRPA1 antagonists inhibit one or more of TRPV2, TRPV4, TRPV3 and/or TRPM8 with an IC₅₀ of 10 micromolar or less.

In certain embodiments, the TRPA1 antagonist has a therapeutic index (T.I.) for treating the condition with the compound of 3 or greater, and more preferably has a T.I. of at least 5, 10, 25, 50 or 100.

In preferred embodiments, the TRPA1 inhibitor has an IC₅₀ for TRPA1 inhibition that, at that concentration, does not cause QT interval elongation in the patient nor alter temperature regulation in the patient.

In certain embodiments, a TRPA1 inhibitor used in the treatment of any of the diseases or indications disclosed herein has one or more of the structural or functional characteristics disclosed herein.

In one aspect, the invention features a compound of formula (I),

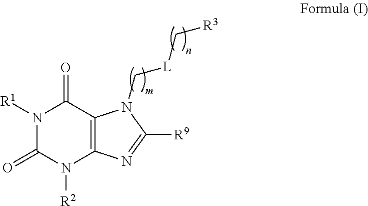

wherein,

R¹ and R² are each independently C₁-C₆ alkyl, C₂-C₆ alkenyl, or C₂-C₆ alkynyl, each of which is optionally substituted with 1-4 R⁵;

L is NR⁶SO₂, SO₂NR⁶, C(O)NR⁶, NR⁶C(O), OC(O)NR⁶, NR⁶C(O)O, NR⁶C(O)NR⁶, S, S(O), S(O)₂, NR⁶, CH₂, O, C(O), C(O)NS(O)₂, S(O)₂NC(O), heteroaryl, or cyclyl;

R³ is cyclyl, heterocyclyl, aryl, heteroaryl, each of which is optionally substituted with 1-4 R⁷;

each R⁵ is independently halo, hydroxyl, alkoxy, amino, alkylamino, dialkylamino, cyano, nitro, amido, alkylamido, dialkylamido, thioyl, sulfonyl, cyclyl, heterocyclyl, aryl, or heteroaryl;

each R⁶ is independently H, C₁-C₆ alkyl, C₁-C₆ alkenyl, hydroxyC₁-C₆ alkyl, alkoxyC₁-C₆ alkyl, cyanoalkyl, haloalkyl, arylalkyl, S(O)alkyl, acyl, amino, amidyl, or S(O)₂H, aryl, alkoxyaryl;

each R⁷ is independently C₁-C₆ alkyl, C₂-C₆ alkenyl, C₂-C₆ alkynyl, cyclyl, heterocyclyl, aryl, heteroaryl, cyclylalkyl, heterocyclylalkyl, arylalkyl, heteroarylalkyl, halo, hydroxyl, alkoxy, aryloxy, arylalkoxy, amino, alkylamino, dialkylamino, thioyl, alkylthioyl, sulfonyl, sulfonamidyl (e.g., where the nitrogen of the sulfonamide is substituted by an alkyl, or where the nitrogen of the sulfonamide together with two carbons to which it is attached, forms a ring), amido (e.g., where the nitrogen of the amide is substituted by an alkyl, or where the nitrogen of the amide together with two carbons to which it is attached, forms a ring), urea, sulfonylurea, acyl, —C(O)aryl, —NHC(O)aryl, —C(O)NHaryl, —C(O)OH, —C(O)Oalkyl, nitro, cyano, each of which is optionally substituted with 1-3 R⁸;

each R⁸ is independently C₁-C₆ alkyl, C₂-C₆ alkenyl, or C₂-C₆ alkynyl, halo, C₁-C₆ haloalkyl, hydroxyl, alkoxy, aryloxy, amino, alkylamino, dialkylamino, thioyl, sulfonyl, sulfonamidyl, amido(e.g., where the nitrogen of the amide is substituted by an alkyl, or where the nitrogen of the amide together with two carbons to which it is attached, forms a ring), C(O)OH, —C(O)Oalkyl, urea, sulfonylurea acyl, nitro, cyano, cyclyl, heterocyclyl, aryl, or heteroaryl; optionally substituted with 1-3 C₁-C₆ alkyl, C₁-C₆ haloalkyl, or halo;

R⁹ is independently H, C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸;

each m and n are independently 0, 1, 2, 3, 4, 5, or 6.

In some embodiments, when L is heteroaryl or cyclopropyl, n is at least 1.

In some embodiments, R¹ is C₁-C₆ alkyl, for example, methyl.

In some embodiments, R¹ is further substituted by a dialkyl amine, for example, a dimethyl amine.

In some embodiments, wherein R¹ is

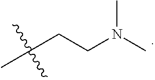

In some embodiments, R¹ is C₁-C₆ alkyl substituted by heterocyclyl, for example a nitrogen containing heterocyclyl such as morpholinyl.

In some embodiments, R² is C₁-C₆ alkyl, for example, methyl.

In some embodiments, R² is further substituted by a dialkyl amine, for example, a dimethyl amine.

In some embodiments, wherein R² is

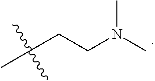

In some embodiments, R² is C₁-C₆ alkyl substituted by heterocyclyl, for example, a nitrogen containing heterocyclyl such as morpholinyl.

In some embodiments, both R¹ and R² are C₁-C₆ alkyl, for example, both R¹ and R² are methyl.

In some embodiments, R³ is monocyclic, for example a monocyclic cyclyl, a monocyclic aryl, a monocyclic heterocyclyl, or a monocyclic heteroaryl.

In some embodiments, R³ is aryl, for example, phenyl.

In some embodiments, R³ is phenyl substituted by 1-3 R⁷. In some embodiments, R⁷ is Me, OMe, or halo. In some embodiments, at least 1 R⁷ is positioned in the para position.

In some embodiments, R³ is phenyl substituted by 1 R⁷. In some embodiments, R⁷ is Me, OMe, or halo. In some embodiments, R⁷ is positioned in the para position, for example, when R⁷ is Me, OMe, or halo, e.g., R⁷ is methyl.

In some embodiments, R³ is

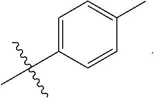

In some embodiments, R³ is heterocyclyl, for example, a nitrogen containing heterocyclyl and/or a 5 membered heterocyclyl. In some embodiments, R³ is substituted by 1-3 R⁷. In some embodiments, at least 1 R⁷ is in the 3 position of the 5 membered ring. In some embodiments, R⁷ is Me, OMe, or halo.

In some embodiments, R³ is substituted by 1 R⁷, for example, Me, OMe, or halo. In some embodiments, R⁷ is in the 3 position of the 5 membered ring, for example, when R⁷ is Me, OMe, or halo.

In some embodiments, R³ is a 6 membered heterocyclyl, for example, R³ is

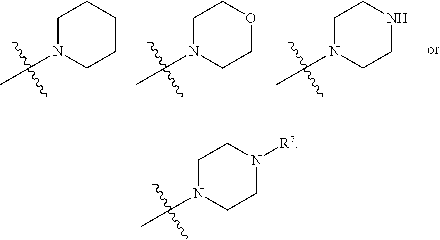

In some embodiments, R³ is substituted by 1-4 R⁷.

In some embodiments, R³ is heteroaryl, for example, a 5 or 6 membered heteroaryl, e.g., a 5 membered heteroaryl. In some embodiments, R³ is substituted by 1-3 R⁷, for example, Me, OMe, or halo. In some embodiments, at least 1 R⁷ is in the 3 position of the 5 membered ring, for example when R⁷ is Me, OMe, or halo. In some embodiments, R³ is substituted by 1 R⁷, for example, when R⁷ is Me, OMe, or halo. In some embodiments, R⁷ is in the 3 position of the 5 membered ring, for example, when R⁷ is Me, OMe, or halo. In some embodiments, R⁷ is in the 4 position of the 5 membered ring, for example, when R⁷ is Me, OMe, or halo.

In some embodiments, R³ is a nitrogen containing heteroaryl, for example,

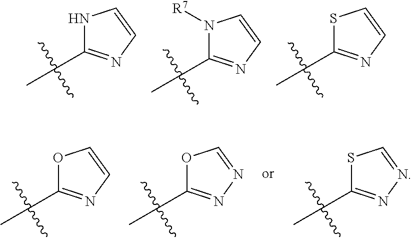

In some embodiments, the 5 membered heteroaryl is substituted by at least 1 R⁷ (e.g., one or two), for example, is in the 3 or 4 position of the 5 membered ring.

In some embodiments, R³ is a 6 membered heteroaryl, for example, substituted by 1-3 R⁷ such as Me, OMe, or halo. In some embodiments, at least 1 R⁷ is positioned in the para position, for example, when R⁷ is Me, OMe, or halo. In some embodiments, R³ is substituted by 1 R⁷, for example, when R⁷ is Me, OMe, or halo. In some embodiments, R⁷ is positioned in the para position, for example, when R⁷ is Me, OMe, or halo.

In some embodiments, R³ is a 6 membered, nitrogen containing heteroaryl.

In some embodiments, R³ is

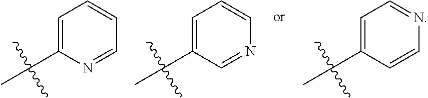

In some embodiments, R³ is substituted by 1-4 R⁷.

In some embodiments, R³ is a 6 membered heteroaryl containing 2 nitrogens, e.g.,

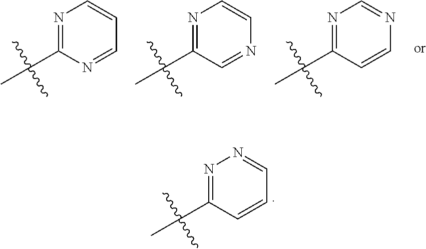

In some embodiments, R³ is substituted by 1-3 R⁷.

In some embodiments, R³ is a heteroaryl or heterocycyl having two fused rings. In some embodiments, R³ is substituted by 1-4 R⁷.

In some embodiments, R³ is a heteroaryl or heterocycyl having three fused rings. In some embodiments, R³ is substituted by 1-4 R⁷.

In some embodiments, each R⁷ is independently C₁-C₆ alkyl, C₂-C₆ alkenyl, C₂-C₆ alkynyl, halo, hydroxyl, alkoxy, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, each of which is optionally substituted with 1-3 R⁸;

In some embodiments, L is L is NR⁶SO₂, SO₂NR⁶, C(O)NR⁶, NR⁶C(O), OC(O)NR⁶, NR⁶C(O)O, NR⁶C(O)NR⁶, S, S(O), S(O)₂, C(O)NS(O)₂, S(O)₂NC(O), heteroaryl, or cyclyl.

In some embodiments, L is L is NR⁶SO₂, SO₂NR₆, OC(O)NR₆, NR₆C(O)O, NR⁶C(O)NR⁶, S, S(O), S(O)₂, C(O)NS(O)₂, S(O)₂NC(O), heteroaryl, or cyclyl.

In some embodiments, L is NR⁶SO₂ or SO₂NR⁶, OC(O)NR⁶, NR⁶C(O)O, NR⁶C(O)NR⁶.

In some embodiments, R⁶ is H.

In some embodiments, L is OC(O)NR⁶ or NR⁶C(O)O. In some embodiments, R⁶ is H.

In some embodiments, L is NR⁶C(O)NR⁶. In some embodiments, R⁶ is H.

In some embodiments, L is cyclyl or heterocyclyl, for example, cyclopropyl.

In some embodiments, L is C(O).

In some embodiments, R⁹ is H.

In some embodiments, R⁹ is halo, for example, chloro.

In some embodiments, m is 1.

In some embodiments, n is 2.

In some embodiments, m is 1 and n is 2.

In some embodiments, n is 0.

In some embodiments, m is 1 and n is 0.

In some embodiments, m+n≦6.

In some embodiments, the compound has one of the formula below:

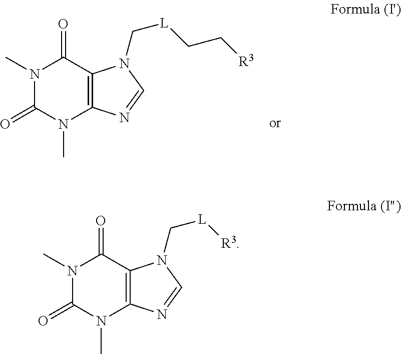

In one aspect, the compound is a compound of formula (Ia)

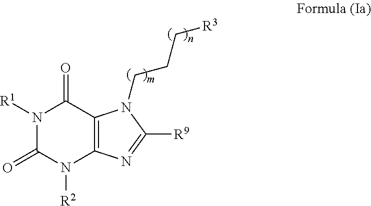

wherein,

R¹ and R² are each independently C₁-C₆ alkyl, C₂-C₆ alkenyl, or C₂-C₆ alkynyl, each of which is optionally substituted with 1-4 R⁵;

L is NR₆C(O) or C(O)NR₆;

R³ is cyclyl, heterocyclyl, aryl, heteroaryl, each of which is optionally substituted with 1-4 R⁷;

each R⁵ is independently halo, hydroxyl, alkoxy, amino, alkylamino, dialkylamino, cyano, nitro, amido(e.g., where the nitrogen of the amide is substituted by an alkyl, or where the nitrogen of the amide together with two carbons to which it is attached, forms a ring), alkylamido, dialkylamido, thioyl, sulfonyl, cyclyl, heterocyclyl, aryl, or heteroaryl;

each R⁶ is independently H, C₁-C₆ alkyl, arylalkyl, S(O)alkyl, acetyl, or S(O)H;

each R⁷ is independently C₁-C₆ alkyl, C₂-C₆ alkenyl, C₂-C₆ alkynyl, cyclyl, heterocyclyl, aryl, heteroaryl, cyclylalkyl, heterocyclylalkyl, arylalkyl, heteroarylalkyl, halo, hydroxyl, alkoxy, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, —C(O)aryl, —NHC(O)aryl, —C(O)NHaryl, nitro, cyano, each of which is optionally substituted with 1-3 R⁸;

each R⁸ is independently C₁-C₆ alkyl, C₂-C₆ alkenyl, or C₂-C₆ alkynyl, halo, C₁-C₆ haloalkyl, hydroxyl, alkoxy, aryloxy, amino, alkylamino, dialkylamino, thioyl, sulfonyl, sulfonamidyl, amido(e.g., where the nitrogen of the amide is substituted by an alkyl, or where the nitrogen of the amide together with two carbons to which it is attached, forms a ring), urea, sulfonylurea acyl, nitro, cyano, cyclyl, heterocyclyl, aryl, or heteroaryl; optionally substituted with 1-3 C₁-C₆ alkyl, C₁-C₆ haloalkyl, or halo;

each R⁹ is independently C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸;

m is 0, 1, 2, 3, 4, 5, or 6; and
n is 2, 3, 4, 5, or 6.

In some embodiments, when m is 1, n is 2, L is C(O)NH, and R¹ and R² are both methyl, R³ is not phenyl. In some embodiments, when L is NR₆C(O), m is at least 2.

In some embodiments, R¹ is C₁-C₆ alkyl, for example, methyl.

In some embodiments, R¹ is further substituted by a dialkyl amine, for example, a dimethyl amine.

In some embodiments, wherein R¹ is

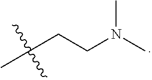

In some embodiments, R¹ is C₁-C₆ alkyl substituted by heterocyclyl, for example a nitrogen containing heterocyclyl such as morpholinyl.

In some embodiments, R² is C₁-C₆ alkyl, for example, methyl.

In some embodiments, R² is further substituted by a dialkyl amine, for example, a dimethyl amine.

In some embodiments, wherein R² is

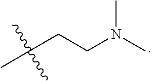

In some embodiments, R² is C₁-C₆ alkyl substituted by heterocyclyl, for example, a nitrogen containing heterocyclyl such as morpholinyl.

In some embodiments, both R¹ and R² are C₁-C₆ alkyl, for example, both R¹ and R² are methyl.

In some embodiments, R³ is monocyclic, for example a monocyclic cyclyl, a monocyclic aryl, a monocyclic heterocyclyl, or a monocyclic heteroaryl.

In some embodiments, R³ is aryl, for example, phenyl.

In some embodiments, R³ is phenyl substituted by 1-3 R⁷. In some embodiments, R⁷ is Me, OMe, or halo. In some embodiments, at least 1 R⁷ is positioned in the para position.

In some embodiments, R³ is phenyl substituted by 1 R⁷. In some embodiments, R⁷ is Me, OMe, or halo. In some embodiments, R⁷ is positioned in the para position, for example, when R⁷ is Me, OMe, or halo, e.g., R⁷ is methyl.

In some embodiments, R³ is

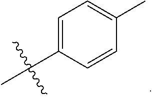

In some embodiments, R³ is heterocyclyl, for example, a nitrogen containing heterocyclyl and/or a 5 membered heterocyclyl. In some embodiments, R³ is substituted by 1-3 R⁷. In some embodiments, at least 1 R⁷ is in the 3 position of the 5 membered ring. In some embodiments, R⁷ is Me, OMe, or halo.

In some embodiments, R³ is substituted by 1 R⁷, for example, Me, OMe, or halo. In some embodiments, R⁷ is in the 3 position of the 5 membered ring, for example, when R⁷ is Me, OMe, or halo.

In some embodiments, R³ is a 6 membered heterocyclyl, for example, R³ is

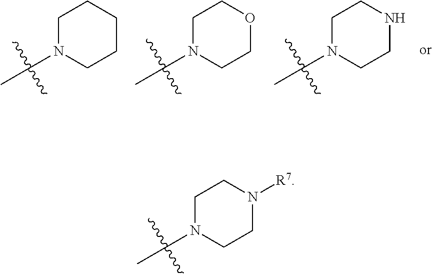

In some embodiments, R³ is substituted by 1-4 R⁷.

In some embodiments, R³ is heteroaryl, for example, a 5 or 6 membered heteroaryl, e.g., a 5 membered heteroaryl. In some embodiments, R³ is substituted by 1-3 R⁷, for example, Me, OMe, or halo. In some embodiments, at least 1 R⁷ is in the 3 position of the 5 membered ring, for example when R⁷ is Me, OMe, or halo. In some embodiments, R³ is substituted by 1 R⁷, for example, when R⁷ is Me, OMe, or halo. In some embodiments, R⁷ is in the 3 position of the 5 membered ring, for example, when R⁷ is Me, OMe, or halo. In some embodiments, R⁷ is in the 4 position of the 5 membered ring, for example, when R⁷ is Me, OMe, or halo.

In some embodiments, R³ is a nitrogen containing heteroaryl, for example,

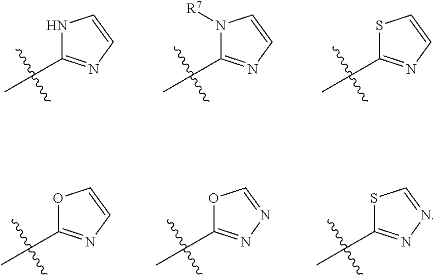

In some embodiments, the 5 membered heteroaryl is substituted by at least 1 R⁷ (e.g., one or two), for example, in the 3 or 4 position of the 5 membered ring.

In some embodiments, R³ is a 6 membered heteroaryl, for example, substituted by 1-3 R⁷ such as Me, OMe, or halo. In some embodiments, at least 1 R⁷ is positioned in the para position, for example, when R⁷ is Me, OMe, or halo. In some embodiments, R³ is substituted by 1 R⁷, for example, when R⁷ is Me, OMe, or halo. In some embodiments, R⁷ is positioned in the para position, for example, when R⁷ is Me, OMe, or halo.

In some embodiments, R³ is

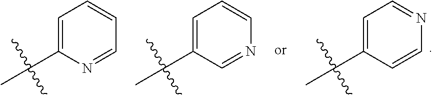

In some embodiments, R³ is substituted by 1-4 R⁷.

In some embodiments, R³ is a 6 membered heteroaryl containing 2 nitrogens, e.g.,

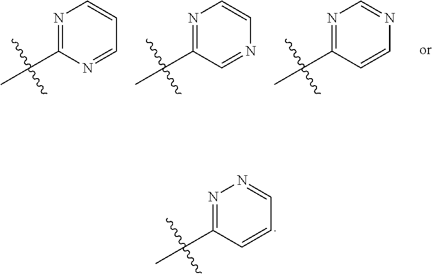

In some embodiments, R³ is substituted by 1-3 R⁷.

In some embodiments, R³ is a heteroaryl or heterocycyl having two fused rings. In some embodiments, R³ is substituted by 1-4 R⁷. In some embodiments, R³ is a heteroaryl or heterocycyl having three fused rings. In some embodiments, R³ is substituted by 1-4 R⁷.

In some embodiments, each R⁷ is independently C₁-C₆ alkyl, C₂-C₆ alkenyl, C₂-C₆ alkynyl, halo, hydroxyl, alkoxy, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, each of which is independently substituted with 1-3 R⁸.

In some embodiments, L is C(O)NR⁶.

In some embodiments, L is NR⁶C(O).

In some embodiments, R⁹ is H.

In some embodiments, R⁹ is halo, for example, chloro.

In some embodiments, m is 1.

In some embodiments, n is 2.

In some embodiments, m is 1 and n is 2.

In some embodiments, n is 0.

In some embodiments, m is 1 and n is 0.

In some embodiments, m+n≦6.

In some preferred embodiments, the compound is a compound of formula (Ia′)

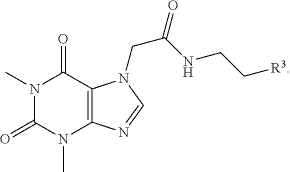

In one aspect, the invention features a compound of formula (Ib)

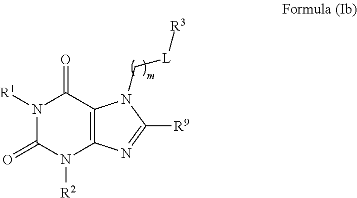

wherein,

R¹ and R² are each independently C₁-C₆ alkyl, C₂-C₆ alkenyl, or C₂-C₆ alkynyl, each of which is optionally substituted with 1-4 R⁵;

L is NR₆C(O) or C(O)NR₆;

R³ is cyclyl, heterocyclyl, aryl, heteroaryl, each of which is optionally substituted with 1-4 R⁷;

each R⁵ is independently halo, hydroxyl, alkoxy, amino, alkylamino, dialkylamino, cyano, nitro, amido (e.g., where the nitrogen of the amide is substituted by an alkyl, or where the nitrogen of the amide together with two carbons to which it is attached, forms a ring), alkylamido, dialkylamido, thioyl, sulfonyl, cyclyl, heterocyclyl, aryl, or heteroaryl;

each R⁶ is independently H, C₁-C₆ alkyl, arylalkyl, S(O)alkyl, acetyl, or S(O)H;

each R⁷ is independently C₁-C₆ alkyl, C₂-C₆ alkenyl, or C₂-C₆ alkynyl, halo, cyclylalkyl, heterocyclylalkyl, arylalkyl, heteroarylalkyl, hydroxyl, alkoxy, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, —C(O)aryl, —NHC(O)aryl, —C(O)NHaryl, nitro, cyano, each of which is optionally substituted with 1-3 R⁸;

each R⁸ is independently C₁-C₆ alkyl, C₂-C₆ alkenyl, or C₂-C₆ alkynyl, halo, C₁-C₆ haloalkyl, hydroxyl, alkoxy, aryloxy, amino, alkylamino, dialkylamino, thioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, cyclyl, heterocyclyl, aryl, or heteroaryl; optionally substituted with 1-3 C₁-C₆ alkyl, C₁-C₆ haloalkyl, or halo;

each R⁹ is independently C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸;

m is 0, 1, 2, 3, 4, 5, or 6.

In some embodiments, when L is NR₆C(O), m is at least 2.

In some embodiments, R¹ is C₁-C₆ alkyl, for example, methyl.

In some embodiments, R¹ is further substituted by a dialkyl amine, for example, a dimethyl amine.

In some embodiments, wherein R¹ is

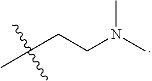

In some embodiments, R¹ is C₁-C₆ alkyl substituted by heterocyclyl, for example a nitrogen containing heterocyclyl such as morpholinyl.

In some embodiments, R² is C₁-C₆ alkyl, for example, methyl.

In some embodiments, R² is further substituted by a dialkyl amine, for example, a dimethyl amine.

In some embodiments, wherein R² is

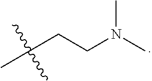

In some embodiments, R² is C₁-C₆ alkyl substituted by heterocyclyl, for example, a nitrogen containing heterocyclyl such as morpholinyl.

In some embodiments, both R¹ and R² are C₁-C₆ alkyl, for example, both R¹ and R² are methyl.

In some embodiments, R³ is monocyclic, for example a monocyclic cyclyl, a monocyclic aryl, a monocyclic heterocyclyl, or a monocyclic heteroaryl.

In some embodiments, R³ is aryl, for example, phenyl.

In some embodiments, R³ is phenyl substituted by 1-3 R⁷. In some embodiments, R⁷ is Me, OMe, or halo. In some embodiments, at least 1 R⁷ is positioned in the para position.

In some embodiments, R³ is phenyl substituted by 1 R⁷. In some embodiments, R⁷ is Me, OMe, or halo. In some embodiments, R⁷ is positioned in the para position, for example, when R⁷ is Me, OMe, or halo, e.g., R⁷ is methyl.

In some embodiments, R³ is

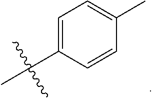

In some embodiments, R³ is heterocyclyl, for example, a nitrogen containing heterocyclyl and/or a 5 membered heterocyclyl. In some embodiments, R³ is substituted by 1-3 R⁷. In some embodiments, at least 1 R⁷ is in the 3 position of the 5 membered ring. In some embodiments, R⁷ is Me, OMe, or halo.

In some embodiments, R³ is substituted by 1 R⁷, for example, Me, OMe, or halo. In some embodiments, R⁷ is in the 3 position of the 5 membered ring, for example, when R⁷ is Me, OMe, or halo.

In some embodiments, R³ is a 6 membered heterocyclyl, for example, R³ is

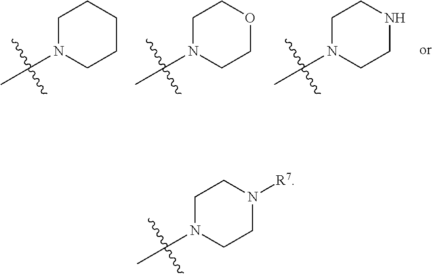

In some embodiments, R³ is substituted by 1-4 R⁷.

In some embodiments, R³ is heteroaryl, for example, a 5 or 6 membered heteroaryl, e.g., a 5 membered heteroaryl. In some embodiments, R³ is substituted by 1-3 R⁷, for example, Me, OMe, or halo. In some embodiments, at least 1 R⁷ is in the 3 position of the 5 membered ring, for example when R⁷ is Me, OMe, or halo. In some embodiments, R³ is substituted by 1 R⁷, for example, when R⁷ is Me, OMe, or halo. In some embodiments, R⁷ is in the 3 position of the 5 membered ring, for example, when R⁷ is Me, OMe, or halo. In some embodiments, R⁷ is in the 4 position of the 5 membered ring, for example, when R⁷ is Me, OMe, or halo.

In some embodiments, R³ is a nitrogen containing heteroaryl, for example,

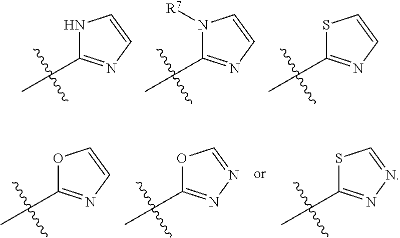

In some embodiments, the 5 membered heteroaryl is substituted by at least 1 R⁷ (e.g., one or two), for example, in the 3 or 4 position of the 5 membered ring.

In some embodiments, R³ is a 6 membered heteroaryl, for example, substituted by 1-3 R⁷ such as Me, OMe, or halo. In some embodiments, at least 1 R⁷ is positioned in the para position, for example, when R⁷ is Me, OMe, or halo. In some embodiments, R³ is substituted by 1 R⁷, for example, when R⁷ is Me, OMe, or halo. In some embodiments, R⁷ is positioned in the para position, for example, when R⁷ is Me, OMe, or halo.

In some embodiments, R³ is

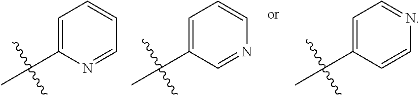

In some embodiments, R³ is substituted by 1-4 R⁷.

In some embodiments, R³ is a 6 membered heteroaryl containing 2 nitrogens, e.g.,

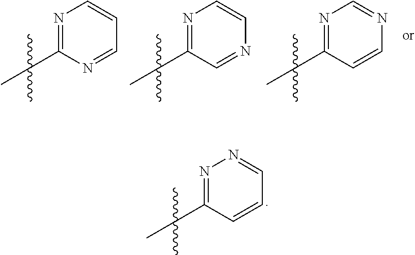

In some embodiments, R³ is substituted by 1-4 R⁷.

In some embodiments, R³ is a heteroaryl or heterocycyl having two fused rings. In some embodiments, R³ is substituted by 1-4 R⁷. In some embodiments, R³ is a heteroaryl or heterocycyl having three fused rings. In some embodiments, R³ is substituted by 1-4 R⁷.

In some embodiments, each R⁷ is independently C₁-C₆ alkyl, C₂-C₆ alkenyl, C₂-C₆ alkynyl, halo, hydroxyl, alkoxy, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, each of which is optionally substituted with 1-3 R⁸.

In some embodiments, L is C(O)NR⁶.

In some embodiments, L is NR⁶C(O).

In some embodiments, R⁹ is H.

In some embodiments, R⁹ is halo, for example, chloro.

In some embodiments, m is 1.

In one aspect, the invention features a compound of formula (Ic)

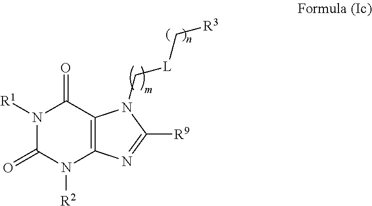

wherein,

R¹ and R² are each independently C₁-C₆ alkyl, C₂-C₆ alkenyl, or C₂-C₆ alkynyl, each of which is optionally substituted with 1-4 R⁵;

L is NR⁶, CH₂, or O;

R³ is cyclyl, heterocyclyl, aryl, heteroaryl, each of which is optionally substituted with 1-4 R⁷;

each R⁵ is independently halo, hydroxyl, alkoxy, amino, alkylamino, dialkylamino, cyano, nitro, amido (e.g., where the nitrogen of the amide is substituted by an alkyl, or where the nitrogen of the amide together with two carbons to which it is attached, forms a ring), alkylamido, dialkylamido, thioyl, sulfonyl, cyclyl, heterocyclyl, aryl, or heteroaryl;

each R⁶ is independently H, C₁-C₆ alkyl, arylalkyl, S(O)alkyl, acetyl, or S(O)H;

each R⁷ is independently C₁-C₆ alkyl, C₂-C₆ alkenyl, or C₂-C₆ alkynyl, halo, hydroxyl, alkoxy, aryloxy, arylalkoxy, cyclylalkyl, heterocyclylalkyl, arylalkyl, heteroarylalkyl, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, each of which is optionally substituted with 1-3 R⁸;

each R⁸ is independently C₁-C₆ alkyl, C₂-C₆ alkenyl, or C₂-C₆ alkynyl, halo, C₁-C₆ haloalkyl, hydroxyl, alkoxy, aryloxy, amino, alkylamino, dialkylamino, thioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea acyl, —C(O)aryl, —NHC(O)aryl, —C(O)NHaryl, nitro, cyano, cyclyl, heterocyclyl, aryl, or heteroaryl; optionally substituted with 1-3 C₁-C₆ alkyl, C₁-C₆ haloalkyl, or halo;

each R⁹ is independently C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸;

m is 1, 2, 3, 4, 5, or 6; and

n is 1, 2, 3, 4, 5, or 6.

In some embodiments, when L is CH₂ and R³ is phenyl, m and n together do not equal 2, 3, or 4. In some embodiments, when L is NR⁶, R³ is not unsubstituted phenyl or phenyl substituted with OMe or C₁-C₆ alkyl further substituted with C(O)Ar. In some embodiments when L is NR₆ or O, m is at least 2.

In some embodiments, R¹ is C₁-C₆ alkyl, for example, methyl.

In some embodiments, R¹ is further substituted by a dialkyl amine, for example, a dimethyl amine.

In some embodiments, wherein R¹ is

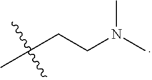

In some embodiments, R¹ is C₁-C₆ alkyl substituted by heterocyclyl, for example a nitrogen containing heterocyclyl such as morpholinyl.

In some embodiments, R² is C₁-C₆ alkyl, for example, methyl.

In some embodiments, R² is further substituted by a dialkyl amine, for example, a dimethyl amine.

In some embodiments, wherein R² is

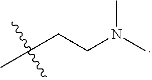

In some embodiments, R² is C₁-C₆ alkyl substituted by heterocyclyl, for example, a nitrogen containing heterocyclyl such as morpholinyl.

In some embodiments, both R¹ and R² are C₁-C₆ alkyl, for example, both R¹ and R² are methyl.

In some embodiments, R³ is monocyclic, for example a monocyclic cyclyl, a monocyclic aryl, a monocyclic heterocyclyl, or a monocyclic heteroaryl.

In some embodiments, R³ is aryl, for example, phenyl.

In some embodiments, R³ is phenyl substituted by 1-3 R⁷. In some embodiments, R⁷ is Me, OMe, or halo. In some embodiments, at least 1 R⁷ is positioned in the para position.

In some embodiments, R³ is phenyl substituted by 1 R⁷. In some embodiments, R⁷ is Me, OMe, or halo. In some embodiments, R⁷ is positioned in the para position, for example, when R⁷ is Me, OMe, or halo, e.g., R⁷ is methyl.

In some embodiments, R³ is

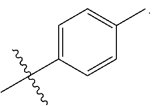

In some embodiments, R³ is heterocyclyl, for example, a nitrogen containing heterocyclyl and/or a 5 membered heterocyclyl. In some embodiments, R³ is substituted by 1-3 R⁷. In some embodiments, at least 1 R⁷ is in the 3 position of the 5 membered ring. In some embodiments, R⁷ is Me, OMe, or halo.

In some embodiments, R³ is substituted by 1 R⁷, for example, Me, OMe, or halo. In some embodiments, R⁷ is in the 3 position of the 5 membered ring, for example, when R⁷ is Me, OMe, or halo.

In some embodiments, R³ is a 6 membered heterocyclyl, for example, R³ is

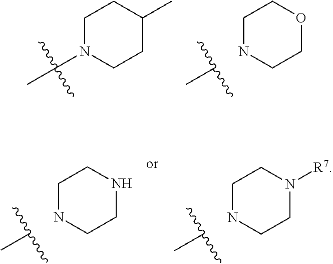

In some embodiments, R³ is substituted by 1-4 R⁷.

In some embodiments, R³ is heteroaryl, for example, a 5 or 6 membered heteroaryl, e.g., a 5 membered heteroaryl. In some embodiments, R³ is substituted by 1-3 R⁷, for example, Me, OMe, or halo. In some embodiments, at least 1 R⁷ is in the 3 position of the 5 membered ring, for example when R⁷ is Me, OMe, or halo. In some embodiments, R³ is substituted by 1 R⁷, for example, when R⁷ is Me, OMe, or halo. In some embodiments, R⁷ is in the 3 position of the 5 membered ring, for example, when R⁷ is Me, OMe, or halo. In some embodiments, R⁷ is in the 4 position of the 5 membered ring, for example, when R⁷ is Me, OMe, or halo.

In some embodiments, R³ is a nitrogen containing heteroaryl, for example,

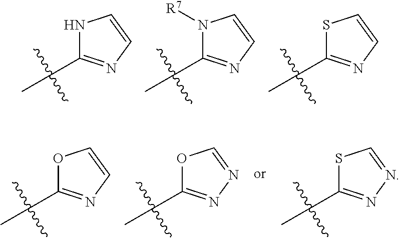

In some embodiments, the 5 membered heteroaryl is substituted by at least 1 R⁷ (e.g., one or two), for example, in the 3 or 4 position of the 5 membered ring.

In some embodiments, R³ is a 6 membered heteroaryl, for example, substituted by 1-3 R⁷ such as Me, OMe, or halo. In some embodiments, at least 1 R⁷ is positioned in the para position, for example, when R⁷ is Me, OMe, or halo. In some embodiments, R³ is substituted by 1 R⁷, for example, when R⁷ is Me, OMe, or halo. In some embodiments, R⁷ is positioned in the para position, for example, when R⁷ is Me, OMe, or halo.

In some embodiments, R³ is

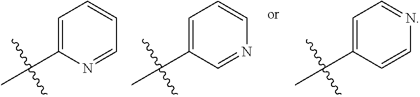

In some embodiments, R³ is substituted by 1-4 R⁷.

In some embodiments, R³ is a 6 membered heteroaryl containing 2 nitrogens, e.g.,

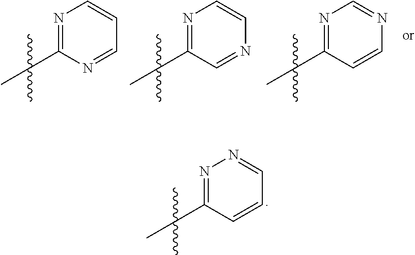

In some embodiments, R³ is substituted by 1-3 R⁷.
In some embodiments, R³ is a heteroaryl or heterocycyl having two fused rings. In some embodiments, R³ is substituted by 1-4 R⁷. In some embodiments, R³ is a heteroaryl or heterocycyl having three fused rings. In some embodiments, R³ is substituted by 1-4 R⁷.

In some embodiments, each R⁷ is independently C₁-C₆ alkyl, C₂-C₆ alkenyl, C₂-C₆ alkynyl, halo, hydroxyl, alkoxy, aryloxy, arylalkoxy, amino, alkylamino, dialkylamino, thioyl, alkylthioyl, sulfonyl, sulfonamidyl, amido(e.g., where the nitrogen of the amide is substituted by an alkyl, or where the nitrogen of the amide together with two carbons to which it is attached, forms a ring), urea, sulfonylurea, acyl, nitro, cyano, each of which is optionally substituted with 1-3 R⁸;

In some embodiments, L is NR⁶.

In some embodiments, L is O.

In some embodiments, L is CH₂.

In some embodiments, R⁹ is H.

In some embodiments, R⁹ is halo, for example, chloro.

In some embodiments, m is 1.

In some embodiments, n is 2.

In some embodiments, m is 1 and n is 2.

In some embodiments, n is 0.

In some embodiments, m is 1 and n is 0.

In one aspect, the invention features a compound of formula (Id)

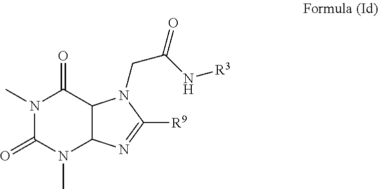

wherein

R³ is a 3 membered ring fused heteroaryl, optionally substituted with 1-4 R⁷;

each R⁷ is independently C₁-C₆ alkyl, C₂-C₆ alkenyl, C₂-C₆ alkynyl, cyclyl, heterocyclyl, aryl, heteroaryl, cyclylalkyl, heterocyclylalkyl, arylalkyl, heteroarylalkyl, halo, hydroxyl, alkoxy, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, each of which is optionally substituted with 1-3 R⁸;

each R⁸ is independently C₁-C₆ alkyl, C₂-C₆ alkenyl, or C₂-C₆ alkynyl, halo, C₁-C₆ haloalkyl, hydroxyl, alkoxy, aryloxy, amino, alkylamino, dialkylamino, thioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea acyl, —C(O)aryl, —NHC(O)aryl, —C(O)NHaryl, nitro, cyano, cyclyl, heterocyclyl, aryl, or heteroaryl; optionally substituted with 1-3 C₁-C₆ alkyl, C₁-C₆ haloalkyl, or halo;

R⁹ is independently H, C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸.

In some embodiments, R³ is substituted with 0, 1 or 3 R⁷, each of which is independently C₁-C₆ alkyl, C₂-C₆ alkenyl, C₂-C₆ alkynyl, halo, hydroxyl, alkoxy, acyl, nitro, or cyano.

In one aspect, the compound is a formula (II):

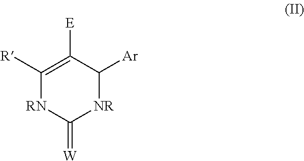

wherein
W represents O or S, preferably S;
R, independently for each occurrence, represents H or lower alkyl, preferably H;
R′ represents substituted or unsubstituted alkyl or substituted or unsubstituted aryl;
E represents carboxylic acid (CO₂H), ester or amide; and
Ar represents a substituted or unsubstituted aryl ring; and
wherein said compound inhibits TRPA1 with an with an IC₅₀ of 10 micromolar or less.

In one aspect, the compound is a formula (III):

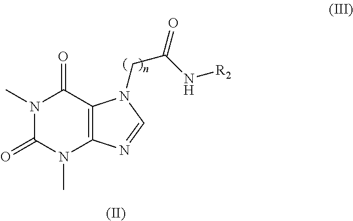

wherein
n is an integer from 1 to 3; and
R₂ represents a substituent; and
wherein said compound inhibits TRPA1 with an with an IC₅₀ of 10 micromolar or less.

In certain embodiments, R₂ represents optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

In certain embodiments, R₂ is not

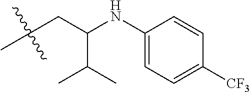

when n=1.

In certain embodiments, the present invention provides a method for treating or preventing a condition involving activation of TRPA1 or for which reduced TRPA1 activity can reduce the severity, comprising administering an effective amount of a compound of Formula IV or a salt thereof, or a solvate, hydrate, oxidative metabolite or prodrug of the compound or its salt:

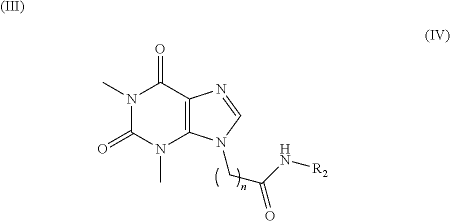

wherein
n is an integer from 1 to 3; and
R₂ represents a substituent; and
wherein said compound inhibits TRPA1 with an with an IC₅₀ of 10 micromolar or less.

In certain embodiments, R₂ represents optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

In certain embodiments, the present invention provides a method for treating or preventing a condition involving activation of TRPA1 or for which reduced TRPA1 activity can reduce the severity, comprising administering an effective amount of a compound of Formula V or a salt thereof, or a solvate, hydrate, oxidative metabolite or prodrug of the compound or its salt:

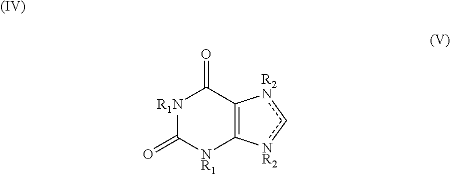

wherein
R₁, independently for each occurrence, represents H or lower alkyl;
one occurrence of R₂ is absent and one occurrence of R₂ is MₘR₃;
R₃ represents substituted or unsubstituted aryl;
M, independently for each occurrence, represents a substituted or unsubstituted methylene group (e.g., substituted with lower alkyl, oxo, hydroxyl, etc.), NR₁, O, S, S(O), or S(O₂), preferably selected such that no two heteroatoms are adjacent to each other; and
m is an integer from 0-10; and
wherein said compound inhibits TRPA1 with an with an IC₅₀ of 10 micromolar or less.

In certain embodiments, MₘR₃ represents

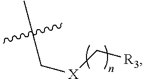

wherein
n is an integer between 0 and 4; and
X is —C(═O)O— or —C(═O)NR₄— wherein R₄ is H or lower alkyl, preferably —C(═O)NH—.

In one aspect, the invention features a compound of formula (VIII),

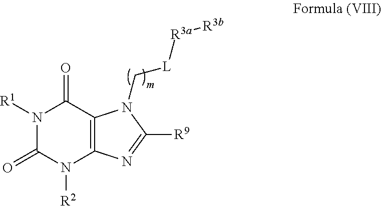

wherein,

R¹ and R² are each independently C₁-C₆ alkyl, C₂-C₆ alkenyl, or C₂-C₆ alkynyl, each of which is optionally substituted with 1-4 R⁵;

L is NR⁶SO₂, SO₂NR⁶, OC(O)NR⁶, NR⁶C(O)O, NR⁶C(O)NR⁶, NR⁶C(O), C(O)NR⁶, O, C(O), S, S(O), S(O)₂, NR₆, or CH₂,

each of R^{3a} and R^{3b} is independently cyclyl, heterocyclyl, aryl, heteroaryl, each of which is optionally substituted with 1-4 R⁷;

each R⁵ is independently halo, hydroxyl, alkoxy, amino, alkylamino, dialkylamino, cyano, nitro, amido(e.g., where the nitrogen of the amide is substituted by an alkyl, or where the nitrogen of the amide together with two carbons to which it is attached, forms a ring), alkylamido, dialkylamido, thioyl, sulfonyl, cyclyl, heterocyclyl, aryl, or heteroaryl;

each R⁶ is independently H, C₁-C₆ alkyl, C₁-C₆ alkenyl, hydroxyC₁-C₆ alkyl, alkoxyC₁-C₆ alkyl, cyanoalkyl, haloalkyl, arylalkyl, S(O)alkyl, acyl, amino, amidyl, or S(O)₂H, aryl, alkoxyaryl;

each R⁷ is independently C₁-C₆ alkyl, C₂-C₆ alkenyl, or C₂-C₆ alkynyl, halo, hydroxyl, alkoxy, oxo, aryl, heteroaryl, cyclyl, heterocyclyl, arylalkyl, heteroarylalkyl, cyclylalkyl, heterocyclylalkyl, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), hydroxyl alkoxyl, alkoxy —C(O)OH, —C(O)Oalkyl, urea, sulfonylurea, acyl, nitro, cyano, each of which is optionally substituted with 1-3 R⁸;

each R⁸ is independently C₁-C₆ alkyl, C₂-C₆ alkenyl, or C₂-C₆ alkynyl, aryl, heteroaryl, cyclyl, halo, hydroxyl, alkoxy, oxo, aryloxy, amino, alkylamino, dialkylamino, C(O)OH, —C(O)Oalkyl, thioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, cyclyl, heterocyclyl, aryl, or heteroaryl;

R⁹ is H, C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸;

m is 1, 2, 3, 4, 5, or 6.

In some embodiments, m is at least 2 when L is connected to the methylene carbon via a heteroatom.

In some embodiments, when L is CH₂, S, C(O)NR⁶ or NR⁶C(O), R^{3a} is not a 5-membered heterocyclyl, 5-membered heteroaryl, or piperazine.

In some embodiments, when L is C(O)NH, R^{3a} and R^{3b} are not both phenyl.

In some embodiments, m is at least 2 when L is connected to the methylene carbon via a heteroatom.

In some embodiments, R¹ is C₁-C₆ alkyl, for example, methyl.

In some embodiments, R¹ is further substituted by a dialkyl amine, for example, a dimethyl amine.

In some embodiments, wherein R¹ is

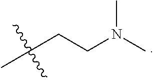

In some embodiments, R¹ is C₁-C₆ alkyl substituted by heterocyclyl, for example a nitrogen containing heterocyclyl such as morpholinyl.

In some embodiments, R² is C₁-C₆ alkyl, for example, methyl.

In some embodiments, R² is further substituted by a dialkyl amine, for example, a dimethyl amine.

In some embodiments, wherein R² is

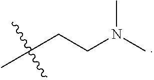

In some embodiments, R² is C₁-C₆ alkyl substituted by heterocyclyl, for example, a nitrogen containing heterocyclyl such as morpholinyl.

In some embodiments, R^{3a} is monocyclic, for example a monocyclic cyclyl, a monocyclic aryl, a monocyclic heterocyclyl, or a monocyclic heteroaryl.

In some embodiments, R^{3a} is aryl, for example, phenyl.

In some embodiments, R^{3a} is

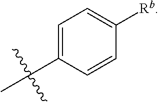

In some embodiments, R^{3a} and/or R^{3b} is substituted by 1-4 R⁷.

In some embodiments, R^{3a} is heterocyclyl, for example, a nitrogen containing heterocyclyl and/or a 5 membered heterocyclyl. In some embodiments, at least 1 R^{3b} is in the 3 position of the 5 membered ring.

In some embodiments, R^{3a} is substituted by 1 R⁷, for example, Me, OMe, or halo. In some embodiments, R⁷ is in a position other than the 3 position of the 5 membered ring, for example, when R⁷ is Me, OMe, or halo.

In some embodiments, R^{3a} is a 6 membered heterocyclyl, for example, R³ is

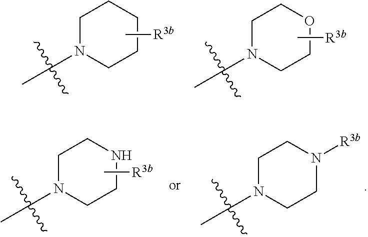

In some embodiments, R^{3a} and/or R^{3b} is substituted by 1-4 R⁷.

In some embodiments, R^{3a} is heteroaryl, for example, a 5 or 6 membered heteroaryl, e.g., a 5 membered heteroaryl. In some embodiments, R^{3a} is substituted by 1-3 R⁷, for example, Me, OMe, or halo. In some embodiments, at least 1 R⁷ is in the 2 or 3 position of the 5 membered ring, for example when R⁷ is Me, OMe, or halo. In some embodiments, R³ is substituted by 1 R⁷, for example, when R⁷ is Me, OMe, or halo. In some embodiments, R⁷ is in the 2 or 3 position of the 5 membered ring, for example, when R⁷ is Me, OMe, or halo. In some embodiments, R⁷ is in the 4 position of the 5 membered ring, for example, when R⁷ is Me, OMe, or halo.

In some embodiments, R^{3a} is a nitrogen containing heteroaryl, for example,

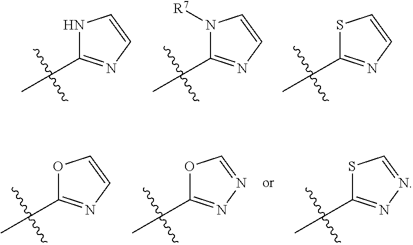

In some embodiments, the 5 membered heteroaryl is substituted by at least one R⁷ (e.g., one or two), for example, in the 3 or 4 position of the 5 membered ring. In some embodiments, R^{3a} and/or R^{3b} is further substituted by a cyclyl, heterocyclyl, aryl, heteroaryl (e.g., phenyl, or thiophenyl, each of which is independently optionally substituted with 1-4 R⁷). In some embodiments, R^{3a} is

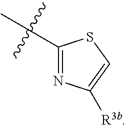

In some embodiments, R^{3b} is phenyl, optionally substituted by 1-4 R⁷. For example, 1 or 2 halo, Me, OMe, or amino.

In some embodiments, R^{3a} is a nitrogen containing heteroaryl, for example,

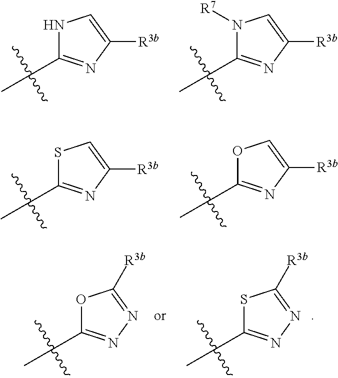

In some embodiments, R^{3a} and/or R^{3b} is further substituted by 1-4 R⁷.

In some embodiments, R^{3a} and R^{3b} together form

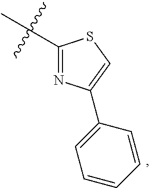

wherein R^{3a} and/or R^{3b} is optionally further substituted by 1-4 R⁷. In some embodiments, the phenyl is further substituted by 1-3 R⁷. In some embodiments, at least one R⁷ is amino, alkylamino, dialkylamino or heterocycyl (e.g., a nitrogen containing heterocyclyl such as pyrrolidine or piperidine). In some embodiments, at least 1 R⁷ is positioned in the para position of the phenyl ring.

In some embodiments, R^{3a} is a 6 membered heteroaryl, for example, substituted by 1-3 R⁷ such as Me, OMe, or halo. In some embodiments, at least 1 R⁷ is positioned in the para position, for example, when R⁷ is Me, OMe, or halo. In some embodiments, R^{3a} is substituted by 1 R⁷, for example, when R⁷ is Me, OMe, or halo. In some embodiments, R⁷ is positioned in the para position, for example, when R⁷ is Me, OMe, or halo.

In some embodiments, R^{3b} is a 6 membered, nitrogen containing heteroaryl.

In some embodiments, R^{3a} is

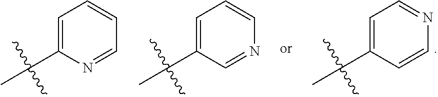

In some embodiments, R^{3a} is substituted by 1-4 R⁷.

In some embodiments, R^{3a} is a 6 membered heteroaryl containing 2 nitrogens, e.g.,

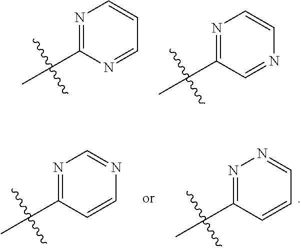

In some embodiments, R^{3a} is substituted by 1-4 R⁷.

In some embodiments, R^{3a} is a heteroaryl or heterocycyl having two fused rings. In some embodiments, R^{3a} is substituted by 1-4 R⁷. In some embodiments, R^{3a} is a heteroaryl or heterocycyl having three fused rings. In some embodiments, R^{3a} is substituted by 1-4 R⁷.

In some embodiments, R^{3b} is phenyl. In some embodiments, the phenyl is further substituted with 1-3 R⁷.

In some embodiments, R^{3b} is a heteroaryl or heterocycyl having two fused rings. In some embodiments, R^{3b} is substituted by 1-4 R⁷.

In some embodiments, each R⁷ is independently C₁-C₆ alkyl, C₂-C₆ alkenyl, C₂-C₆ alkynyl, halo, hydroxyl, alkoxy, oxo, aryl, heteroaryl, cyclyl, heterocyclyl, aryloxy, arylalkoxy, amino, alkylamino, dialkylamino, thioyl, alkylthioyl, sulfonyl, sulfonamidyl, amido, urea, sulfonylurea, acyl, nitro, or cyano, each of which is optionally substituted with 1-3 R⁸.

In some embodiments, L is L is NR⁶SO₂, SO₂NR⁶, OC(O)NR⁶, NR⁶C(O)O, NR⁶C(O)NR⁶, S, S(O), S(O)₂, C(O)NS(O)₂, S(O)₂NC(O), heteroaryl, or cyclyl.

In some embodiments, L is NR⁶SO₂ or SO₂NR⁶, OC(O)NR⁶, NR⁶C(O)O, NR⁶C(O)NR⁶. In some embodiments, R⁶ is H.

In some embodiments, L is OC(O)NR⁶ or NR⁶C(O)O. In some embodiments, R⁶ is H.

In some embodiments, L is NR⁶C(O)NR⁶. In some embodiments, R⁶ is H.

In some embodiments, L is cyclyl or heterocyclyl, for example, cyclopropyl.

In some embodiments, L is C(O)NR⁶ or NR⁶C(O). In some embodiments, R⁶ is H.

In some embodiments, L is C(O).

In some embodiments, R⁹ is H.

In some embodiments, R⁹ is halo, for example, chloro.

In some embodiments, m is 1.

In some embodiments, n is 2.

In some embodiments, m is 1 and n is 2, for example, where L is C(O)NR⁶.

In some embodiments, n is 0.

In some embodiments, m is 1 and n is 0, for example, where R³ is aryl or heteroaryl (e.g., further substituted by at least one R⁷).

In some embodiments, m+n≦6.

In some embodiments, the compound is of formula (VIII′)

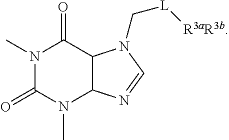

Formula (VIII′). In some embodiments, L is C(O)NR⁶.

In one aspect, the compound is a compound of Formula (VIII″)

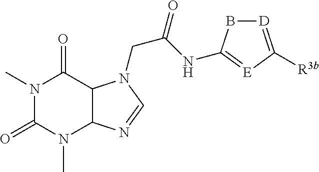

Formula (VIII″), wherein B is O, S, or NR⁶; D and E are independently CH, CR⁷ or N. In some embodiments, R^{3b} is phenyl, for example, a phenyl optionally substituted with 1-4 R⁷. In some embodiments, at least one R⁷ is amino, alkylamino, dialkylamino or heterocycyl (e.g., a nitrogen containing heterocyclyl such as pyrrolidine or piperidine). In some embodiments, at least 1 R⁷ is positioned in the para position of the phenyl ring.

In some embodiments, D and E are independently O, S, or NR⁶.

In some embodiments, B is S, D is CH, and E is N.

In one aspect, the compound is a compound of Formula (VIII′″)

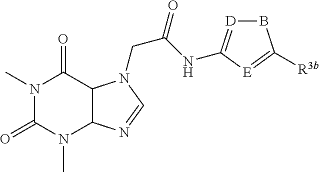

Formula (VIII′″), wherein B is O, S, or NR⁶; D and E are independently CH, CR⁷ or N. In some embodiments, R^{3b} is phenyl, for example, a phenyl optionally substituted with 1-4 R⁷. In some embodiments, at least one R⁷ is amino, alkylamino, dialkylamino or heterocycyl (e.g., a nitrogen containing heterocyclyl such as pyrrolidine or piperidine). In some embodiments, at least 1 R⁷ is positioned in the para position of the phenyl ring.

In some embodiments, D and E are independently O, S, or NR⁶.

In some embodiments, B is S, D is CH, and E is N.

In one aspect, the invention features a compound of formula (VIIIa)

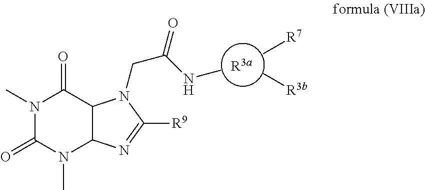

wherein

R^{3a} cyclyl, heterocyclyl, aryl, heteroaryl,

R^{3b} is cyclyl, heterocyclyl, aryl, heteroaryl; optionally substituted with 1-3 R⁷;

each R⁷ is independently C₁-C₆ alkyl, C₂-C₆ alkenyl, or C₂-C₆ alkynyl, halo, oxo, hydroxyl, alkoxy, aryl, heteroaryl, cyclyl, heterocyclyl, aryloxy, arylalkoxy, amino, alkylamino, dialkylamino, thioyl, alkylthioyl, sulfonyl, sulfonamidyl (e.g., where the nitrogen of the sulfonamide is substituted by an alkyl, or where the nitrogen of the sulfonamide together with two carbons to which it is attached, forms a ring), amido(e.g., where the nitrogen of the amide is substituted by an alkyl, or where the nitrogen of the amide together with two carbons to which it is attached, forms a ring), hydroxyl alkoxyl, alkoxy alkoxyl, urea, sulfonylurea, acyl, nitro, cyano, each of which is optionally substituted with 1-3 R⁸;

each R⁸ is independently C₁-C₆ alkyl, C₂-C₆ alkenyl, or C₂-C₆ alkynyl, aryl, heteroaryl, cyclyl, halo, hydroxyl, alkoxy, aryloxy, amino, alkylamino, dialkylamino, thioyl, sulfonyl, sulfonamidyl, amido, urea, sulfonylurea acyl, nitro, cyano, cyclyl, heterocyclyl, aryl, or heteroaryl; and

R⁹ is H or halo.

In some embodiments, R^{3a} is aryl or heteroaryl.

In some embodiments, R^{3a} is heteroaryl.

In some embodiments, R^{3a} is thiazoyl.

In some embodiments, R⁷ is C₁-C₆ alkyl, halo, aryl, or heteroaryl, for example, optionally substituted with 1-3 R⁸

In some embodiments, R^{3b} is aryl or heteroaryl.

In one aspect, the invention features a compound of formula (VIIIb)

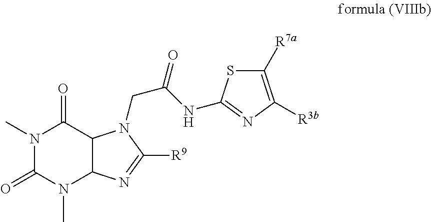

wherein R^{3b} is aryl or heteroaryl; optionally substituted with 1-3 R⁷;

each R⁷ is independently C₁-C₆ alkyl, C₂-C₆ alkenyl, or C₂-C₆ alkynyl, halo, hydroxyl, alkoxy, aryl, heteroaryl, cyclyl, heterocyclyl, aryloxy, arylalkoxy, amino, alkylamino, dialkylamino, thioyl, alkylthioyl, sulfonyl, sulfonamidyl, amido, hydroxyl alkoxyl, alkoxy alkoxyl, urea, sulfonylurea, acyl, nitro, cyano, each of which is optionally substituted with 1-3 R⁸;

R^{7a} is H, C₁-C₆ alkyl, C₂-C₆ alkenyl, or C₂-C₆ alkynyl, halo, hydroxyl, alkoxy, aryl, heteroaryl, cyclyl, heterocyclyl, aryloxy, arylalkoxy, amino, alkylamino, dialkylamino, thioyl, alkylthioyl, sulfonyl, sulfonamidyl, amido, hydroxyl alkoxyl, alkoxy alkoxyl, urea, sulfonylurea, acyl, nitro, cyano, each of which is optionally substituted with 1-3 R⁸;

each R⁸ is independently C₁-C₆ alkyl, C₂-C₆ alkenyl, or C₂-C₆ alkynyl, aryl, heteroaryl, cyclyl, halo, hydroxyl, alkoxy, aryloxy, amino, alkylamino, dialkylamino, thioyl, sulfonyl, sulfonamidyl, amido, urea, sulfonylurea acyl, nitro, cyano, cyclyl, heterocyclyl, aryl, or heteroaryl; and

R⁹ is H or halo.

In some embodiments, R^{3b} is phenyl.

In some embodiments, R^{3b} is phenyl substituted with at least 1 R⁷ and wherein at least 1 R⁷ is amino, alkylamino, dialkylamino or heterocycyl (e.g., a nitrogen containing heterocyclyl such as pyrrolidine or piperidine).

In some embodiments, R^{3b} is

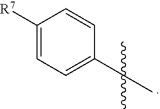

In some embodiments, R^{3b} is further substituted by at least one additional R⁷ (e.g., a halo).

In some embodiments, R⁷ is amino, alkylamino, dialkylamino or heterocycyl (e.g., a nitrogen containing heterocyclyl such as pyrrolidine or piperidine).

In some embodiments, R⁷ is diethylamino.

In some embodiments, R⁷ is pyrrolidinyl.

In some embodiments, R^{3b} is further substituted by at least one additional R⁷ (e.g., a halo).

In some embodiments, at least 1 R⁷ is positioned in the para position of the phenyl ring.

In some embodiments, R^{3b} is a bicyclic fused aryl or heteroaryl, for example, optionally substituted with 1-3 R⁷.

In some embodiments, R⁷ is H.

In some embodiments, R⁷ is C₁-C₆ alkyl.

In some embodiments, R⁹ is H.

In some embodiments, a compound is a substantially pure stereoisomer. For example, in some embodiments, a compound described herein has been purified to provide a substantially chiraly enriched compound (e.g., wherein the compound is substantially free of other stereoisomers). In some embodiments, the compound is at least about 60% pure, e.g., at least about 65%, at least about 70%, at least about 75%, at least about 80%, at least about 85%, at least about 90%, at least about 95%, at least about 97%, at least about 98%, or at least about 99%.

In some embodiments, the compound is a compound depicted in Table 2 (e.g., is any one of the individual compounds) having a formula described herein. For example, the compounds of the invention are a compound described in Table 2 of U.S. Ser. No. 11/645,307 filed Dec. 12, 2006, which is incorporated herein by reference in its entirety.

One aspect of the present invention provides a pharmaceutical preparation suitable for use in a human patient, or for veterinary use, comprising an effective amount of any of the compounds shown above (e.g., a compound of described herein or a salt thereof, or a solvate, hydrate, oxidative metabolite or prodrug of the compound or its salt), and one or more pharmaceutically acceptable excipients. In certain embodiments, the pharmaceutical preparations may be for use in treating or preventing a condition involving activation of TRPA1 or for which reduced TRPA1 activity can reduce the severity. In certain embodiments, the pharmaceutical preparations have a low enough pyrogen activity to be suitable for use in a human patient, or for veterinary use. In certain embodiments, the pharmaceutical preparation comprises an effective amount of any of the compounds shown above, wherein the compound inhibits TRPA1 (e.g., a TRPA1-mediated current and/or TRPA1-mediated ion flux) with an IC₅₀ of 10 micromolar or less. In certain embodiments, the pharmaceutical preparation comprises a compound which inhibits TRPA1 with an IC₅₀ of 5 micromolar or less, 2 micromolar or less, 1 micromolar or less, or with an IC₅₀ of 500 nM or less, 250 nM or less, 200 nM or less, or 100 nM or less.

TRPA1 antagonists of the subject invention can be used as part of a prophylaxis or treatment for a variety of disorders and conditions, including, but not limited to, respiratory conditions as described herein. The invention contemplates the use of compounds having any of the structures provided in the specification in the treatment of or to reduce the symptoms of any of the diseases or conditions disclosed in the application. The invention further contemplates the use of compounds having any of the structures provided in the specification in the manufacture of a medicament or pharmaceutical preparation to treat or reduce the symptoms of any of the diseases or conditions provided in the specification. Compounds for use in treating a particular disease or condition can be formulated for administration via a route appropriate for the particular disease or condition.

Individuals that may be treated with the present compounds are identified using any standard techniques known in the art for diagnosing respiratory conditions including, for example, X-rays, pulmonary function test, computed tomography scan, culture of microorganisms from bodily fluids (e.g., blood, sputum, urine, tears, and saliva), bronchoscopy, biopsy (e.g., of the lung or pleura), ventilation test or perfusion scan, breathing devices such as a peak flow meter or by spirometry, and ultrasound scan.

TRPA1 antagonists can be administered alone or in combination with other therapeutic agents including corticosteroids, bronchodilators, antibiotics, anticoagulants, chemotherapy, immunosuppressants, physiotherapy, oxygen, mechanical ventilation, radiotherapy, surgery, removal of a cancer (e.g. lobectomy and pneumonectomy), pleurodesis, lung volume reduction surgery, and lung transplantation. The TRPA1 antagonist may also be administered conjointly with one or more of an anti-inflammatory agent, anti-proliferative agent, anti-fungal agent, anti-viral agent, or anti-septic agent.

TRPA1 antagonists can be administered topically, orally, transdermally, rectally, vaginally, parentally, intranasally, intrapulmonary, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally or by inhalation.

In certain preferred embodiments, a TRPA1 antagonist is administered topically.

In certain preferred embodiments, a TRPA1 antagonist is administered orally.

In certain preferred embodiments, a TRPA1 antagonist is administered parentally.

In certain preferred embodiments, a TRPA1 antagonist is administered to prevent, treat or alleviate signs and symptoms of cough.

Still another aspect of the present invention relates to the use of a TRPA1 antagonist, e.g., a small molecule agent that inhibits inward TRPA1-mediated current with an IC₅₀ of 1 micromolar or less, in the manufacture of a medicament to prevent, treat or alleviate symptoms of a disease, disorder or condition involving activation of TRPA1, or for which reduced TRPA1 activity can reduce the severity, in a patient.

Yet another aspect of the present invention relates to a pharmaceutical preparation comprising an agent that inhibits inward TRPA1-mediated current with an IC₅₀ of 1 micromolar or less; and a pharmaceutically acceptable excipient or solvent wherein the agent is provided in a dosage form providing an amount effective to prevent, treat or alleviate symptoms of a disease, disorder or condition involving activation of TRPA1, or for which reduced TRPA1 activity can reduce the severity, in a patient. In certain preferred embodiments, the pharmaceutical preparation does not cause QT interval elongation in the patient.

In certain illustrative embodiments, the pharmaceutical preparation comprises an agent that inhibits TRPA1-mediated current with an IC₅₀ of at least one order of magnitude lower than its IC₅₀ for inhibition of NaV 1.2 function, TRPV1 function, TRPV5 function, TRPV6 function, mitochondrial uniporter function and HERG function; and a pharmaceutically acceptable excipient or solvent, wherein the agent is provided in a dosage form providing an amount effective to prevent, treat or alleviate symptoms of a disease, disorder or condition involving activation of TRPA1, or for which reduced TRPA1 activity can reduce the severity, in a patient, but which does not cause QT interval elongation.

In another illustrative embodiment, the pharmaceutical preparation comprises an agent that inhibits a TRPA1-mediated current with an IC₅₀ of 1 micromolar or less; and a pharmaceutically acceptable excipient or solvent, wherein the agent is provided in a dosage form providing an amount effective to prevent, treat or alleviate symptoms of a disease, disorder or condition involving activation of TRPA1, or for which reduced TRPA1 activity can reduce the severity, in a patient, but which does not cause QT interval elongation.

One preferred preparation is a topical formulation for reducing TRPA1 activity in skin or mucosa, comprising an agent that inhibits a TRPA1-mediated current with an IC₅₀ of 1 micromolar or less.

Another preferred preparation is a removable patch or bandage, comprising: (i) a polymeric base; and (ii) an agent that inhibits a TRPA1-mediated current with an IC₅₀ of 1 micromolar or less.

Yet another embodiment is an antitussive composition for peroral administration comprising an agent that inhibits both a TRPA1-mediated current with an IC₅₀ of 1 micromolar or less, and an orally-acceptable pharmaceutical carrier in the form of an aqueous-based liquid, or solid dissolvable in the mouth, selected from the group consisting of syrup, elixer, suspension, spray, lozenge, chewable lozenge, powder, and chewable tablet. Such antitussive compositions can include one or more additional agents for treating cough, allergy or asthma symptom selected from the group consisting of: antihistamines, 5-lipoxygenase inhibitors, leukotriene inhibitors, H3 inhibitors, β-adrenergic receptor agonists, xanthine derivatives, α-adrenergic receptor agonists, mast cell stabilizers, expectorants, NK1, NK2 and NK3 tachykinin receptor antagonists, and GABA_{B} agonists.

Still another embodiment is a metered dose aerosol dispenser containing an aerosol pharmaceutical composition for pulmonary or nasal delivery comprising an agent that inhibits a TRPA1-mediated current with an IC₅₀ of 1 micromolar or less. For instance, it can be a metered dose inhaler, a dry powder inhaler or an air-jet nebulizer.

In another aspect, the invention contemplates that any of the TRPA1 inhibitors of the present invention, including inhibitors having one or more of the characteristics disclosed herein, can be used to inhibit a function of TRPA1, for example a TRPA1-mediated current and/or a TRPA1-mediated ion flux. In some embodiments, the compounds can be used to inhibit a TRPA1 mediated current in vitro, for example in cells in culture. In some embodiments, the compounds can be used to inhibit a TRPA1 mediated current in vivo. In certain embodiments, the compounds inhibit both an inward and an outward TRPA1-mediated current. In certain embodiments, the compounds inhibit a TRPA1 mediated ion flux in vitro, for example in cells in culture. In certain other embodiments, the compounds inhibit a TRPA1 mediated in flux in vivo.

The invention contemplates pharmaceutical preparations and uses of TRPA1 antagonists having any combination of the foregoing or following characteristics, as well as any combination of the structural or functional characteristics of the TRPA1 antagonists described herein. Any such antagonists or preparations can be used in the treatment of any of the diseases or conditions described herein. Any such antagonists or preparations can be used to inhibit a function of TRPA1, for example a TRPA1-mediated current and/or a TRPA1-mediated ion flux.

### DESCRIPTION OF THE DRAWINGS

FIGS. 1A and 1B are graphs showing the plasma levels of compounds 7 (intravenous or oral administration) and 8 (oral administration) of Table 3 over time.

FIG. 1C is a table summarizing the pharmacokinetics of compound 7 of Table 3 following oral and intravenous administration.

FIGS. 2A and 2B are graphs showing the plasma levels of compound 7 and compound 8 of Table 3 over time following oral administration.

FIG. 3 is Table 2, showing compounds that are part of the invention.

FIG. 4 is Table 4 a table showing exemplary compounds of the invention.

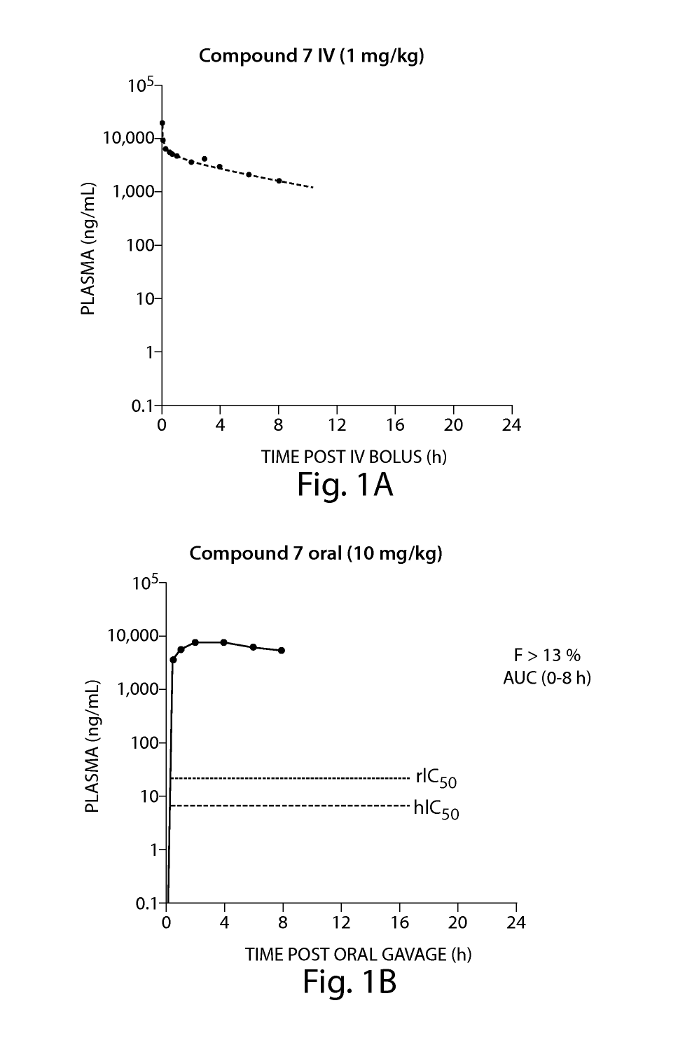

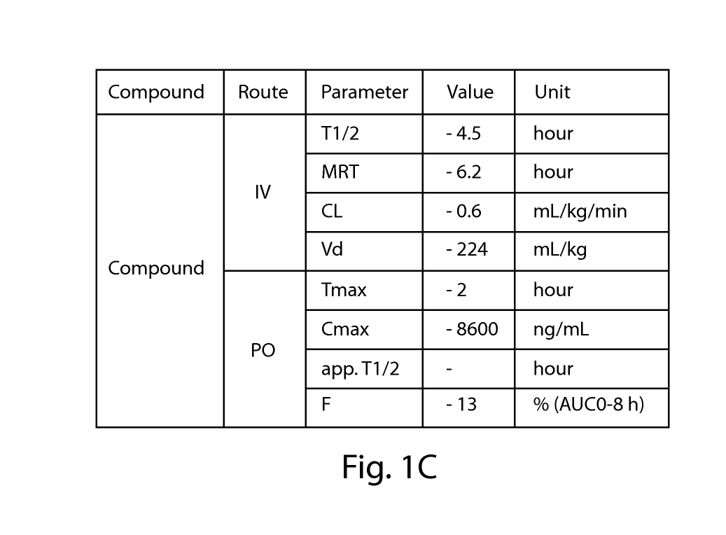

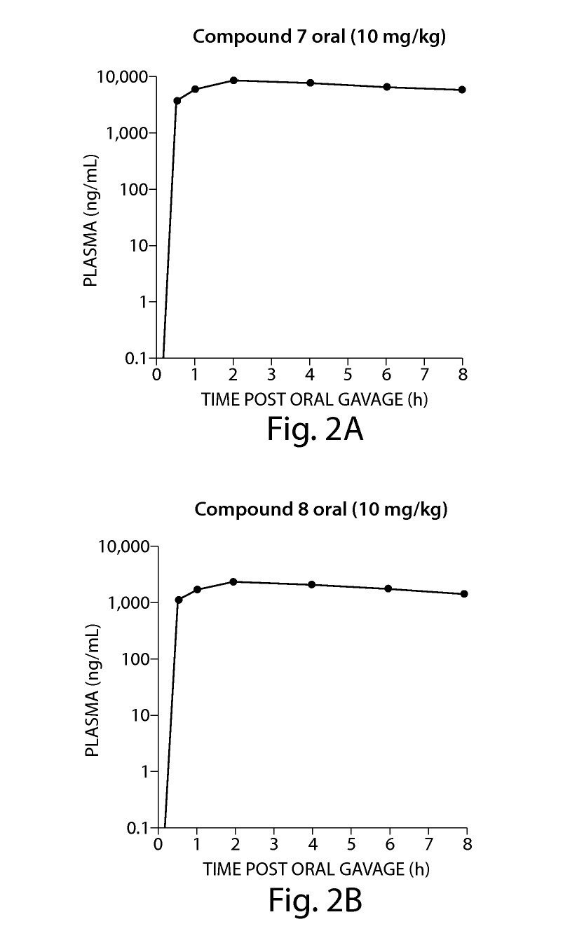

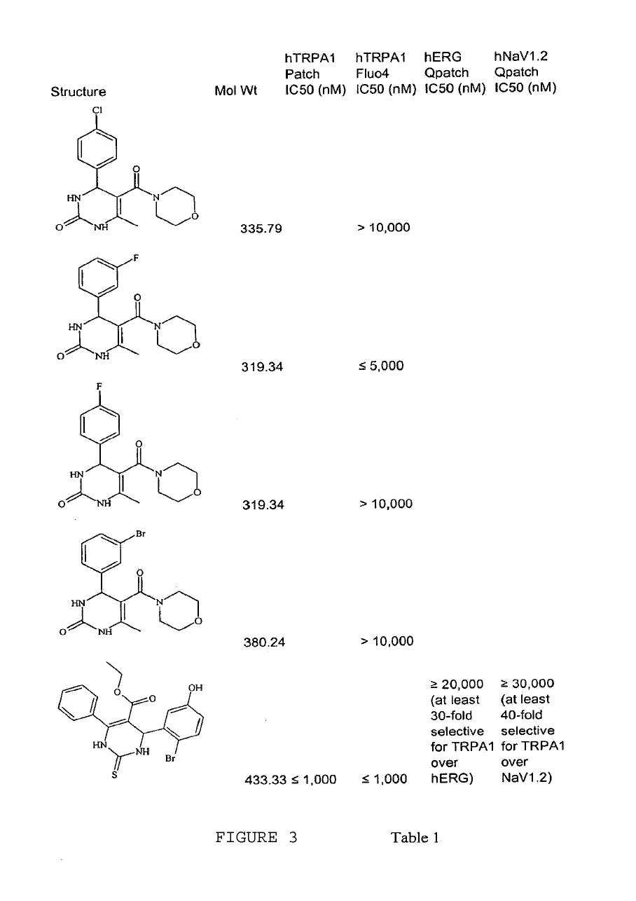

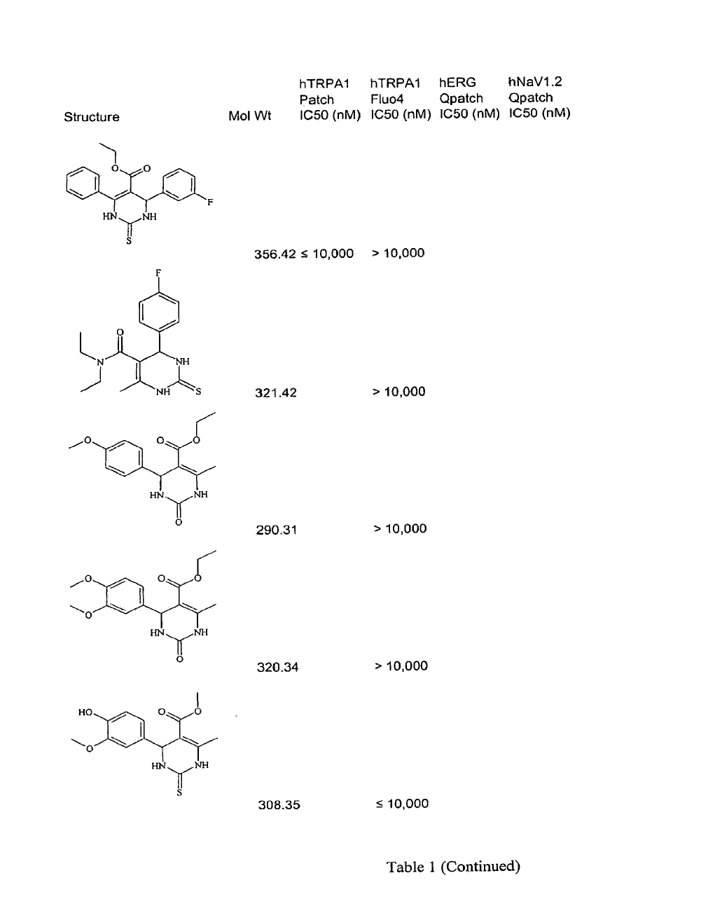

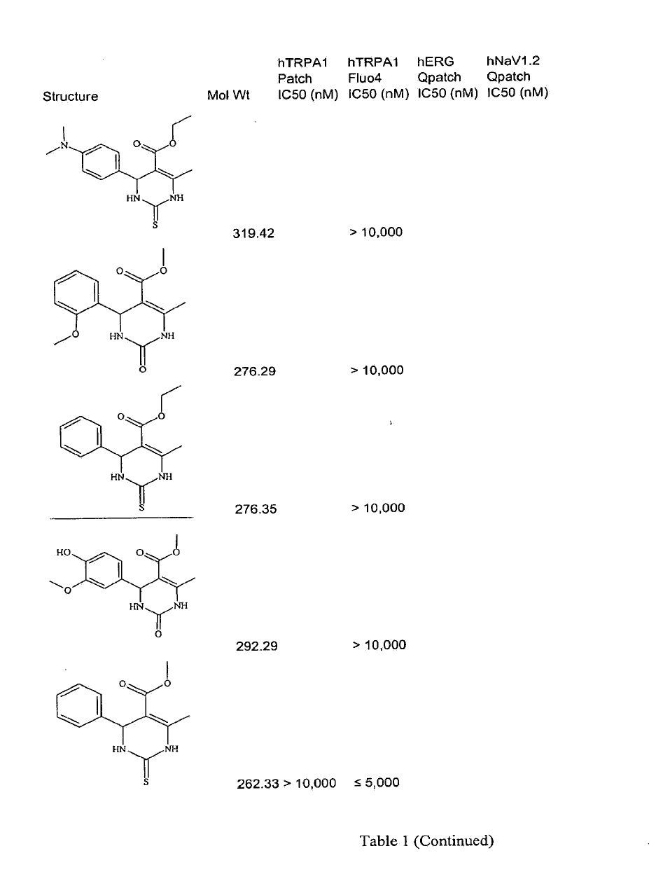

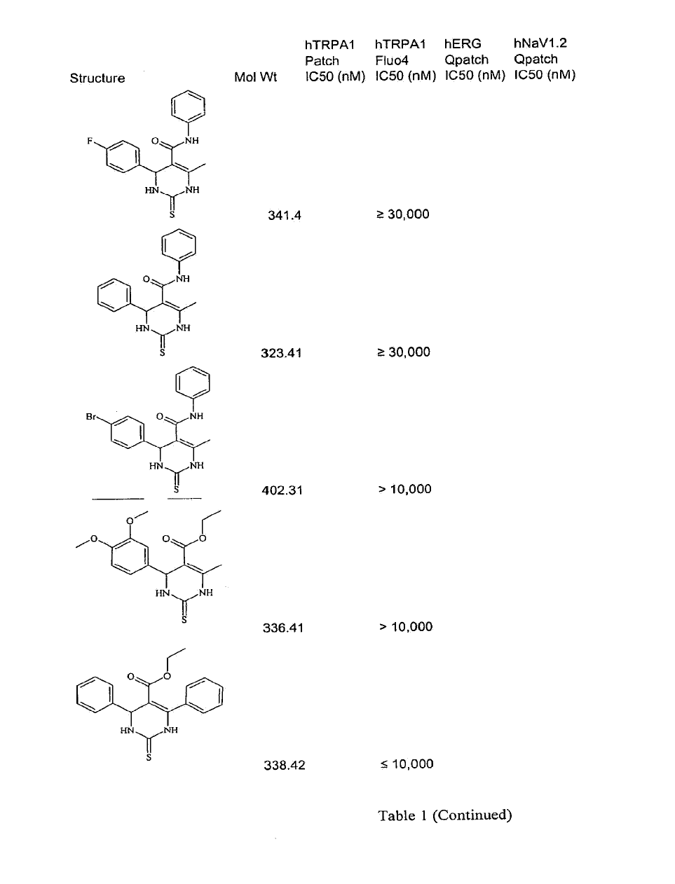

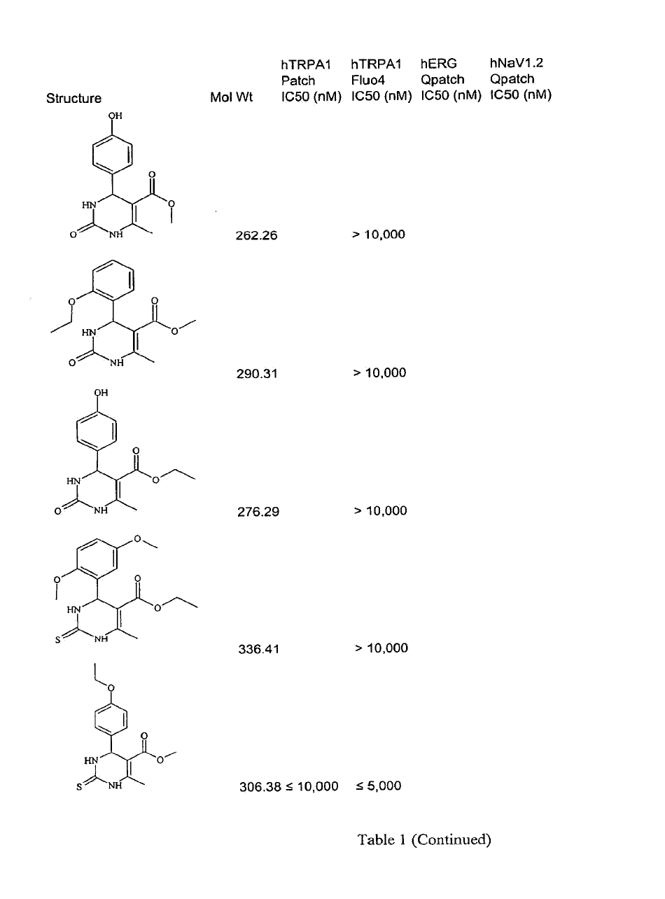

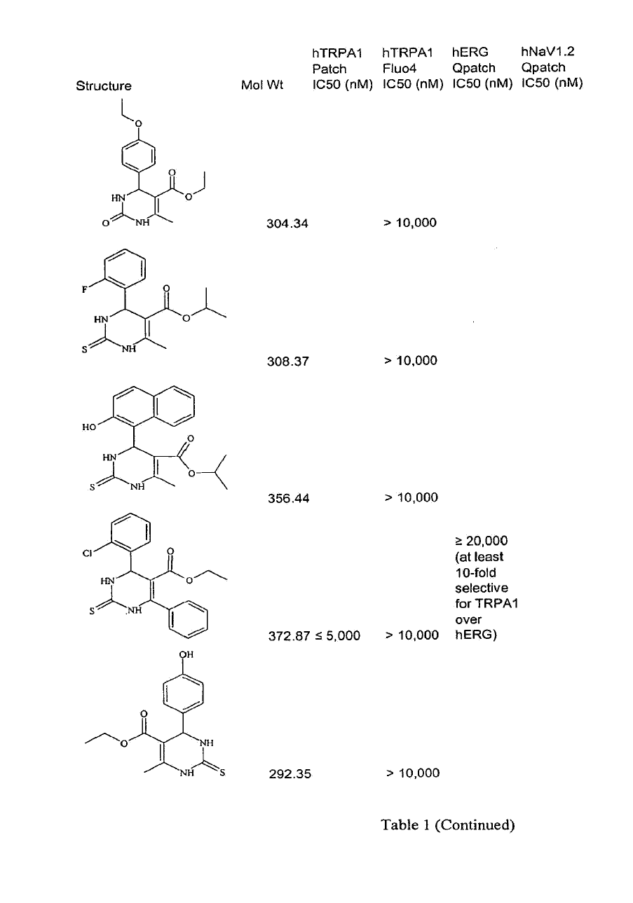

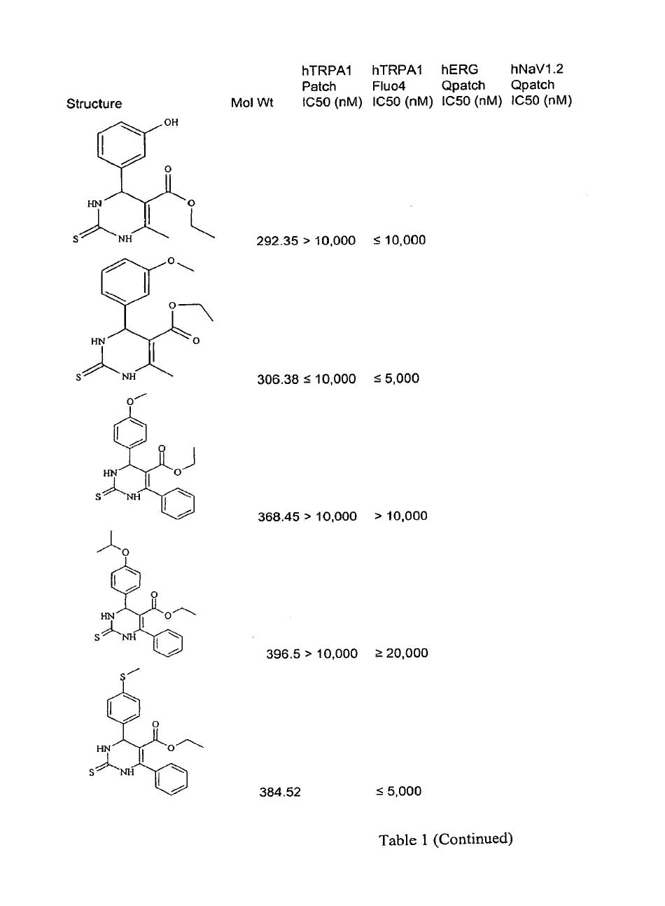

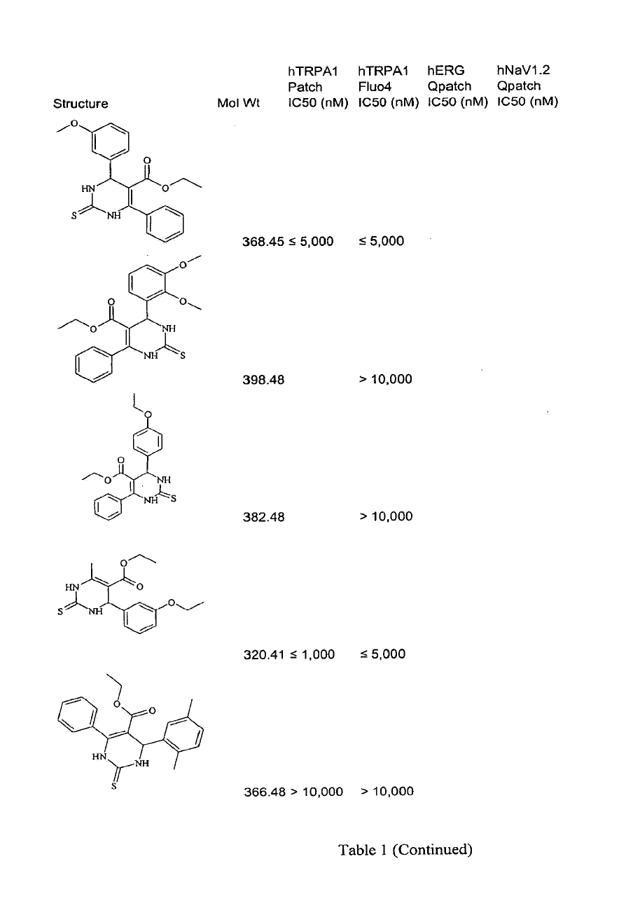

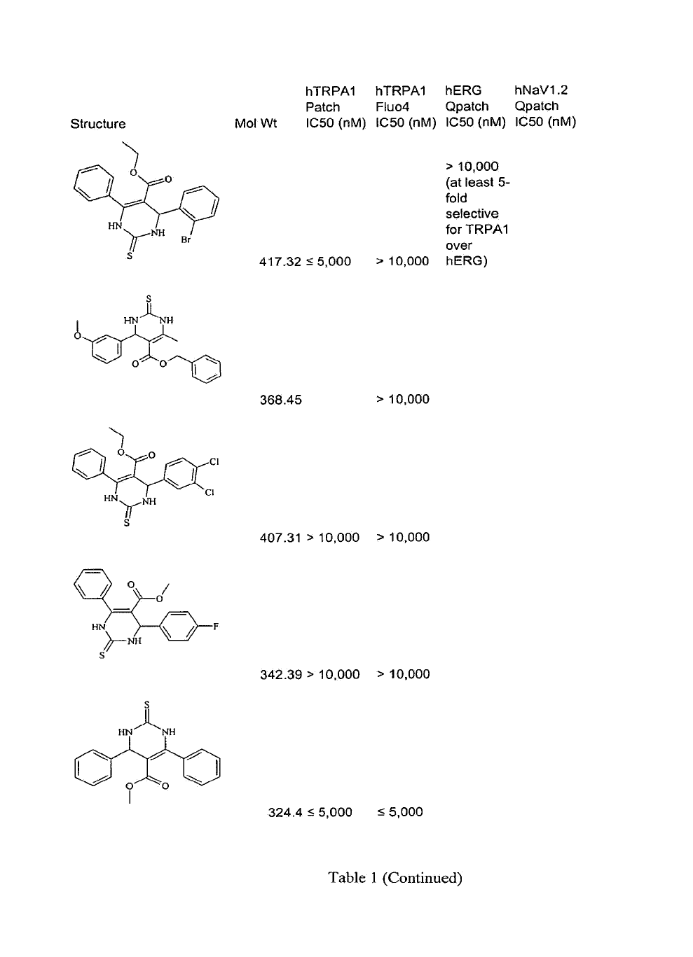

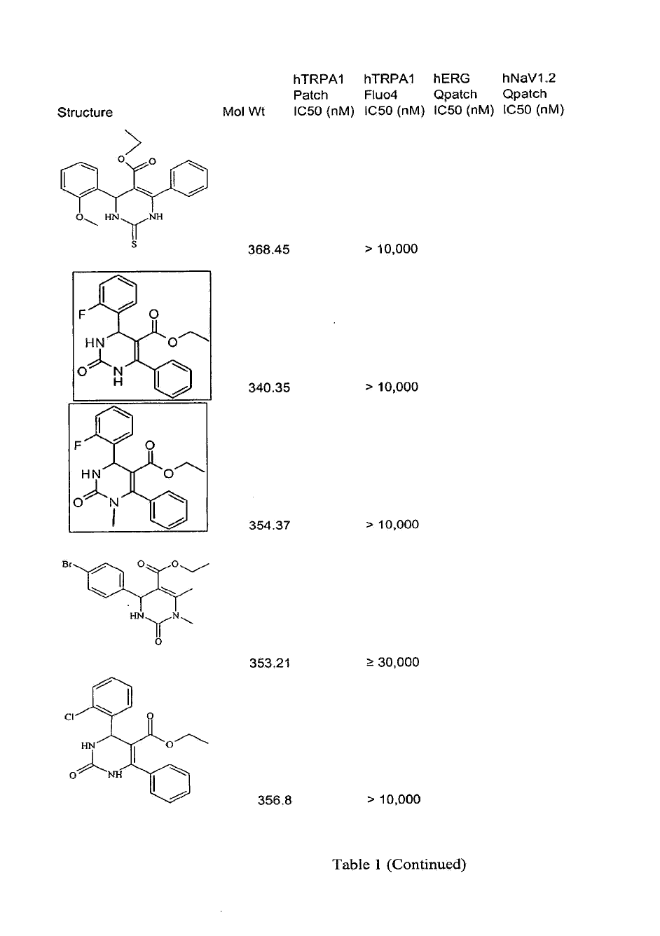

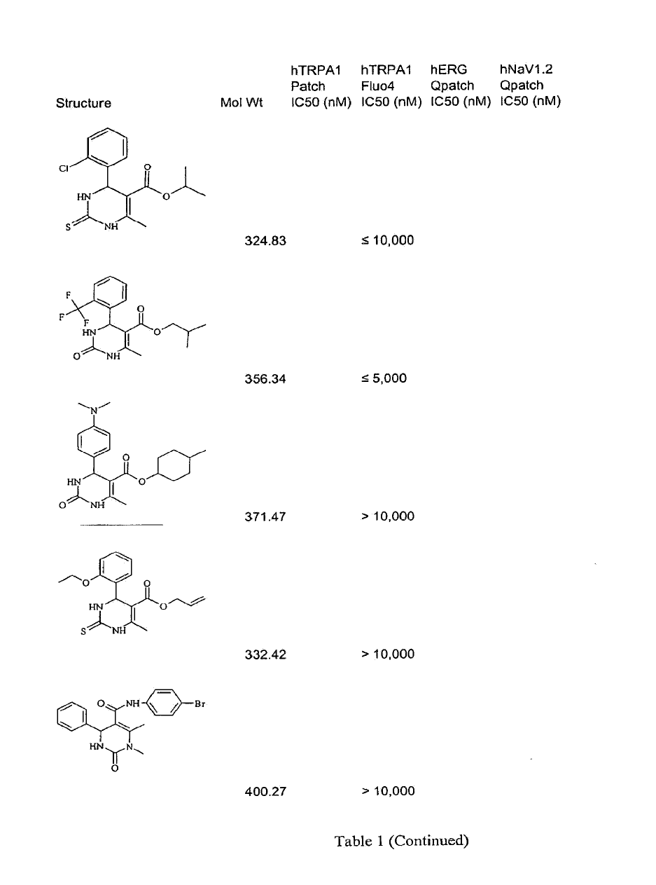

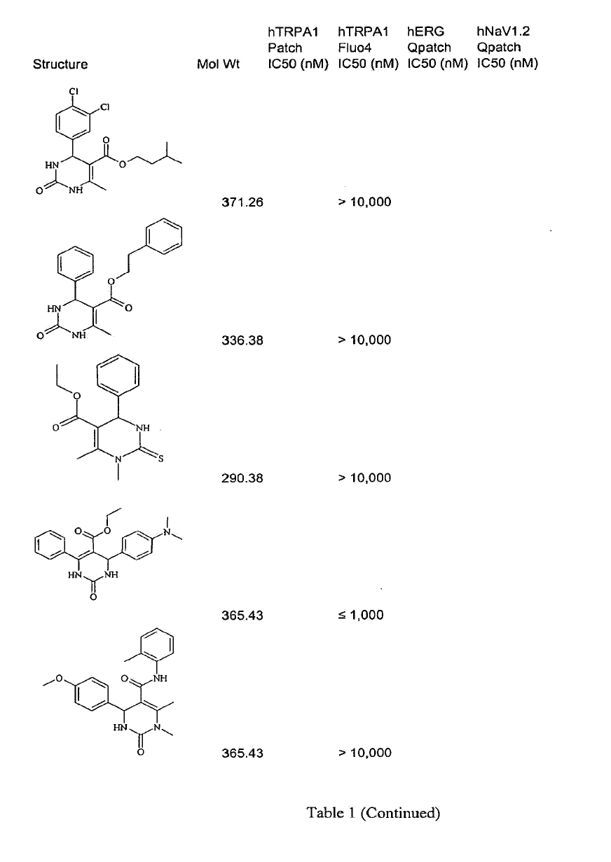

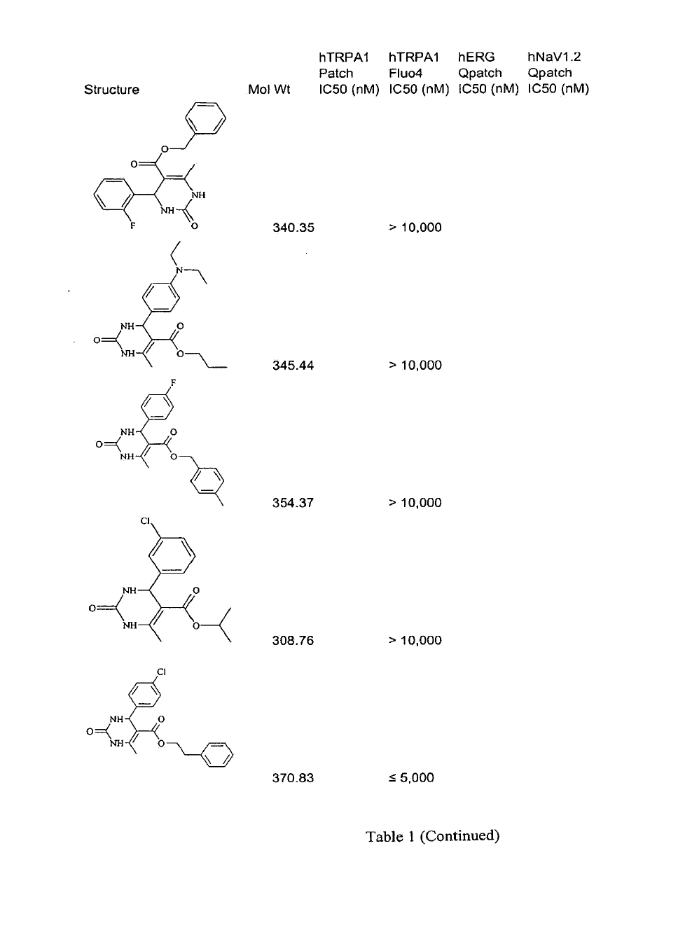

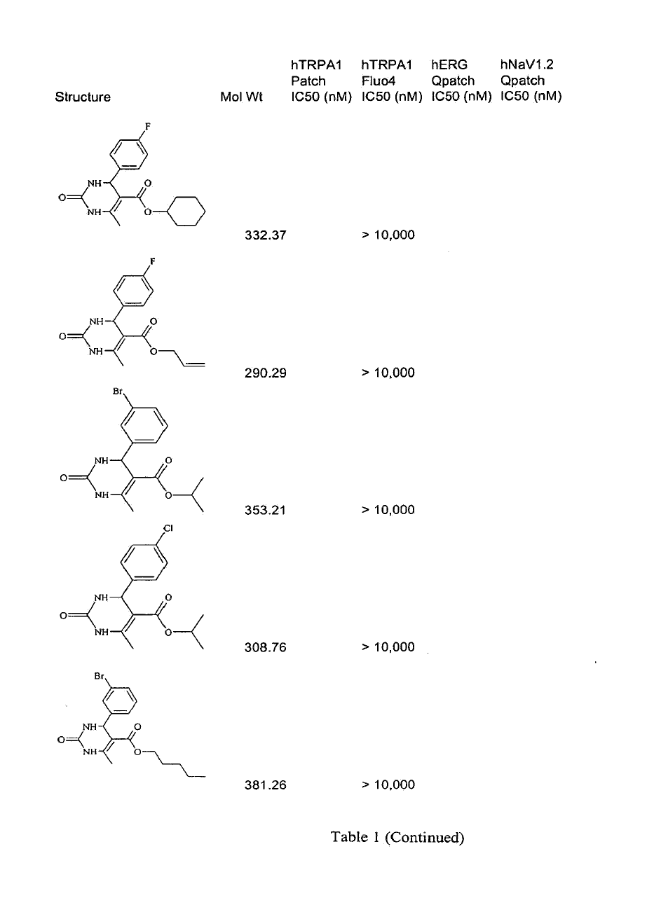

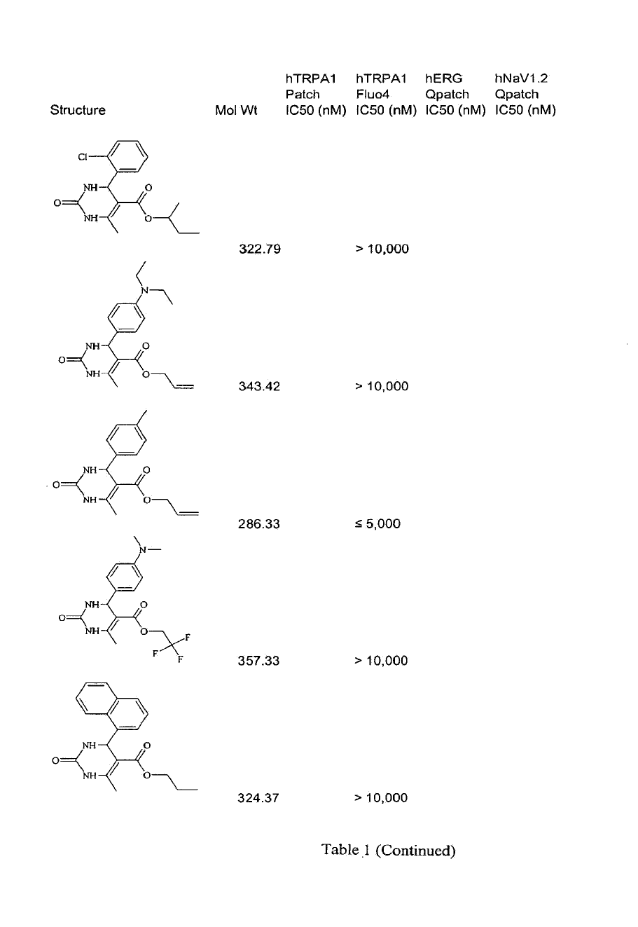

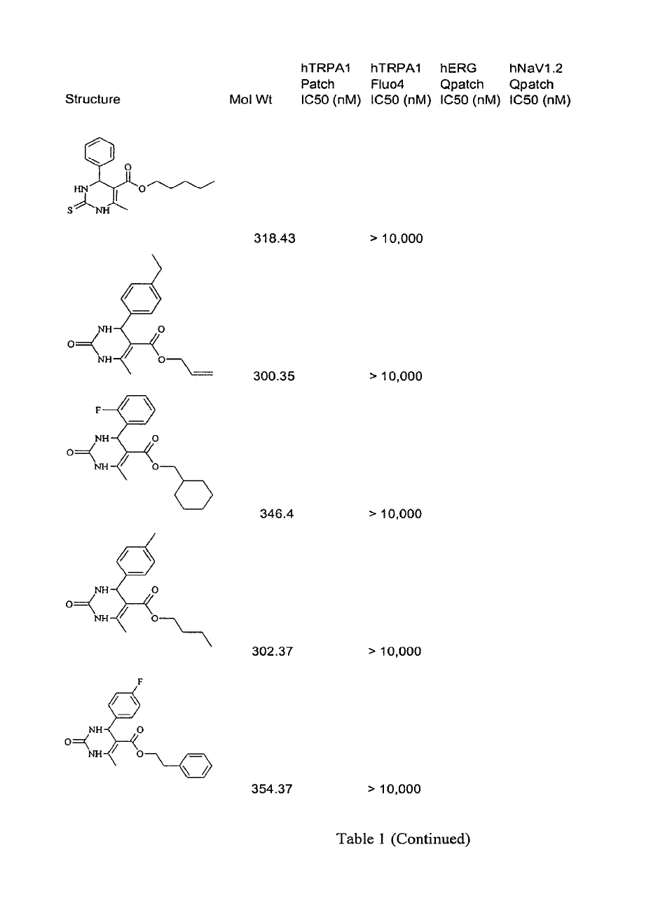

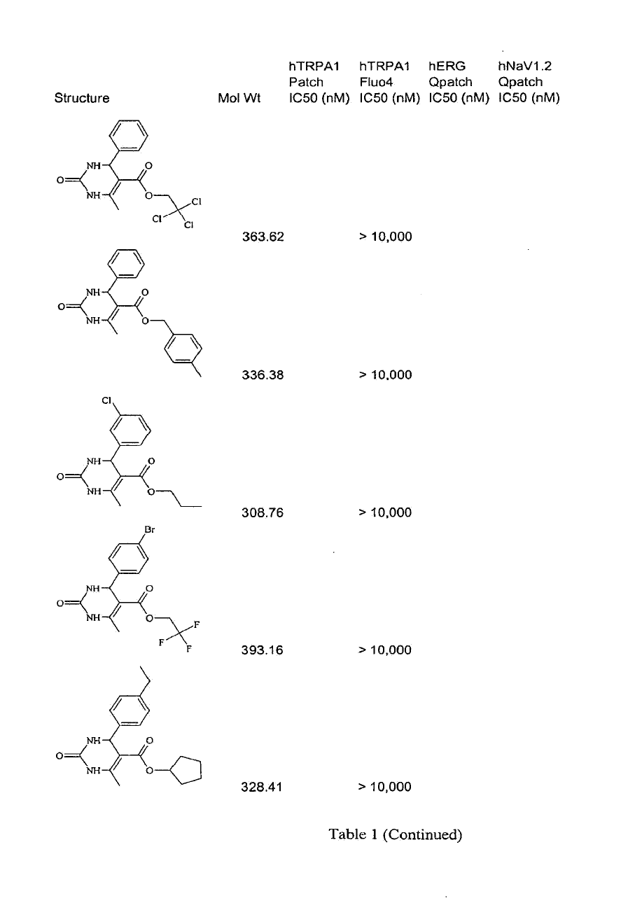

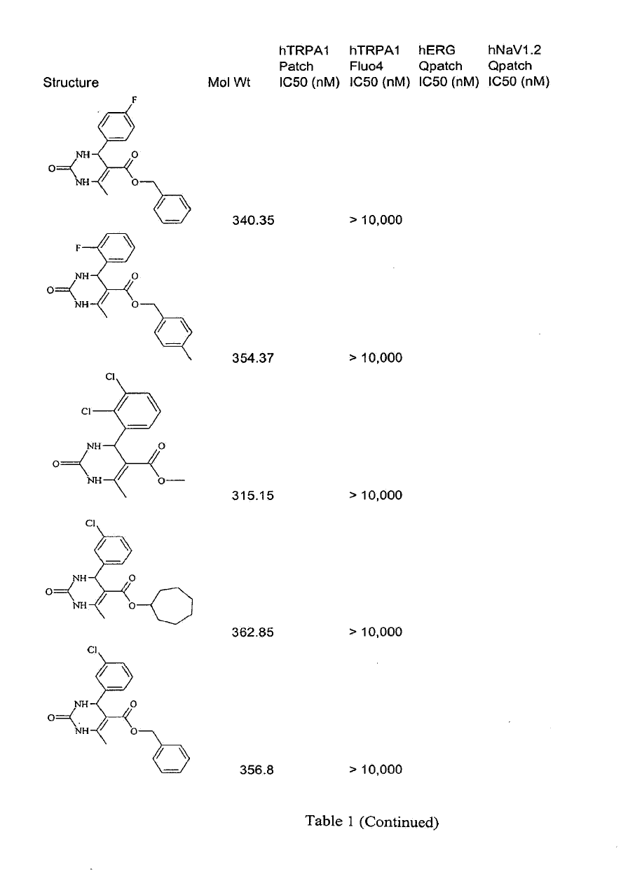

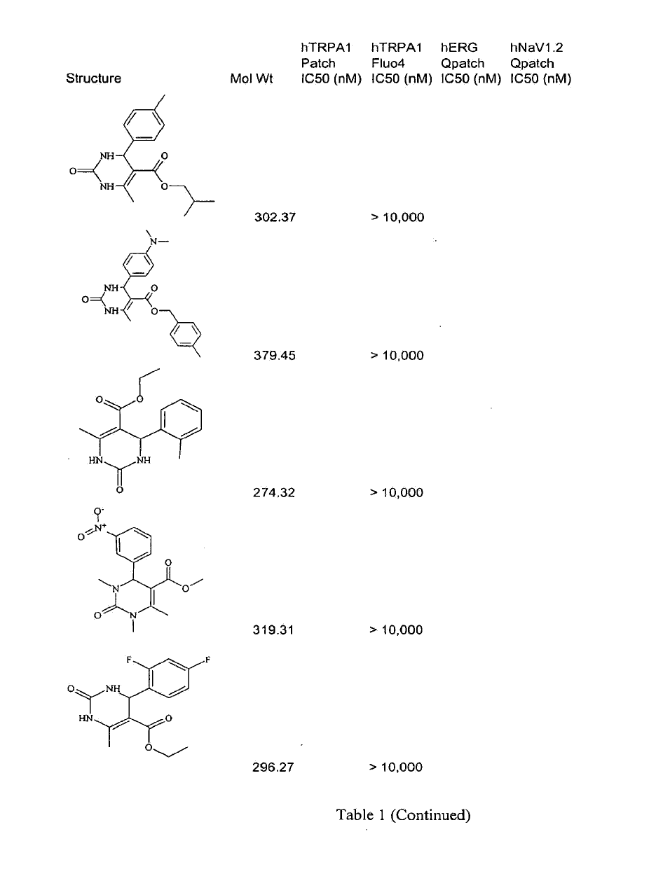

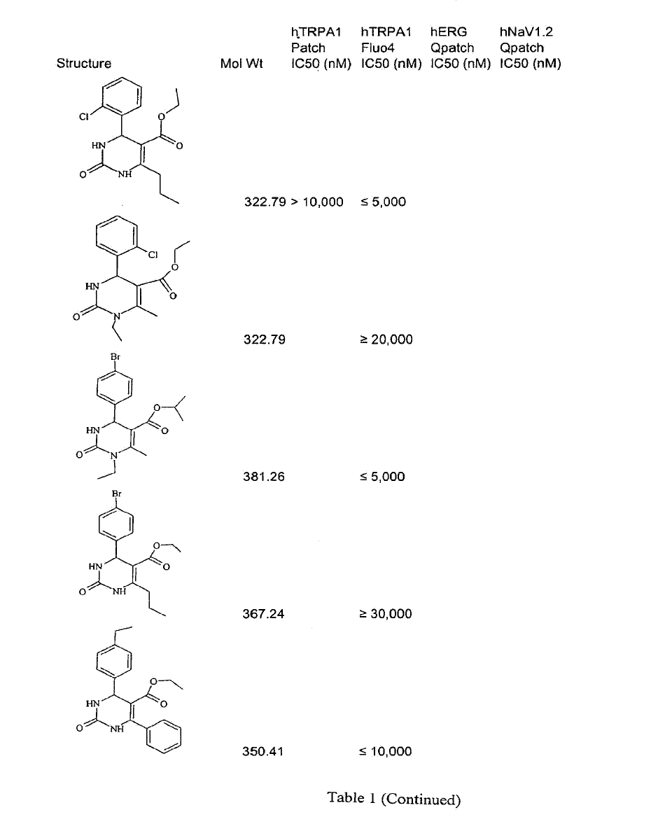

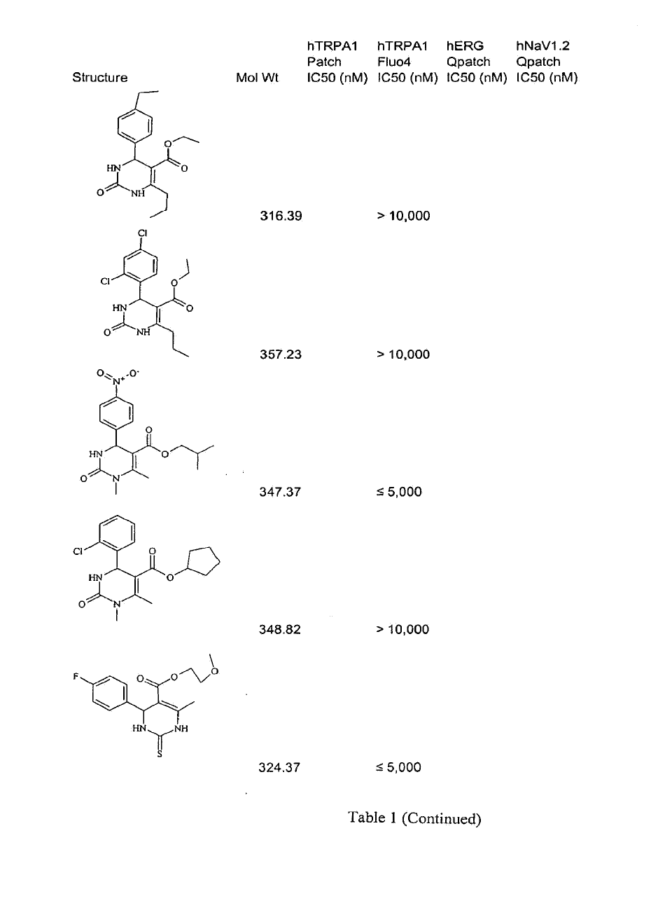

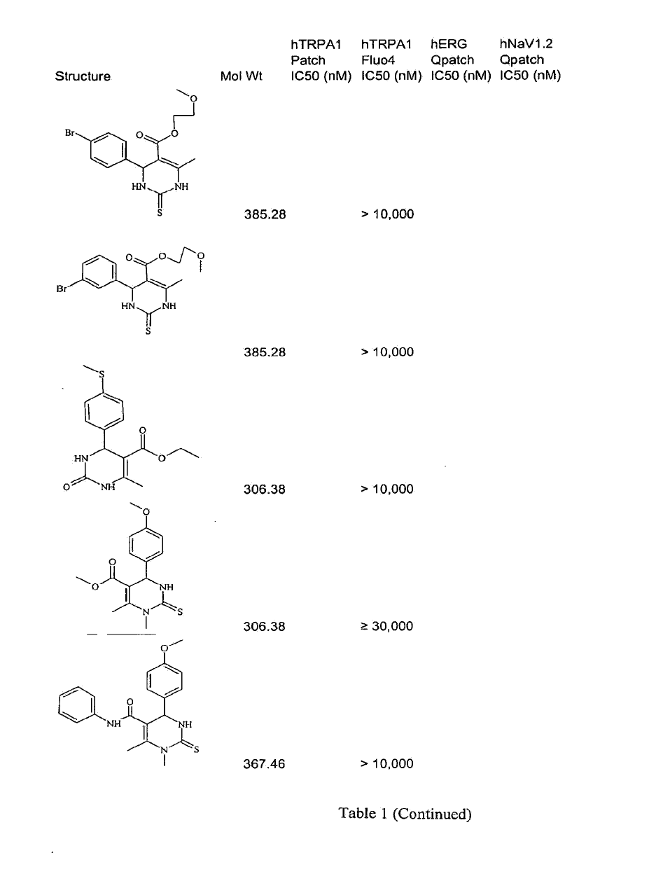

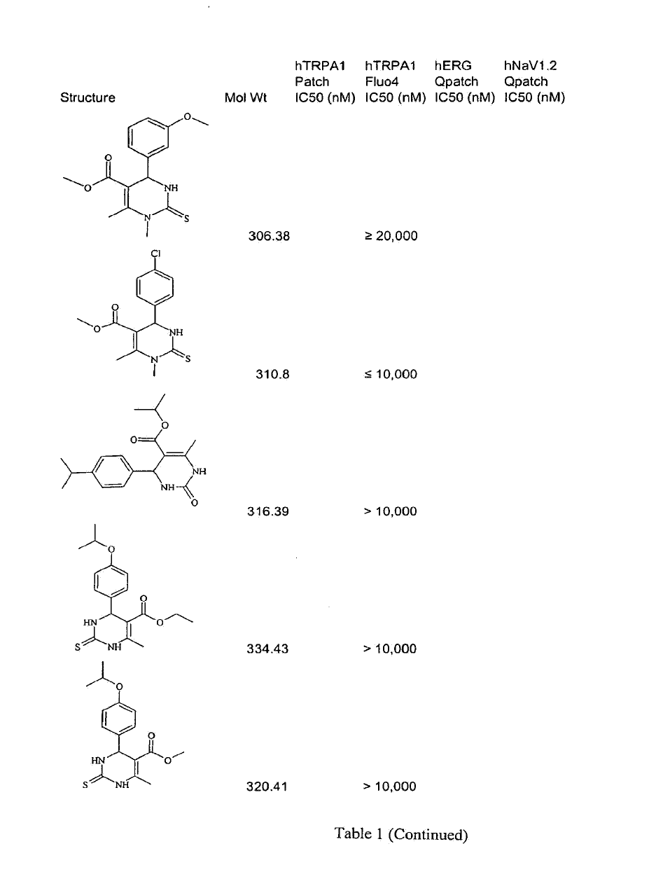

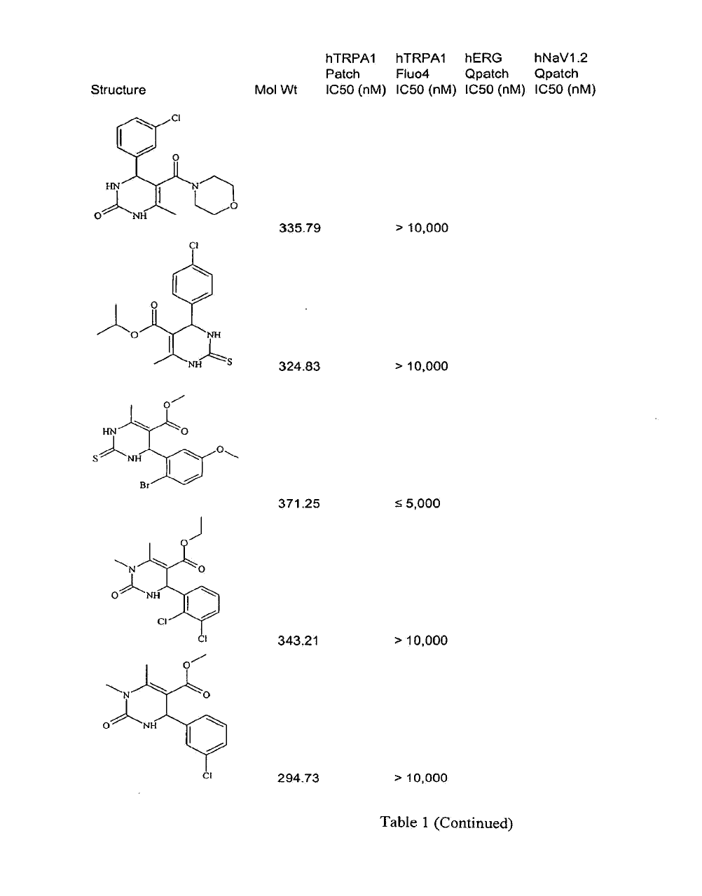

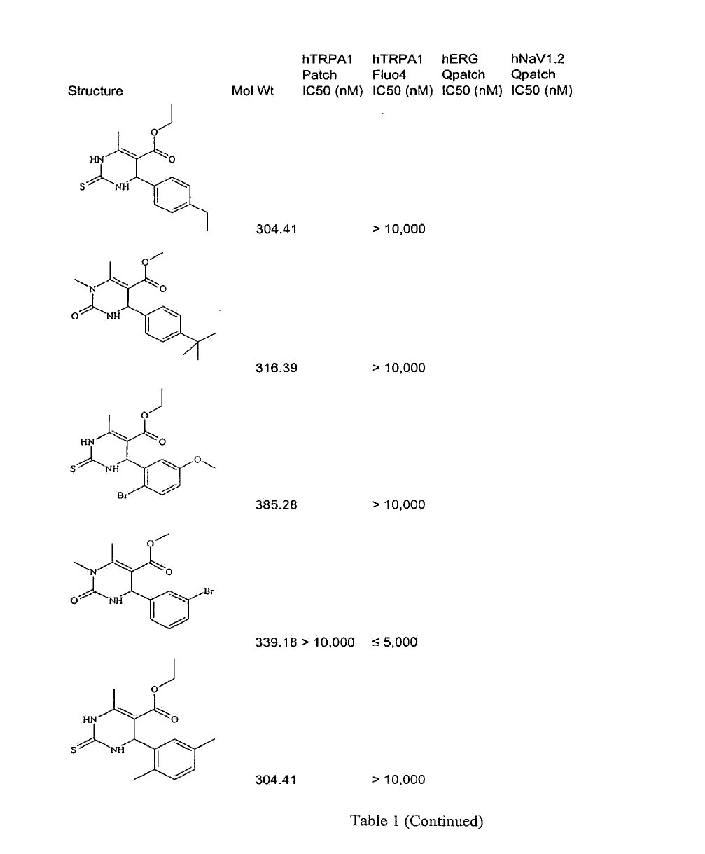

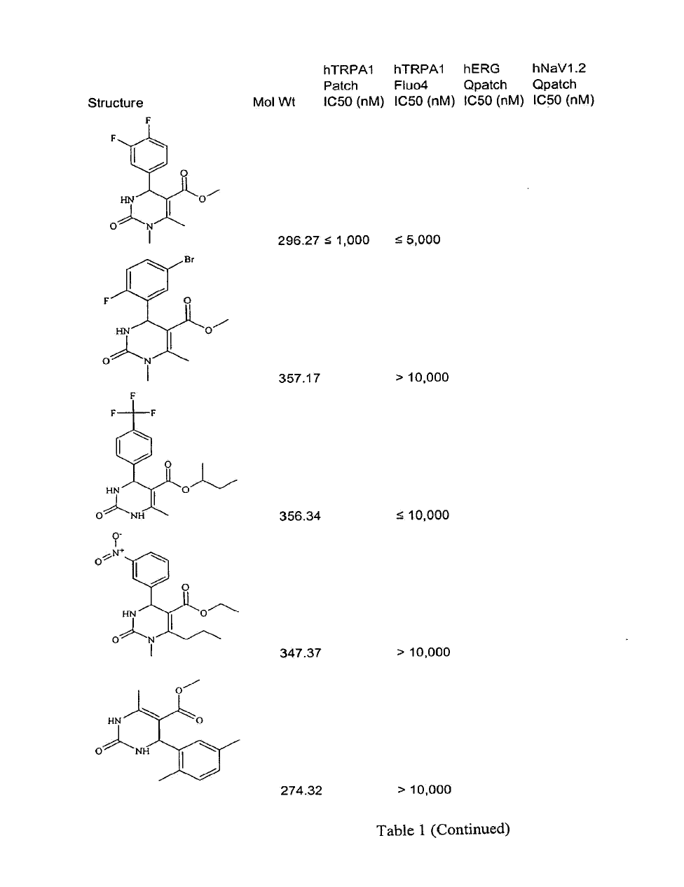

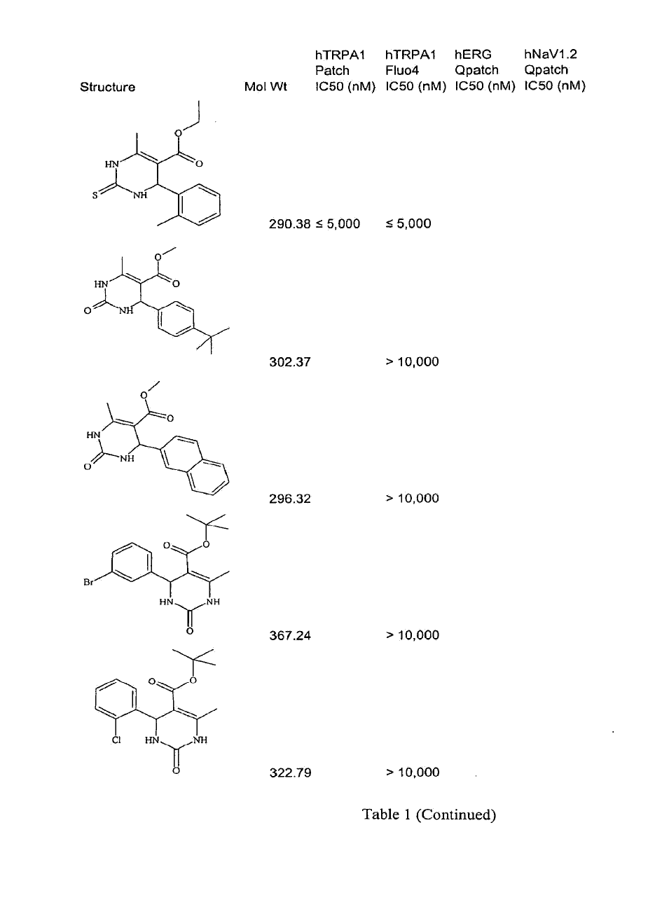

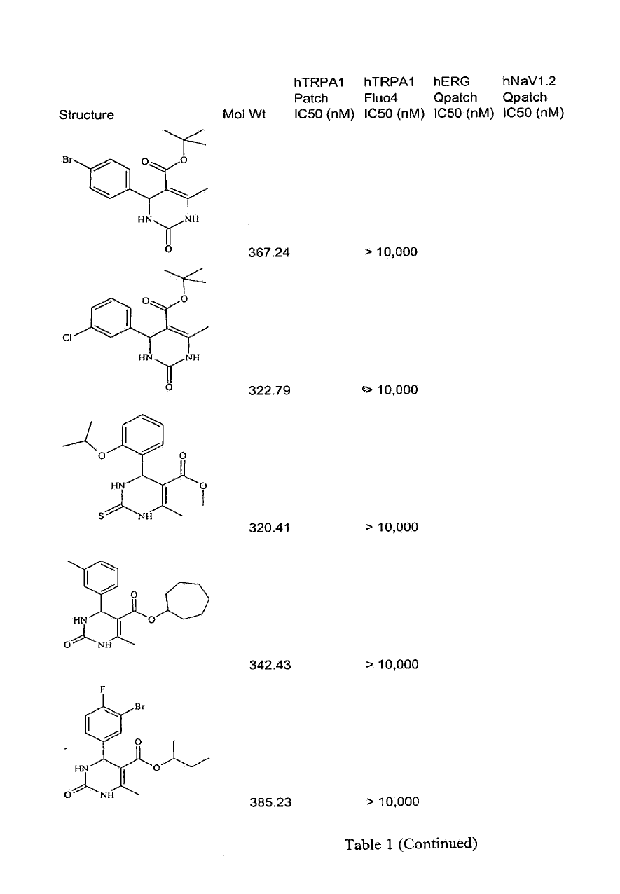

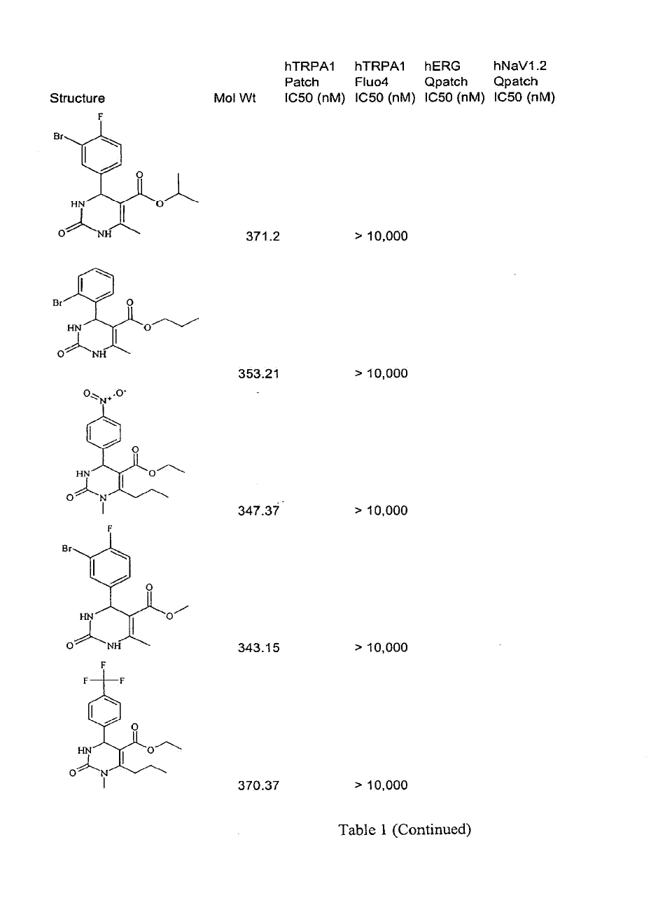

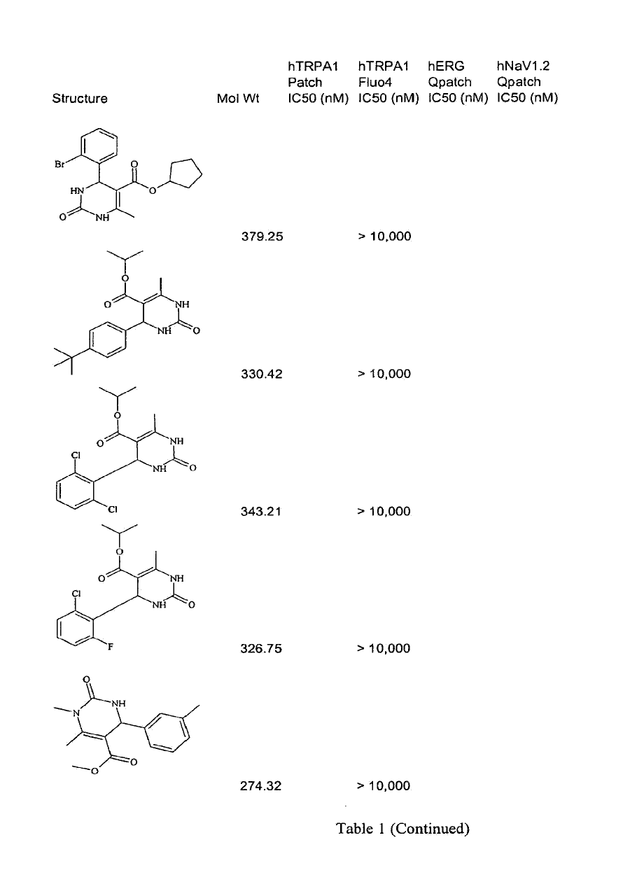

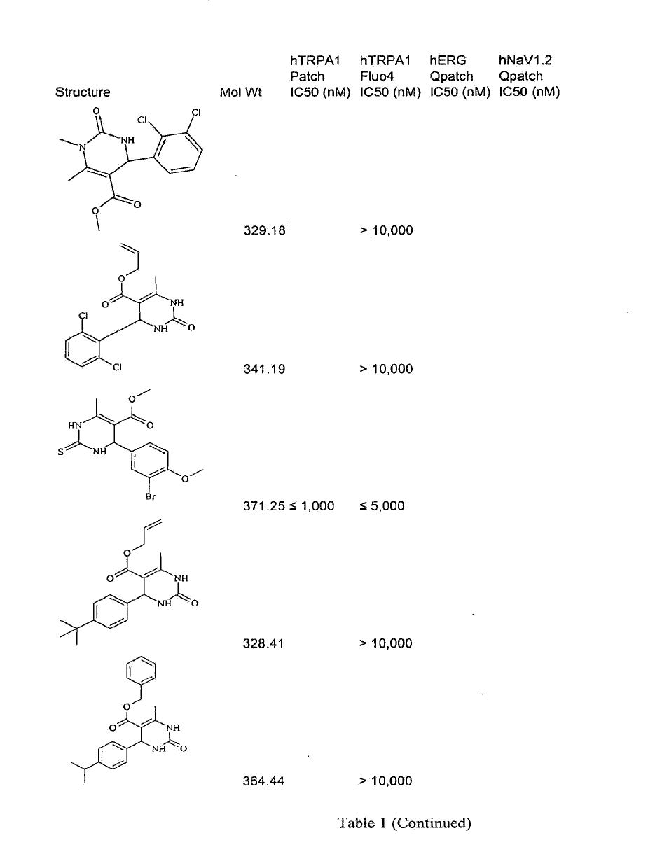

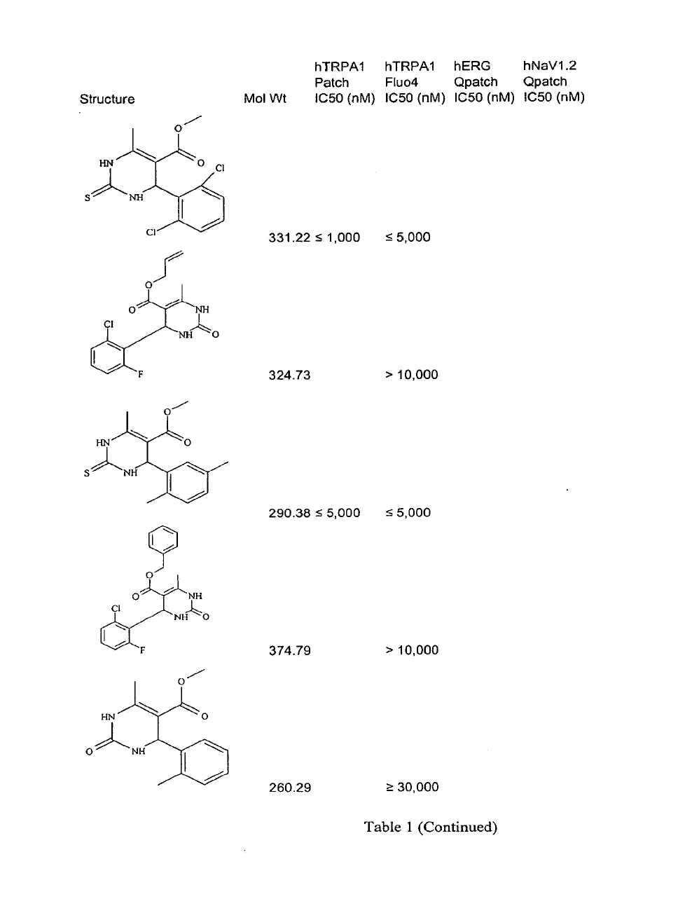

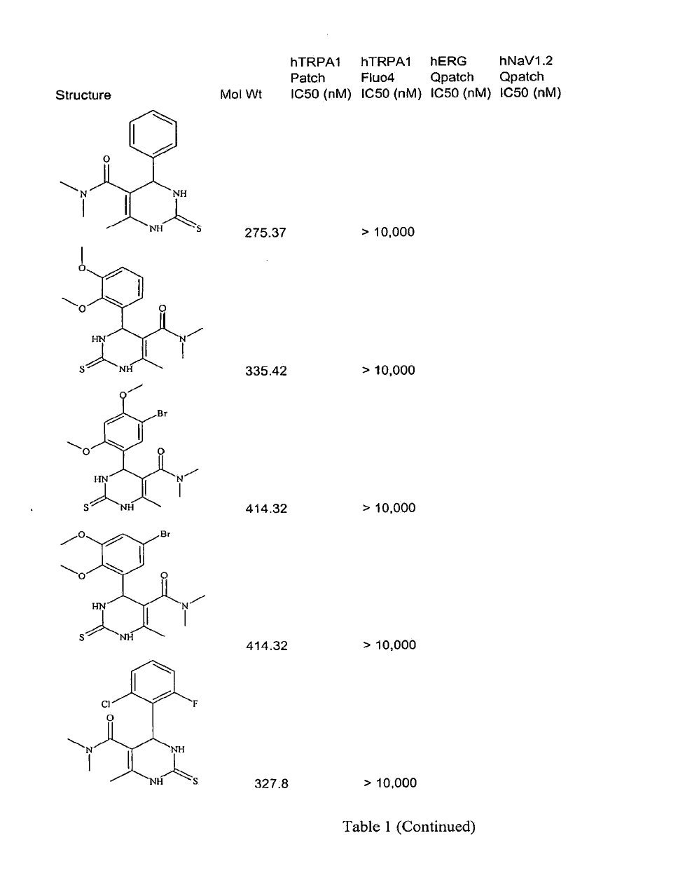

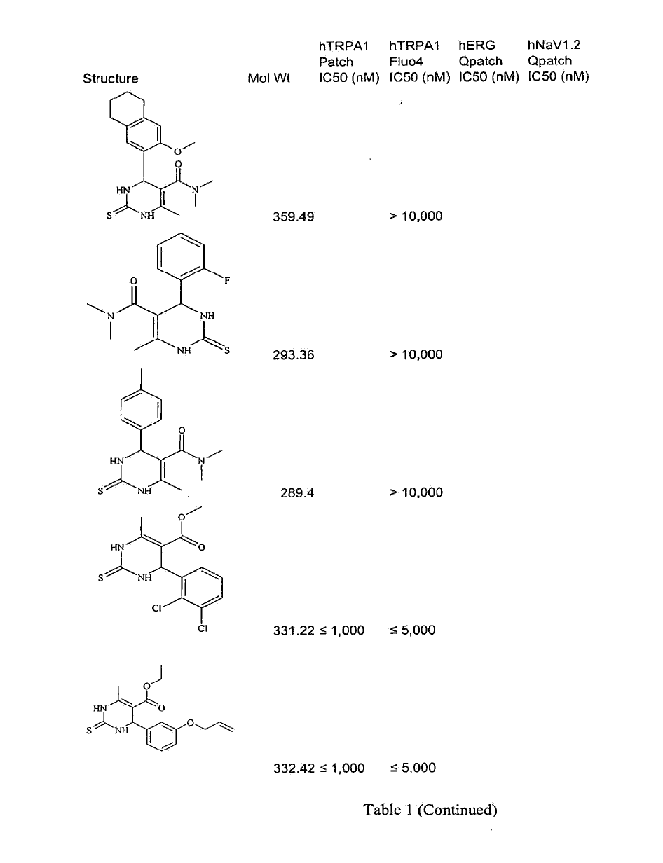

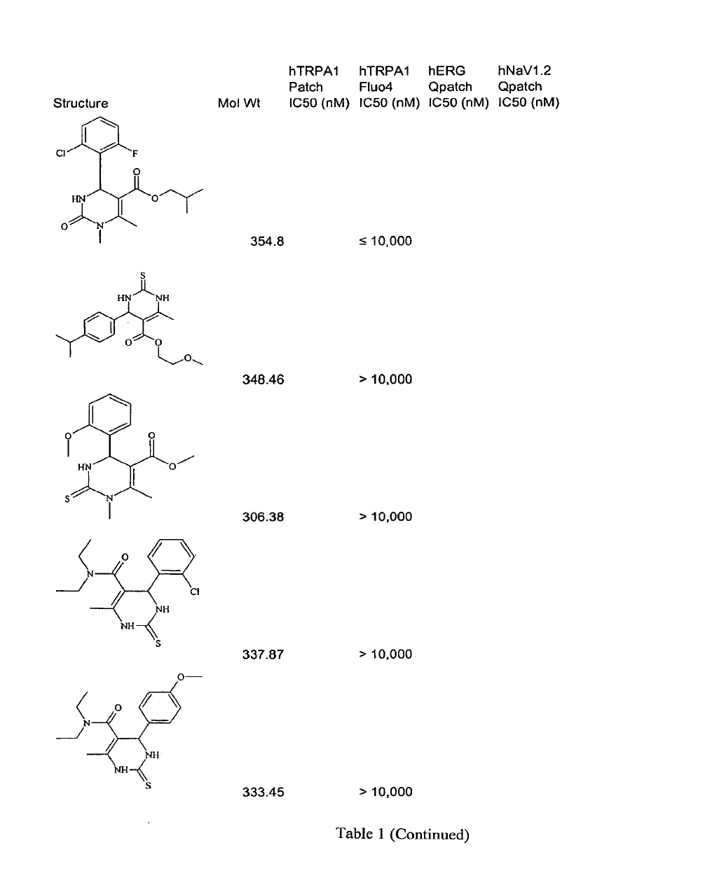

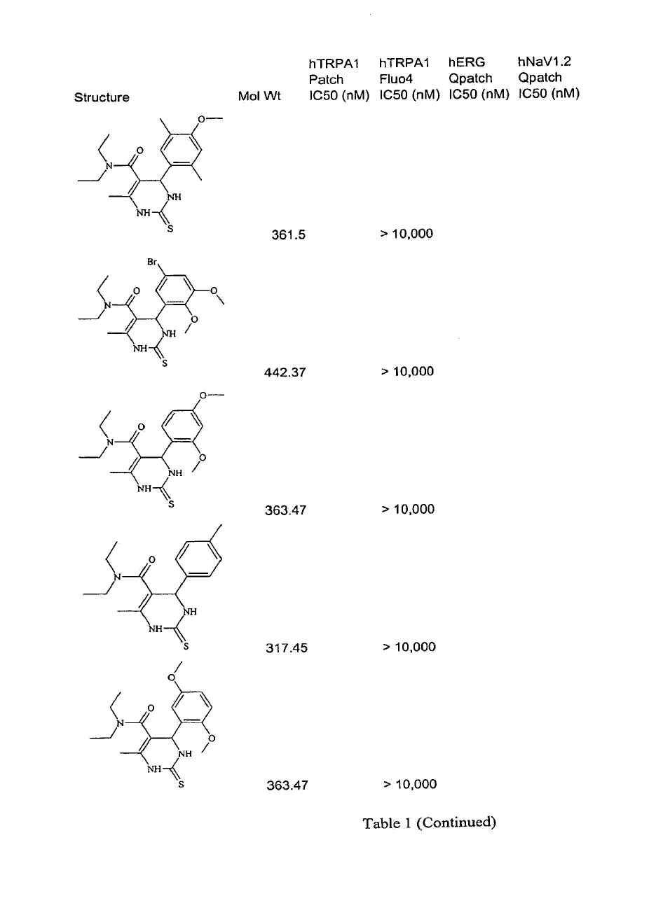

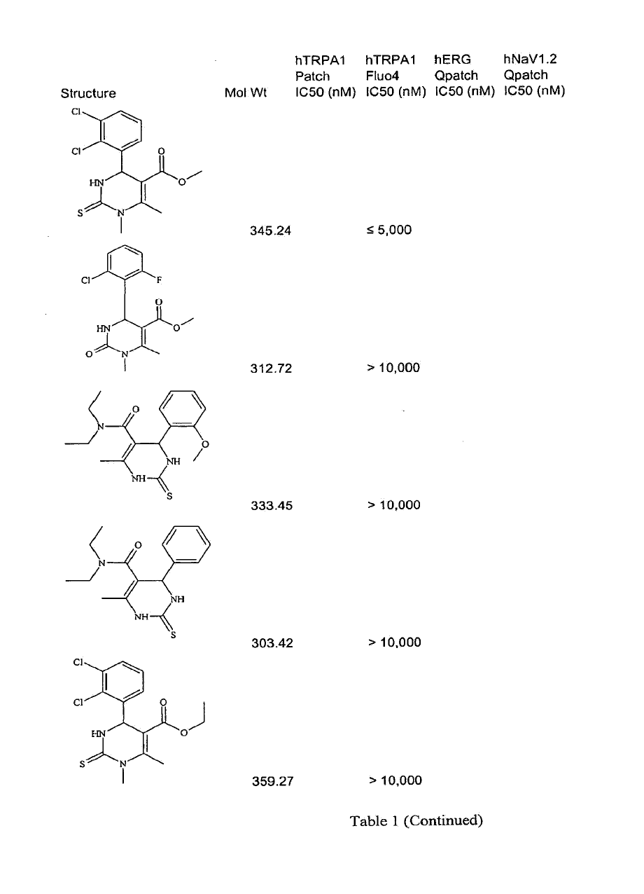

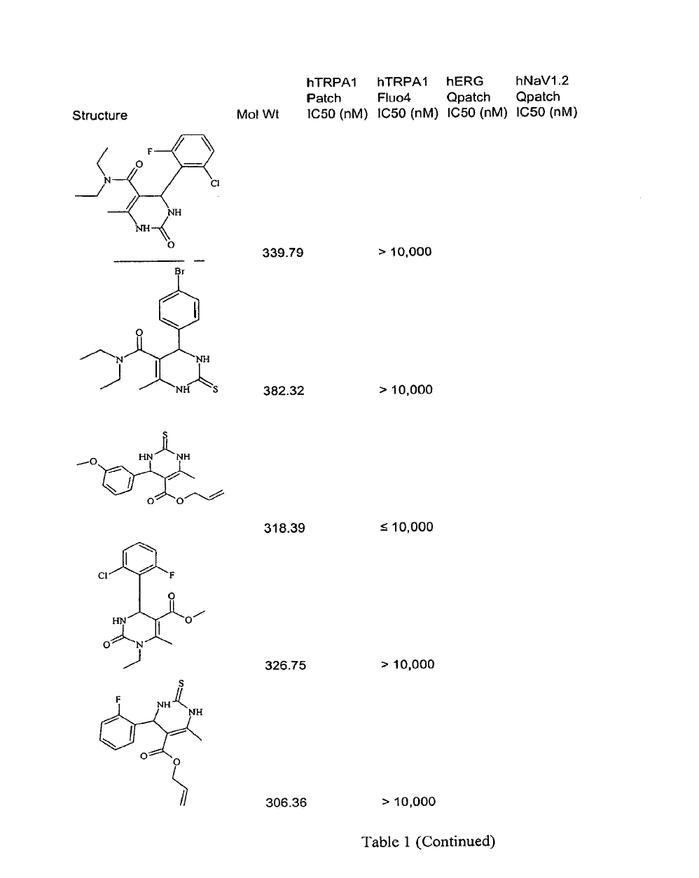

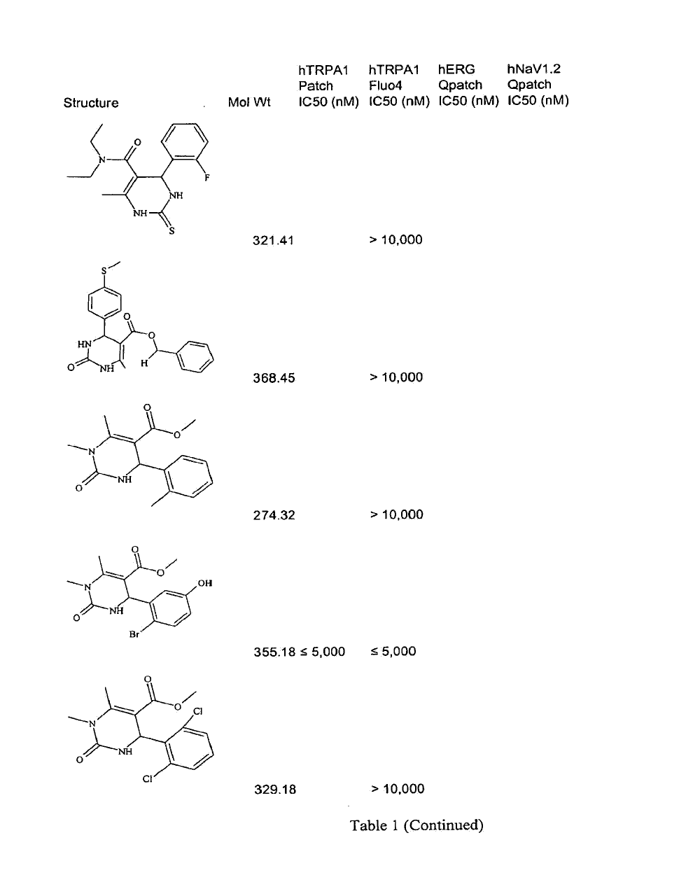

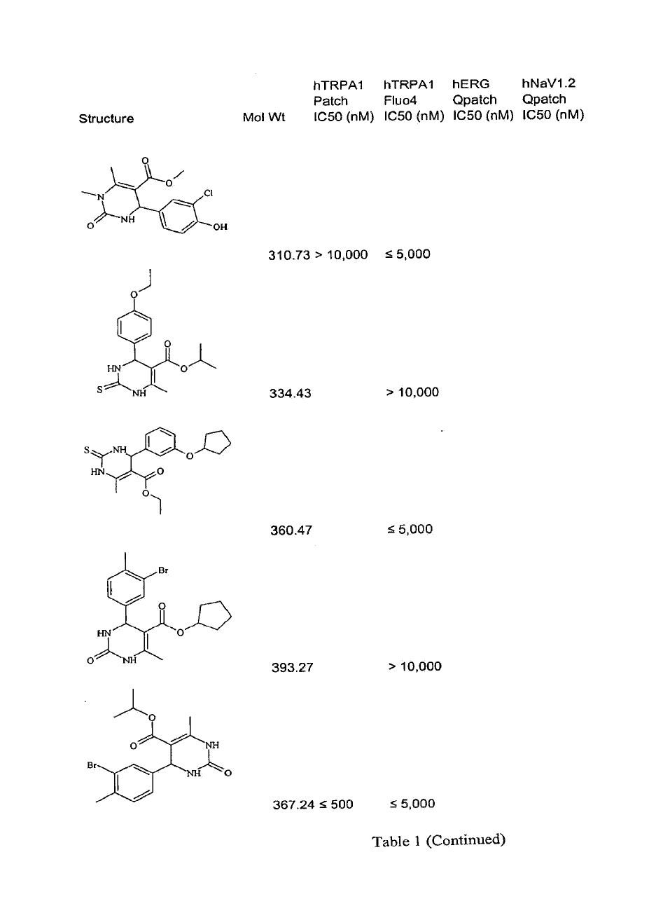

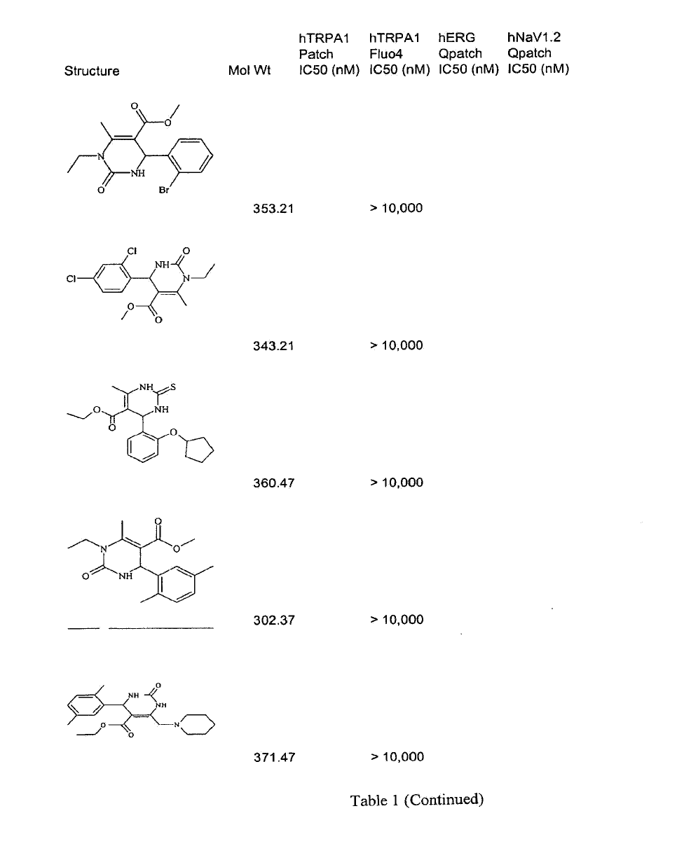

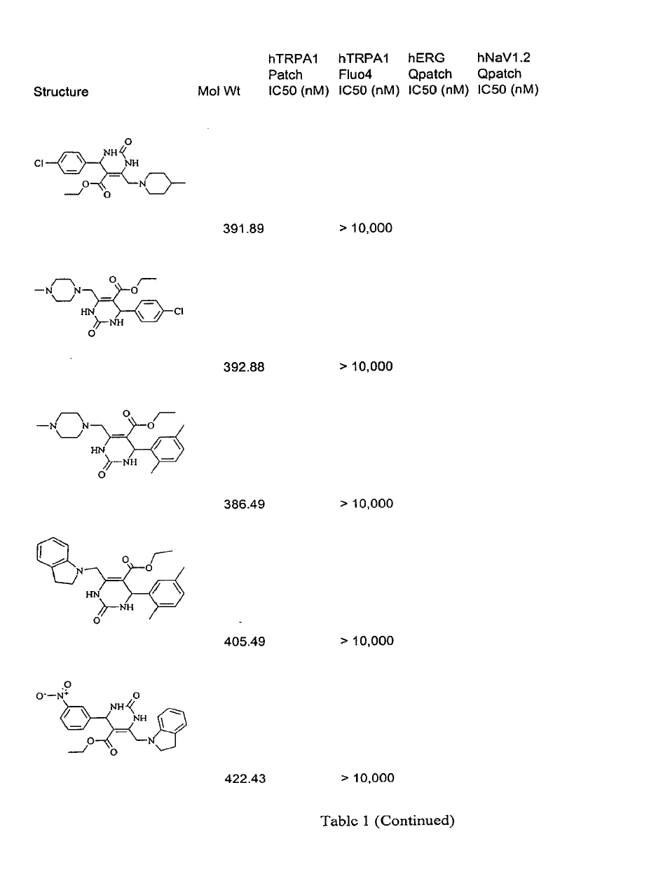

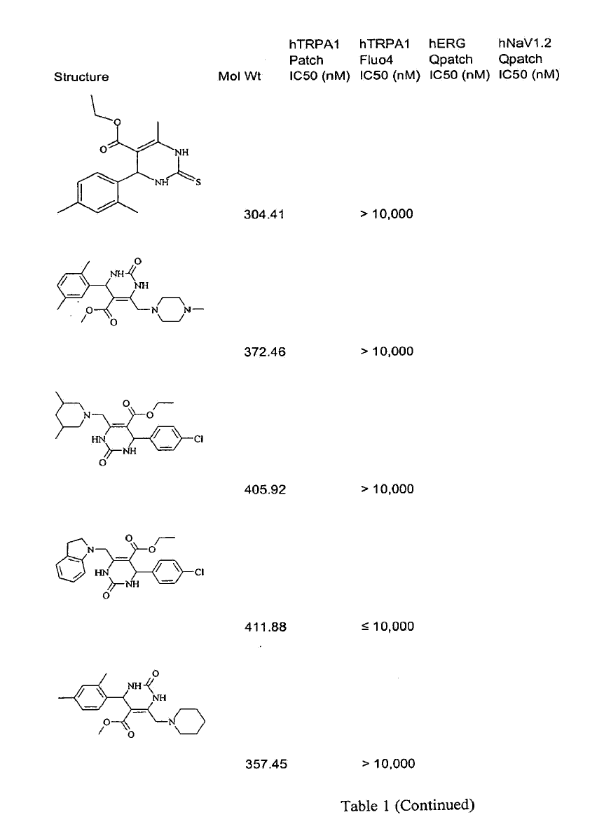

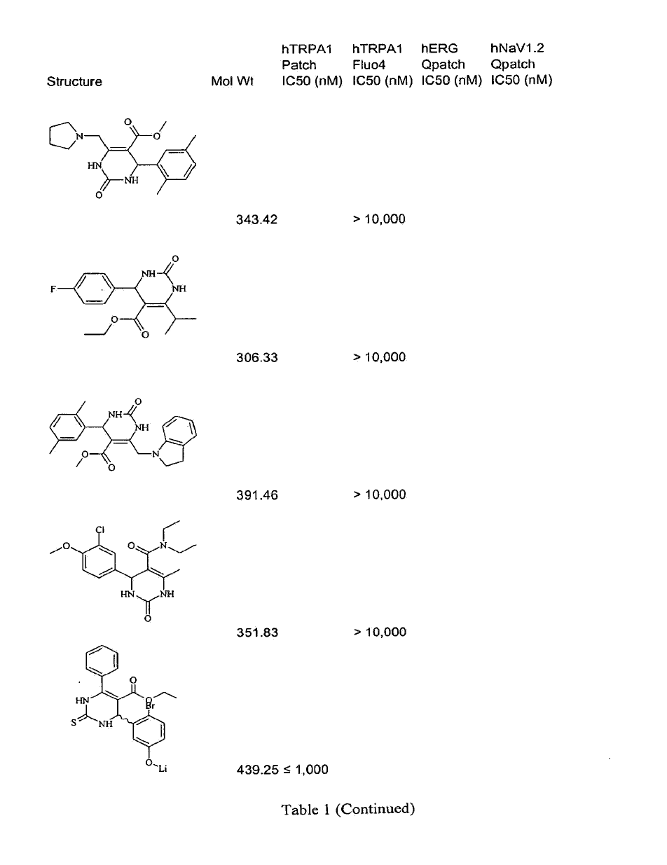

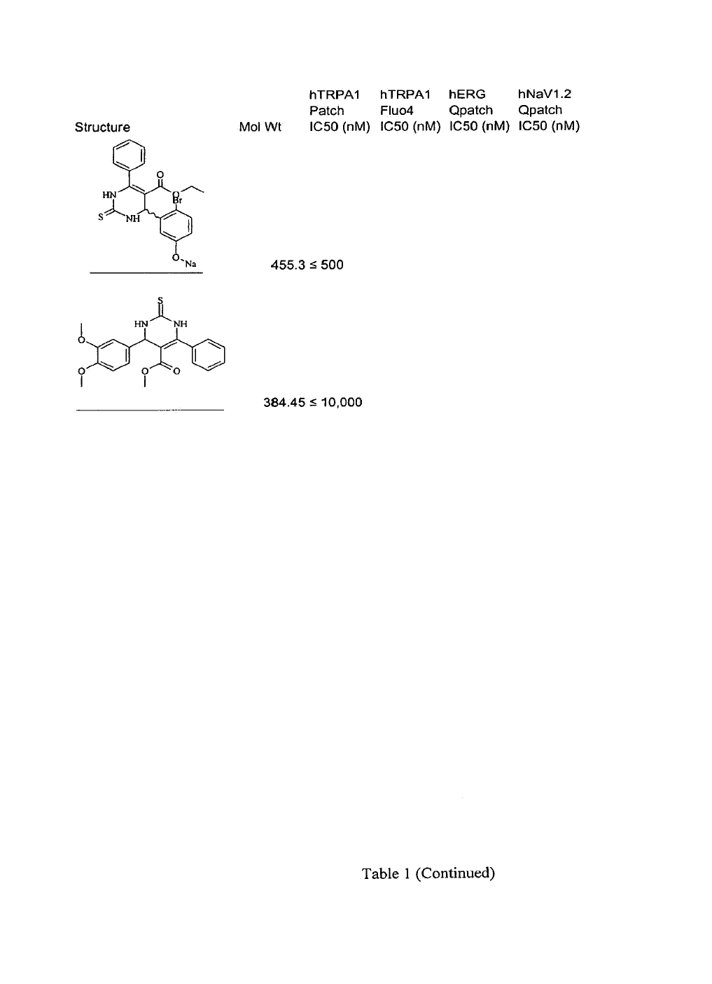

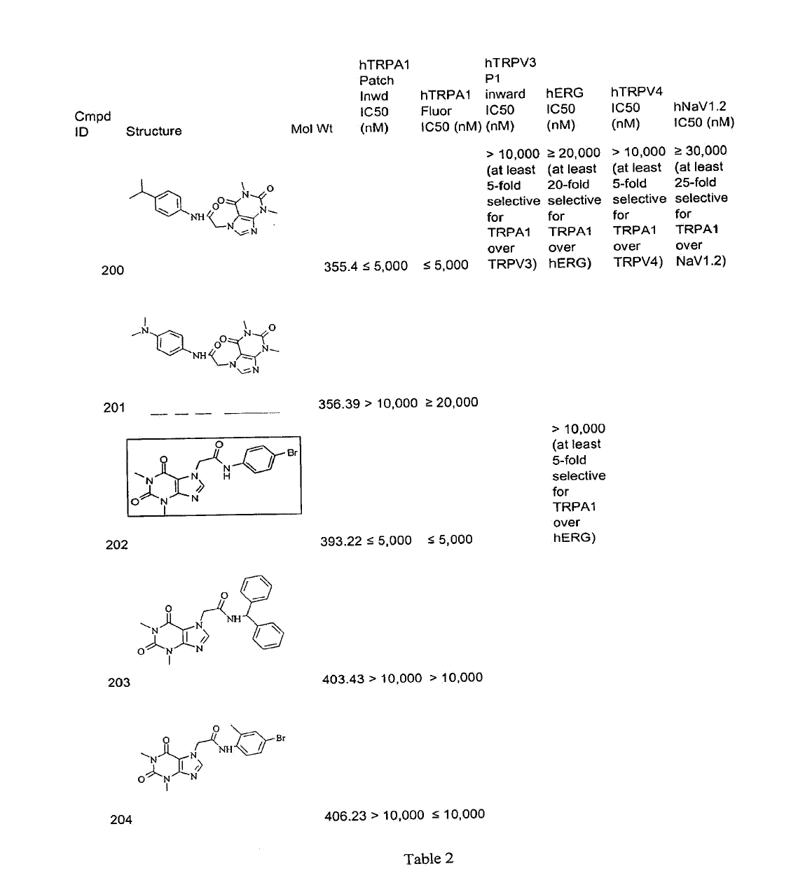

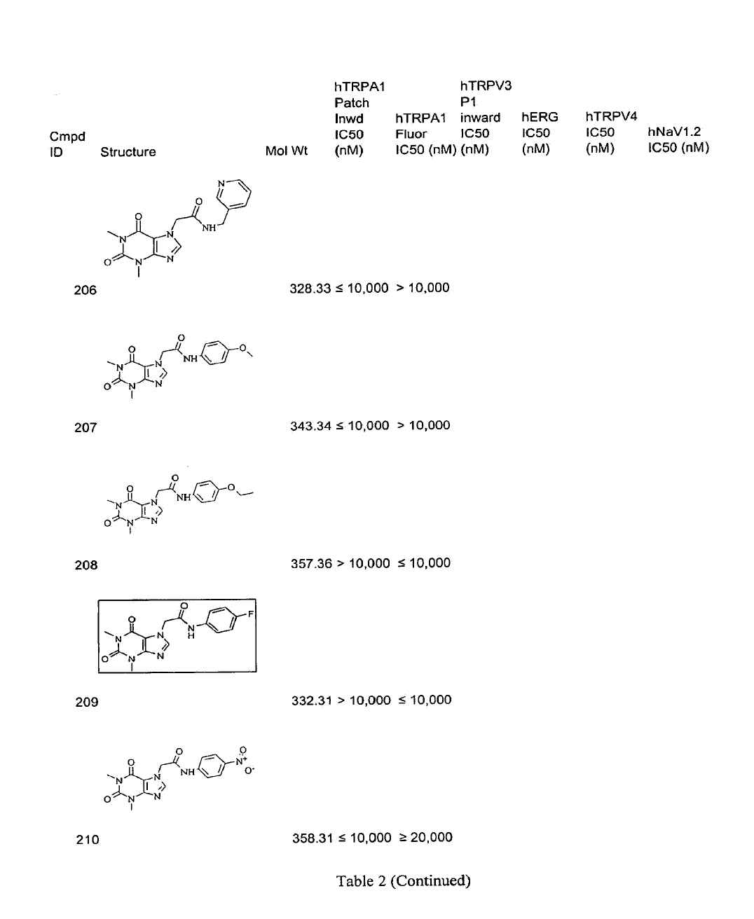

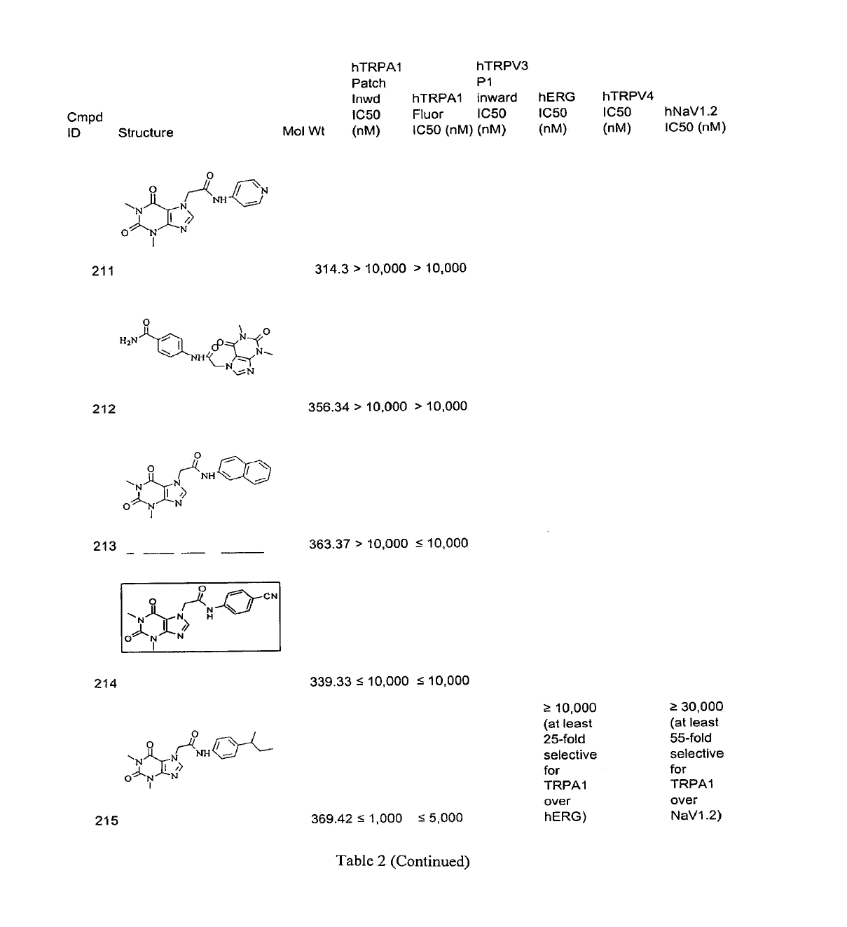

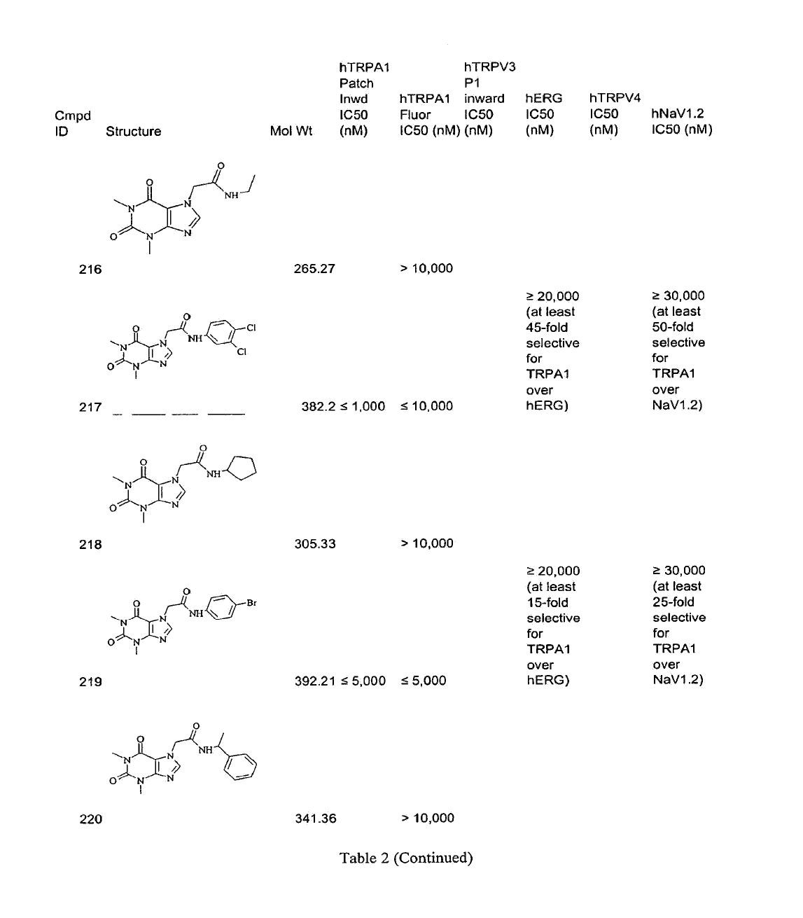

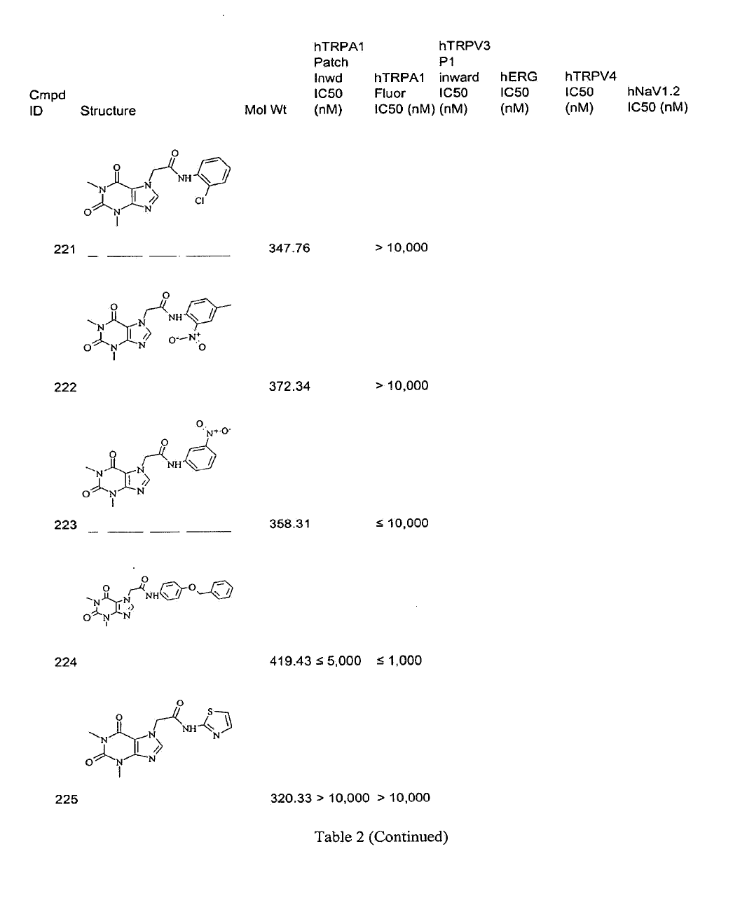

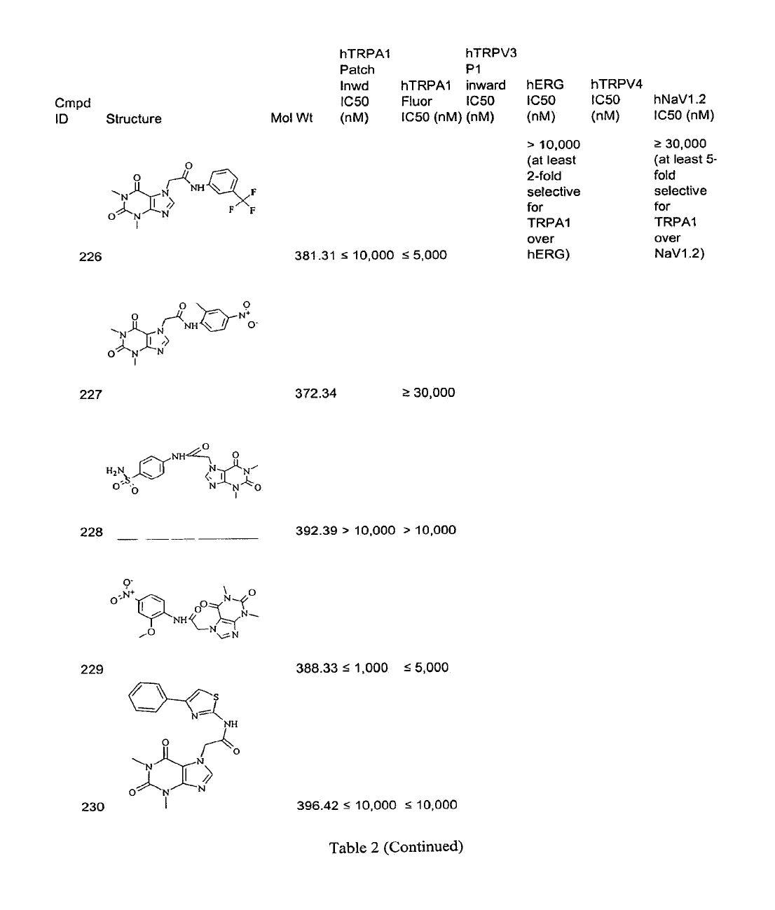

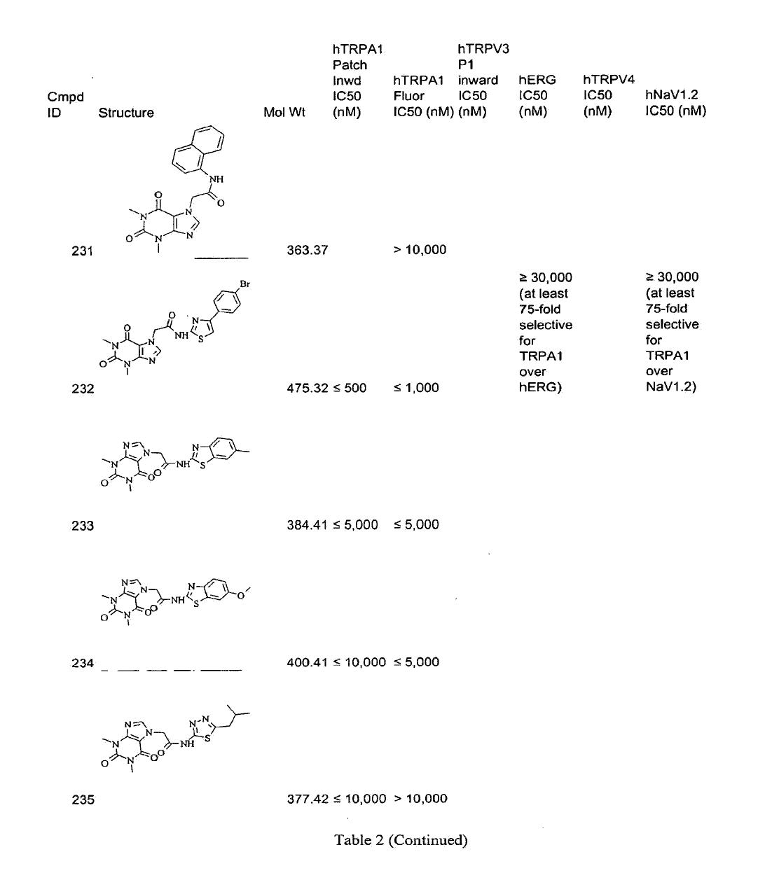

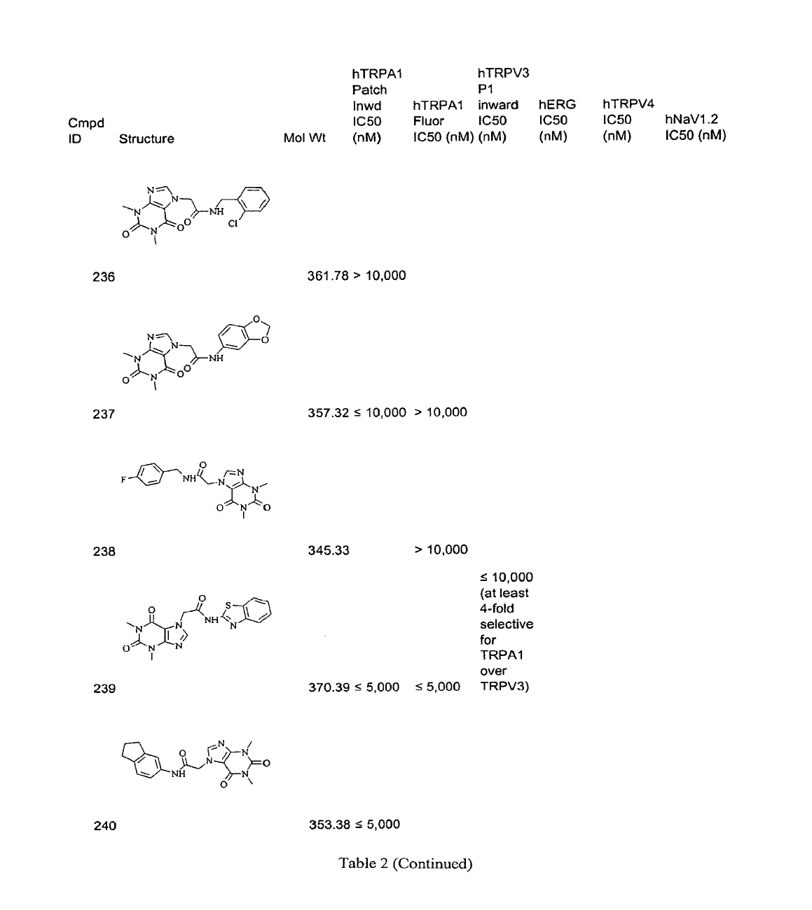

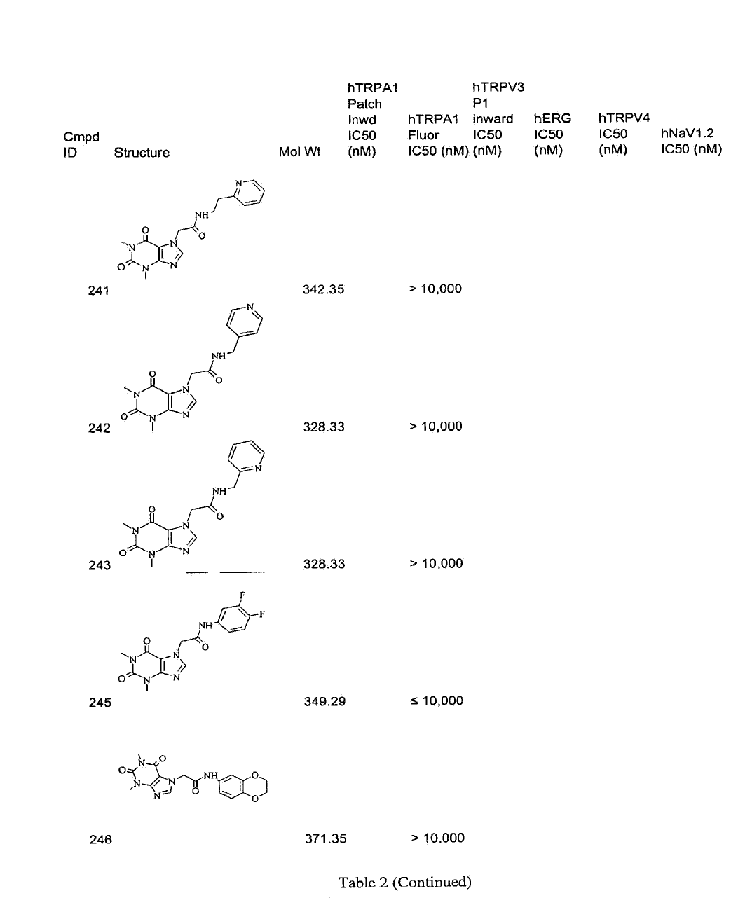

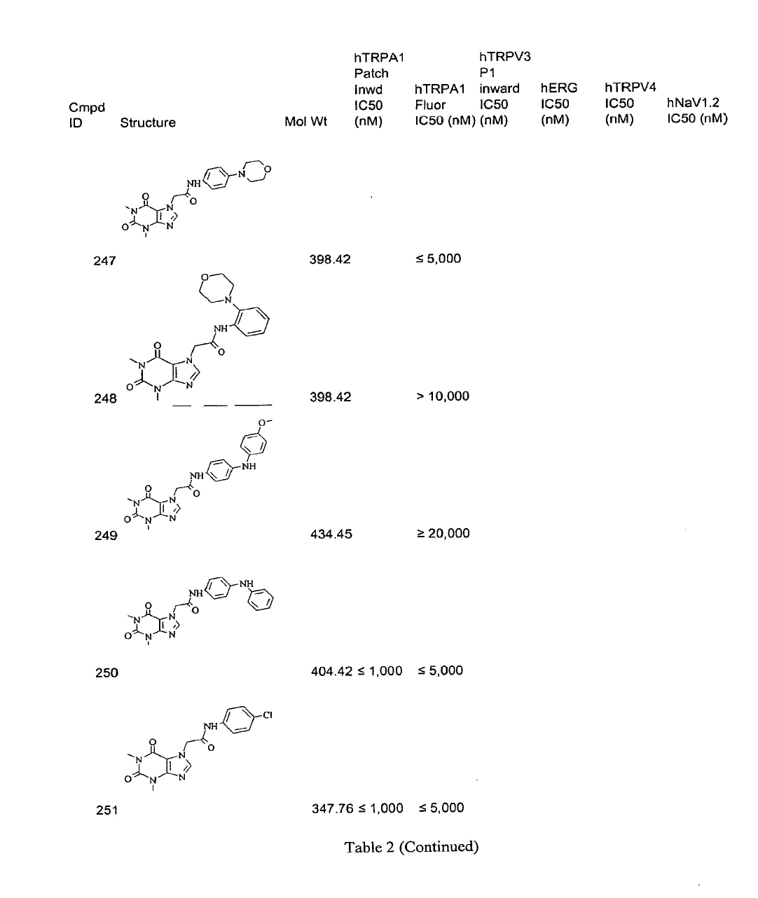

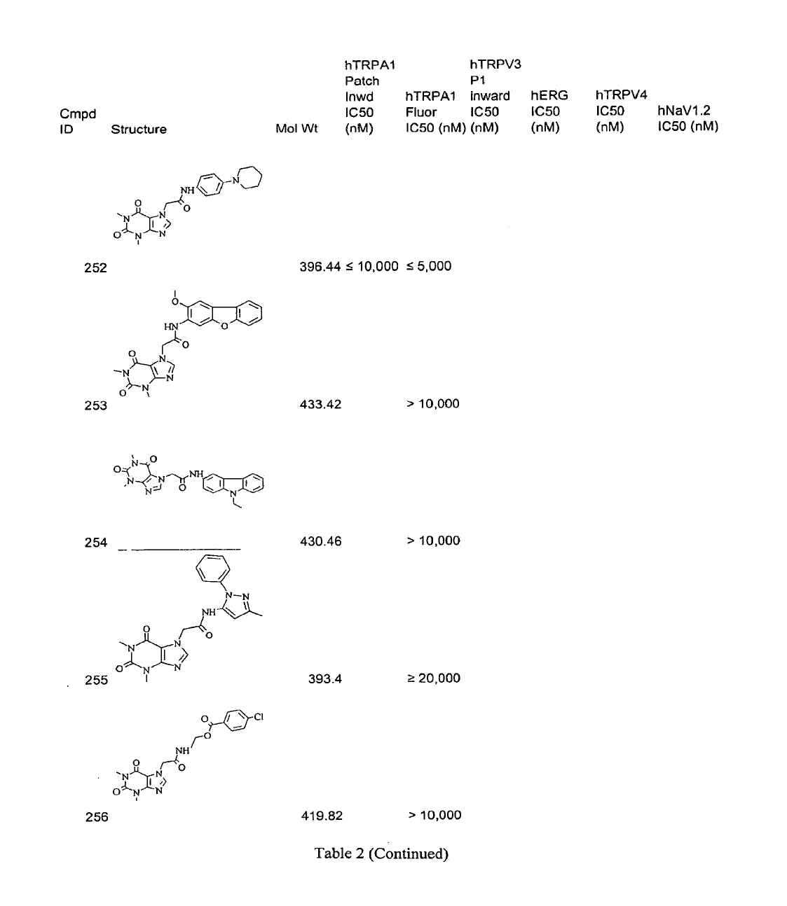

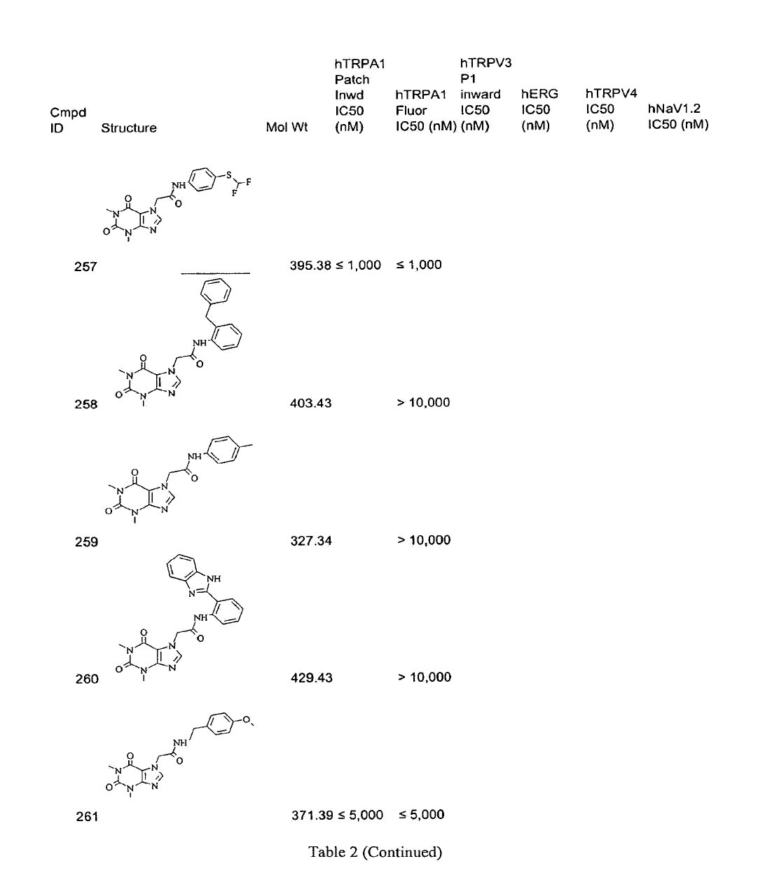

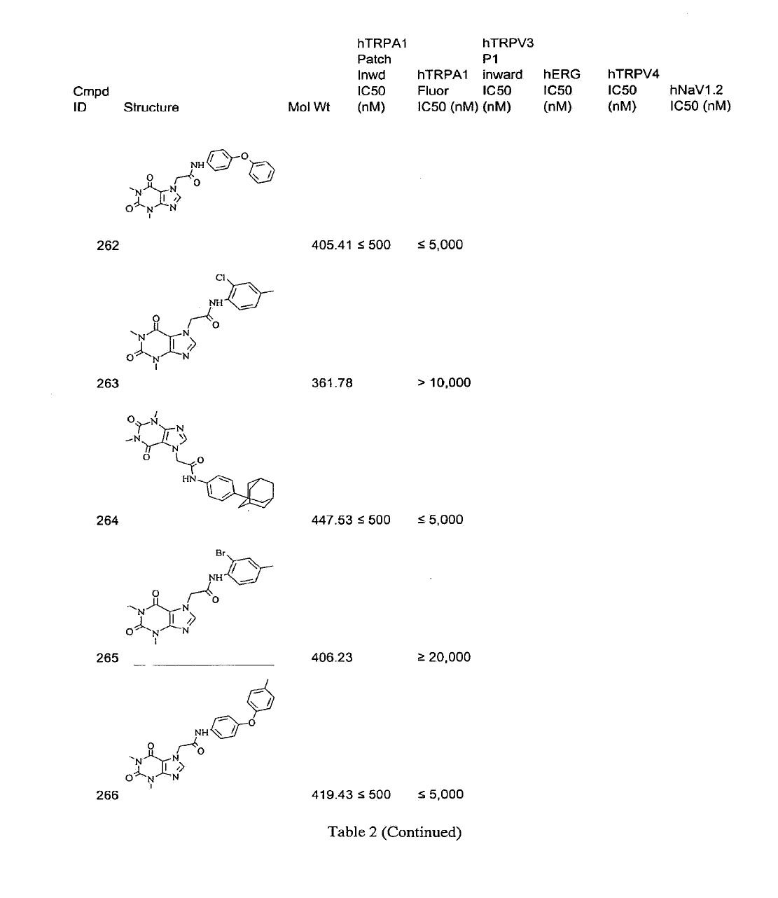

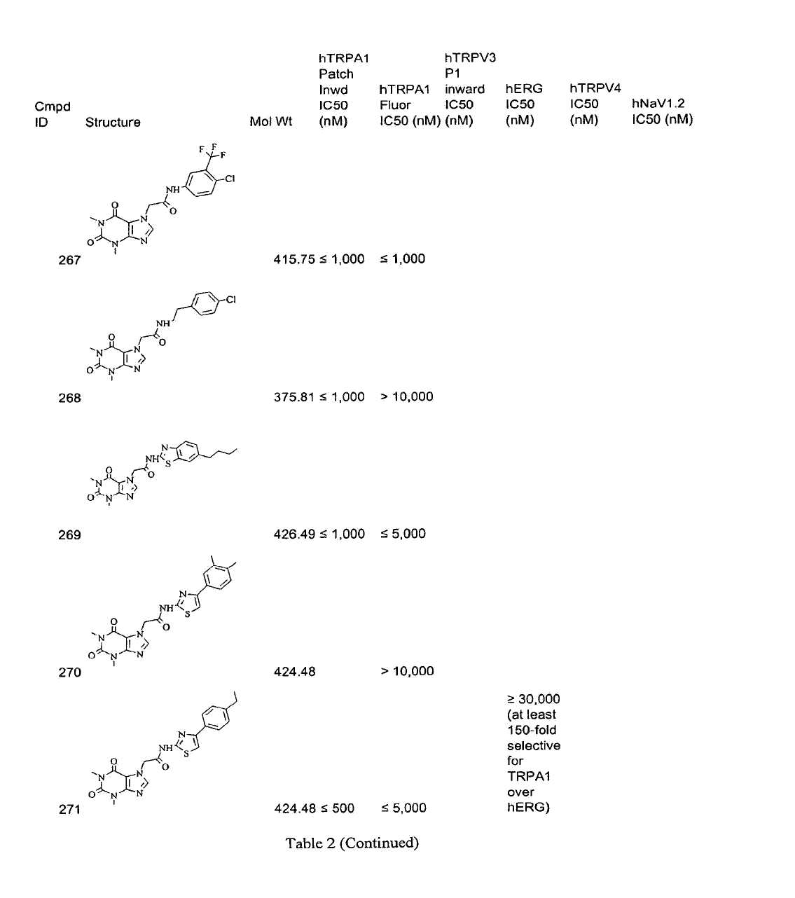

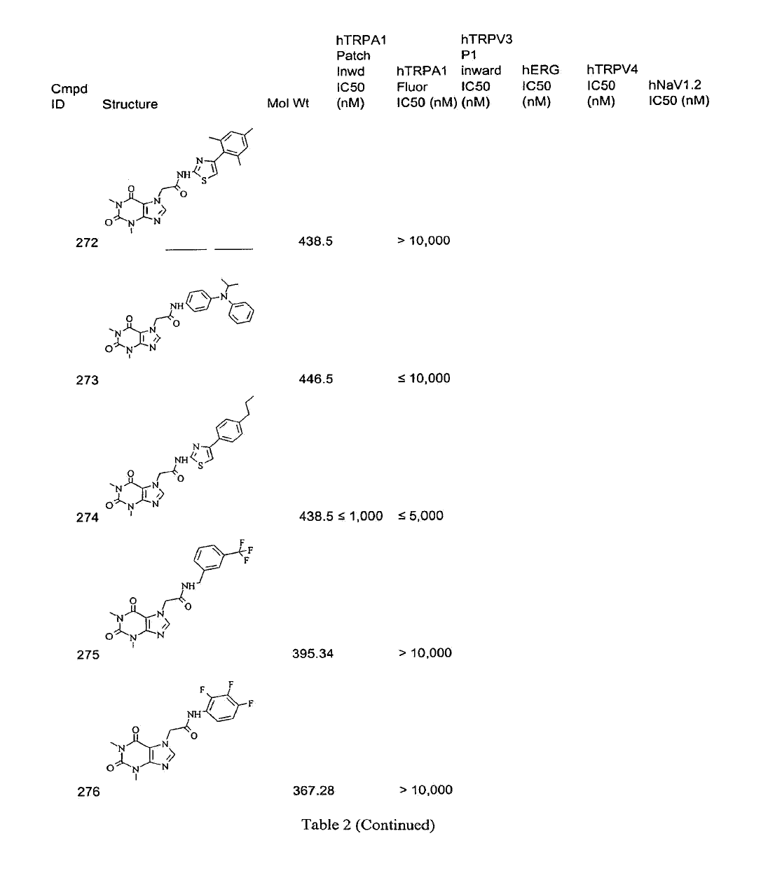

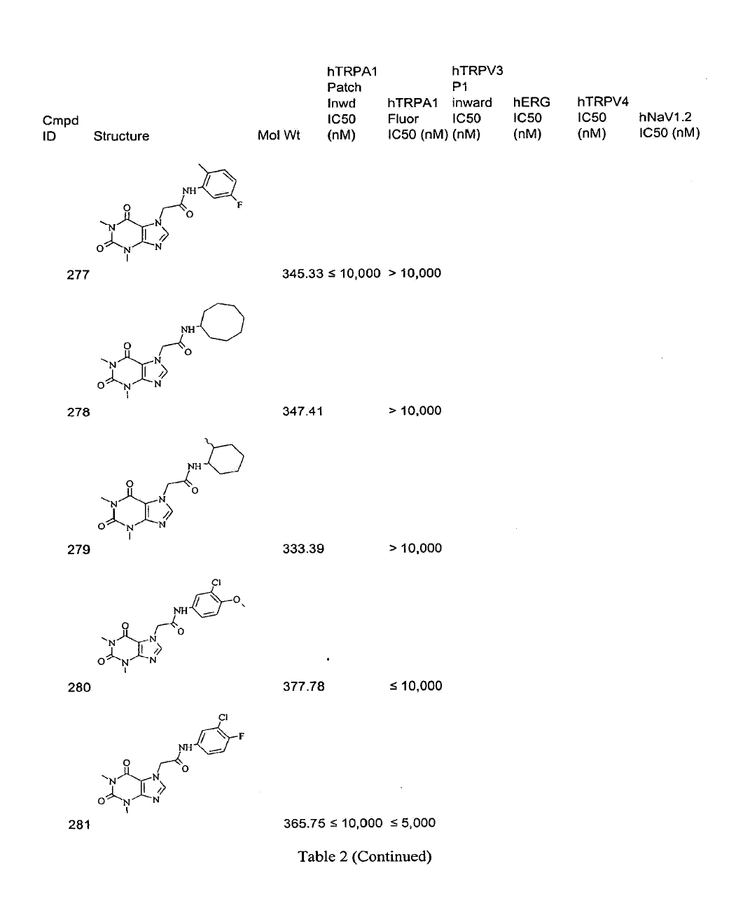

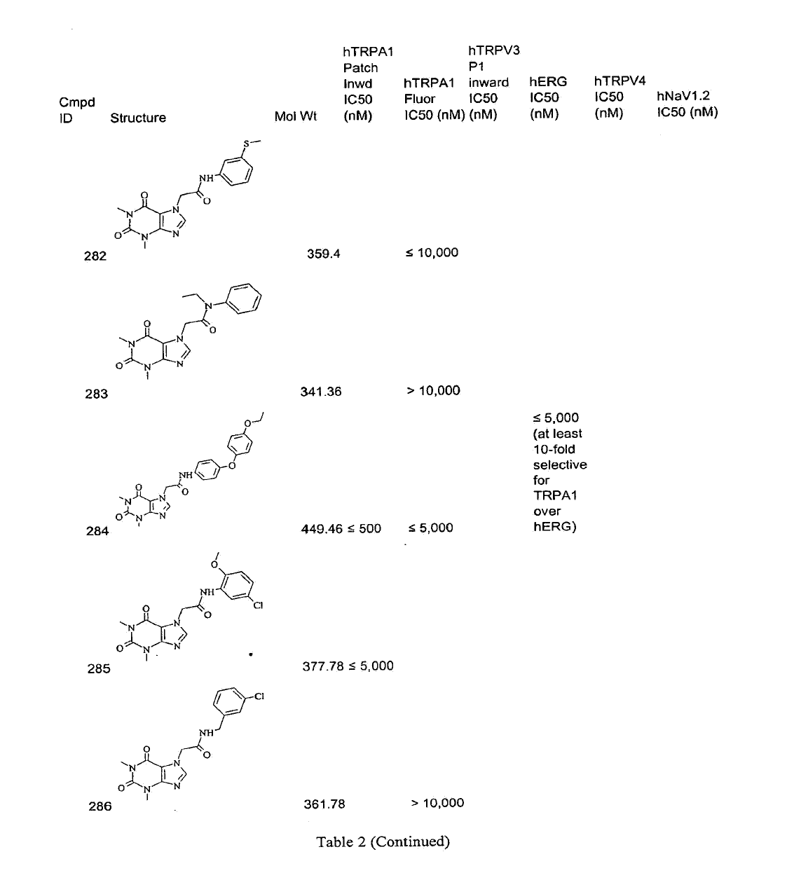

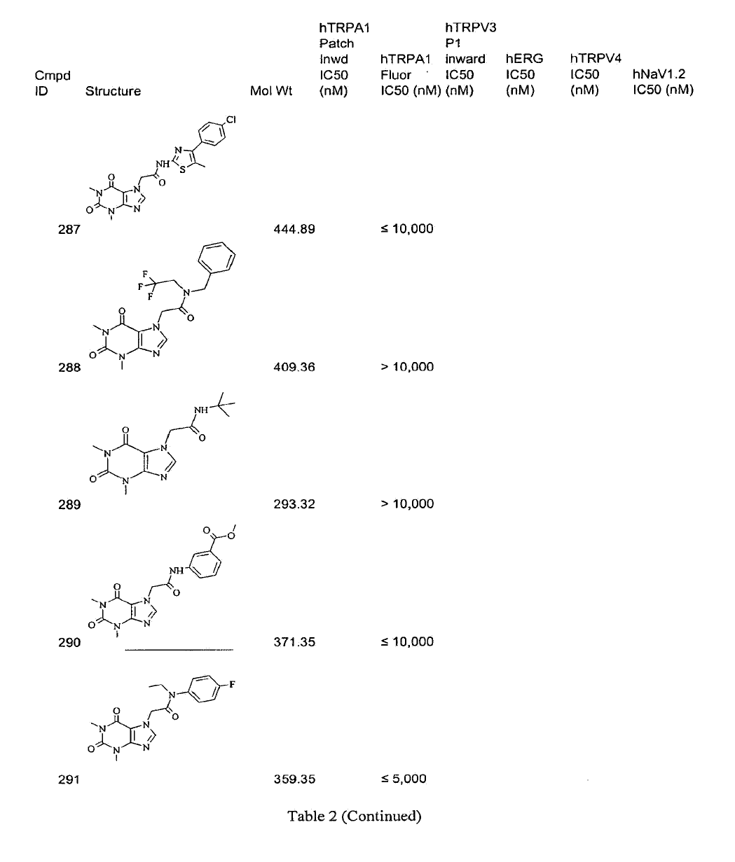

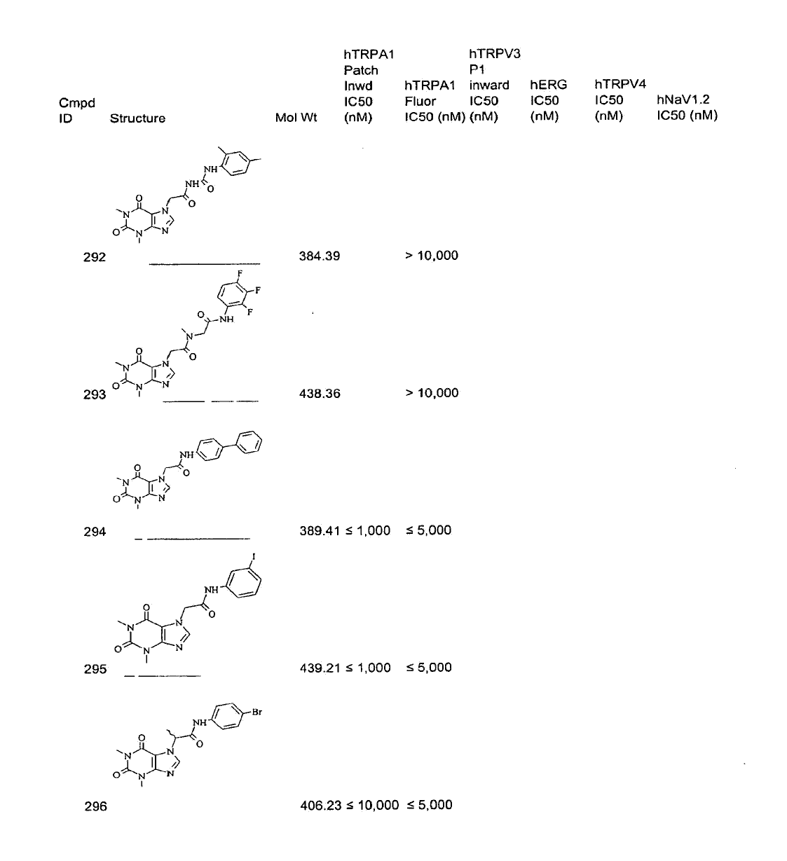

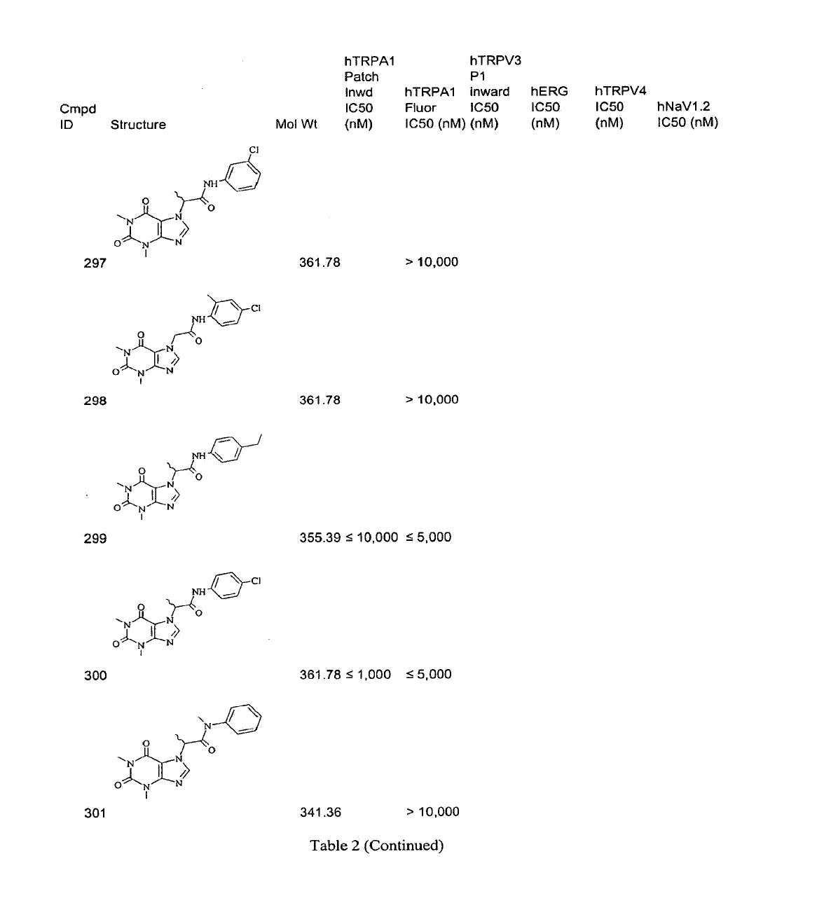

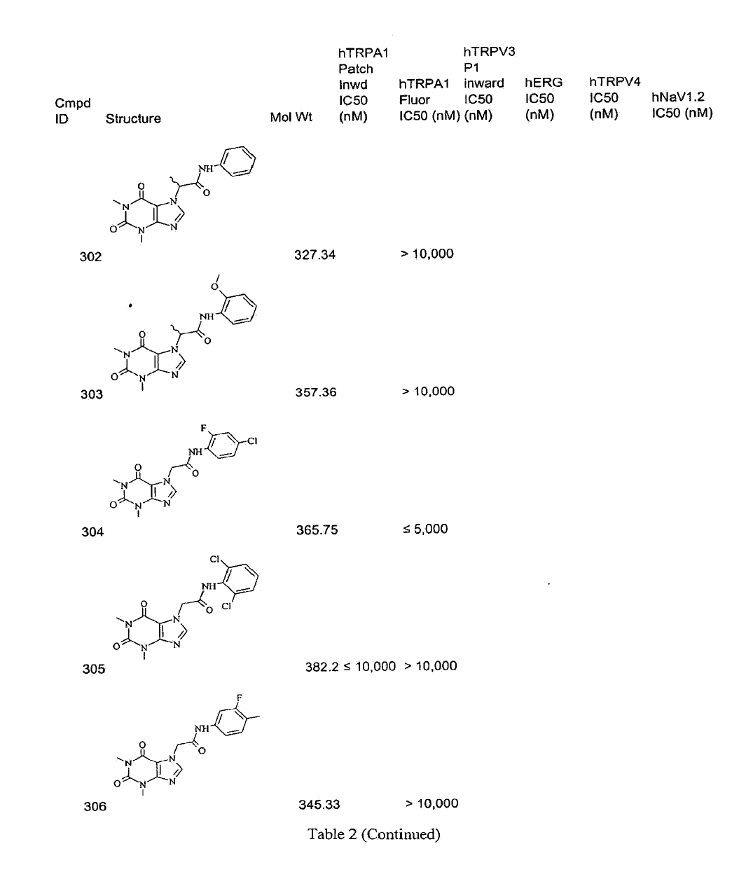

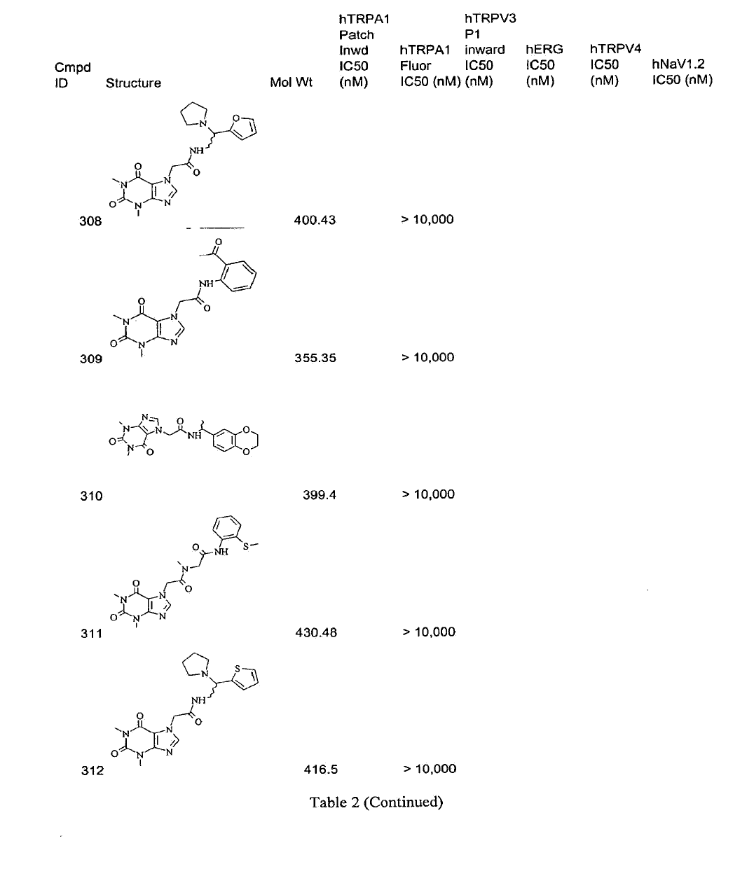

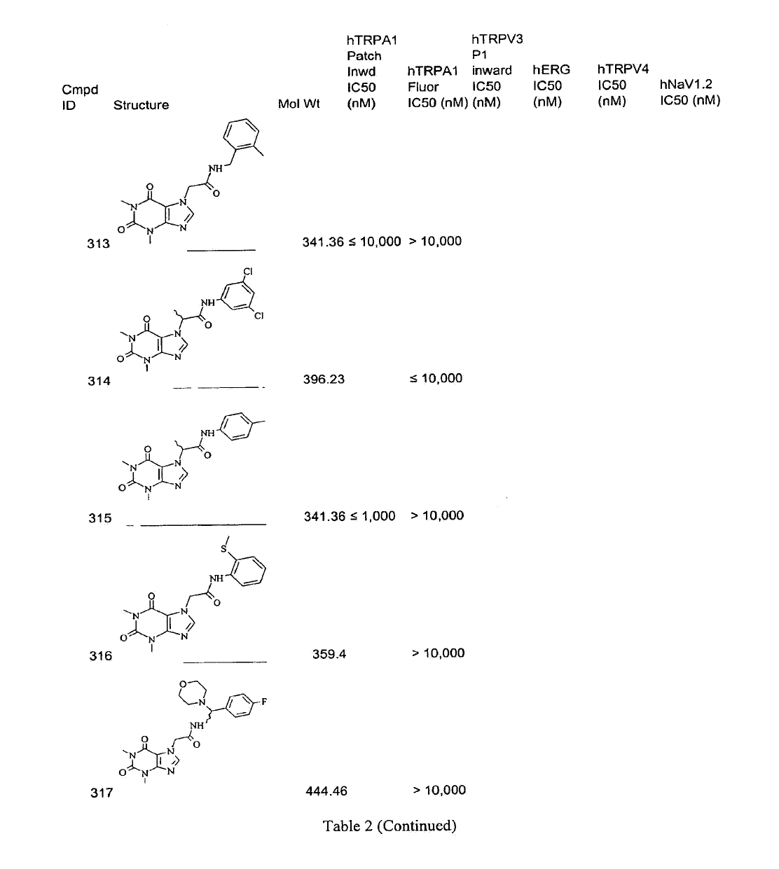

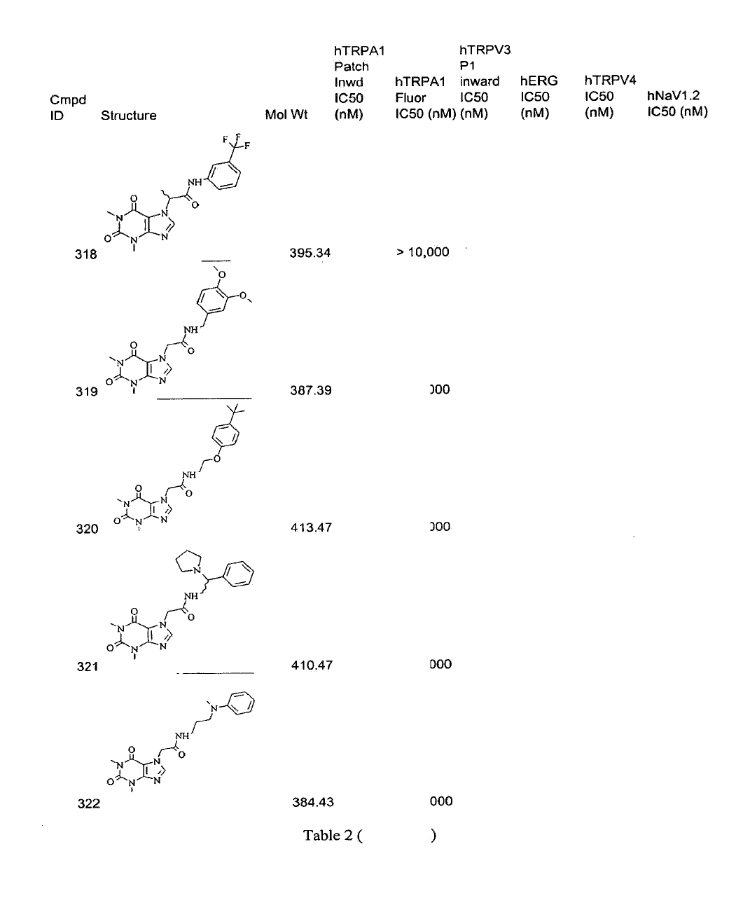

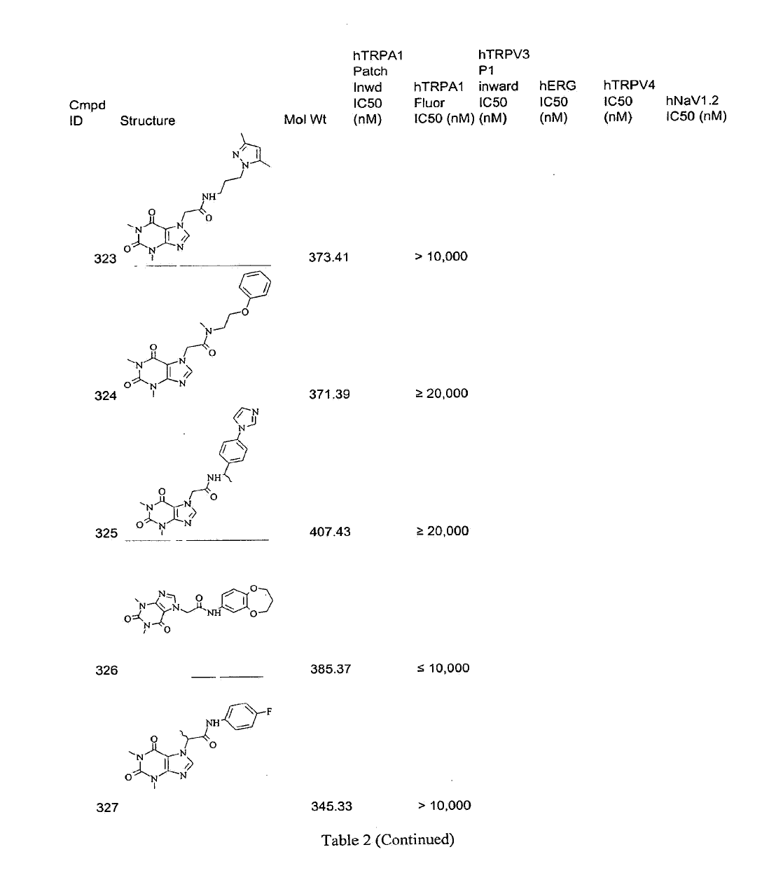

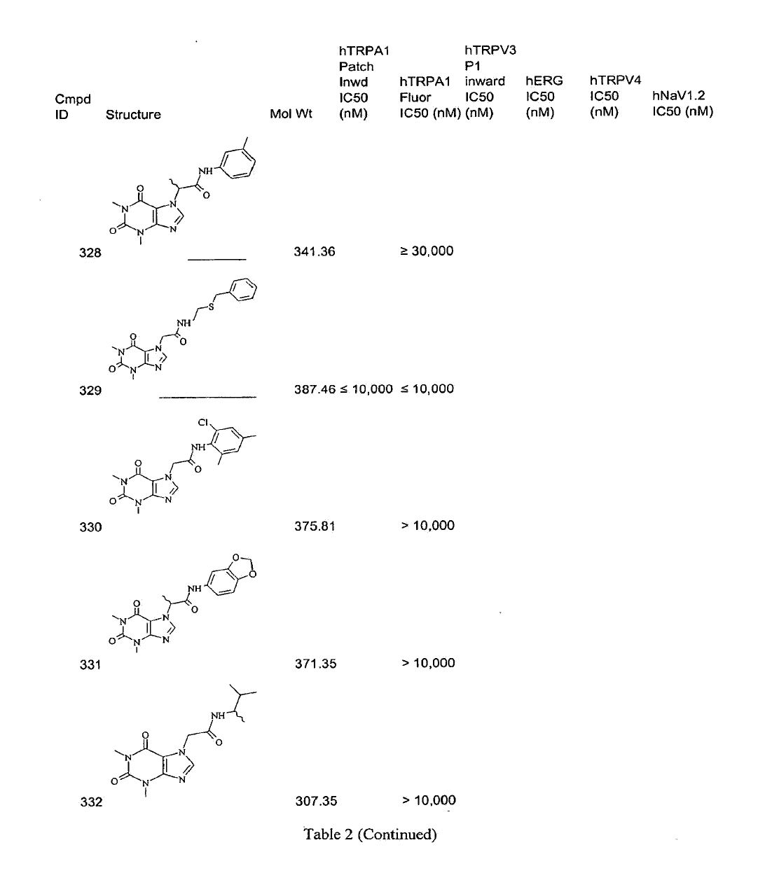

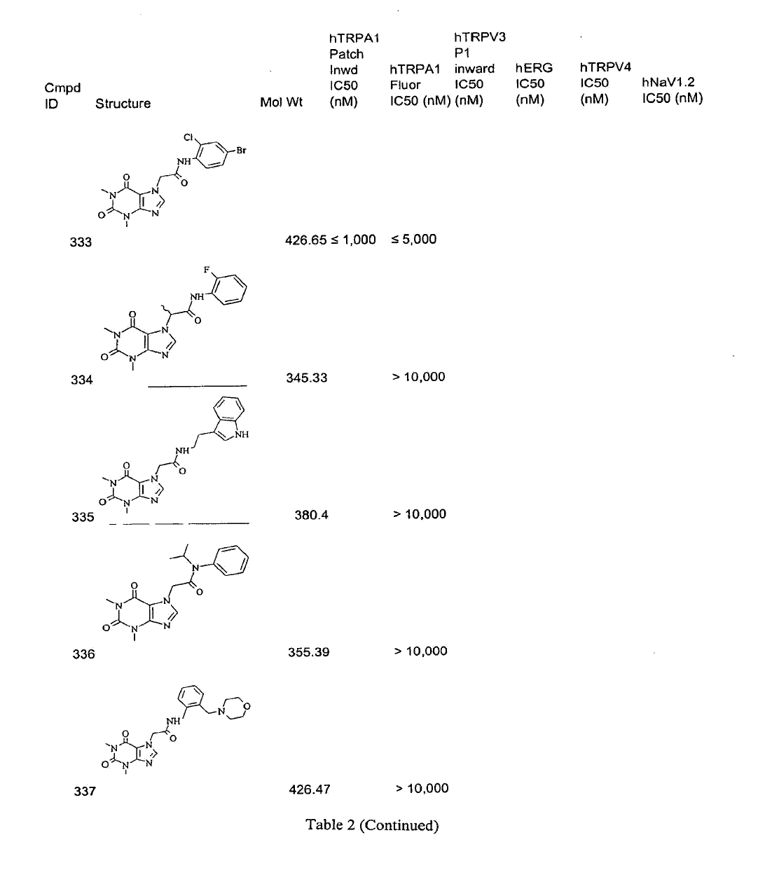

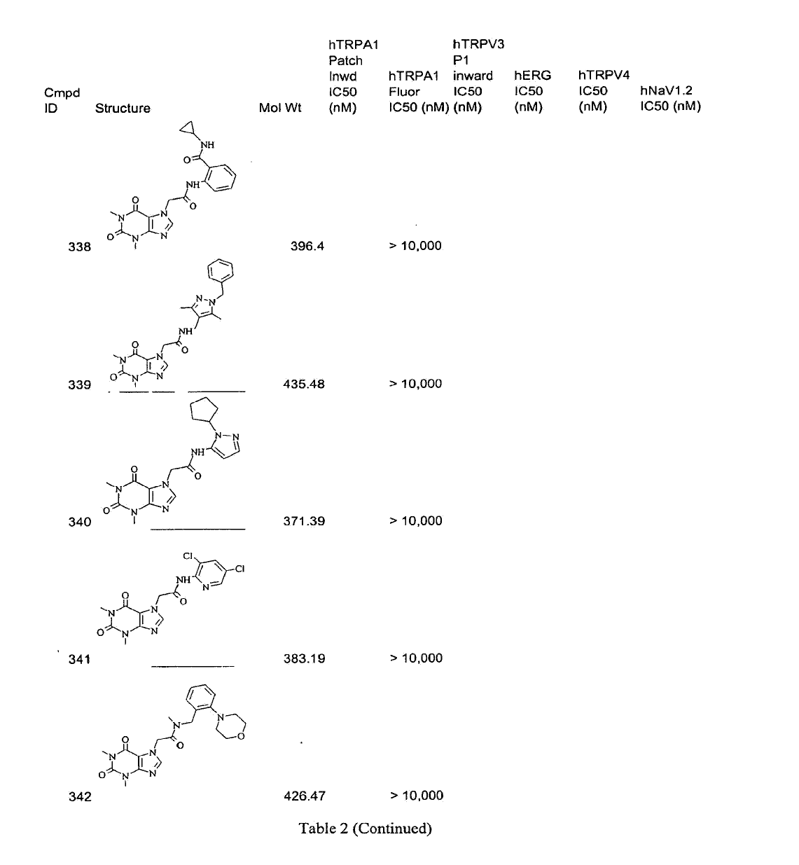

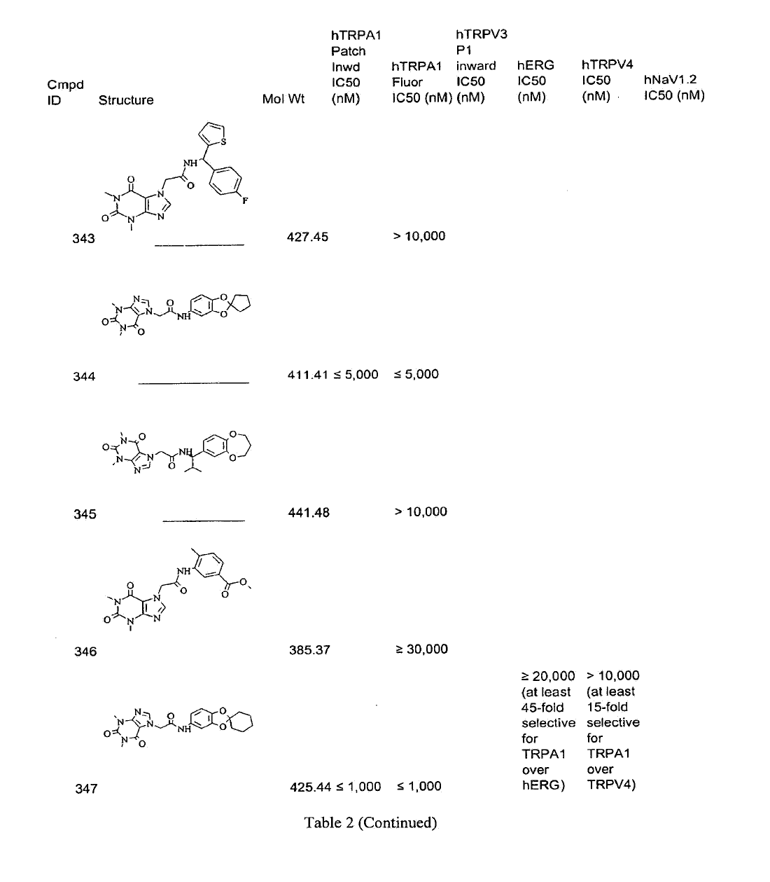

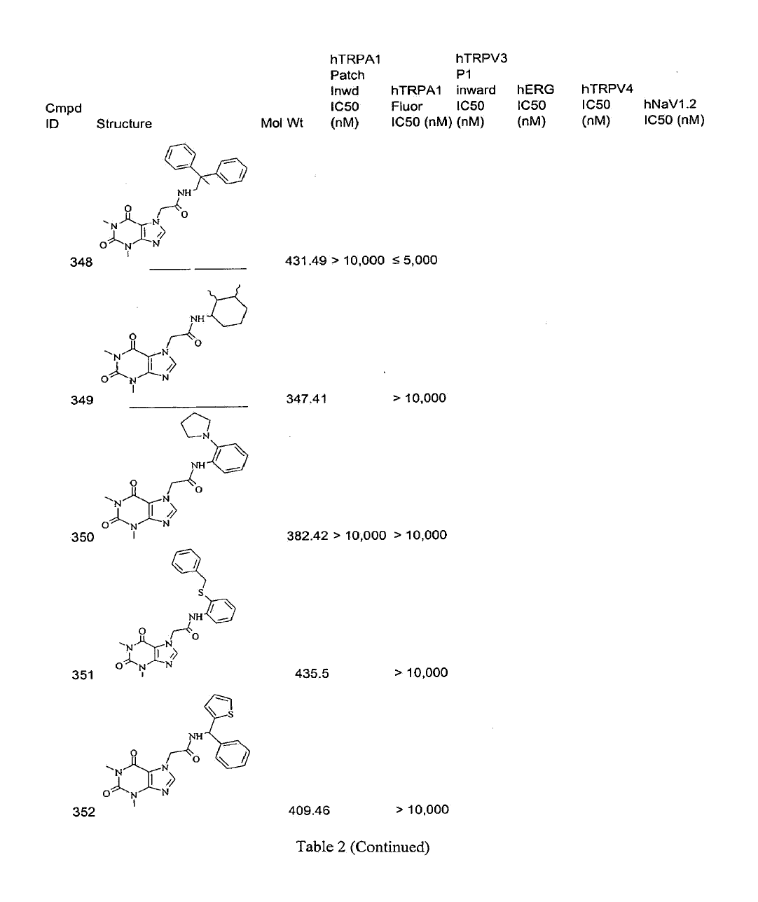

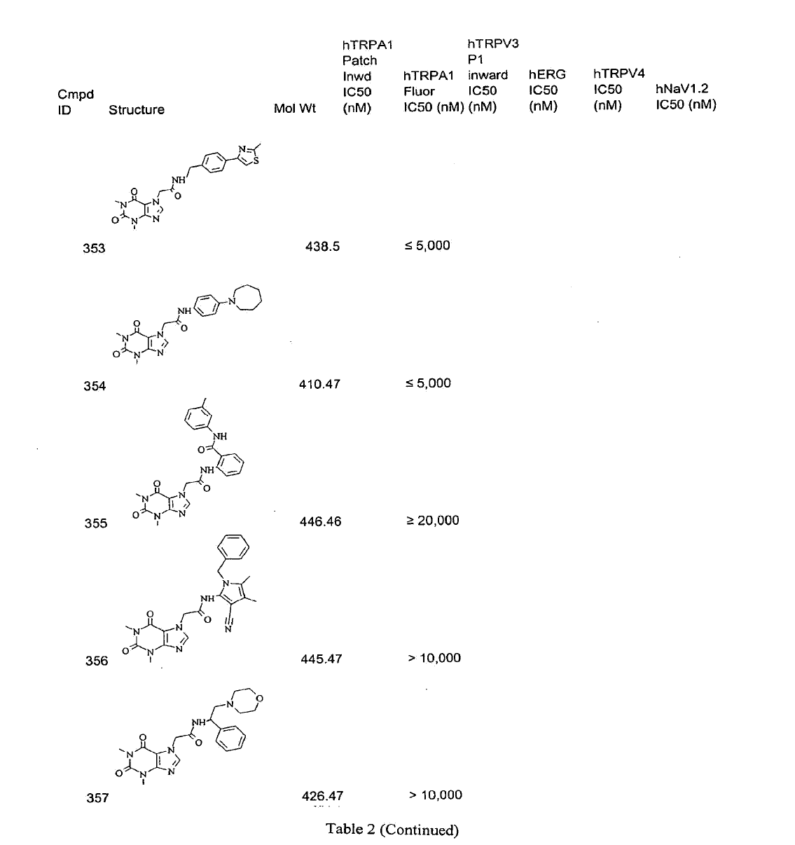

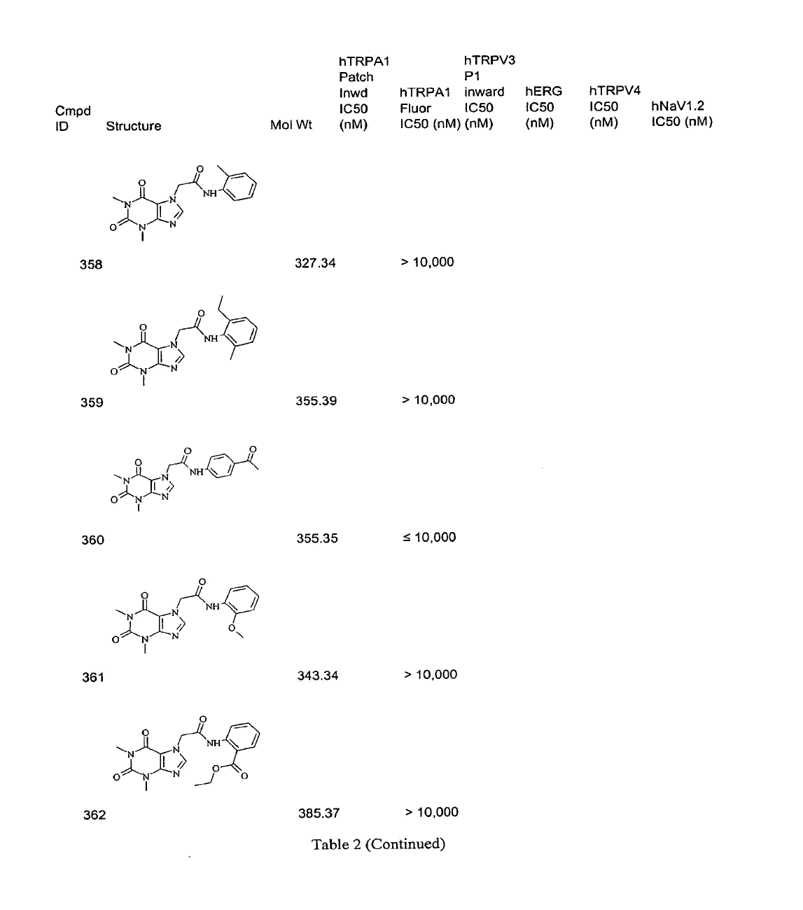

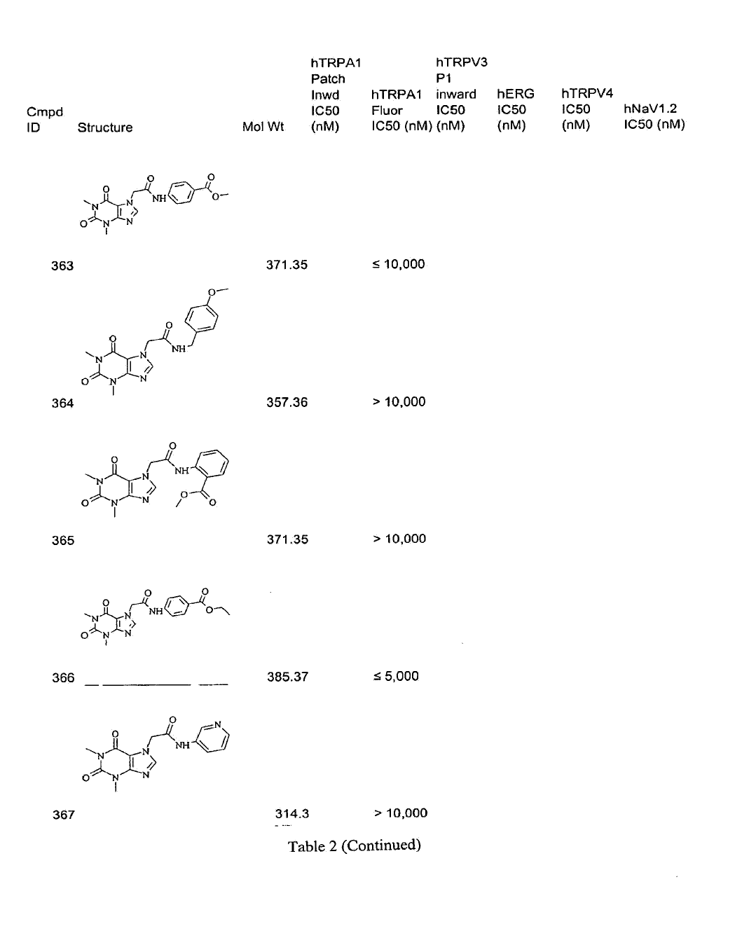

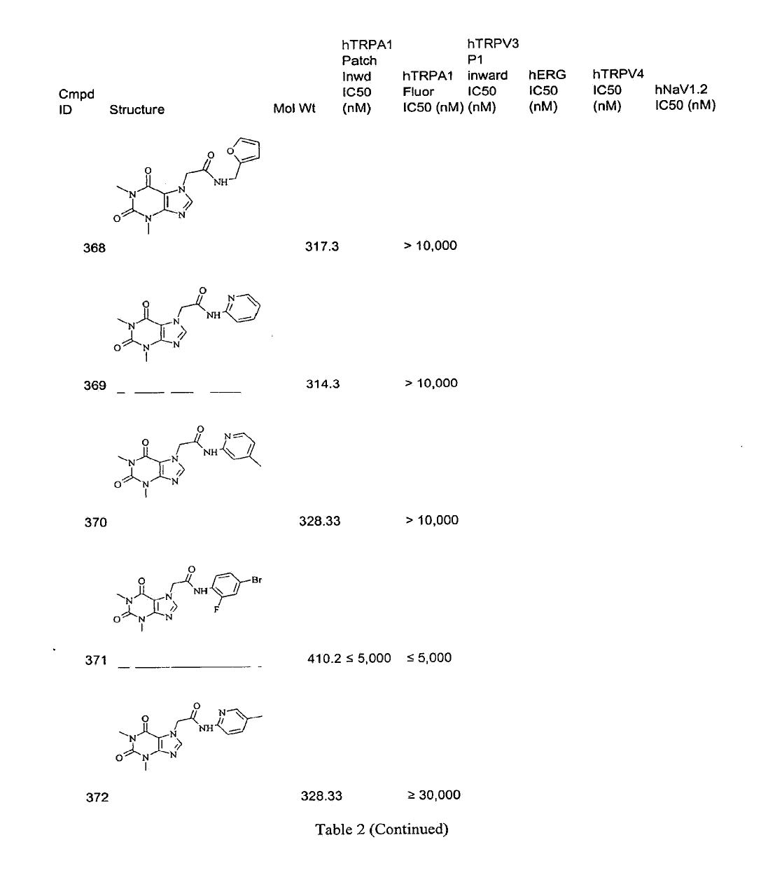

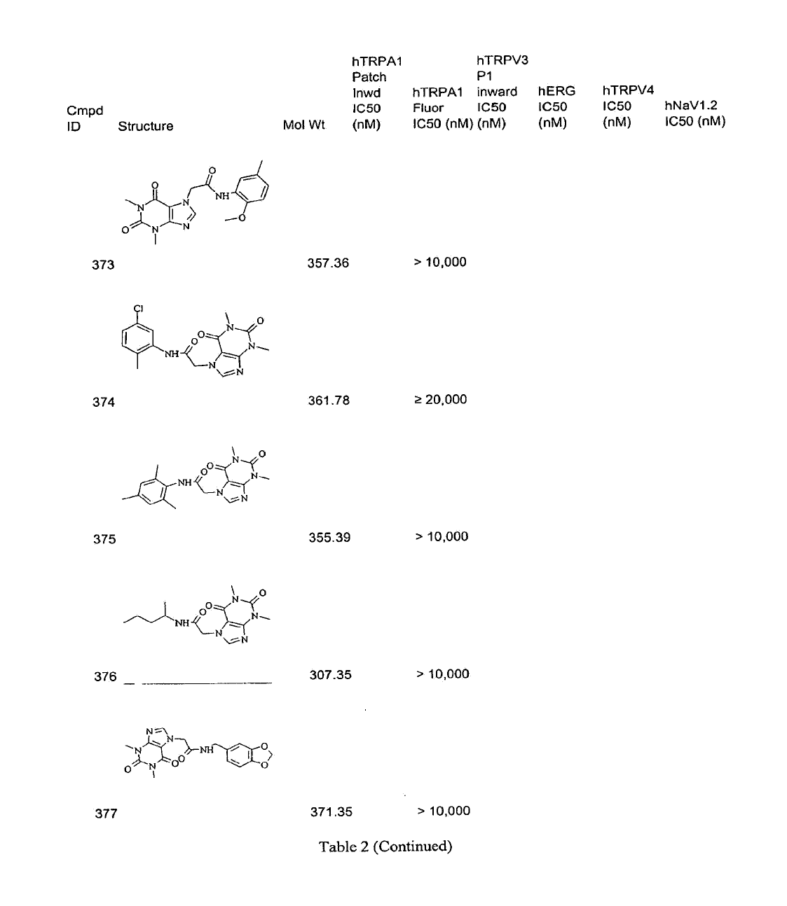

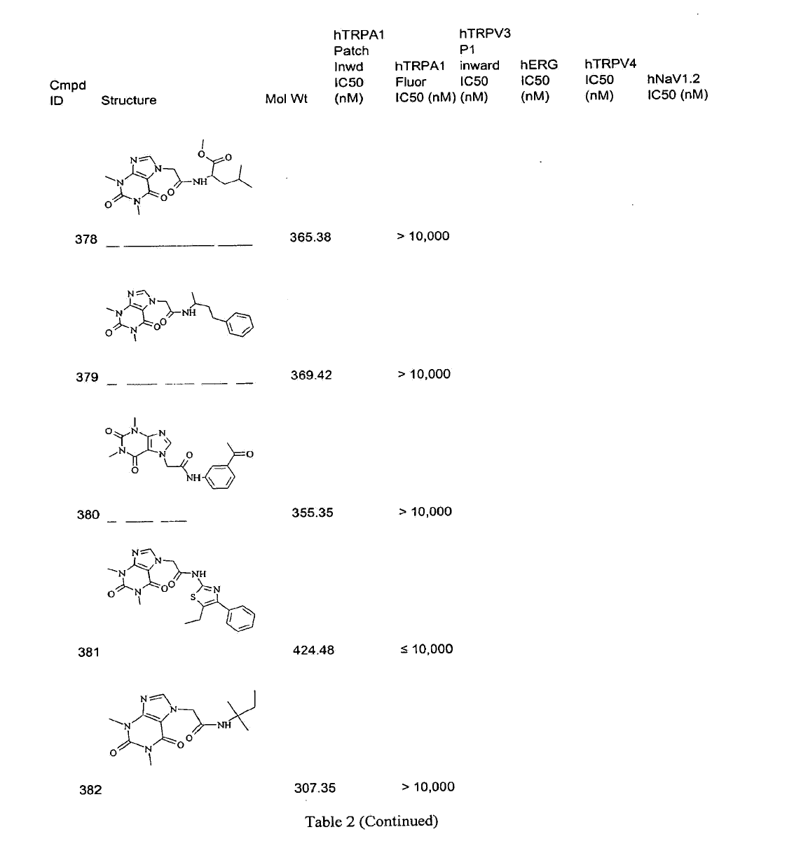

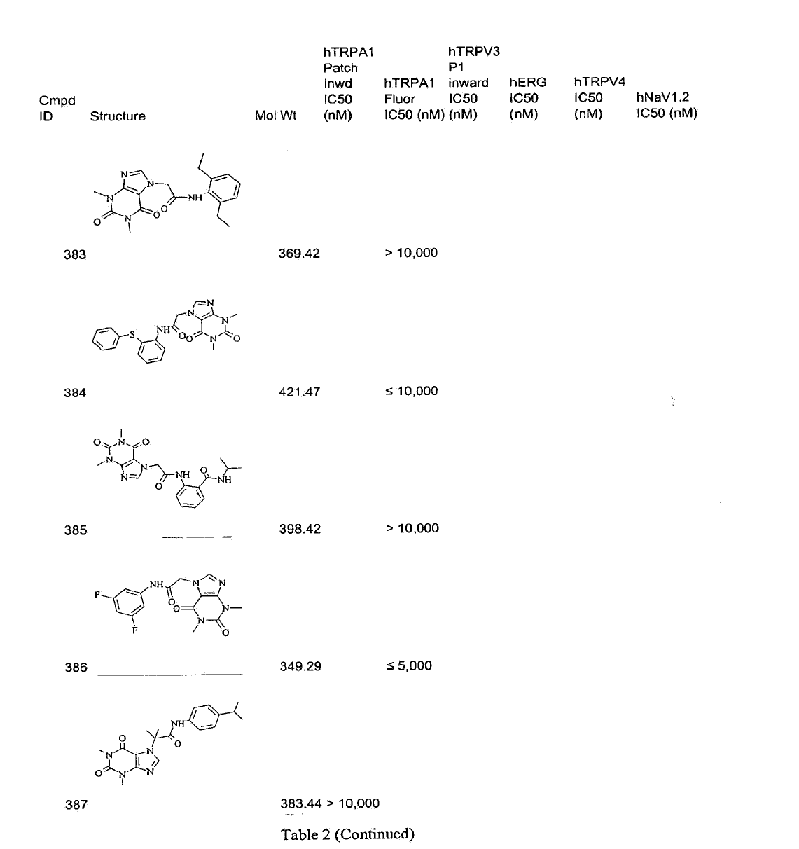

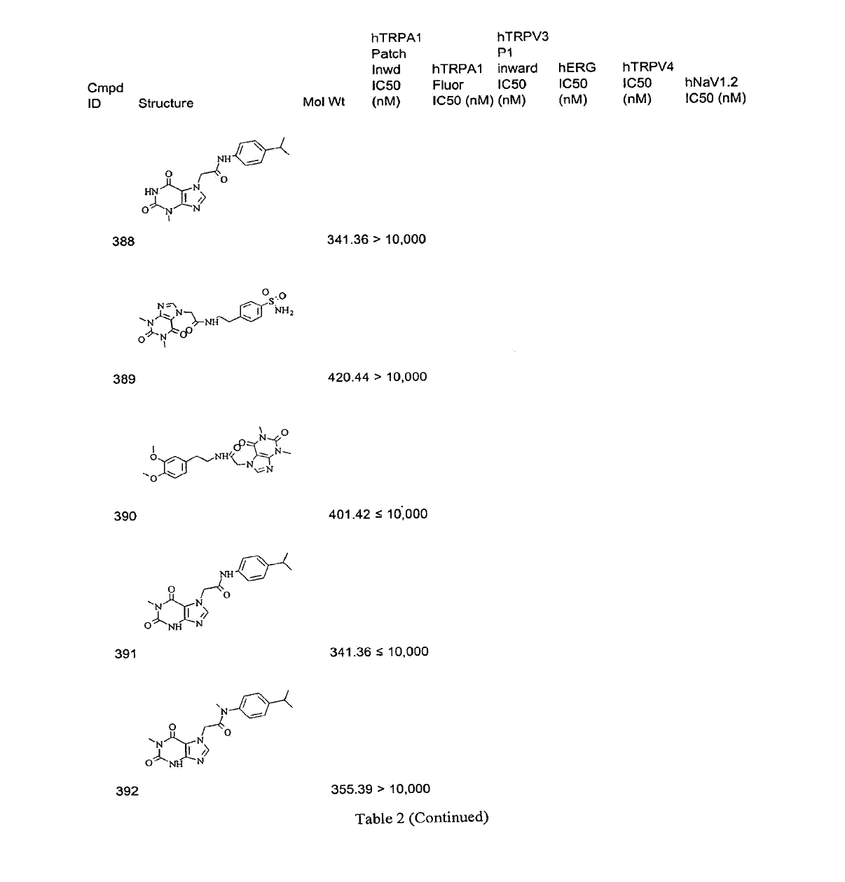

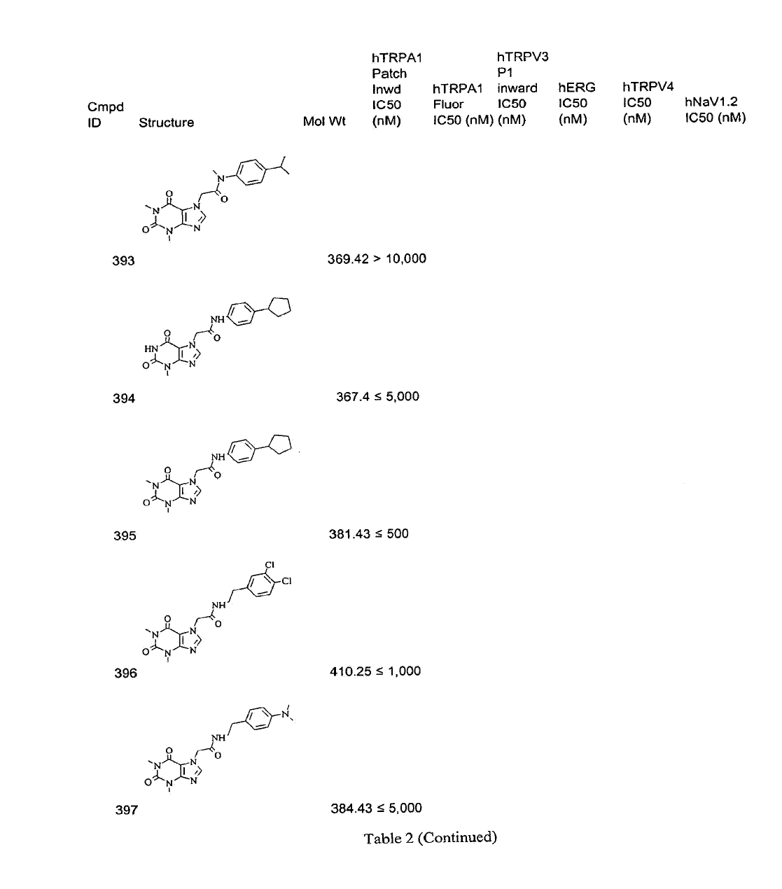

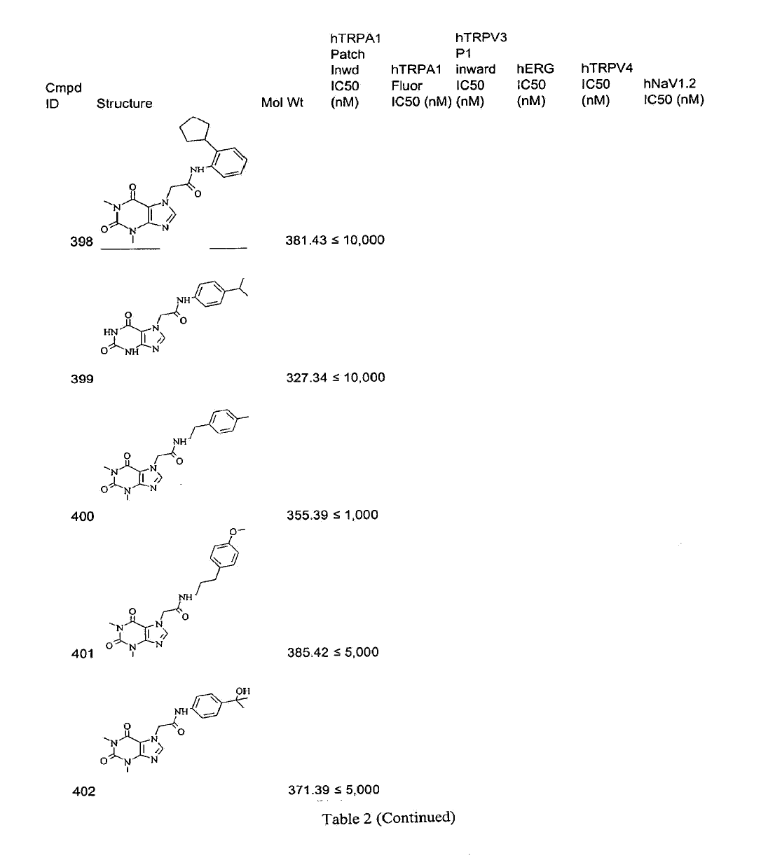

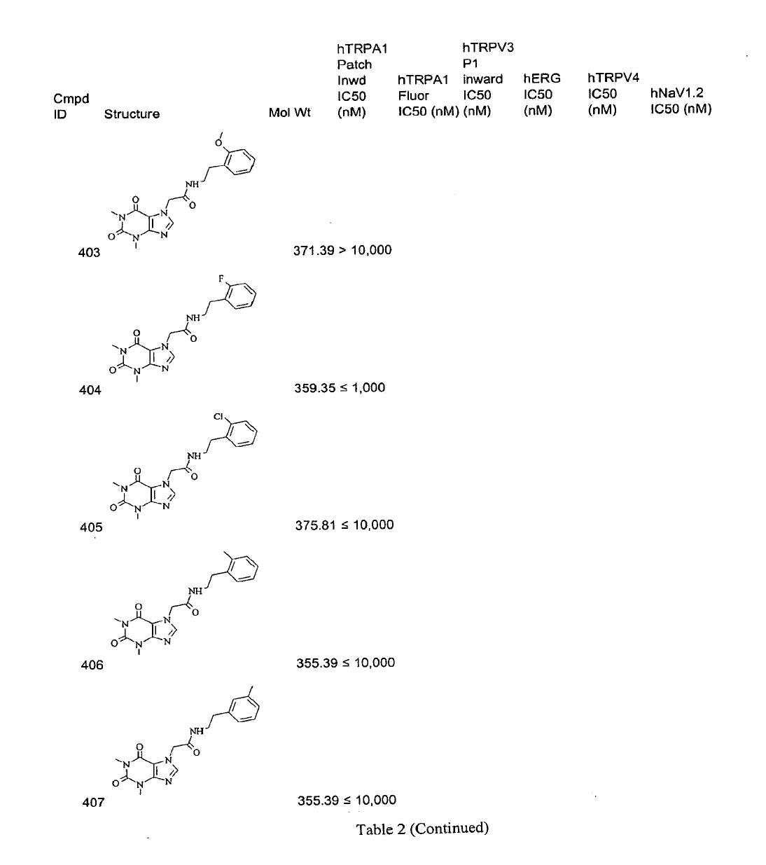

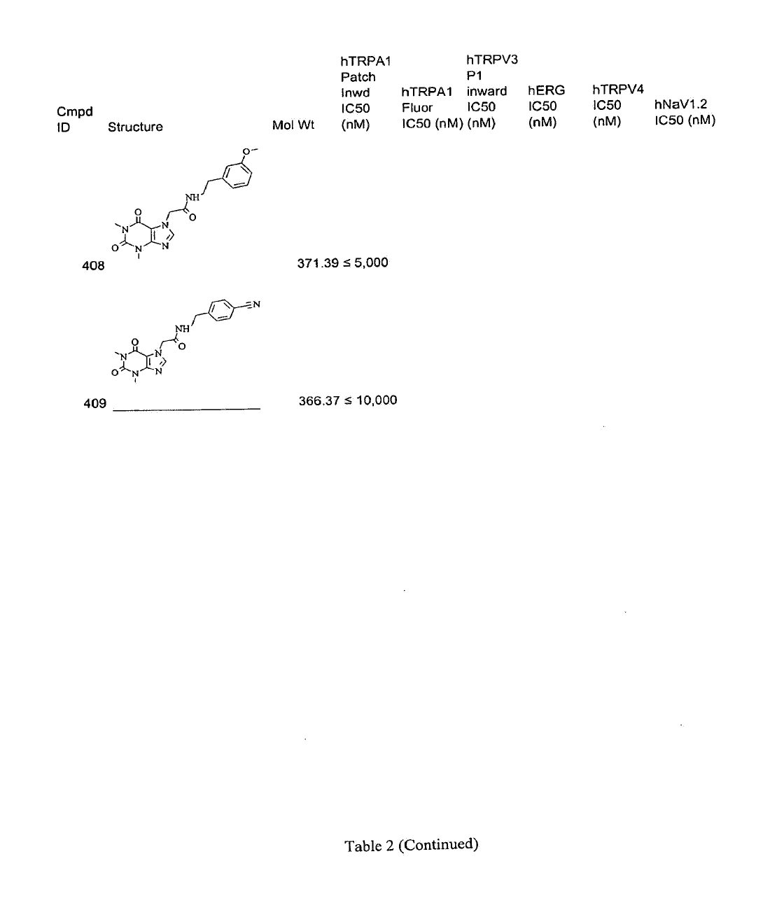

### DETAILED DESCRIPTION OF THE INVENTION

Cellular homeostasis is a result of the summation of regulatory systems involved in, amongst other things, the regulation of ion flux and membrane potential. Cellular homeostasis is achieved, at least in part, by movement of ions into and out of cells across the plasma membrane and within cells by movement of ions across membranes of intracellular organelles including, for example, the endoplasmic reticulum, sarcoplasmic reticulum, mitochondria and endocytic organelles including endosomes and lysosomes.

Movement of ions across cellular membranes is carried out by specialized proteins. TRP channels are one large family of non-selective cation channels that function to help regulate ion flux and membrane potential. TRP channels are subdivided into 6 sub-families including the TRPA (ANKTM1) family. TRPA1 is a member of the TRPA class of TRP channels.

Non-selective cation channels such as TRPA1 modulate the flux of calcium and sodium ions across cellular membranes. Sodium and calcium influx leads to a depolarization of the cell. This increases the probability that voltage-gated ion channels will reach the threshold required for activation. As a result, activation of non-selective cation channels can increase electrical excitability and increase the frequency of voltage-dependent events. Voltage-dependent events include, but are not limited to, neuronal action potentials, cardiac action potentials, smooth muscle contraction, cardiac muscle contraction, and skeletal muscle contraction.

Calcium influx caused by the activation of non-selective cation channels such as TRPA1 also alters the intracellular free calcium concentration. Calcium is a ubiquitous second messenger molecule within the cell. Thus alterations in intracellular calcium levels have profound effects on signal transduction and gene expression. Thus, activation of non-selective cation channels such as TRPA1 can lead to changes in gene expression and cellular phenotype. Gene expression events include, but are not limited to, production of mRNAs encoding cell surface receptors, ion channels, and kinases. These changes in gene expression can lead to hyperexcitability in that cell. Blockers of TRPA1 therefore also have the potential to decrease or prevent pain and/or to decrease overactive bladder.

TRPA1 proteins are receptor operated channels expressed in sensory neurons (see, e.g., Jordt et al. (2004) Nature 427:260-265) including those with cell bodies residing in the dorsal root ganglion, trigeminal ganglion, and nodose ganglia (see Jordt et al. (2004) Nature 427:260-265, Nagata et al. (2005) J. Neurosci 25(16) 4052-61). In addition, low levels of TRPA1 message can be found in some types of fibroblasts (see Jaquemar et al. (1999) JBC 274(11): 7325-33). TRPA1 has also been reported to be expressed in the bladder. Stimulation of a number of extracellular receptors, including, but not limited to, G-protein coupled receptors or receptor tyrosine kinases are sufficient to activate TRPA1.

TRPA1 proteins suitable for use in accordance with the methods provided herein include, for example: human (SEQ ID NO: 1 and SEQ ID NO: 3 amino acid sequences, encoded by SEQ ID NO: 2 and SEQ ID NO: 4 nucleotide sequences respectively) and murine (SEQ ID NO: 5 amino acid sequence, encoded by SEQ ID NO: 6 nucleotide sequence). Particular TRPA1 proteins also include proteins encoded by cDNAs that would hybridize to the TRPA1 sequence (see SEQ ID NO: 2) under stringent conditions.

TRPA1 is the ion channel that responds to mustard oil. The active ingredients in mustard oil (allyl isothiocyanate) and the active ingredient in garlic (allicin) are both capable of activating TRPA1. Other stimuli may also be able to activate TRPA1. It has been reported that severe cold temperatures between 4 and 15° C. activate TRPA1 (see Story et al., (2003) Cell 112(6): 819-829). However, this finding has been controversial (see Jordt et al. (2004) Nature 427:260-265; Nagata et al. (2005) J. Neurosci 25(16): 4052-61). In addition, TRPA1 shares many structural similarities with TRP channels (i.e., TRPN1, Drosophila TRPA1) in lower animals that respond to mechanical stimulation.

Modulating the function of TRPA1 proteins provides a means of modulating calcium homeostasis, sodium homeostasis, membrane polarization, and/or intracellular calcium levels, and compounds that can modulate TRPA1 function are useful in many aspects, including, but not limited to, maintaining calcium homeostasis, modulating intracellular calcium levels, modulating membrane polarization, and treating or preventing diseases, disorders, or conditions associated with calcium and/or sodium homeostasis or dyshomeostasis.

In certain aspects, the present invention provides methods for treating or ameliorating the effects of diseases and conditions using small molecules that inhibit a TRPA1-mediated current and/or a TRPA1-mediated ion flux with an IC₅₀ of less than 10 micromolar. Exemplary suitable compounds for use in any of the methods of the invention (e.g., to treat any of the diseases or conditions disclosed herein) include compounds having one or more of the structural or functional characteristics disclosed herein (e.g., structure, specificity, potency, solubility, etc.).

The present invention contemplates the use of any TRPA1 antagonist possessing one or more of the functional or structural attributes described herein. Additionally, the present invention contemplates the use of TRPA1 antagonists of a compound described herein, as well as the use of any of the particular antagonists provided in Tables 1, 2, 3 or 4. Throughout the application, when particular functional attributes are attributed to TRPA1 antagonists, it is understood that such attributes may characterize TRPA1 inhibitors structurally related to or differing from a compound described herein.

In certain embodiments, a suitable compound inhibits an inward and/or outward TRPA1 mediated current with an IC₅₀ of less than 10 micromolar. In certain embodiments, a suitable compound additionally or alternatively inhibits TRPA1 mediated ion flux with an IC₅₀ of less than 10 micromolar. IC₅₀ can be calculated, for example, in an in vitro assay. For example, IC50 can be calculated using electrophysiological determinations of current, such as standard patch clamp analysis. IC₅₀ can also be evaluated using changes in concentration or flux of ion indicators, such as the calcium flux methods described herein.

In certain embodiments, the invention provides a method for treating or preventing a condition involving activation of TRPA1 or for which reduced TRPA1 activity can reduce the severity, comprising administering an effective amount of a compound described herein or a salt thereof, or a solvate, hydrate, oxidative metabolite or prodrug of the compound or its salt:

Exemplary compounds are provided in Tables 1, 2, 3, and 4.

A represents a compound demonstrating activity of <1 μM as measured in the patch clamp assay. B represents a compound demonstrating activity of >1 μM-<10 μM as measured in the patch clamp assay. C represents a compound demonstrating activity of >10 μM as measured in the patch clamp assay. D represents other exemplary compounds.

TABLE 1

| | | |
|---|---|---|
| 1 | 2-(1,3-dimethyl-2,6-dioxo-2,3-dihydro-1H-purin-7(6H)-yl)-N-(3- | D |
| | methoxyphenyl)acetamide | |
| 2 | 2-(1,3-dimethyl-2,6-dioxo-2,3-dihydro-1H-purin-7(6H)-yl)-N-(4- | C |
| | fluorophenyl)acetamide | |
| 3 | 2-(8-chloro-1,3-dimethyl-2,6-dioxo-2,3-dihydro-1H-purin-7(6H)-yl)-N- | C |
| | p-tolylacetamide | |
| 4 | (E)-2-(1,3-dimethyl-2,6-dioxo-2,3-dihydro-1H-purin-7(6H)-yl)-N′-(4- | B |
| | (trifluoromethyl)benzylidene)acetohydrazide | |
| 5 | 2-(1,3-dimethyl-2,6-dioxo-2,3-dihydro-1H-purin-7(6H)-yl)-N-methyl- | D |
| | N-(2-(pyridin-2-yl)ethyl)acetamide | |
| 6 | 2-(1,3-dimethyl-2,6-dioxo-2,3-dihydro-1H-purin-7(6H)-yl)-N-(2- | D |
| | (pyridin-2-yl)ethyl)acetamide | |
| 7 | 2-(1,3-dimethyl-2,6-dioxo-2,3-dihydro-1H-purin-7(6H)-yl)-N-(3- | D |
| | methoxyphenyl)acetamide | |
| 8 | 2-(1,3-dimethyl-2,6-dioxo-2,3-dihydro-1H-purin-7(6H)-yl)-N-(2- | A |
| | adamantylethyl)acetamide | |
| 9 | 2-(1,3-dimethyl-2,6-dioxo-2,3-dihydro-1H-purin-7(6H)-yl)-N-(5- | B |
| | fluoro-2-methylphenyl)acetamide | |
| 10 | N-cyclooctyl-2-(1,3-dimethyl-2,6-dioxo-2,3-dihydro-1H-purin-7(6H)- | D |
| | yl)acetamide | |
| 11 | 2-(8-chloro-1,3-dimethyl-2,6-dioxo-2,3-dihydro-1H-purin-7(6H)-yl)-N- | D |
| | (adamantylmethyl)acetamide | |
| 12 | 2-(1,3-dimethyl-2,6-dioxo-2,3-dihydro-1H-purin-7(6H)-yl)-N- | D |
| | (thiochroman-4-yl)acetamide | |
| 13 | 2-(1,3-dimethyl-2,6-dioxo-2,3-dihydro-1H-purin-7(6H)-yl)-N-(2- | D |
| | fluorophenyl)acetamide | |
| 14 | 2-(1,3-dimethyl-2,6-dioxo-2,3-dihydro-1H-purin-7(6H)-yl)-N-(2- | D |
| | (furan-2-yl)-2-(pyrrolidin-1-yl)ethyl)acetamide | |
| 15 | 2-(8-chloro-1,3-dimethyl-2,6-dioxo-2,3-dihydro-1H-purin-7(6H)-yl)-N- | D |
| | (3-fluoro-4-methoxybenzyl)-N-methylacetamide | |
| 16 | 2-(8-chloro-1,3-dimethyl-2,6-dioxo-2,3-dihydro-1H-purin-7(6H)-yl)-N- | D |
| | (3-methoxybenzyl)acetamide | |
| 17 | 2-(8-chloro-1,3-dimethyl-2,6-dioxo-2,3-dihydro-1H-purin-7(6H)-yl)-N- | D |
| | (4-fluorophenethyl)acetamide | |
| 18 | 2-(1,3-dimethyl-2,6-dioxo-2,3-dihydro-1H-purin-7(6H)-yl)-N-p- | A |
| | tolylpropanamide | |
| 19 | N-((2,3-dihydrobenzo[b][1,4]dioxin-2-yl)methyl)-2-(1,3-dimethyl-2,6- | D |
| | dioxo-2,3-dihydro-1H-purin-7(6H)-yl)acetamide | |
| 20 | 2-(8-chloro-1,3-dimethyl-2,6-dioxo-2,3-dihydro-1H-purin-7(6H)-yl)-N- | A |
| | (2,3-dihydro-1H-inden-5-yl)acetamide | |
| 21 | 2-(8-chloro-1,3-dimethyl-2,6-dioxo-2,3-dihydro-1H-purin-7(6H)-yl)-N- | D |
| | (2,3-dimethylcyclohexyl)acetamide | |
| 22 | methyl 4-((2-(8-chloro-1,3-dimethyl-2,6-dioxo-2,3-dihydro-1H-purin- | D |
| | 7(6H)-yl)acetamido)methyl)benzoate | |
| 23 | N-(3,4-dimethoxybenzyl)-2-(1,3-dimethyl-2,6-dioxo-2,3-dihydro-1H- | D |
| | purin-7(6H)-yl)acetamide | |
| 24 | 2-(8-chloro-1,3-dimethyl-2,6-dioxo-2,3-dihydro-1H-purin-7(6H)-yl)-N- | D |
| | isobutyl-N-(dioxytetrahydrothiophen-3-yl)acetamide | |
| 25 | N-((3,5-dimethyl-1-phenyl-1H-pyrazol-4-yl)methyl)-2-(1,3-dimethyl- | D |
| | 2,6-dioxo-2,3-dihydro-1H-purin-7(6H)-yl)-N-methylacetamide | |
| 26 | 2-(8-chloro-1,3-dimethyl-2,6-dioxo-2,3-dihydro-1H-purin-7(6H)-yl)-N- | D |
| | methyl-N-(4-methylbenzyl)acetamide | |
| 27 | 2-(8-chloro-1,3-dimethyl-2,6-dioxo-2,3-dihydro-1H-purin-7(6H)-yl)-N- | D |
| | cyclohexyl-N-ethylacetamide | |
| 28 | 2-(8-chloro-1,3-dimethyl-2,6-dioxo-2,3-dihydro-1H-purin-7(6H)-yl)-N- | D |
| | (3,4-dihydro-2H-benzo[b][1,4]dioxepin-7-yl)acetamide | |
| 29 | 2-(8-chloro-1,3-dimethyl-2,6-dioxo-2,3-dihydro-1H-purin-7(6H)-yl)-N- | D |
| | (1-cyclopentyl-1H-pyrazol-5-yl)acetamide | |
| 30 | 2-(8-chloro-1,3-dimethyl-2,6-dioxo-2,3-dihydro-1H-purin-7(6H)-yl)-N- | D |
| | methyl-N-(1,2,3,4-tetrahydronaphthalen-1-yl)acetamide | |
| 31 | 2-(8-chloro-1,3-dimethyl-2,6-dioxo-2,3-dihydro-1H-purin-7(6H)-yl)-N- | D |
| | (4-phenylbutan-2-yl)acetamide | |
| 32 | N-cyclohexyl-2-(1,3-dimethyl-2,6-dioxo-2,3-dihydro-1H-purin-7(6H)- | D |
| | yl)-N-ethylacetamide | |
| 33 | 2-(8-chloro-1,3-dimethyl-2,6-dioxo-2,3-dihydro-1H-purin-7(6H)-yl)-N- | A |
| | (2-(4-chlorophenylthio)ethyl)acetamide | |
| 34 | N-((2,3-dihydrobenzo[b][1,4]dioxin-2-yl)methyl)-2-(1,3-dimethyl-2,6- | D |
| | dioxo-2,3-dihydro-1H-purin-7(6H)-yl)-N-methylacetamide | |
| 35 | N-(2-cyclohexenylethyl)-2-(1,3-dimethyl-2,6-dioxo-2,3-dihydro-1H- | D |
| | purin-7(6H)-yl)acetamide | |
| 36 | 2-(8-chloro-1,3-dimethyl-2,6-dioxo-2,3-dihydro-1H-purin-7(6H)-yl)-N- | D |
| | (1-(5,6,7,8-tetrahydronaphthalen-2-yl)ethyl)acetamide | |
| 37 | N-(3,5-dichloropyridin-2-yl)-2-(1,3-dimethyl-2,6-dioxo-2,3-dihydro- | D |
| | 1H-purin-7(6H)-yl)propanamide | |
| 38 | N-(5-chloropyridin-2-yl)-2-(1,3-dimethyl-2,6-dioxo-2,3-dihydro-1H- | D |
| | purin-7(6H)-yl)propanamide | |
| 39 | N-((4-chlorophenyl)(cyclopropyl)methyl)-2-(1,3-dimethyl-2,6-dioxo- | D |
| | 2,3-dihydro-1H-purin-7(6H)-yl)acetamide | |
| 40 | N-(1-(3,4-dihydro-2H-benzo[b][1,4]dioxepin-7-yl)-2-methylpropyl)-2- | D |
| | (1,3-dimethyl-2,6-dioxo-2,3-dihydro-1H-purin-7(6H)-yl)acetamide | |
| 41 | N-((1-benzyl-1H-pyrazol-4-yl)methyl)-2-(1,3-dimethyl-2,6-dioxo-2,3- | D |
| | dihydro-1H-purin-7(6H)-yl)-N-methylacetamide | |
| 42 | 2-(1,3-dimethyl-2,6-dioxo-2,3-dihydro-1H-purin-7(6H)-yl)-N-(2,2- | C |
| | diphenylpropyl)acetamide | |
| 43 | 2-(1,3-dimethyl-2,6-dioxo-2,3-dihydro-1H-purin-7(6H)-yl)-N-((2- | D |
| | ethylbenzofuran-3-yl)methyl)-N-methylacetamide | |
| 44 | N-(cyclohexylmethyl)-2-(1,3-dimethyl-2,6-dioxo-2,3-dihydro-1H- | B |
| | purin-7(6H)-yl)acetamide | |
| 45 | N-cyclohexyl-2-(1,3-dimethyl-2,6-dioxo-2,3-dihydro-1H-purin-7(6H)- | D |
| | yl)-N-methylpropanamide | |
| 46 | 3-(1,3-dimethyl-2,6-dioxo-2,3-dihydro-1H-purin-7(6H)-yl)-N-(4- | C |
| | methoxybenzyl)propanamide | |
| 47 | 2-(1,3-diethyl-2,6-dioxo-2,3-dihydro-1H-purin-7(6H)-yl)-N-(4- | C |
| | methoxyphenethyl)acetamide | |
| 48 | 1-(1,3-dimethyl-2,6-dioxo-2,3-dihydro-1H-purin-7(6H)-yl)-N-(4- | C |
| | methoxyphenethyl)methanesulfonamide | |
| 49 | N-(2-cyclohexylethyl)-2-(1,3-dimethyl-2,6-dioxo-2,3-dihydro-1H- | C |
| | purin-7(6H)-yl)acetamide | |
| 50 | 2-(1,3-dimethyl-2,6-dioxo-2,3-dihydro-1H-purin-7(6H)-yl)-N-(2-(4- | C |
| | hydroxycyclohexyl)ethyl)acetamide | |
| 51 | 2-(1,3-dimethyl-2,6-dioxo-2,3-dihydro-1H-purin-7(6H)-yl)-N-(3,4- | B |
| | dimethylphenethyl)acetamide | |
| 52 | N-(2-(biphenyl-4-yl)ethyl)-2-(1,3-dimethyl-2,6-dioxo-2,3-dihydro-1H- | A |
| | purin-7(6H)-yl)acetamide | |
| 53 | N-(4-(benzyloxy)phenethyl)-2-(1,3-dimethyl-2,6-dioxo-2,3-dihydro- | B |
| | 1H-purin-7(6H)-yl)acetamide | |
| 54 | 2-(1,3-dimethyl-2,6,8-trioxo-2,3,7,8-tetrahydro-1H-purin-9(6H)-yl)-N- | C |
| | (4-methoxyphenethyl)acetamide | |
| 55 | 2-(1,3-dimethyl-2,6-dioxo-2,3-dihydro-1H-purin-7(6H)-yl)-N- | C |
| | ((3R,4R)-quinuclidin-3-yl)acetamide | |
| 56 | N-(4-butoxyphenethyl)-2-(1,3-dimethyl-2,6-dioxo-2,3-dihydro-1H- | B |
| | purin-7(6H)-yl)acetamide | |
| 57 | N-(4-cyclohexylphenethyl)-2-(1,3-dimethyl-2,6-dioxo-2,3-dihydro-1H- | A |
| | purin-7(6H)-yl)acetamide | |
| 58 | 2-(1,3-dimethyl-2,6-dioxo-2,3-dihydro-1H-purin-7(6H)-yl)-N-((1S,2R)- | B |
| | 2-(4-methoxyphenyl)cyclopropyl)acetamide | |
| 59 | N-(4-tert-butylphenethyl)-2-(1,3-dimethyl-2,6-dioxo-2,3-dihydro-1H- | B |
| | purin-7(6H)-yl)acetamide | |
| 60 | 2-(1,3-dimethyl-2,6-dioxo-2,3-dihydro-1H-purin-7(6H)-yl)-N-(4- | B |
| | methoxybenzylsulfonyl)acetamide | |
| 61 | N-(4-methoxyphenethyl)-2-(1,3,9-trimethyl-2,6,8-trioxo-2,3-dihydro- | B |
| | 1H-purin-7(6H,8H,9H)-yl)acetamide | |
| 62 | 4-methylphenethyl 2-(1,3-dimethyl-2,6-dioxo-2,3-dihydro-1H-purin- | B |
| | 7(6H)-yl)acetate | |
| 63 | 2-(1,3-dimethyl-2,6-dioxo-2,3-dihydro-1H-purin-7(6H)-yl)-N-(2- | C |
| | (piperidin-1-yl)ethyl)acetamide | |
| 64 | 2-(1,3-dimethyl-2,6-dioxo-2,3-dihydro-1H-purin-7(6H)-yl)-N-(2- | C |
| | morpholinoethyl)acetamide | |
| 65 | 2-(1,3-dimethyl-2,6-dioxo-2,3-dihydro-1H-purin-7(6H)-yl)-N-((1S,2S)- | B |
| | 2-(4-methoxyphenyl)cyclopropyl)acetamide | |
| 66 | 2-(1,3-dicyclopropyl-2,6-dioxo-2,3-dihydro-1H-purin-7(6H)-yl)-N-(4- | D |
| | methoxyphenethyl)acetamide | |
| 67 | 2-(1,3-dimethyl-2,6-dioxo-2,3-dihydro-1H-purin-7(6H)-yl)-N-(2-(4- | D |
| | methoxycyclohexyl)ethyl)acetamide | |
| 68 | 2-(1,3-dimethyl-2,6,8-trioxo-2,3-dihydro-1H-purin-7(6H,8H,9H)-yl)-N- | C |
| | (4-methoxyphenethyl)acetamide | |
| 69 | 2-(1,3-dimethyl-2,6-dioxo-2,3-dihydro-1H-purin-7(6H)-yl)-N-(2- | C |
| | (pyridin-4-yl)ethyl)acetamide | |
| 70 | 2-(1,3-dimethyl-2,6-dioxo-2,3-dihydro-1H-purin-7(6H)-yl)-N-(2-(4- | B |
| | methylpiperazin-1-yl)ethyl)acetamide | |
| 71 | 1,3-dimethyl-7-(2-(4-methylphenethylamino)ethyl)-1H-purine- | B |
| | 2,6(3H,7H)-dione | |
| 72 | N-(2-(1,3-dimethyl-2,6-dioxo-2,3-dihydro-1H-purin-7(6H)-yl)ethyl)-N- | B |
| | (4-methylphenethyl)methanesulfonamide | |
| 73 | 2-(3-methyl-2,6-dioxo-1-propyl-2,3-dihydro-1H-purin-7(6H)-yl)-N-(4- | C |
| | methylphenethyl)acetamide | |
| 74 | 1,3-dimethyl-7-(2-(methyl(4-methylphenethyl)amino)ethyl)-1H-purine- | B |
| | 2,6(3H,7H)-dione | |
| 75 | 2-(1-methyl-2,6-dioxo-3-propyl-2,3-dihydro-1H-purin-7(6H)-yl)-N-(4- | B |
| | methylphenethyl)acetamide | |
| 76 | N-(2-(1,3-dimethyl-2,6-dioxo-2,3-dihydro-1H-purin-7(6H)-yl)ethyl)-N- | A |
| | (4-methylphenethyl)acetamide | |
| 77 | 2-(1-methyl-2,6-dioxo-3-propyl-2,3-dihydro-1H-purin-7(6H)-yl)-N- | A |
| | phenethylacetamide | |
| 78 | 2-(1,3-dimethyl-2,6-dioxo-2,3-dihydro-1H-purin-7(6H)-yl)-N-(2- | B |
| | phenylpropyl)acetamide | |
| 79 | 2-(1,3-dimethyl-2,6-dioxo-2,3-dihydro-1H-purin-7(6H)-yl)-N-(3- | B |
| | fluorophenethyl)acetamide | |
| 80 | 2-(1,3-dimethyl-2,6-dioxo-2,3-dihydro-1H-purin-7(6H)-yl)-N-(4- | B |
| | ethylphenethyl)acetamide | |
| 81 | (S)-2-(1,3-dimethyl-2,6-dioxo-2,3-dihydro-1H-purin-7(6H)-yl)-N-(1- | B |
| | hydroxy-3-phenylpropan-2-yl)acetamide | |
| 82 | N-(2,3-dimethoxyphenethyl)-2-(1,3-dimethyl-2,6-dioxo-2,3-dihydro- | B |
| | 1H-purin-7(6H)-yl)acetamide | |
| 83 | N-(2,3-dichlorophenethyl)-2-(1,3-dimethyl-2,6-dioxo-2,3-dihydro-1H- | B |
| | purin-7(6H)-yl)acetamide | |
| 84 | N-(2-(benzo[d][1,3]dioxol-5-yl)ethyl)-2-(1,3-dimethyl-2,6-dioxo-2,3- | B |
| | dihydro-1H-purin-7(6H)-yl)acetamide | |
| 85 | (R)-2-(1,3-dimethyl-2,6-dioxo-2,3-dihydro-1H-purin-7(6H)-yl)-N-(1- | B |
| | hydroxy-3-phenylpropan-2-yl)acetamide | |
| 86 | N-(2,5-dimethoxyphenethyl)-2-(1,3-dimethyl-2,6-dioxo-2,3-dihydro- | B |
| | 1H-purin-7(6H)-yl)acetamide | |
| 87 | N-(2,4-dichlorophenethyl)-2-(1,3-dimethyl-2,6-dioxo-2,3-dihydro-1H- | B |
| | purin-7(6H)-yl)acetamide | |
| 88 | N-(2,6-dichlorophenethyl)-2-(1,3-dimethyl-2,6-dioxo-2,3-dihydro-1H- | B |
| | purin-7(6H)-yl)acetamide | |
| 89 | N-(2-bromophenethyl)-2-(1,3-dimethyl-2,6-dioxo-2,3-dihydro-1H- | B |
| | purin-7(6H)-yl)acetamide | |
| 90 | 2-(1,3-dimethyl-2,6-dioxo-2,3-dihydro-1H-purin-7(6H)-yl)-N-(2- | D |
| | (quinuclidin-3-yl)ethyl)acetamide | |
| 91 | 2-(3-(2-(dimethylamino)ethyl)-1-methyl-2,6-dioxo-2,3-dihydro-1H- | D |
| | purin-7(6H)-yl)-N-(4-methylphenethyl)acetamide | |
| 92 | 2-(1-(2-(dimethylamino)ethyl)-3-methyl-2,6-dioxo-2,3-dihydro-1H- | D |
| | purin-7(6H)-yl)-N-(4-methylphenethyl)acetamide | |
| 93 | N-(6-chlorobenzo[d]thiazol-2-yl)-2-(1,3-dimethyl-2,6-dioxo-2,3- | D |
| | dihydro-1H-purin-7(6H)-yl)acetamide | |
| 94 | N-(6-bromobenzo[d]thiazol-2-yl)-2-(1,3-dimethyl-2,6-dioxo-2,3- | A |
| | dihydro-1H-purin-7(6H)-yl)acetamide | |
| 95 | N-(4-chlorobenzo[d]thiazol-2-yl)-2-(1,3-dimethyl-2,6-dioxo-2,3- | D |
| | dihydro-1H-purin-7(6H)-yl)acetamide | |
| 96 | 2-(1,3-dimethyl-2,6-dioxo-2,3-dihydro-1H-purin-7(6H)-yl)-N-(5,6- | D |
| | dimethylbenzo[d]thiazol-2-yl)acetamide | |
| 97 | 2-(1,3-dimethyl-2,6-dioxo-2,3-dihydro-1H-purin-7(6H)-yl)-N-(6- | A |
| | ethoxybenzo[d]thiazol-2-yl)acetamide | |
| 98 | 2-(1,3-dimethyl-2,6-dioxo-2,3-dihydro-1H-purin-7(6H)-yl)-N-(4- | D |
| | methoxybenzo[d]thiazol-2-yl)acetamide | |
| 99 | 2-(1,3-dimethyl-2,6-dioxo-2,3-dihydro-1H-purin-7(6H)-yl)-N-(4- | D |
| | methylbenzo[d]thiazol-2-yl)acetamide | |
| 100 | 2-(1,3-dimethyl-2,6-dioxo-2,3-dihydro-1H-purin-7(6H)-yl)-N-(6- | D |
| | (methylsulfonyl)benzo[d]thiazol-2-yl)acetamide | |
| 101 | 2-(1,3-dimethyl-2,6-dioxo-2,3-dihydro-1H-purin-7(6H)-yl)-N-(2- | D |
| | hydroxy-2-phenylethyl)acetamide | |
| 102 | N-(3-chlorophenethyl)-2-(1,3-dimethyl-2,6-dioxo-2,3-dihydro-1H- | D |
| | purin-7(6H)-yl)acetamide | |
| 103 | (S)-2-(2-(1,3-dimethyl-2,6-dioxo-2,3-dihydro-1H-purin-7(6H)- | D |
| | yl)acetamido)-3-phenylpropanamide | |
| 104 | N-(2-chloro-6,7-dimethoxyquinazolin-4-yl)-2-(1,3-dimethyl-2,6-dioxo- | D |
| | 2,3-dihydro-1H-purin-7(6H)-yl)acetamide | |
| 105 | N-(4-bromophenethyl)-2-(1,3-dimethyl-2,6-dioxo-2,3-dihydro-1H- | D |
| | purin-7(6H)-yl)acetamide | |
| 106 | 2-(1,3-dimethyl-2,6-dioxo-2,3-dihydro-1H-purin-7(6H)-yl)-N-(2,2- | D |
| | diphenylethyl)acetamide | |
| 107 | (S)-methyl 2-(2-(1,3-dimethyl-2,6-dioxo-2,3-dihydro-1H-purin-7(6H)- | D |
| | yl)acetamido)-3-phenylpropanoate | |
| 108 | N-(3,5-dimethoxyphenethyl)-2-(1,3-dimethyl-2,6-dioxo-2,3-dihydro- | D |
| | 1H-purin-7(6H)-yl)acetamide | |
| 109 | (R)-2-(2-(1,3-dimethyl-2,6-dioxo-2,3-dihydro-1H-purin-7(6H)- | D |
| | yl)acetamido)-3-phenylpropanamide | |
| 110 | 2-(1,3-dimethyl-2,6-dioxo-2,3-dihydro-1H-purin-7(6H)-yl)-N-(4,5,6,7- | D |
| | tetrahydrobenzo[d]thiazol-2-yl)acetamide | |
| 111 | ethyl 2-(2-(1,3-dimethyl-2,6-dioxo-2,3-dihydro-1H-purin-7(6H)- | D |
| | yl)acetamido)-5-methylthiazole-4-carboxylate | |
| 112 | N-(2-(1,4-diazabicyclo[2.2.2]octan-2-yl)ethyl)-2-(1,3-dimethyl-2,6- | D |
| | dioxo-2,3-dihydro-1H-purin-7(6H)-yl)acetamide | |
| 113 | ethyl 2-(2-(1,3-dimethyl-2,6-dioxo-2,3-dihydro-1H-purin-7(6H)- | D |
| | yl)acetamido)benzo[d]thiazole-6-carboxylate | |
| 114 | 2-(1,3-dimethyl-2,6-dioxo-2,3-dihydro-1H-purin-7(6H)-yl)-N-methyl- | D |
| | N-phenethylacetamide | |
| 115 | 1,3-dimethyl-7-((5-(4-methylbenzyl)-1,3,4-oxadiazol-2-yl)methyl)-1H- | D |
| | purine-2,6(3H,7H)-dione | |
| 116 | 2-(1,3-dimethyl-2,6-dioxo-2,3-dihydro-1H-purin-7(6H)-yl)-N-(2-(6- | D |
| | methylpyridin-3-yl)ethyl)acetamide | |
| 117 | 2-(1,3-dimethyl-2,6-dioxo-2,3-dihydro-1H-purin-7(6H)-yl)-N-(2-(1- | D |
| | methyl-1H-imidazol-2-yl)ethyl)acetamide | |
| 118 | 1,3-dimethyl-7-(3-(6-methyl-1H-indol-2-yl)propyl)-1H-purine- | D |
| | 2,6(3H,7H)-dione | |
| 119 | 2-(1,3-dimethyl-2,6-dioxo-2,3-dihydro-1H-purin-7(6H)-yl)-N-ethyl-N- | D |
| | (pyridin-4-ylmethyl)acetamide | |
| 120 | N-(4,5-dihydrothiazol-2-yl)-2-(1,3-dimethyl-2,6-dioxo-2,3-dihydro-1H- | D |
| | purin-7(6H)-yl)acetamide | |
| 121 | ethyl 2-(2-(1,3-dimethyl-2,6-dioxo-2,3-dihydro-1H-purin-7(6H)- | D |
| | yl)acetamido)-4-methylthiazole-5-carboxylate | |
| 122 | N-(4H-chromeno[4,3-d]thiazol-2-yl)-2-(1,3-dimethyl-2,6-dioxo-2,3- | D |
| | dihydro-1H-purin-7(6H)-yl)acetamide | |
| 123 | ethyl 2-(2-(2-(1,3-dimethyl-2,6-dioxo-2,3-dihydro-1H-purin-7(6H)- | D |
| | yl)acetamido)thiazol-4-yl)acetate | |
| 124 | 2-(1,3-dimethyl-2,6-dioxo-2,3-dihydro-1H-purin-7(6H)-yl)-N-(4- | D |
| | methylthiazol-2-yl)acetamide | |
| 125 | 2-(1,3-dimethyl-2,6-dioxo-2,3-dihydro-1H-purin-7(6H)-yl)-N-(1,2,3,4- | D |
| | tetrahydronaphthalen-1-yl)acetamide | |
| 126 | N-(2,3-dihydro-1H-inden-1-yl)-2-(1,3-dimethyl-2,6-dioxo-2,3-dihydro- | D |
| | 1H-purin-7(6H)-yl)acetamide | |
| 127 | N′-(2-(1,3-dimethyl-2,6-dioxo-2,3-dihydro-1H-purin-7(6H)-yl)acetyl)- | D |
| | 4-methylbenzohydrazide | |
| 128 | 2-(1,3-dimethyl-2,6-dioxo-2,3-dihydro-1H-purin-7(6H)-yl)-N-(2-(5- | D |
| | methylpyridin-2-yl)ethyl)acetamide | |
| 129 | 1-(2-(1,3-dimethyl-2,6-dioxo-2,3-dihydro-1H-purin-7(6H)-yl)ethyl)-1- | D |
| | (4-methylphenethyl)urea | |
| 130 | 1,3-dimethyl-7-((5-p-tolyl-1,3,4-oxadiazol-2-yl)methyl)-1H-purine- | D |
| | 2,6(3H,7H)-dione | |
| 131 | N-(2-(1,3-dimethyl-2,6-dioxo-2,3-dihydro-1H-purin-7(6H)-yl)ethyl)-N- | D |
| | (4-methylphenethyl)propionamide | |
| 132 | 1,3-dimethyl-7-((2-(4-methylphenethyl)cyclopropyl)methyl)-1H- | D |
| | purine-2,6(3H,7H)-dione | |
| 133 | N-(3-bromo-4-methoxyphenethyl)-2-(1,3-dimethyl-2,6-dioxo-2,3- | D |
| | dihydro-1H-purin-7(6H)-yl)acetamide | |
| 134 | N-((1R,2R)-1,3-dihydroxy-1-phenylpropan-2-yl)-2-(1,3-dimethyl-2,6- | D |
| | dioxo-2,3-dihydro-1H-purin-7(6H)-yl)acetamide | |
| 135 | 2-(1,3-dimethyl-2,6-dioxo-2,3-dihydro-1H-purin-7(6H)-yl)-N- | D |
| | (piperidin-4-yl)acetamide | |
| 136 | 2-(1,3-dimethyl-2,6-dioxo-2,3-dihydro-1H-purin-7(6H)-yl)-N-(2,2,6,6- | D |
| | tetramethylpiperidin-4-yl)acetamide | |
| 137 | 2-(1,3-dimethyl-2,6-dioxo-2,3-dihydro-1H-purin-7(6H)-yl)-N-(2- | D |
| | hydroxy-2-phenylethyl)-N-methylacetamide | |
| 138 | 2-(1,3-dimethyl-2,6-dioxo-2,3-dihydro-1H-purin-7(6H)-yl)-N-(4,6- | D |
| | dimethylpyrimidin-2-yl)acetamide | |
| 139 | N-benzyl-2-(1,3-dimethyl-2,6-dioxo-2,3-dihydro-1H-purin-7(6H)-yl)- | D |
| | N-(4,5,6,7-tetrahydrobenzo[d]thiazol-2-yl)acetamide | |
| 140 | N-benzyl-2-(8-chloro-1,3-dimethyl-2,6-dioxo-2,3-dihydro-1H-purin- | D |
| | 7(6H)-yl)-N-(4,5,6,7-tetrahydrobenzo[d]thiazol-2-yl)acetamide | |
| 141 | N-(4-acetylthiazol-2-yl)-2-(1,3-dimethyl-2,6-dioxo-2,3-dihydro-1H- | D |
| | purin-7(6H)-yl)acetamide | |
| 142 | 2-(8-chloro-1,3-dimethyl-2,6-dioxo-1,2,3,6-tetrahydropurin-7-yl)-N-(4- | D |
| | methylphenethyl)acetamide; | |
| 143 | N-(6-butylbenzo[d]thiazol-2-yl)-2-(8-chloro-1,3-dimethyl-2,6-dioxo- | D |
| | 1,2,3,6-tetrahydropurin-7-yl)acetamide; | |
| 144 | 2-(8-chloro-1,3-dimethyl-2,6-dioxo-1,2,3,6-tetrahydropurin-7-yl)-N-(2- | D |
| | (adamant-1-yl)ethyl)acetamide; | |
| 145 | 1-(2-(1,3-dimethyl-2,6-dioxo-1,2,3,6-tetrahydropurin-7-yl)ethyl)-1-(4- | D |
| | methylphenethyl)urea; | |
| 146 | N-(2-(1,3-dimethyl-2,6-dioxo-1,2,3,6-tetrahydropurin-7-yl)ethyl)-N-(4- | D |
| | methylphenethyl)propionamide; | |
| 147 | N-(2-(1,3-dimethyl-2,6-dioxo-1,2,3,6-tetrahydropurin-7-yl)ethyl)-N-(4- | D |
| | methylphenethyl)pentanamide; | |
| 148 | 2-(3-(2-hydroxyethyl)-1-methyl-2,6-dioxo-1,2,3,6-tetrahydropurin-7- | D |
| | yl)-N-(4-methylphenethyl)acetamide; | |
| 149 | 2-(3-(2-amino-2-oxoethyl)-1-methyl-2,6-dioxo-1,2,3,6-tetrahydropurin- | D |
| | 7-yl)-N-(4-methylphenethyl)acetamide; | |
| 150 | N-(2-(1,3-dimethyl-2,6-dioxo-1,2,3,6-tetrahydropurin-7-yl)ethyl)-4- | D |
| | oxo-4-phenylbutanamide; | |
| 151 | N-(2-(1,3-dimethyl-2,6-dioxo-1,2,3,6-tetrahydropurin-7-yl)ethyl)-5-(4- | D |
| | fluorophenyl)-5-oxopentanamide; | |
| 152 | 2-(3-chloro-4-(trifluoromethyl)phenyl)-N-(2-(1,3-dimethyl-2,6-dioxo- | D |
| | 1,2,3,6-tetrahydropurin-7-yl)ethyl)acetamide; | |
| 153 | 2-(3-chloro-4-fluorophenyl)-N-(2-(1,3-dimethyl-2,6-dioxo-1,2,3,6- | D |
| | tetrahydropurin-7-yl)ethyl)acetamide; | |
| 154 | 1-benzyl-N-(2-(1,3-dimethyl-2,6-dioxo-1,2,3,6-tetrahydropurin-7- | D |
| | yl)ethyl)-1H-pyrazole-4-carboxamide; | |
| 155 | N-(2-(2-(1,3-dimethyl-2,6-dioxo-1,2,3,6-tetrahydropurin-7- | D |
| | yl)ethylamino)-2-oxoethyl)-4-methylbenzamide; | |
| 156 | N-(2-(1,3-dimethyl-2,6-dioxo-1,2,3,6-tetrahydropurin-7-yl)ethyl)-1- | D |
| | isopropyl-1H-pyrazole-4-carboxamide; | |
| 157 | N-(2-(1,3-dimethyl-2,6-dioxo-1,2,3,6-tetrahydropurin-7-yl)ethyl)-2- | D |
| | phenylacetamide; | |
| 158 | benzyl 2-(1,3-dimethyl-2,6-dioxo-1,2,3,6-tetrahydropurin-7- | D |
| | yl)ethylcarbamate; | |
| 159 | N-(2-(1,3-dimethyl-2,6-dioxo-1,2,3,6-tetrahydropurin-7-yl)ethyl)-4- | D |
| | methylbenzamide; | |
| 160 | N-(2-(1,3-dimethyl-2,6-dioxo-1,2,3,6-tetrahydropurin-7-yl)ethyl)-3,4,5- | D |
| | trimethoxybenzamide; | |
| 161 | 4-chloro-N-(2-(1,3-dimethyl-2,6-dioxo-1,2,3,6-tetrahydropurin-7- | D |
| | yl)ethyl)picolinamide; | |
| 162 | 5-bromo-N-(2-(1,3-dimethyl-2,6-dioxo-1,2,3,6-tetrahydropurin-7- | D |
| | yl)ethyl)furan-2-carboxamide; | |
| 163 | 5-chloro-N-(2-(1,3-dimethyl-2,6-dioxo-1,2,3,6-tetrahydropurin-7- | D |
| | yl)ethyl)thiophene-2-carboxamide; | |
| 164 | N-(2-(1,3-dimethyl-2,6-dioxo-1,2,3,6-tetrahydropurin-7- | D |
| | yl)ethyl)pyrimidine-4-carboxamide; | |
| 165 | N-(2-(1,3-dimethyl-2,6-dioxo-1,2,3,6-tetrahydropurin-7- | D |
| | yl)ethyl)pyrazine-2-carboxamide; | |
| 166 | N-(2-(1,3-dimethyl-2,6-dioxo-1,2,3,6-tetrahydropurin-7- | D |
| | yl)ethyl)cyclopentanecarboxamide; | |
| 167 | N-(4H-chromeno[4,3-d]oxazol-2-yl)-2-(1,3-dimethyl-2,6-dioxo-1,2,3,6- | D |
| | tetrahydropurin-7-yl)acetamide | |
| 168 | N-(3,4-dihydrochromeno[3,4-d]imidazol-2-yl)-2-(1,3-dimethyl-2,6- | D |
| | dioxo-1,2,3,6-tetrahydropurin-7-yl)acetamide | |
| 169 | 2-(1,3-dimethyl-2,6-dioxo-1,2,3,6-tetrahydropurin-7-yl)-N-(3-methyl- | D |
| | 3,4-dihydrochromeno[3,4-d]imidazol-2-yl)acetamide | |
| 170 | 2-(1,3-dimethyl-2,6-dioxo-1,2,3,6-tetrahydropurin-7-yl)-N-(3-ethyl-3,4- | D |
| | dihydrochromeno[3,4-d]imidazol-2-yl)acetamide | |
| 171 | N-(4H-chromeno[4,3-d]thiazol-2-yl)-2-(1,3-dimethyl-2,6-dioxo-1,2,3,6- | D |
| | tetrahydropurin-7-yl)acetamide | |
| 172 | N-(7-chloro-4H-chromeno[4,3-d]thiazol-2-yl)-2-(1,3-dimethyl-2,6- | D |
| | dioxo-1,2,3,6-tetrahydropurin-7-yl)acetamide | |
| 173 | 2-(1,3-dimethyl-2,6-dioxo-1,2,3,6-tetrahydropurin-7-yl)-N-(7-ethoxy- | D |
| | 4H-chromeno[4,3-d]thiazol-2-yl)acetamide | |

In certain embodiments, the invention provides a method for treating or preventing a condition involving activation of TRPA1 or for which reduced TRPA1 activity can reduce the severity, comprising administering an effective amount of a compound of Formula II or a salt thereof, or a solvate, hydrate, oxidative metabolite or prodrug of the compound or its salt:

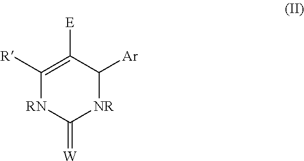

wherein
W represents O or S, preferably S;
R, independently for each occurrence, represents H or lower alkyl, preferably H;
R′ represents substituted or unsubstituted alkyl or substituted or unsubstituted aryl;
E represents carboxylic acid (CO₂H), ester or amide; and
Ar represents a substituted or unsubstituted aryl ring; and
wherein said compound inhibits TRPA1 with an with an IC₅₀ of 10 micromolar or less.

Examples of compounds within the above formula include:

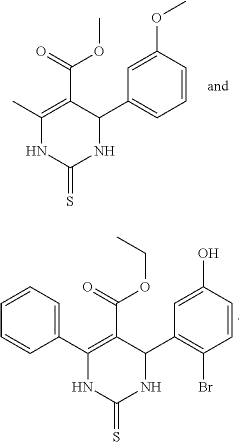

Further examples of compounds within formula I are shown in Table 2 along with their corresponding in vitro data.

In certain embodiments, the invention provides a method for treating or preventing a condition involving activation of TRPA1 or for which reduced TRPA1 activity can reduce the severity, comprising administering an effective amount of a compound of Formula III or a salt thereof, or a solvate, hydrate, oxidative metabolite or prodrug of the compound or its salt:

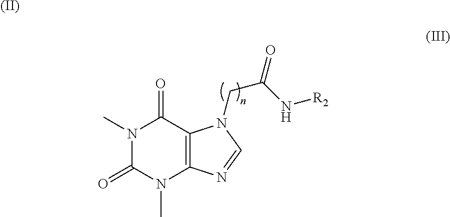

wherein
n is an integer from 1 to 3; and
R₂ represents a substituent; and
wherein said compound inhibits TRPA1 with an with an IC₅₀ of 10 micromolar or less.

In certain embodiments, R₂ represents optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

In certain embodiments, R₂ is not

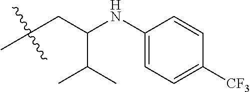

when n=1.

In certain embodiments, a compound of Formula II is 10-fold more selective for its TRPA1 activity than for its kininogenase inhibitory activity.

Examples of compounds of formula III include, but are not limited to, compounds 200-204, 206-243, 245-255, 257-282, 284-287, 289-290, 294-295, 298, 304-306, 308-310, 312-313, 316-317, 319, 321, 323, 325-326, 330, 332-333, 335, 337-341, 343-386, 389-390, 395-398, and 400-409, as are depicted in Table 2 along with their in vitro data.

In certain embodiments, the invention provides a method for treating or preventing a condition involving activation of TRPA1 or for which reduced TRPA1 activity can reduce the severity, comprising administering an effective amount of a compound of Formula IV or a salt thereof, or a solvate, hydrate, oxidative metabolite or prodrug of the compound or its salt:

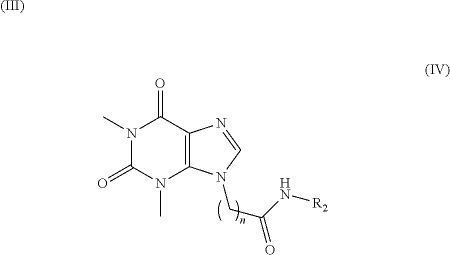

wherein
n is an integer from 1 to 3; and
R₂ represents a substituent; and
wherein said compound inhibits TRPA1 with an with an IC₅₀ of 10 micromolar or less.

In certain embodiments, R₂ represents optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

In certain embodiments, the invention provides a method for treating or preventing a condition involving activation of TRPA1 or for which reduced TRPA1 activity can reduce the severity, comprising administering an effective amount of a compound of Formula V or a salt thereof, or a solvate, hydrate, oxidative metabolite or prodrug of the compound or its salt:

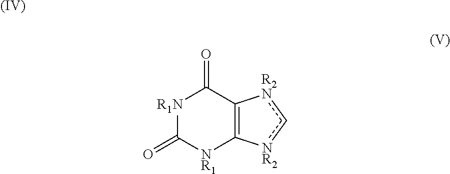

wherein
R₁, independently for each occurrence, represents H or lower alkyl;
one occurrence of R₂ is absent and one occurrence of R₂ is MₘR₃;
R₃ represents substituted or unsubstituted aryl;
M, independently for each occurrence, represents a substituted or unsubstituted methylene group (e.g., substituted with lower alkyl, oxo, hydroxyl, etc.), NR₁, O, S, S(O), or S(O₂), preferably selected such that no two heteroatoms are adjacent to each other; and
m is an integer from 0-10; and
wherein said compound inhibits TRPA1 with an with an IC₅₀ of 10 micromolar or less.

In certain embodiments, MₘR₃ represents

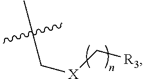

wherein
n is an integer between 0 and 4; and
X is —C(═O)O— or —C(═O)NR₄— wherein R₄ is H or lower alkyl, preferably —C(═O)NH—.

In certain embodiments, a compound of Formula V is 10-fold more selective for its TRPA1 activity than for its kininogenase inhibitory activity.

Examples of compounds of formula V include, but are not limited to, compounds 200-202, 204, 207-210, 212-215, 217, 219-224, 226-229, 231, 236-238, 240, 245-254, 256-268, 273, 275-277, 280-286, 288, 290-306, 309-311, 313-316, 318-320, 322, 324-331, 333-334, 336-338, 342, 344-347, 350-351, 353-355, 358-366, 371, 373-375, 377, 379-380, and 383-409, as are depicted in Table 2 along with their in vitro data.

TABLE 3

| | | |
|---|---|---|
| 1 | 2-(1,3-dimethyl-2,6-dioxo-2,3-dihydro-1H-purin-7(6H)-yl)-N-(4-p- | A |
| | tolylthiazol-2-yl)acetamide | |
| 2 | 2-(1,3-dimethyl-2,6-dioxo-2,3-dihydro-1H-purin-7(6H)-yl)-N-(4-(4- | A |
| | isopropylphenyl)thiazol-2-yl)acetamide | |
| 3 | 2-(1,3-dimethyl-2,6-dioxo-2,3-dihydro-1H-purin-7(6H)-yl)-N-(5- | A |
| | methyl-4-p-tolylthiazol-2-yl)acetamide | |
| 4 | N-(4-(4-tert-butylphenyl)thiazol-2-yl)-2-(1,3-dimethyl-2,6-dioxo-2,3- | A |
| | dihydro-1H-purin-7(6H)-yl)acetamide | |
| 5 | N-(4-(4-cyclohexylphenyl)thiazol-2-yl)-2-(1,3-dimethyl-2,6-dioxo-2,3- | A |
| | dihydro-1H-purin-7(6H)-yl)acetamide | |
| 6 | 2-(1,3-dimethyl-2,6-dioxo-2,3-dihydro-1H-purin-7(6H)-yl)-N-(4-(4- | A |
| | ethoxyphenyl)thiazol-2-yl)acetamide | |
| 7 | N-(4-(3,4-dichlorophenyl)thiazol-2-yl)-2-(1,3-dimethyl-2,6-dioxo-2,3- | A |
| | dihydro-1H-purin-7(6H)-yl)acetamide | |
| 8 | N-(4-(2,4-difluorophenyl)thiazol-2-yl)-2-(1,3-dimethyl-2,6-dioxo-2,3- | A |
| | dihydro-1H-purin-7(6H)-yl)acetamide | |
| 9 | 2-(1,3-dimethyl-2,6-dioxo-2,3-dihydro-1H-purin-7(6H)-yl)-N-(4,5- | A |
| | diphenylthiazol-2-yl)acetamide | |
| 10 | N-(4-(4-chlorophenyl)thiazol-2-yl)-2-(1,3-dimethyl-2,6-dioxo-2,3- | A |
| | dihydro-1H-purin-7(6H)-yl)acetamide | |
| 11 | N-(4-(3,4-difluorophenyl)thiazol-2-yl)-2-(1,3-dimethyl-2,6-dioxo-2,3- | A |
| | dihydro-1H-purin-7(6H)-yl)acetamide | |
| 12 | 2-(1,3-dimethyl-2,6-dioxo-2,3-dihydro-1H-purin-7(6H)-yl)-N-(5-(4- | D |
| | fluorophenyl)-1,3,4-thiadiazol-2-yl)acetamide | |
| 13 | 2-(1,3-dimethyl-2,6-dioxo-2,3-dihydro-1H-purin-7(6H)-yl)-N-(5- | D |
| | phenyl-1,3,4-thiadiazol-2-yl)acetamide | |
| 14 | N-(5-(4-chlorophenyl)-1,3,4-thiadiazol-2-yl)-2-(1,3-dimethyl-2,6-dioxo- | A |
| | 2,3-dihydro-1H-purin-7(6H)-yl)acetamide | |
| 15 | N-(4-(3-bromo-4-methoxyphenyl)thiazol-2-yl)-2-(1,3-dimethyl-2,6- | D |
| | dioxo-2,3-dihydro-1H-purin-7(6H)-yl)acetamide | |
| 16 | 2-(1,3-dimethyl-2,6-dioxo-2,3-dihydro-1H-purin-7(6H)-yl)-N-(4- | D |
| | (naphthalen-1-yl)thiazol-2-yl)acetamide | |
| 17 | 2-(1,3-dimethyl-2,6-dioxo-2,3-dihydro-1H-purin-7(6H)-yl)-N-(4- | A |
| | (naphthalen-2-yl)thiazol-2-yl)acetamide | |
| 18 | N-(4-(2,4-dichlorophenyl)thiazol-2-yl)-2-(1,3-dimethyl-2,6-dioxo-2,3- | D |
| | dihydro-1H-purin-7(6H)-yl)acetamide | |
| 19 | N-(4,5-dip-tolylthiazol-2-yl)-2-(1,3-dimethyl-2,6-dioxo-2,3-dihydro- | D |
| | 1H-purin-7(6H)-yl)acetamide | |
| 20 | N-(4-(2,5-dimethyl-1-phenyl-1H-pyrrol-3-yl)thiazol-2-yl)-2-(1,3- | D |
| | dimethyl-2,6-dioxo-2,3-dihydro-1H-purin-7(6H)-yl)acetamide | |
| 21 | N-(4-(1H-indol-3-yl)thiazol-2-yl)-2-(1,3-dimethyl-2,6-dioxo-2,3- | D |
| | dihydro-1H-purin-7(6H)-yl)acetamid | |
| 22 | 2-(1,3-dimethyl-2,6-dioxo-2,3-dihydro-1H-purin-7(6H)-yl)-N-(4-(5- | B |
| | methylfuran-2-yl)thiazol-2-yl)acetamide | |
| 23 | 2-(1,3-dimethyl-2,6-dioxo-2,3-dihydro-1H-purin-7(6H)-yl)-N-(4- | B |
| | (pyridin-2-yl)thiazol-2-yl)acetamide | |
| 24 | N-(4-(4-bromophenyl)-6-(trifluoromethyl)pyrimidin-2-yl)-2-(1,3- | D |
| | dimethyl-2,6-dioxo-2,3-dihydro-1H-purin-7(6H)-yl)acetamide | |
| 25 | 2-(1,3-dimethyl-2,6-dioxo-2,3-dihydro-1H-purin-7(6H)-yl)-N-(4-(2,4- | D |
| | dimethylphenyl)thiazol-2-yl)acetamide | |
| 26 | 2-(1,3-dimethyl-2,6-dioxo-2,3-dihydro-1H-purin-7(6H)-yl)-N-(4-(4- | D |
| | fluorophenyl)thiazol-2-yl)acetamide | |
| 27 | N-(4-(2,5-dimethoxyphenyl)thiazol-2-yl)-2-(1,3-dimethyl-2,6-dioxo- | D |
| | 2,3-dihydro-1H-purin-7(6H)-yl)acetamide | |
| 28 | N-(4-(2,5-difluorophenyl)thiazol-2-yl)-2-(1,3-dimethyl-2,6-dioxo-2,3- | D |
| | dihydro-1H-purin-7(6H)-yl)acetamide | |
| 29 | N-(4-(2,3-dihydrobenzo[b][1,4]dioxin-6-yl)thiazol-2-yl)-2-(1,3- | D |
| | dimethyl-2,6-dioxo-2,3-dihydro-1H-purin-7(6H)-yl)acetamide | |
| 30 | 2-(1,3-dimethyl-2,6-dioxo-2,3-dihydro-1H-purin-7(6H)-yl)-N-(4-(5- | D |
| | methylthiophen-2-yl)thiazol-2-yl)acetamide | |
| 31 | N-allyl-2-(8-chloro-1,3-dimethyl-2,6-dioxo-2,3-dihydro-1H-purin- | D |
| | 7(6H)-yl)-N-(4-phenylthiazol-2-yl)acetamide | |
| 32 | 2-(1,3-dimethyl-2,6-dioxo-2,3-dihydro-1H-purin-7(6H)-yl)-N-(4- | D |
| | (5,6,7,8-tetrahydronaphthalen-2-yl)thiazol-2-yl)acetamide | |
| 33 | 2-(8-chloro-1,3-dimethyl-2,6-dioxo-1,2,3,6-tetrahydropurin-7-yl)-N-(4- | D |
| | (adamant-1-yl)phenyl)acetamide; | |
| 34 | 2-(8-chloro-1,3-dimethyl-2,6-dioxo-1,2,3,6-tetrahydropurin-7-yl)-N-(4- | D |
| | (4-ethylphenyl)thiazol-2-yl)acetamide; | |
| 35 | N-(2-(1,3-dimethyl-2,6-dioxo-1,2,3,6-tetrahydropurin-7-yl)ethyl)-3-(2- | D |
| | methylpyrimidin-4-yl)benzamide; | |
| 36 | 2-(1,3-dimethyl-2,6-dioxo-1,2,3,6-tetrahydropurin-7-yl)-N-(4-(4- | D |
| | ethoxyphenyl)-1H-imidazol-2-yl)acetamide | |
| 37 | 2-(1,3-dimethyl-2,6-dioxo-1,2,3,6-tetrahydropurin-7-yl)-N-(4-(4- | D |
| | ethoxyphenyl)-5-methyl-1H-imidazol-2-yl)acetamide | |
| 38 | 2-(1,3-dimethyl-2,6-dioxo-1,2,3,6-tetrahydropurin-7-yl)-N-(4-(4- | D |
| | ethoxyphenyl)-5-phenyl-1H-imidazol-2-yl)acetamide | |
| 39 | 2-(1,3-dimethyl-2,6-dioxo-1,2,3,6-tetrahydropurin-7-yl)-N-(4-(4- | D |
| | ethoxyphenyl)-1-methyl-1H-imidazol-2-yl)acetamide | |
| 40 | 2-(1,3-dimethyl-2,6-dioxo-1,2,3,6-tetrahydropurin-7-yl)-N-(4-(4- | D |
| | ethoxyphenyl)-1,5-dimethyl-1H-imidazol-2-yl)acetamide | |
| 41 | 2-(1,3-dimethyl-2,6-dioxo-1,2,3,6-tetrahydropurin-7-yl)-N-(4-(4- | D |
| | ethoxyphenyl)-1-methyl-5-phenyl-1H-imidazol-2-yl)acetamide | |
| 42 | 2-(1,3-dimethyl-2,6-dioxo-1,2,3,6-tetrahydropurin-7-yl)-N-(4-(4- | D |
| | ethoxyphenyl)-1-ethyl-1H-imidazol-2-yl)acetamide | |
| 43 | 2-(1,3-dimethyl-2,6-dioxo-1,2,3,6-tetrahydropurin-7-yl)-N-(4-(4- | D |
| | ethoxyphenyl)-1-ethyl-5-methyl-1H-imidazol-2-yl)acetamide | |
| 44 | 2-(1,3-dimethyl-2,6-dioxo-1,2,3,6-tetrahydropurin-7-yl)-N-(4-(4- | D |
| | ethoxyphenyl)-1-ethyl-5-phenyl-1H-imidazol-2-yl)acetamide | |
| 45 | 2-(1,3-dimethyl-2,6-dioxo-1,2,3,6-tetrahydropurin-7-yl)-N-(4-(4- | D |
| | ethoxyphenyl)-1-hydroxy-1H-imidazol-2-yl)acetamide | |
| 46 | 2-(1,3-dimethyl-2,6-dioxo-1,2,3,6-tetrahydropurin-7-yl)-N-(4-(4- | D |
| | ethoxyphenyl)-1-hydroxy-5-methyl-1H-imidazol-2-yl)acetamide | |
| 47 | 2-(1,3-dimethyl-2,6-dioxo-1,2,3,6-tetrahydropurin-7-yl)-N-(4-(4- | D |
| | ethoxyphenyl)-1-hydroxy-5-phenyl-1H-imidazol-2-yl)acetamide | |
| 48 | 2-(1,3-dimethyl-2,6-dioxo-1,2,3,6-tetrahydropurin-7-yl)-N-(4-(4- | D |
| | methoxyphenyl)thiazol-2-yl)acetamide | |
| 49 | 2-(1,3-dimethyl-2,6-dioxo-1,2,3,6-tetrahydropurin-7-yl)-N-(4-(4- | D |
| | isopropoxyphenyl)thiazol-2-yl)acetamide | |
| 50 | 2-(1,3-dimethyl-2,6-dioxo-1,2,3,6-tetrahydropurin-7-yl)-N-(4-(4- | D |
| | (dimethylamino)phenyl)thiazol-2-yl)acetamide | |
| 51 | N-(4-(4-(diethylamino)phenyl)thiazol-2-yl)-2-(1,3-dimethyl-2,6-dioxo- | D |
| | 1,2,3,6-tetrahydropurin-7-yl)acetamide | |
| 52 | N-(4-(4-acetamidophenyl)thiazol-2-yl)-2-(1,3-dimethyl-2,6-dioxo- | D |
| | 1,2,3,6-tetrahydropurin-7-yl)acetamide | |
| 53 | 2-(1,3-dimethyl-2,6-dioxo-1,2,3,6-tetrahydropurin-7-yl)-N-(4-(4- | D |
| | ureidophenyl)thiazol-2-yl)acetamide | |
| 54 | N-(4-(4-cyanophenyl)thiazol-2-yl)-2-(1,3-dimethyl-2,6-dioxo-1,2,3,6- | D |
| | tetrahydropurin-7-yl)acetamide | |
| 55 | 2-(1,3-dimethyl-2,6-dioxo-1,2,3,6-tetrahydropurin-7-yl)-N-(4-(4-(2- | D |
| | hydroxyethoxy)phenyl)thiazol-2-yl)acetamide | |
| 56 | N-(4-(2-chlorophenyl)thiazol-2-yl)-2-(1,3-dimethyl-2,6-dioxo-1,2,3,6- | D |
| | tetrahydropurin-7-yl)acetamide | |
| 57 | N-(4-(3-chlorophenyl)thiazol-2-yl)-2-(1,3-dimethyl-2,6-dioxo-1,2,3,6- | D |
| | tetrahydropurin-7-yl)acetamide | |
| 58 | 2-(1,3-dimethyl-2,6-dioxo-1,2,3,6-tetrahydropurin-7-yl)-N-(4-(2- | D |
| | ethoxyphenyl)thiazol-2-yl)acetamide | |
| 59 | 2-(1,3-dimethyl-2,6-dioxo-1,2,3,6-tetrahydropurin-7-yl)-N-(4-(3- | D |
| | ethoxyphenyl)thiazol-2-yl)acetamide | |
| 60 | 2-(1,3-dimethyl-2,6-dioxo-1,2,3,6-tetrahydropurin-7-yl)-N-(4-(2- | D |
| | methoxyphenyl)thiazol-2-yl)acetamide | |
| 61 | 2-(1,3-dimethyl-2,6-dioxo-1,2,3,6-tetrahydropurin-7-yl)-N-(4-(3- | D |
| | methoxyphenyl)thiazol-2-yl)acetamide | |
| 62 | N-(4-(2-bromophenyl)thiazol-2-yl)-2-(1,3-dimethyl-2,6-dioxo-1,2,3,6- | D |
| | tetrahydropurin-7-yl)acetamide | |
| 63 | N-(4-(3-bromophenyl)thiazol-2-yl)-2-(1,3-dimethyl-2,6-dioxo-1,2,3,6- | D |
| | tetrahydropurin-7-yl)acetamide | |
| 64 | 2-(1,3-dimethyl-2,6-dioxo-1,2,3,6-tetrahydropurin-7-yl)-N-(4-(2- | D |
| | fluorophenyl)thiazol-2-yl)acetamide | |
| 65 | 2-(1,3-dimethyl-2,6-dioxo-1,2,3,6-tetrahydropurin-7-yl)-N-(4-(3- | D |
| | fluorophenyl)thiazol-2-yl)acetamide | |
| 66 | 2-(1,3-dimethyl-2,6-dioxo-1,2,3,6-tetrahydropurin-7-yl)-N-(4-o- | D |
| | tolylthiazol-2-yl)acetamide | |
| 67 | 2-(1,3-dimethyl-2,6-dioxo-1,2,3,6-tetrahydropurin-7-yl)-N-(4-m- | D |
| | tolylthiazol-2-yl)acetamide | |
| 68 | 2-(1,3-dimethyl-2,6-dioxo-1,2,3,6-tetrahydropurin-7-yl)-N-(4-(2-(2- | D |
| | hydroxyethoxy)phenyl)thiazol-2-yl)acetamide | |
| 69 | 2-(1,3-dimethyl-2,6-dioxo-1,2,3,6-tetrahydropurin-7-yl)-N-(4-(3-(2- | D |
| | hydroxyethoxy)phenyl)thiazol-2-yl)acetamide | |
| 70 | 2-(1,3-dimethyl-2,6-dioxo-1,2,3,6-tetrahydropurin-7-yl)-N-(4-(3,4,5- | D |
| | trichlorophenyl)thiazol-2-yl)acetamide | |
| 71 | 2-(1,3-dimethyl-2,6-dioxo-1,2,3,6-tetrahydropurin-7-yl)-N-(4-(2,3,4- | D |
| | trichlorophenyl)thiazol-2-yl)acetamide | |
| 72 | 2-(1,3-dimethyl-2,6-dioxo-1,2,3,6-tetrahydropurin-7-yl)-N-(4-(2,4,5- | D |
| | trichlorophenyl)thiazol-2-yl)acetamide | |
| 73 | N-(4-(3,4-dimethoxyphenyl)thiazol-2-yl)-2-(1,3-dimethyl-2,6-dioxo- | D |
| | 1,2,3,6-tetrahydropurin-7-yl)acetamide | |
| 74 | N-(4-(3-chloro-4-ethoxyphenyl)thiazol-2-yl)-2-(1,3-dimethyl-2,6-dioxo- | D |
| | 1,2,3,6-tetrahydropurin-7-yl)acetamide | |
| 75 | N-(4-(4-chloro-3-ethoxyphenyl)thiazol-2-yl)-2-(1,3-dimethyl-2,6-dioxo- | D |
| | 1,2,3,6-tetrahydropurin-7-yl)acetamide | |
| 76 | N-(4-(2-chloro-4-ethoxyphenyl)thiazol-2-yl)-2-(1,3-dimethyl-2,6-dioxo- | D |
| | 1,2,3,6-tetrahydropurin-7-yl)acetamide | |
| 77 | N-(4-(2,4-dichlorophenyl)thiazol-2-yl)-2-(1,3-dimethyl-2,6-dioxo- | D |
| | 1,2,3,6-tetrahydropurin-7-yl)acetamide | |
| 78 | 2-(1,3-dimethyl-2,6-dioxo-1,2,3,6-tetrahydropurin-7-yl)-N-(4-(2,4,6- | D |
| | trichlorophenyl)thiazol-2-yl)acetamide | |
| 79 | 2-(1,3-dimethyl-2,6-dioxo-1,2,3,6-tetrahydropurin-7-yl)-N-(4- | D |
| | phenylthiazol-2-yl)acetamide | |
| 80 | 2-(1,3-dimethyl-2,6-dioxo-1,2,3,6-tetrahydropurin-7-yl)-N-(4,5- | D |
| | diphenylthiazol-2-yl)acetamide | |
| 81 | 2-(1,3-dimethyl-2,6-dioxo-1,2,3,6-tetrahydropurin-7-yl)-N-(4,5- | D |
| | di(methylphenyl)thiazol-2-yl)acetamide | |
| 82 | N-(4-(3,4-dichlorophenyl)-5-phenylthiazol-2-yl)-2-(1,3-dimethyl-2,6- | D |
| | dioxo-1,2,3,6-tetrahydropurin-7-yl)acetamide | |
| 83 | N-(4-(3,4-dichlorophenyl)-5-(methylphenyl)thiazol-2-yl)-2-(1,3- | D |
| | dimethyl-2,6-dioxo-1,2,3,6-tetrahydropurin-7-yl)acetamide | |
| 84 | N-(4-(3,4-dichlorophenyl)-5-(4-ethoxyphenyl)thiazol-2-yl)-2-(1,3- | D |
| | dimethyl-2,6-dioxo-1,2,3,6-tetrahydropurin-7-yl)acetamide | |
| 85 | N-(4,5-bis(3,4-dichlorophenyl)thiazol-2-yl)-2-(1,3-dimethyl-2,6-dioxo- | D |
| | 1,2,3,6-tetrahydropurin-7-yl)acetamide | |
| 86 | N-(4-(4-ethoxyphenyl)-5-phenylthiazol-2-yl)-2-(1,3-dimethyl-2,6- | D |
| | dioxo-1,2,3,6-tetrahydropurin-7-yl)acetamide | |
| 87 | N-(4-(4-ethoxyphenyl)-5-(methylphenyl)thiazol-2-yl)-2-(1,3-dimethyl- | D |
| | 2,6-dioxo-1,2,3,6-tetrahydropurin-7-yl)acetamide | |
| 88 | N-(4-(4-ethoxyphenyl)-5-(4-ethoxyphenyl)thiazol-2-yl)-2-(1,3- | D |
| | dimethyl-2,6-dioxo-1,2,3,6-tetrahydropurin-7-yl)acetamide | |
| 89 | N-(5-(3,4-dichlorophenyl)-4-(4-ethoxyphenyl)thiazol-2-yl)-2-(1,3- | D |
| | dimethyl-2,6-dioxo-1,2,3,6-tetrahydropurin-7-yl)acetamide | |
| 90 | 2-(1,3-dimethyl-2,6-dioxo-1,2,3,6-tetrahydropurin-7-yl)-N-(5- | D |
| | phenylthiazol-2-yl)acetamide | |
| 91 | 2-(1,3-dimethyl-2,6-dioxo-1,2,3,6-tetrahydropurin-7-yl)-N-(5- | D |
| | (methylphenyl)thiazol-2-yl)acetamide | |
| 92 | 2-(1,3-dimethyl-2,6-dioxo-1,2,3,6-tetrahydropurin-7-yl)-N-(5- | D |
| | (chlorophenyl)thiazol-2-yl)acetamide | |
| 93 | 2-(1,3-dimethyl-2,6-dioxo-1,2,3,6-tetrahydropurin-7-yl)-N-(5- | D |
| | (methoxyphenyl)thiazol-2-yl)acetamide | |
| 94 | 2-(1,3-dimethyl-2,6-dioxo-1,2,3,6-tetrahydropurin-7-yl)-N-(5- | D |
| | (ethoxyphenyl)thiazol-2-yl)acetamide | |
| 95 | 2-(1,3-dimethyl-2,6-dioxo-1,2,3,6-tetrahydropurin-7-yl)-N-(4-(pyridin- | D |
| | 4-yl)thiazol-2-yl)acetamide | |
| 96 | 2-(1,3-dimethyl-2,6-dioxo-1,2,3,6-tetrahydropurin-7-yl)-N-(4-(pyridin- | D |
| | 3-yl)thiazol-2-yl)acetamide | |
| 97 | 2-(1,3-dimethyl-2,6-dioxo-1,2,3,6-tetrahydropurin-7-yl)-N-(4- | D |
| | (pyrimidin-2-yl)thiazol-2-yl)acetamide | |
| 98 | 2-(1,3-dimethyl-2,6-dioxo-1,2,3,6-tetrahydropurin-7-yl)-N-(4- | D |
| | phenyloxazol-2-yl)acetamide | |
| 99 | 2-(1,3-dimethyl-2,6-dioxo-1,2,3,6-tetrahydropurin-7-yl)-N-(5-ethyl-4- | D |
| | phenyloxazol-2-yl)acetamide | |
| 100 | 2-(1,3-dimethyl-2,6-dioxo-1,2,3,6-tetrahydropurin-7-yl)-N-(4,5- | D |
| | diphenyloxazol-2-yl)acetamide | |
| 101 | N-(4,5-dip-tolyloxazol-2-yl)-2-(1,3-dimethyl-2,6-dioxo-1,2,3,6- | D |
| | tetrahydropurin-7-yl)acetamide | |
| 102 | N-(4,5-bis(4-methoxyphenyl)oxazol-2-yl)-2-(1,3-dimethyl-2,6-dioxo- | D |
| | 1,2,3,6-tetrahydropurin-7-yl)acetamide | |
| 103 | N-(4-(3,4-dichlorophenyl)-5-fluorothiazol-2-yl)-2-(1,3-dimethyl-2,6- | D |
| | dioxo-1,2,3,6-tetrahydropurin-7-yl)acetamide | |
| 104 | N-(5-cyano-4-(3,4-dichlorophenyl)thiazol-2-yl)-2-(1,3-dimethyl-2,6- | D |
| | dioxo-1,2,3,6-tetrahydropurin-7-yl)acetamide | |
| 105 | N-(4-(3,4-dichlorophenyl)-5-methoxythiazol-2-yl)-2-(1,3-dimethyl-2,6- | D |
| | dioxo-1,2,3,6-tetrahydropurin-7-yl)acetamide | |
| 106 | N-(4-(3,4-dichlorophenyl)-5-methylthiazol-2-yl)-2-(1,3-dimethyl-2,6- | D |
| | dioxo-1,2,3,6-tetrahydropurin-7-yl)acetamide | |
| 107 | N-(4-(3,4-dichlorophenyl)-5-ethylthiazol-2-yl)-2-(1,3-dimethyl-2,6- | D |
| | dioxo-1,2,3,6-tetrahydropurin-7-yl)acetamide | |
| 108 | N-(4-(3,4-dichlorophenyl)-5-(trifluoromethyl)thiazol-2-yl)-2-(1,3- | D |
| | dimethyl-2,6-dioxo-1,2,3,6-tetrahydropurin-7-yl)acetamide | |
| 109 | 2-(4-(3,4-dichlorophenyl)-2-(2-(1,3-dimethyl-2,6-dioxo-1,2,3,6- | D |
| | tetrahydropurin-7-yl)acetamido)thiazol-5-yl)ethyl dihydrogen phosphate | |
| 110 | N-(4-(3,4-dichlorophenyl)-5-methylthiazol-2-yl)-2-(1,3-dimethyl-2,6- | D |
| | dioxo-1,2,3,6-tetrahydropurin-7-yl)acetamide | |
| 111 | N-(4-(3,4-dichlorophenyl)-5-ethylthiazol-2-yl)-2-(1,3-dimethyl-2,6- | D |
| | dioxo-1,2,3,6-tetrahydropurin-7-yl)acetamide | |
| 112 | N-(4-(3,4-dichlorophenyl)-5-isopropylthiazol-2-yl)-2-(1,3-dimethyl-2,6- | D |
| | dioxo-1,2,3,6-tetrahydropurin-7-yl)acetamide | |
| 113 | N-(4-(3,4-dichlorophenyl)-5-(2-hydroxyethyl)thiazol-2-yl)-2-(1,3- | D |
| | dimethyl-2,6-dioxo-1,2,3,6-tetrahydropurin-7-yl)acetamide | |

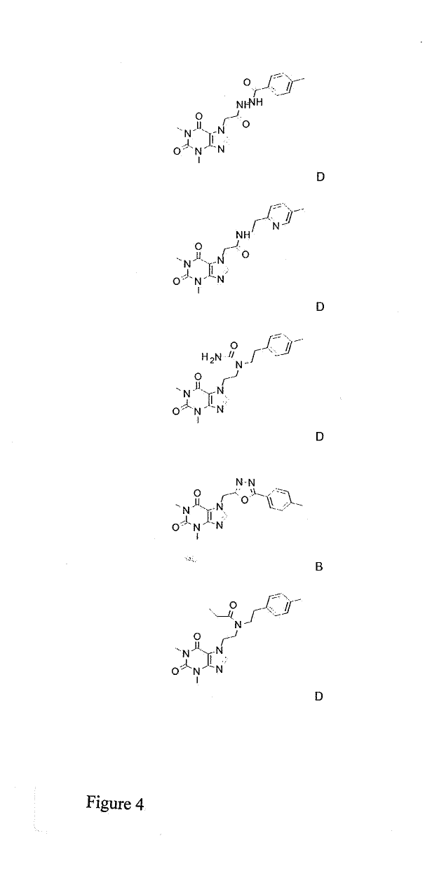

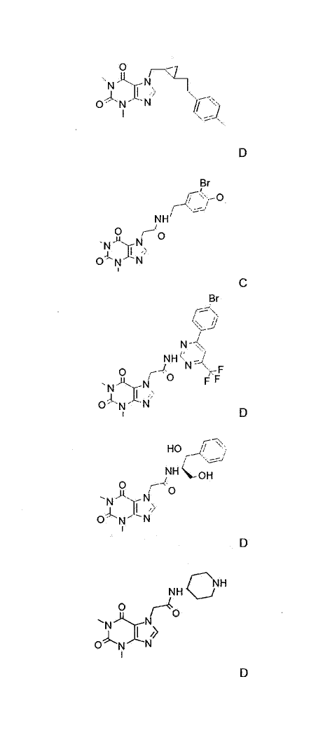

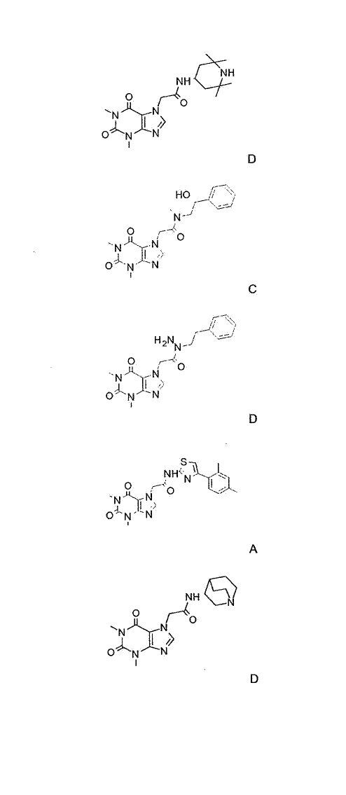

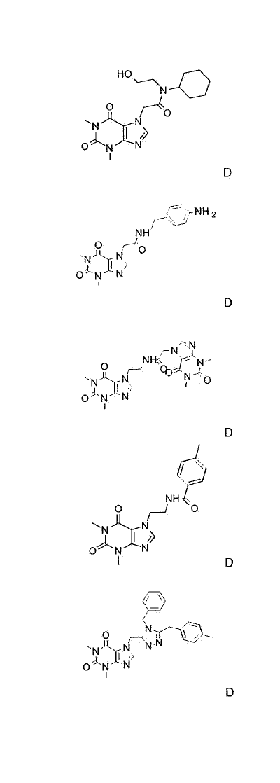

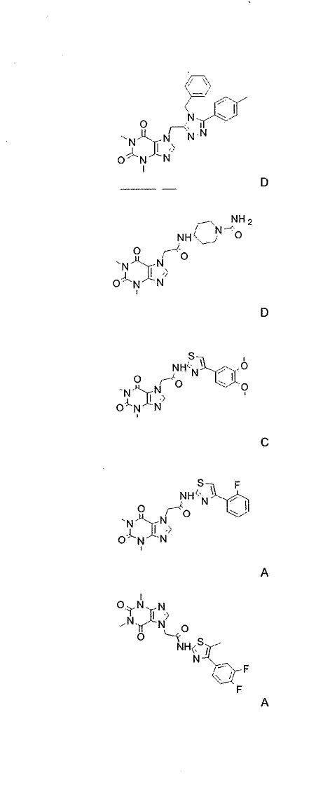

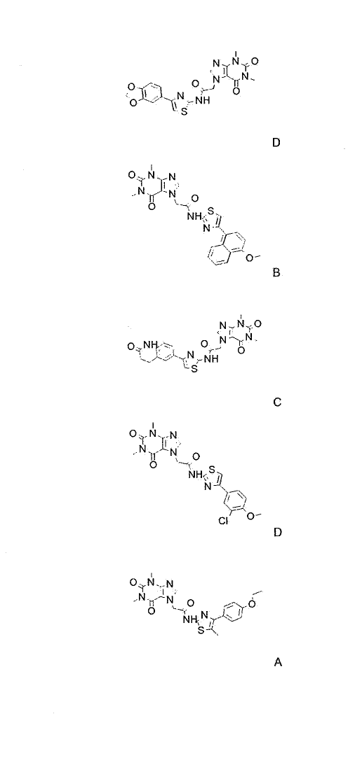

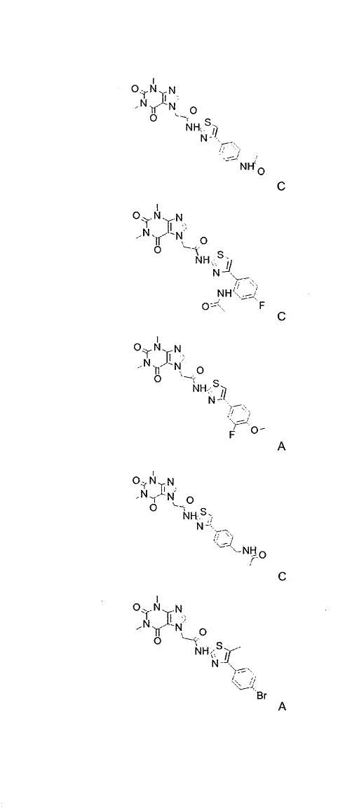

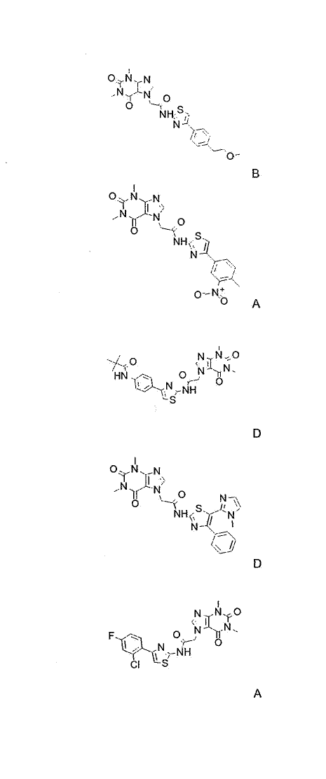

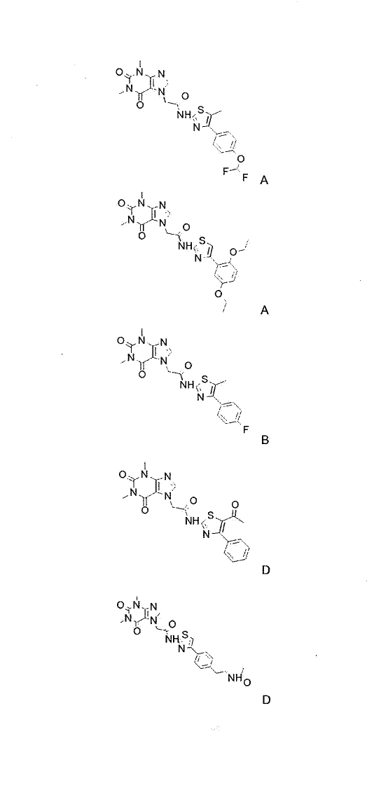

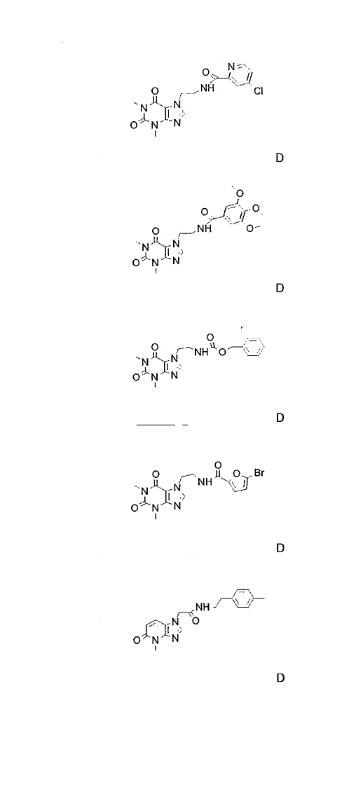

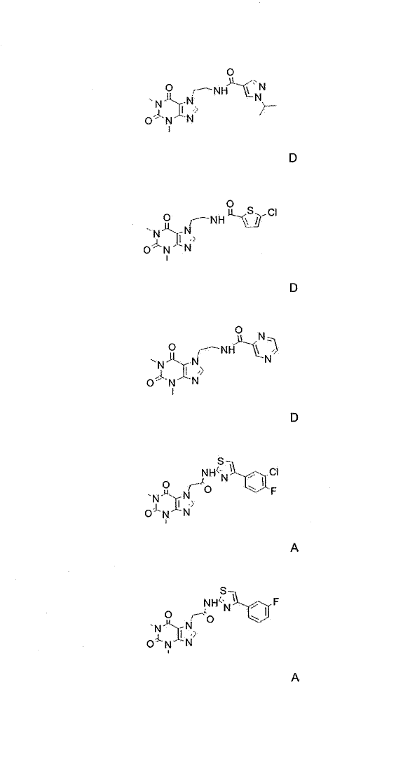

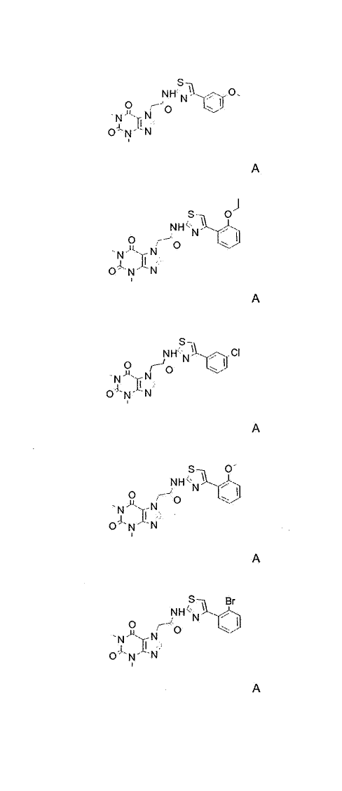

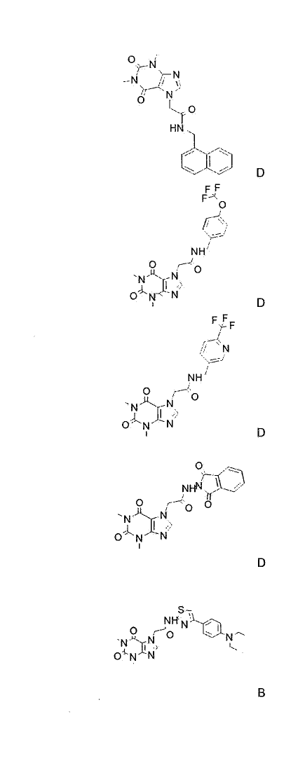

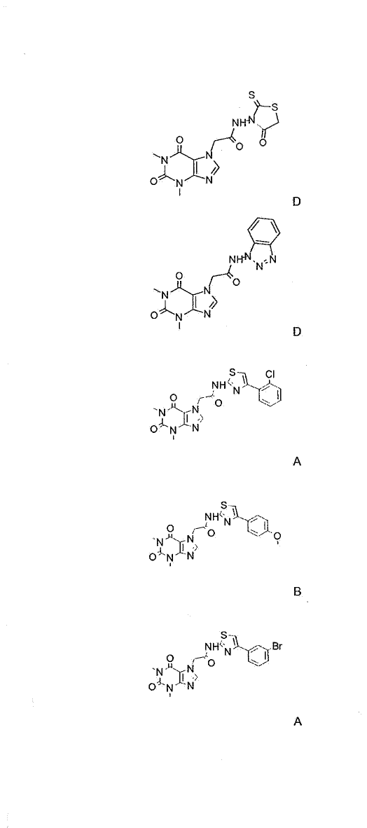

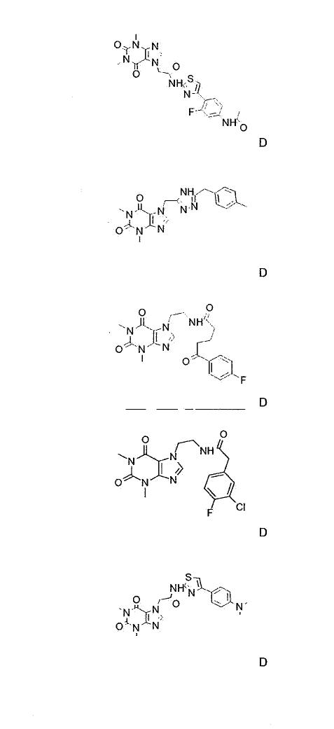

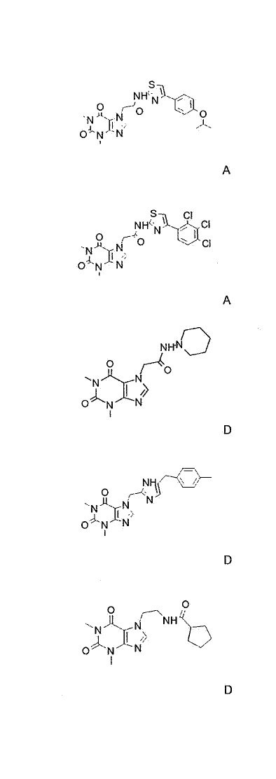

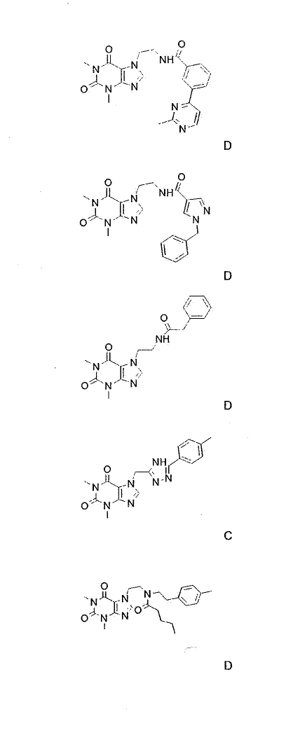

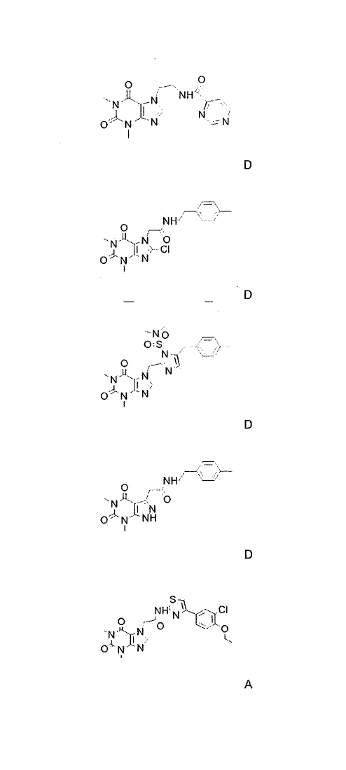

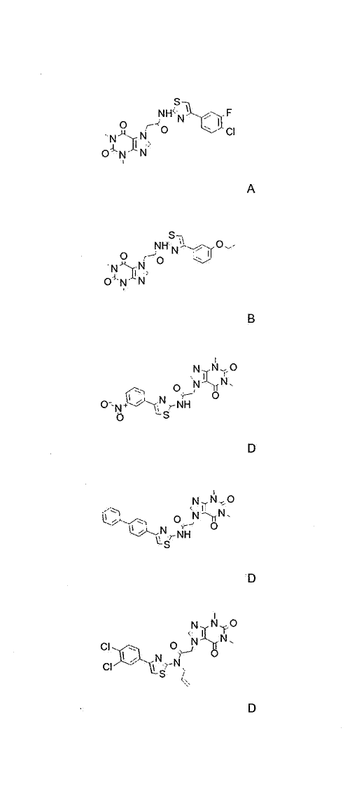

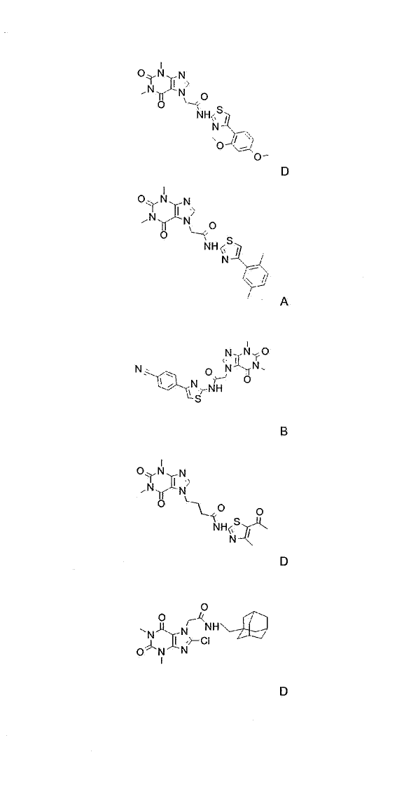

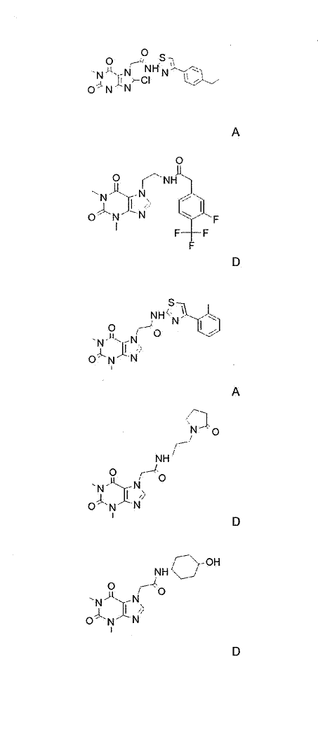

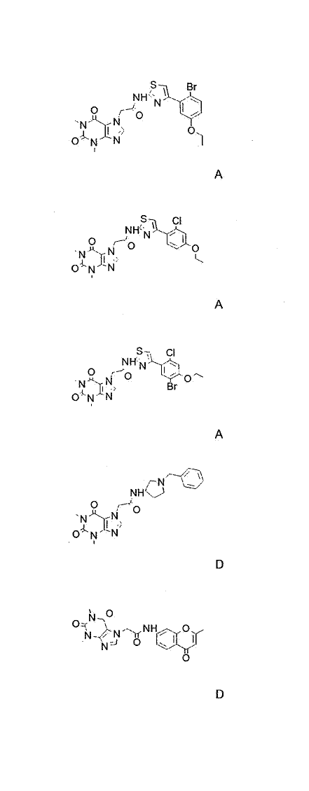

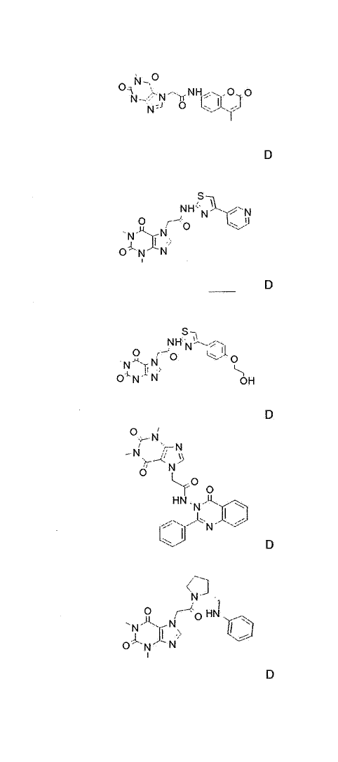

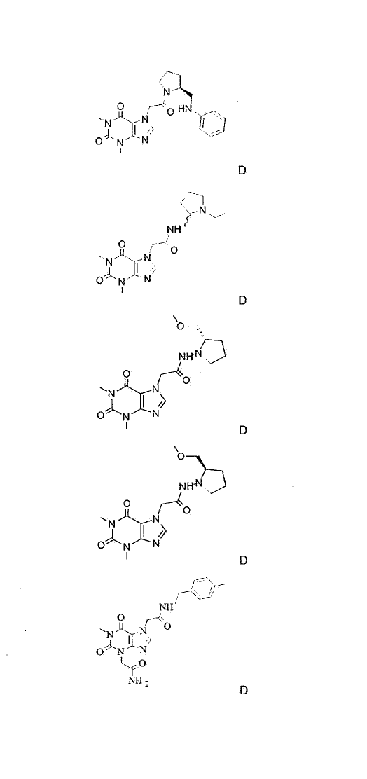

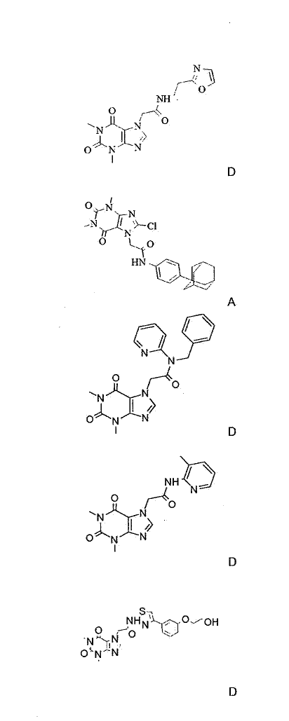

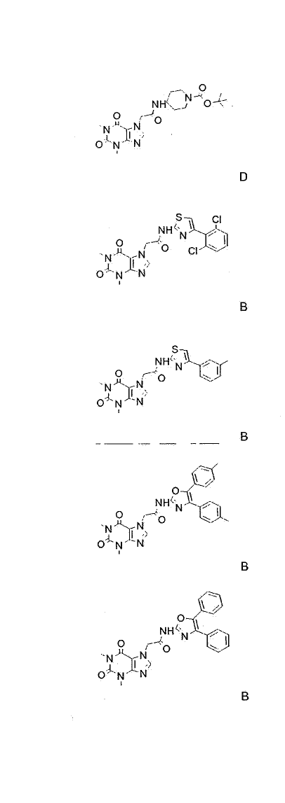

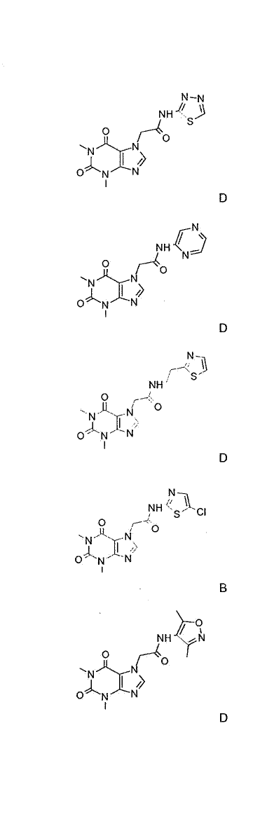

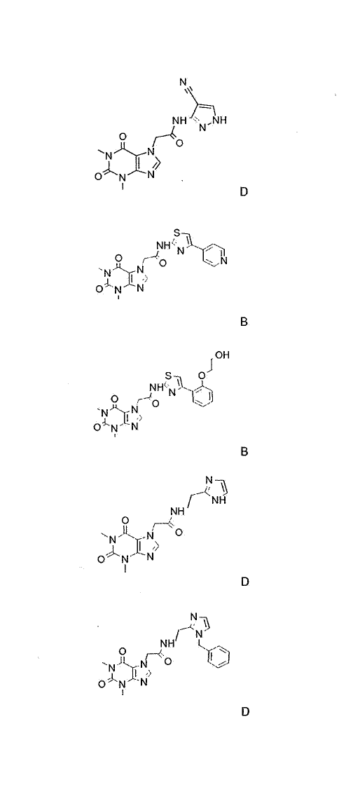

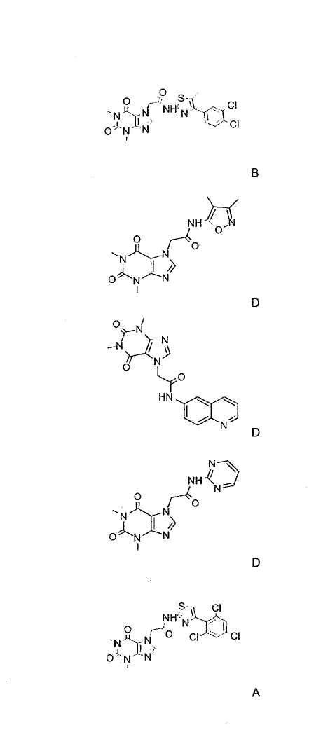

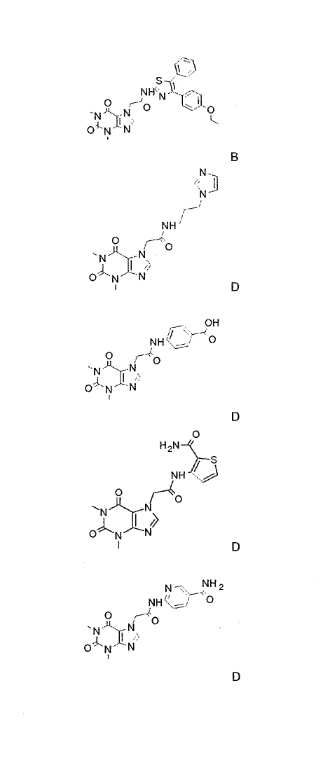

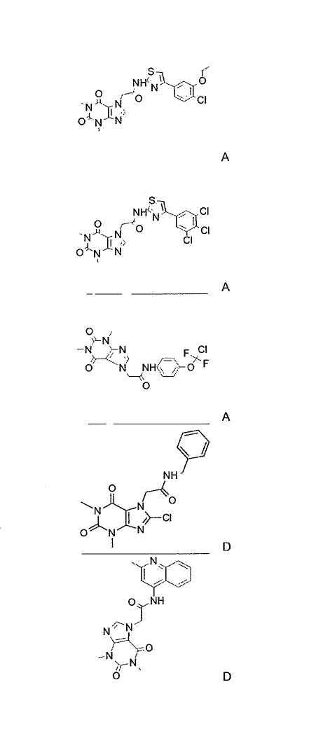

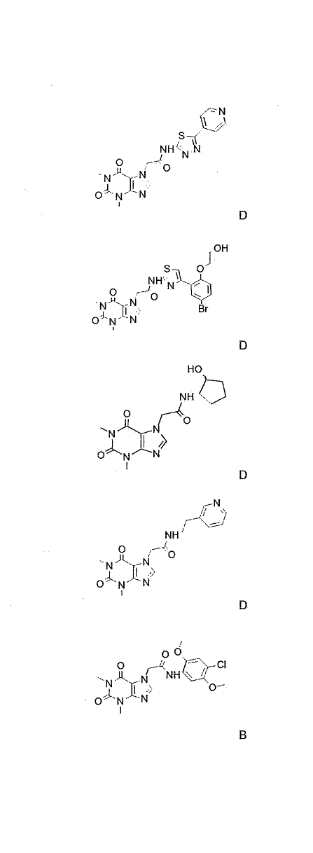

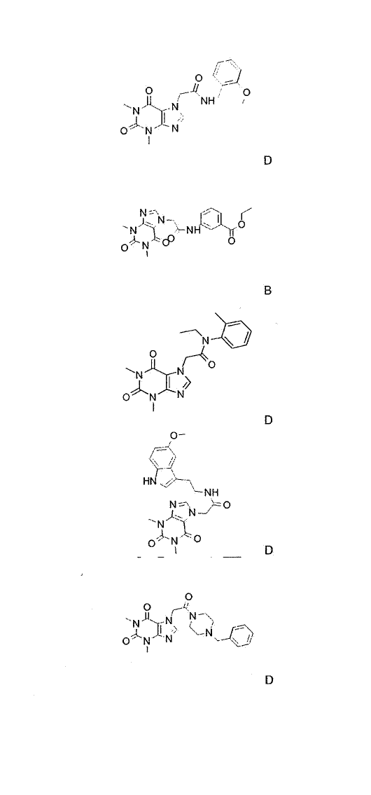

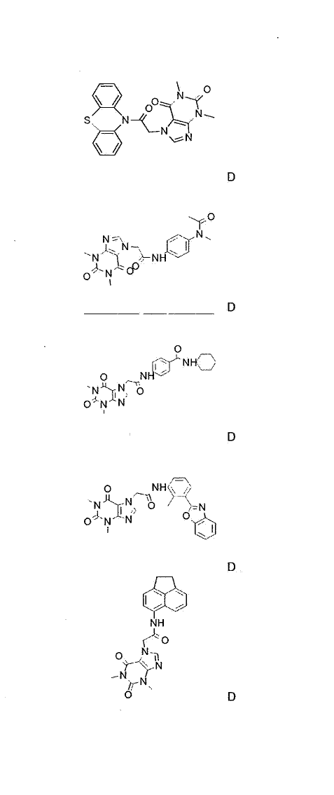

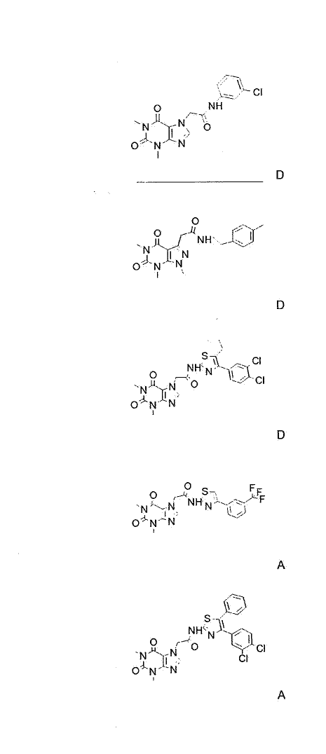

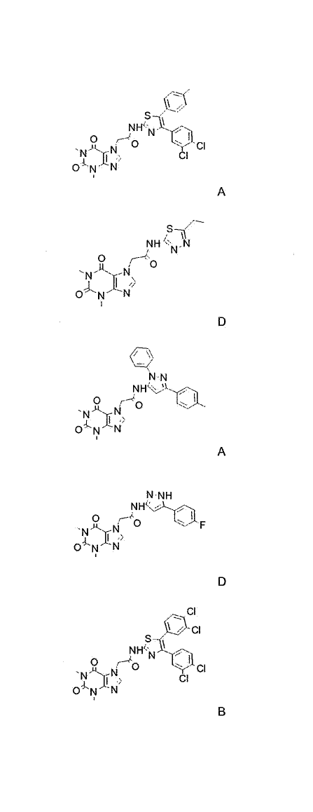

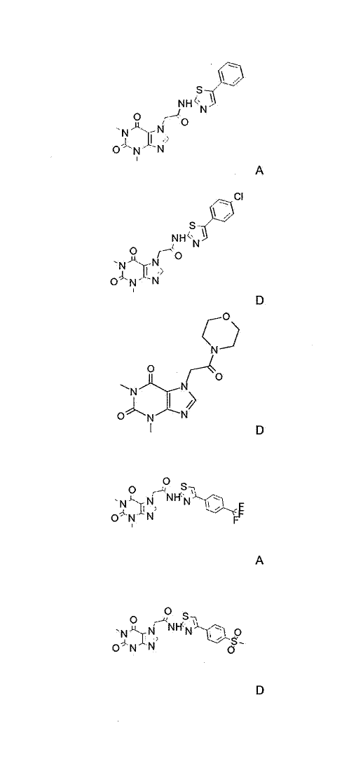

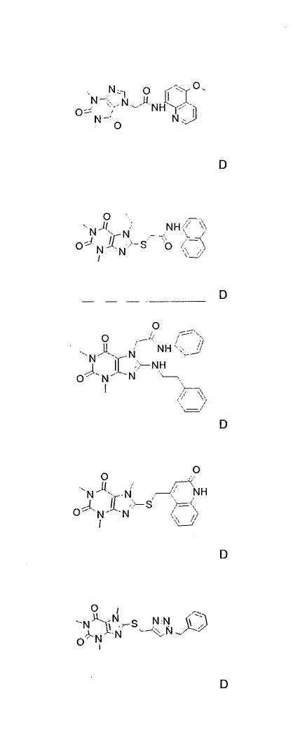

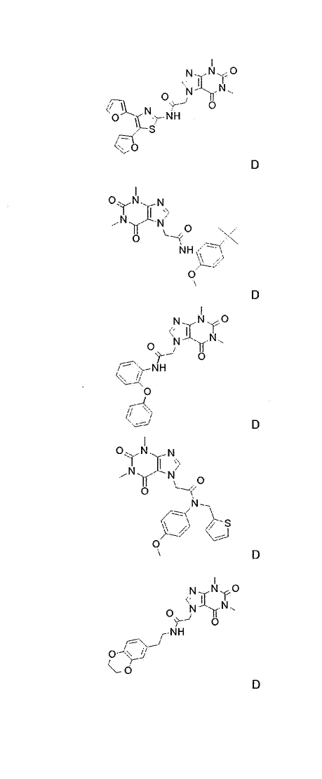

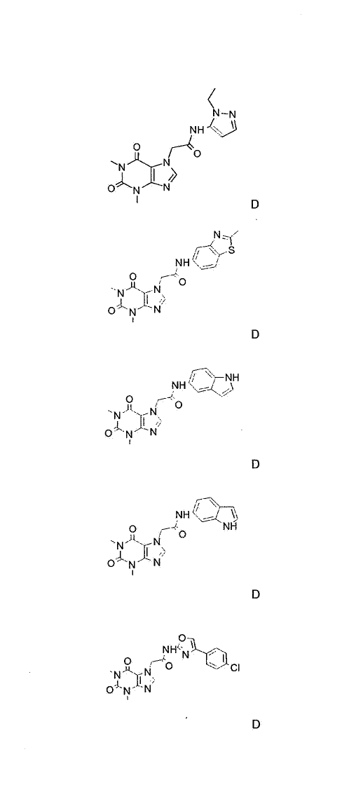

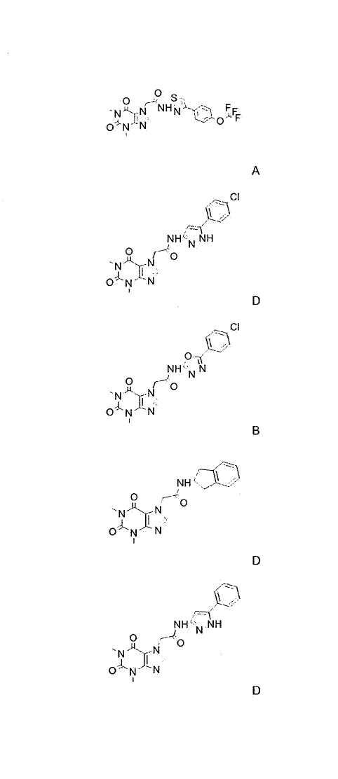

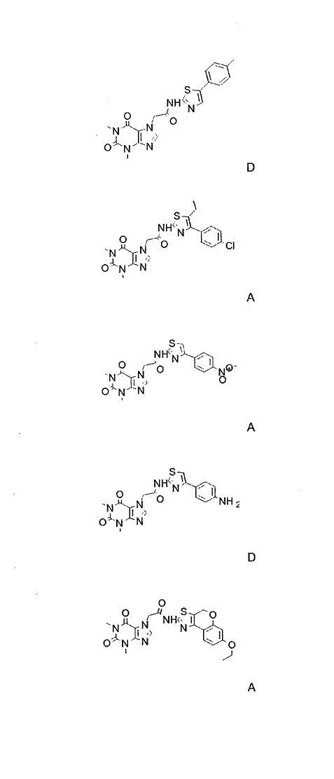

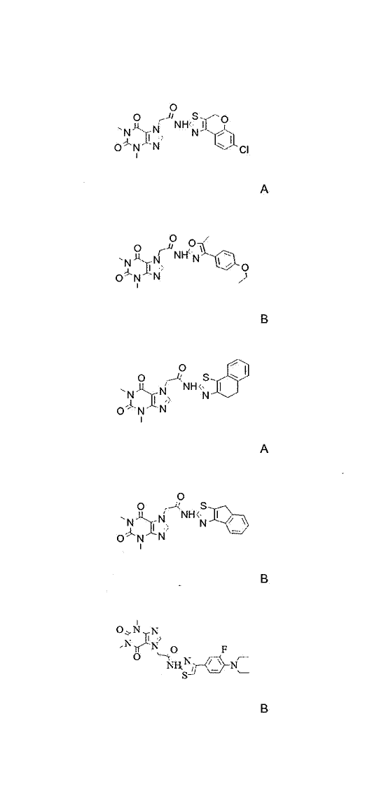

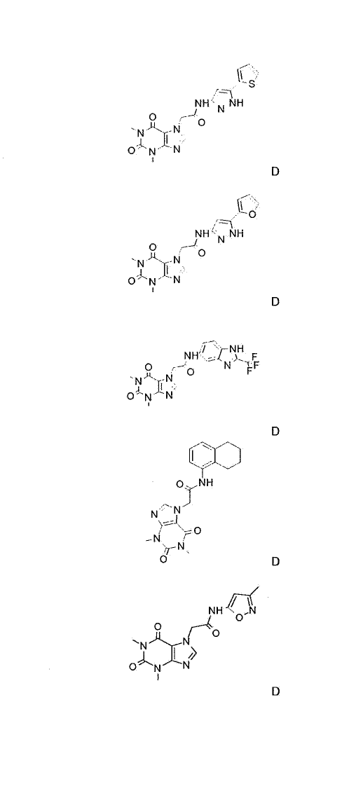

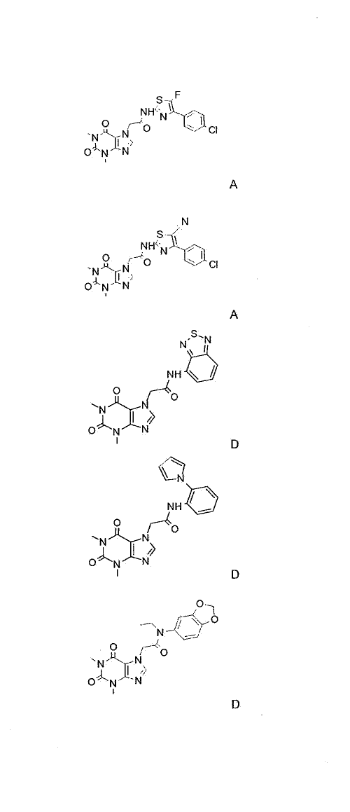

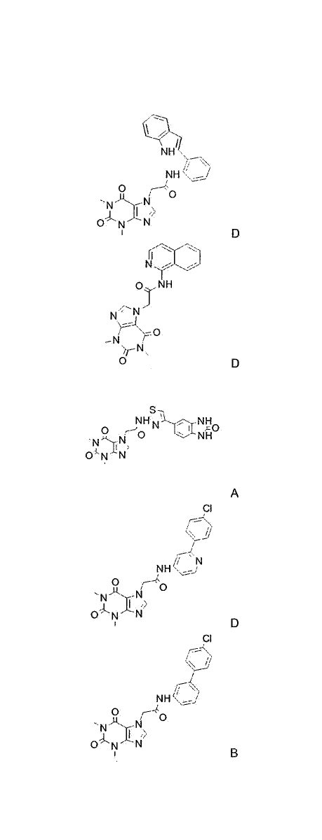

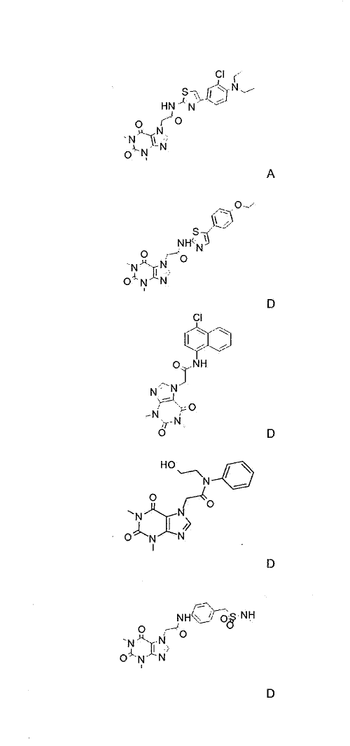

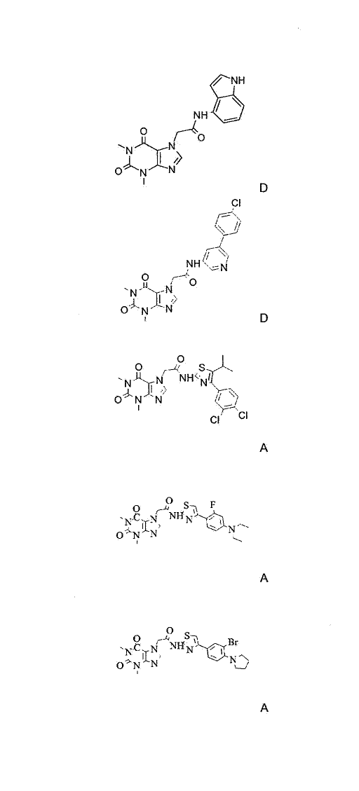

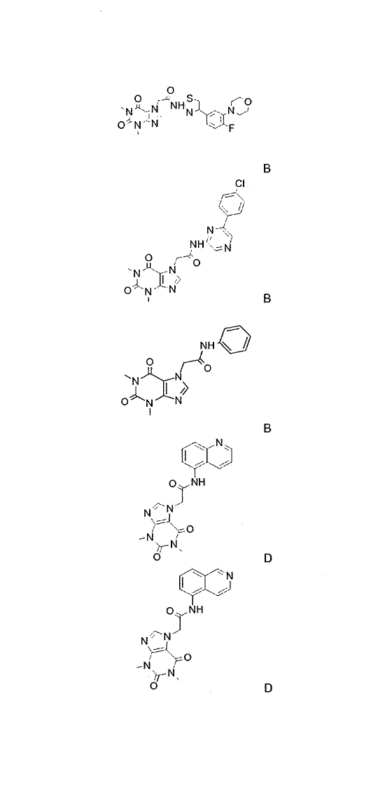

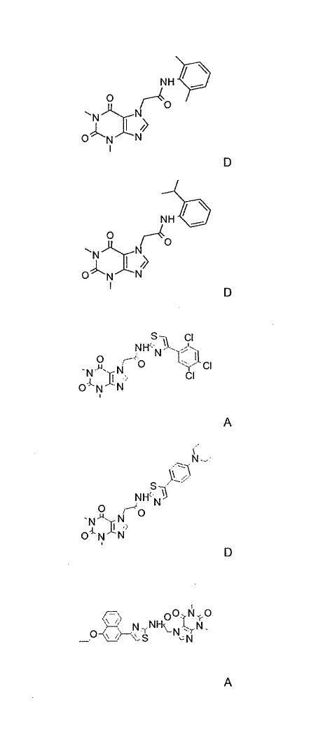

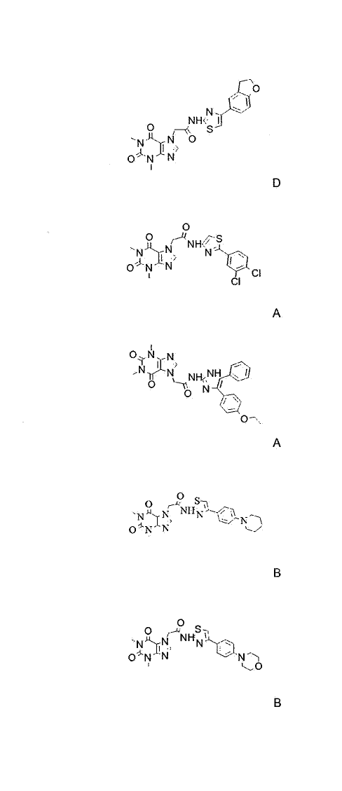

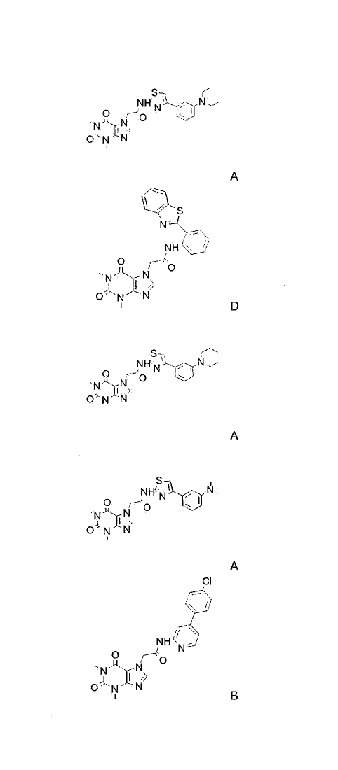

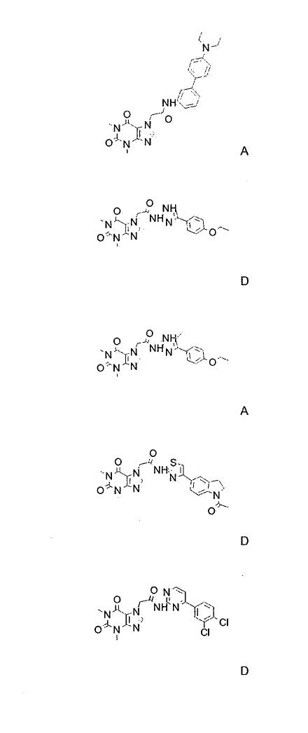

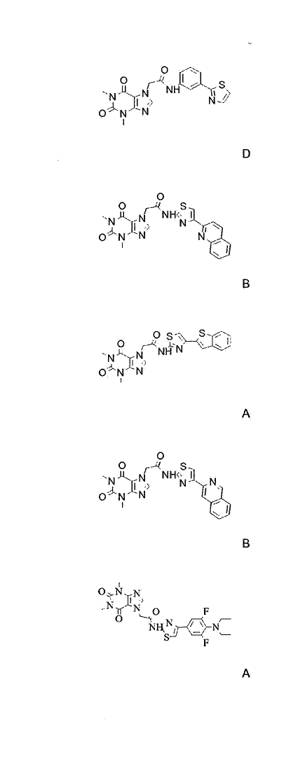

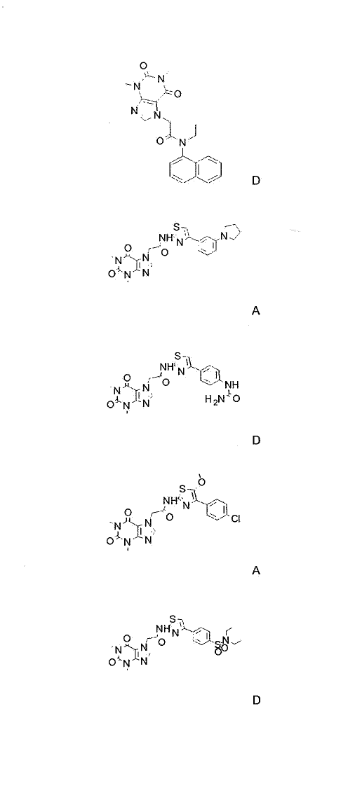

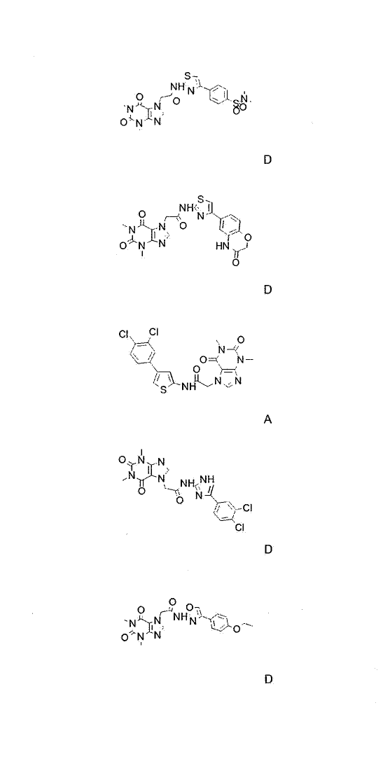

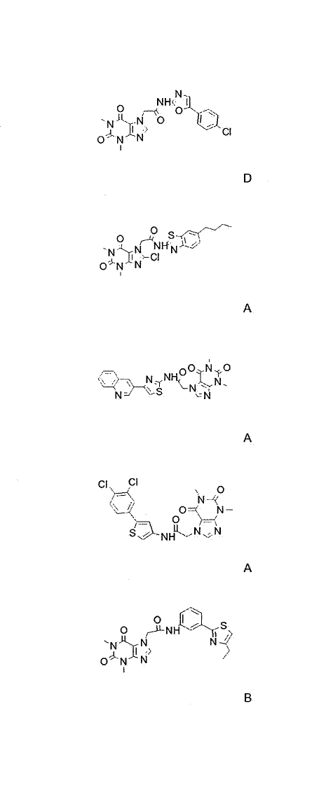

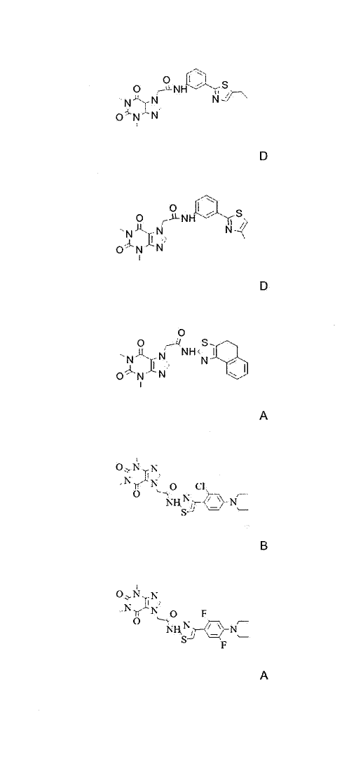

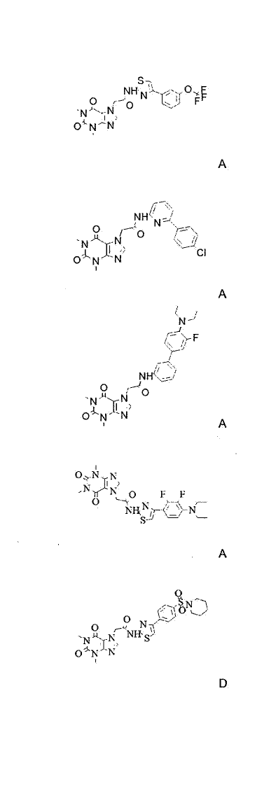

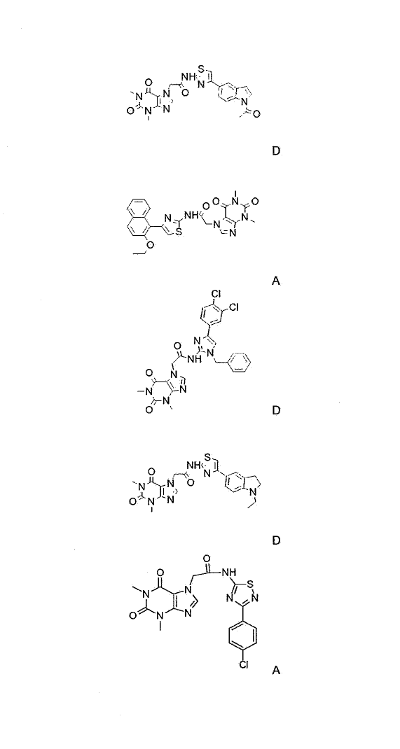

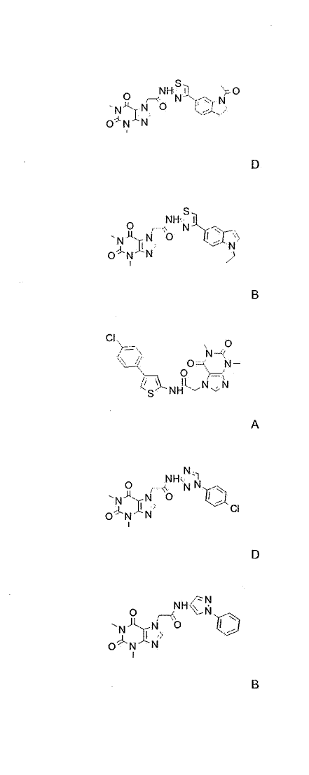

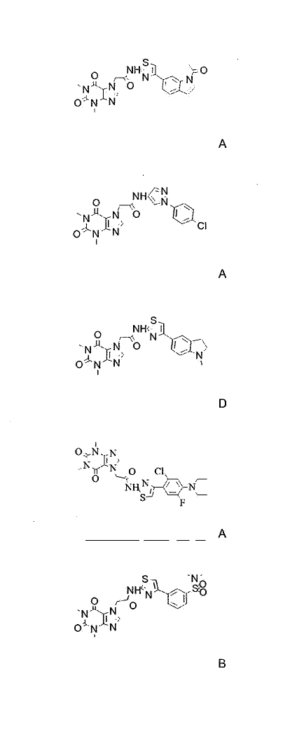

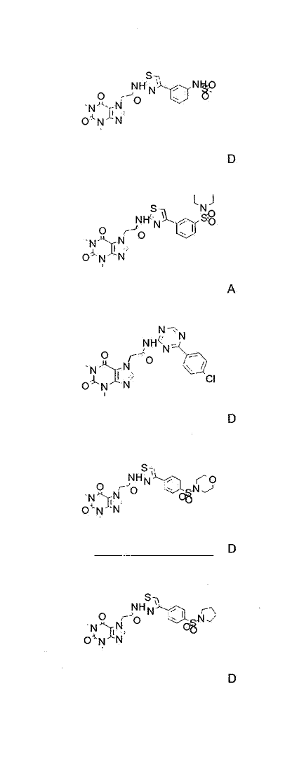

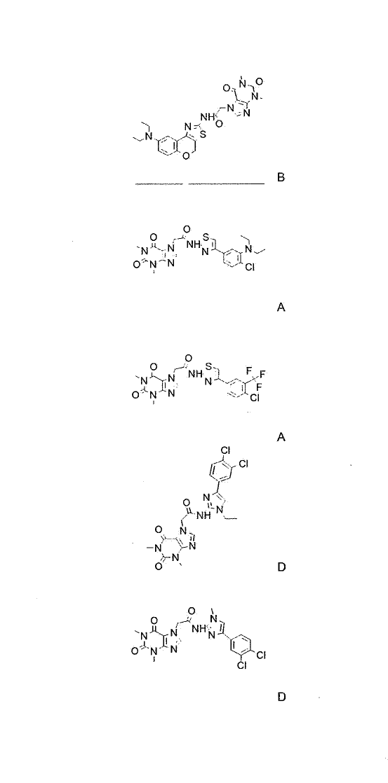

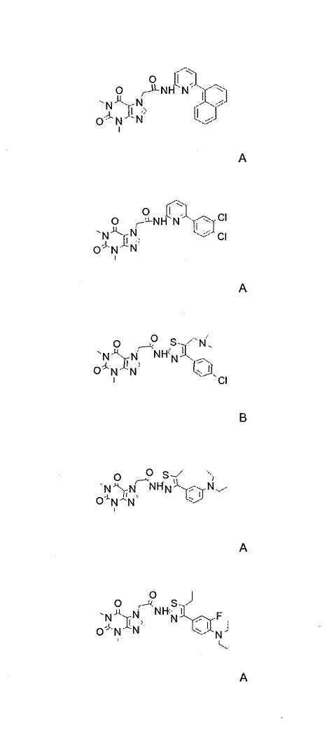

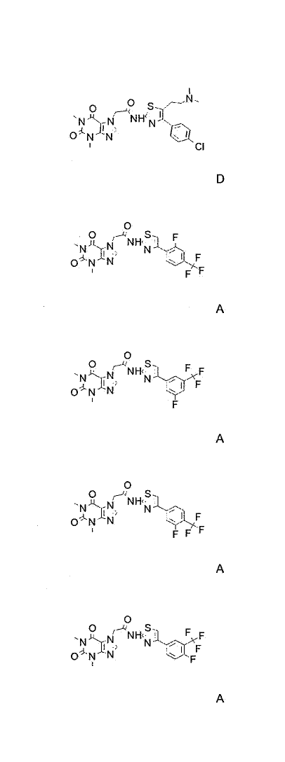

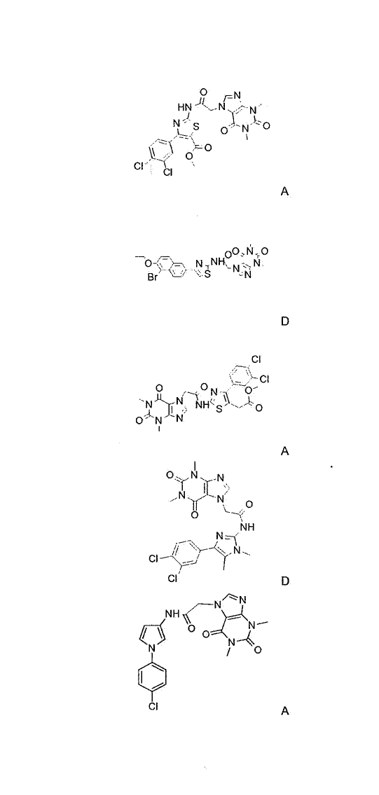

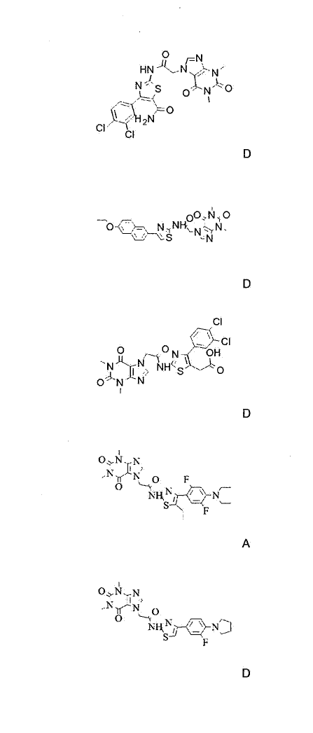

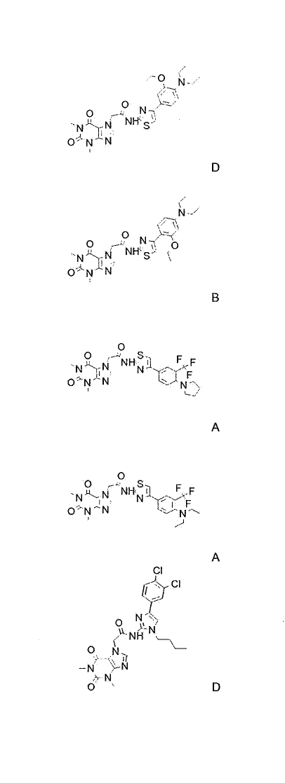

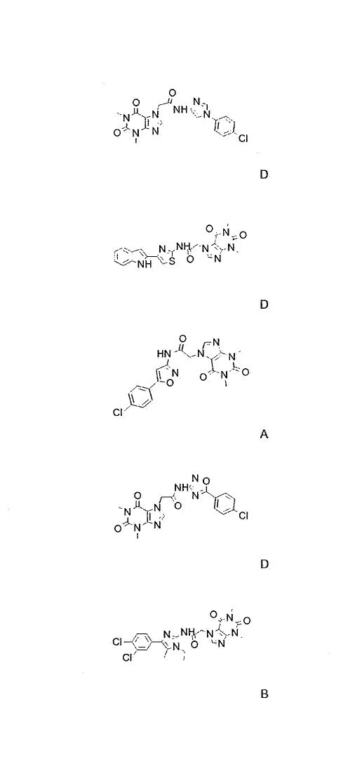

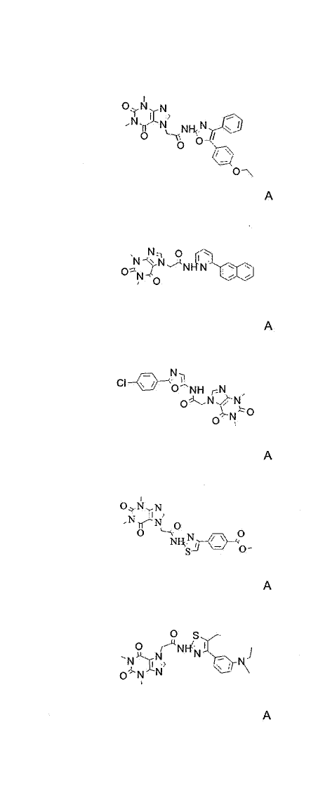

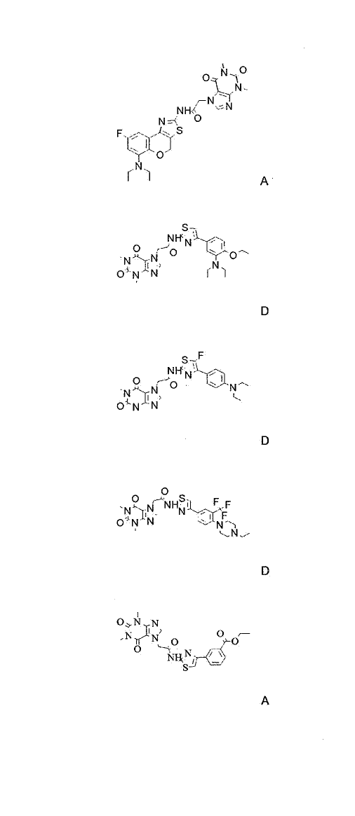

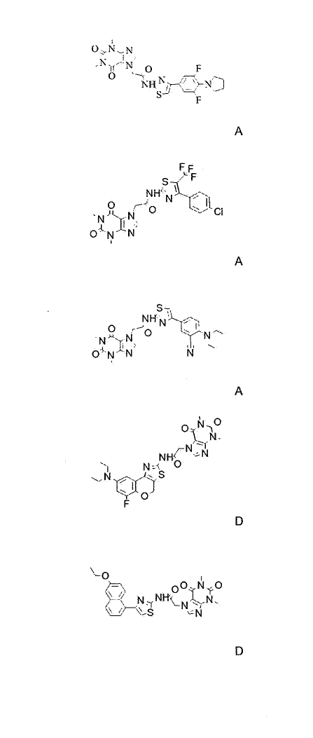

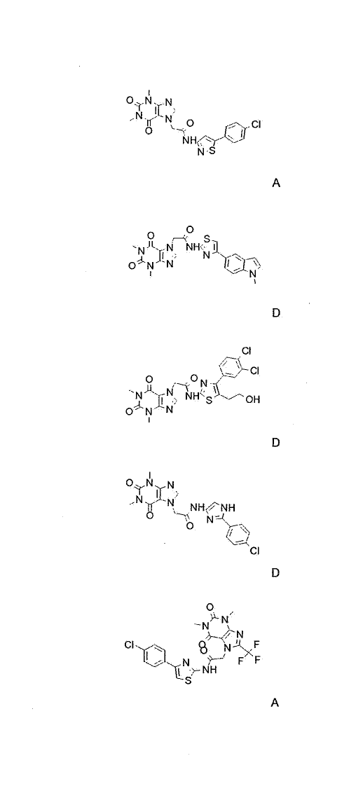

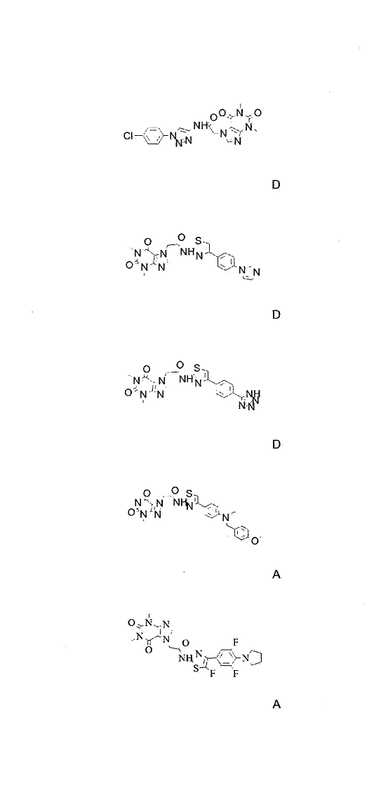

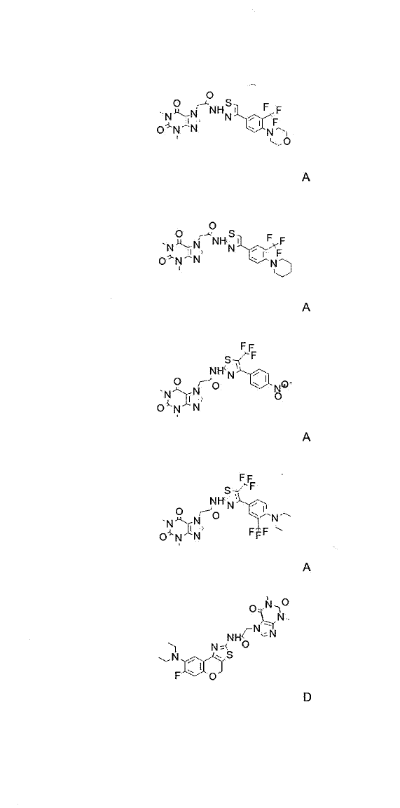

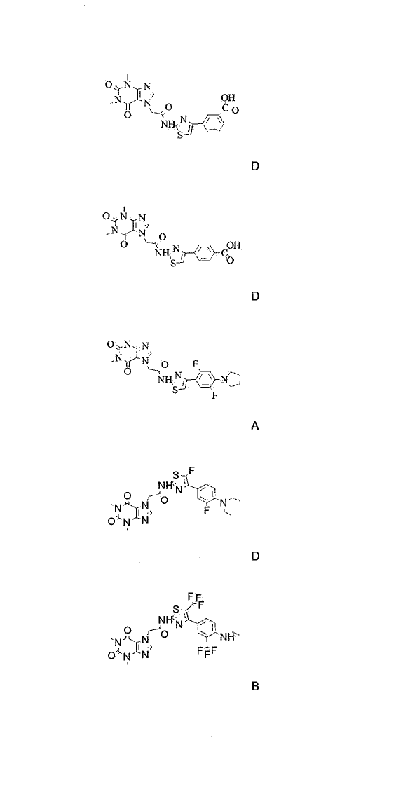

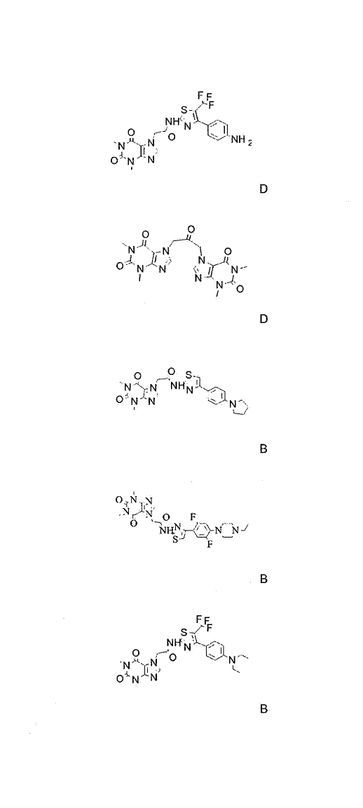

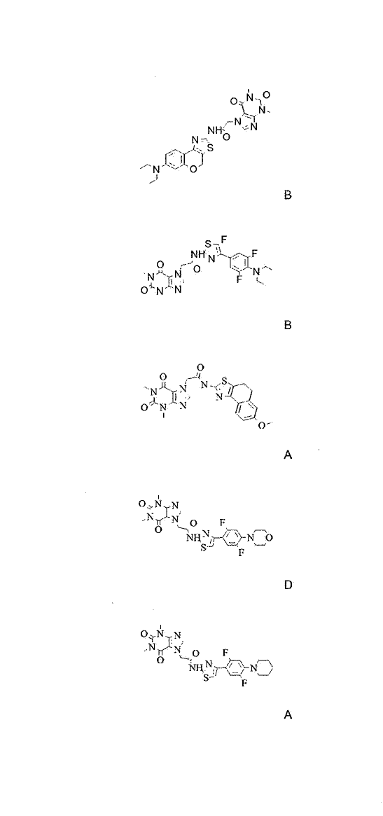

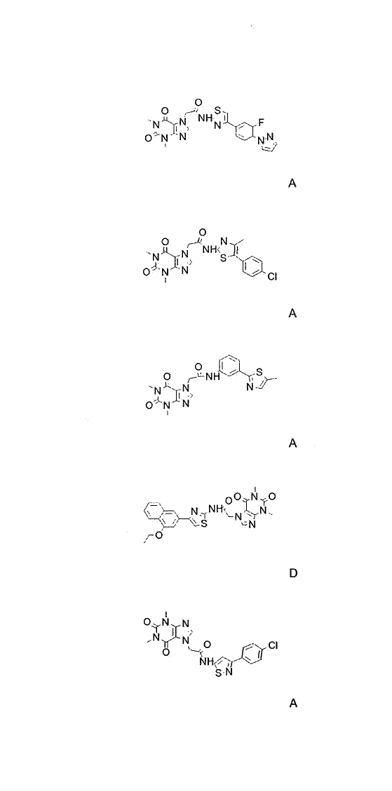

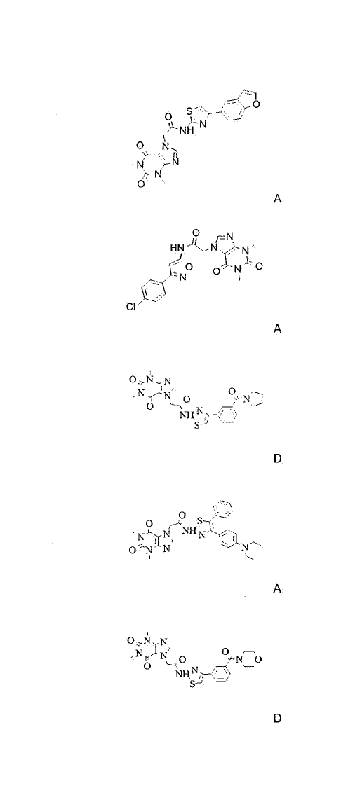

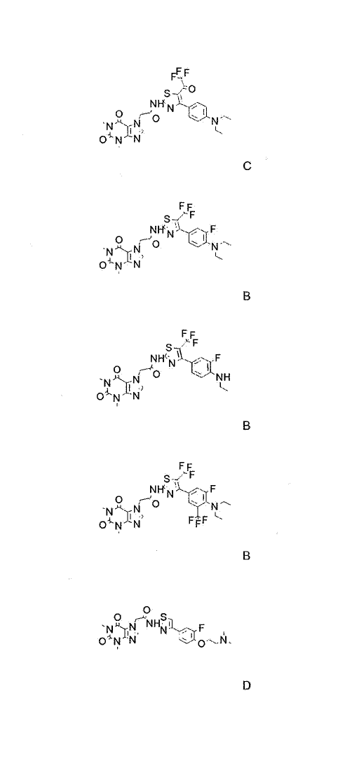

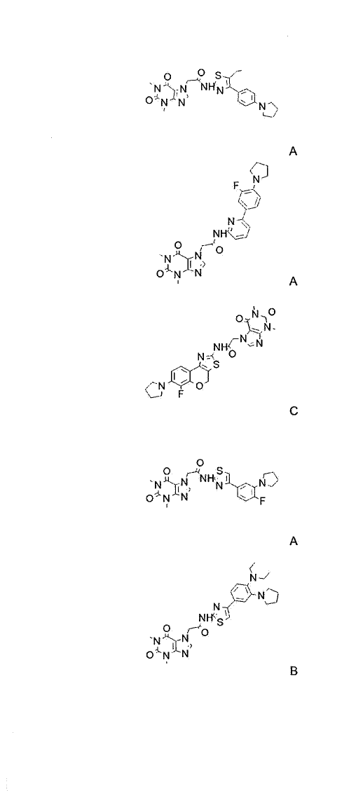

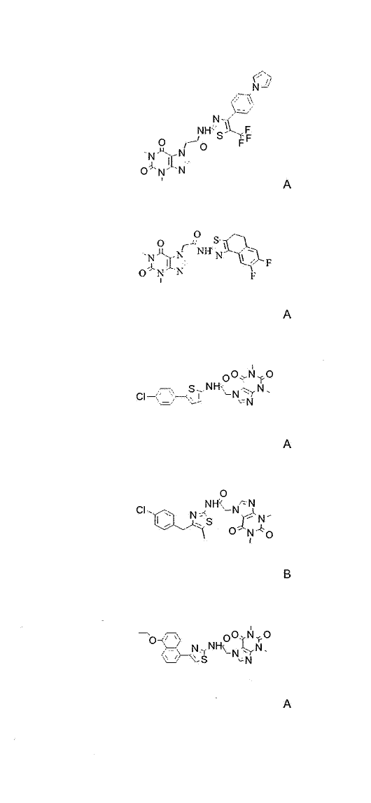

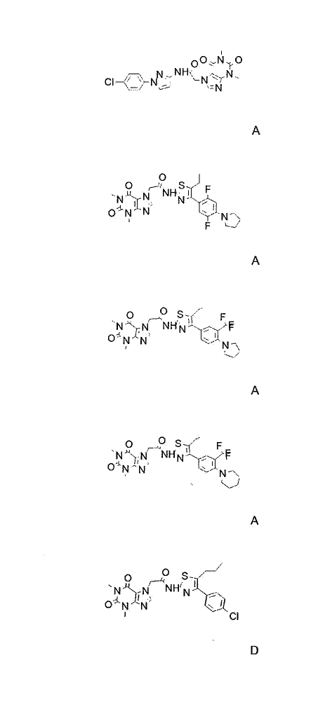

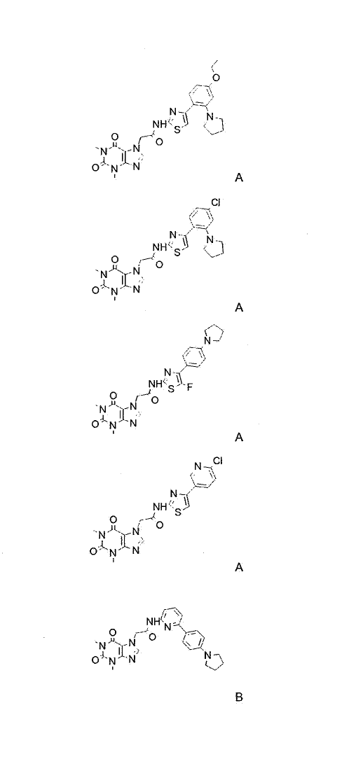

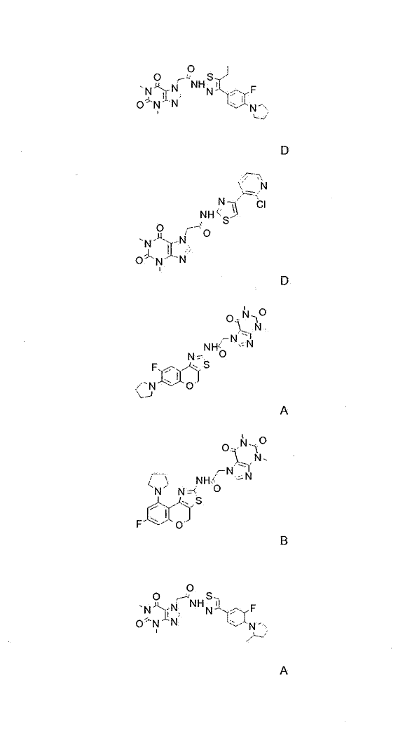

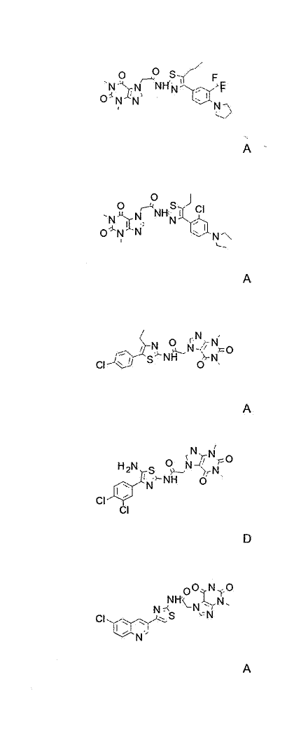

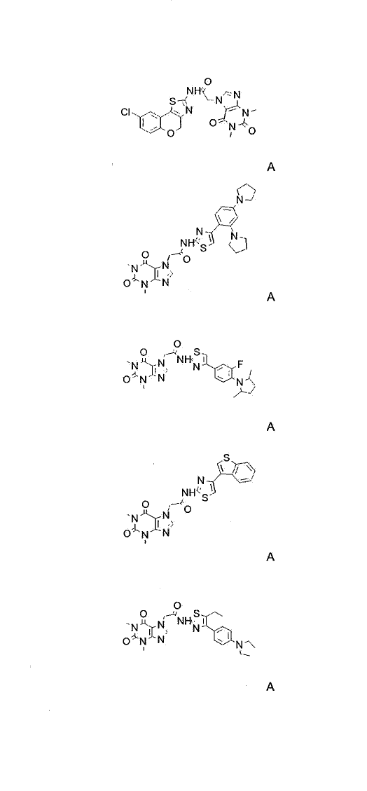

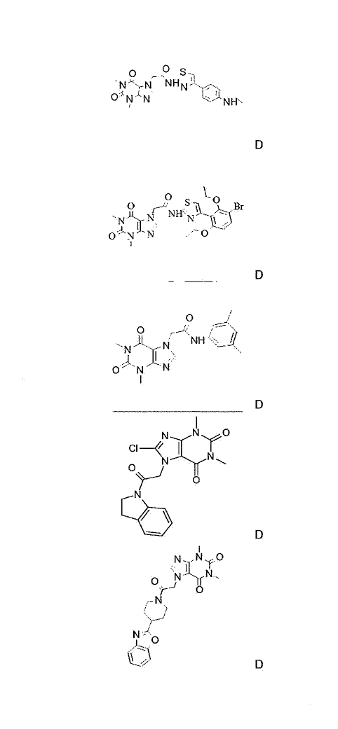

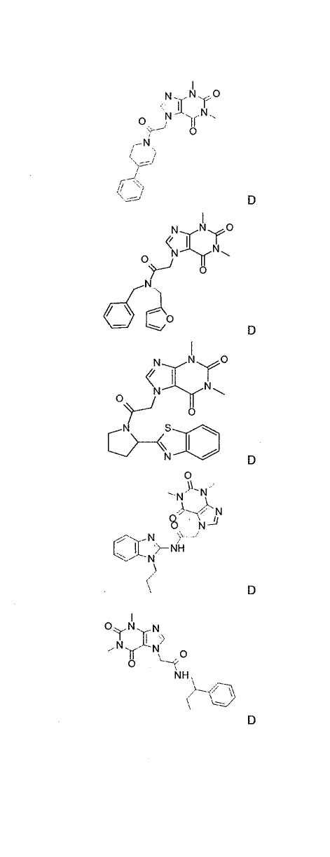

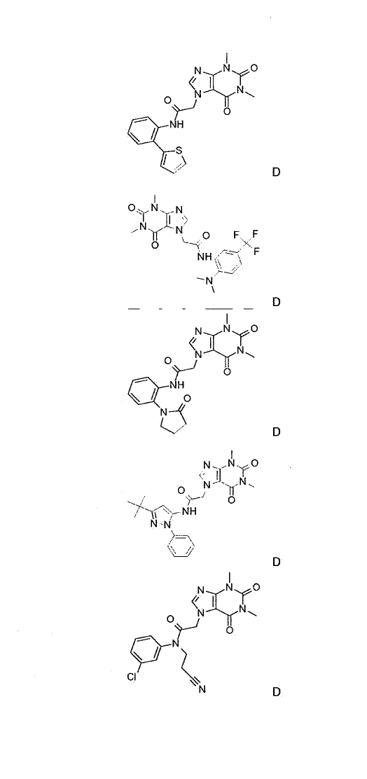

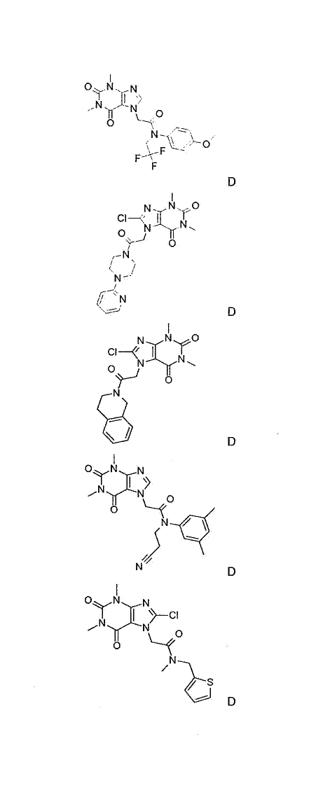

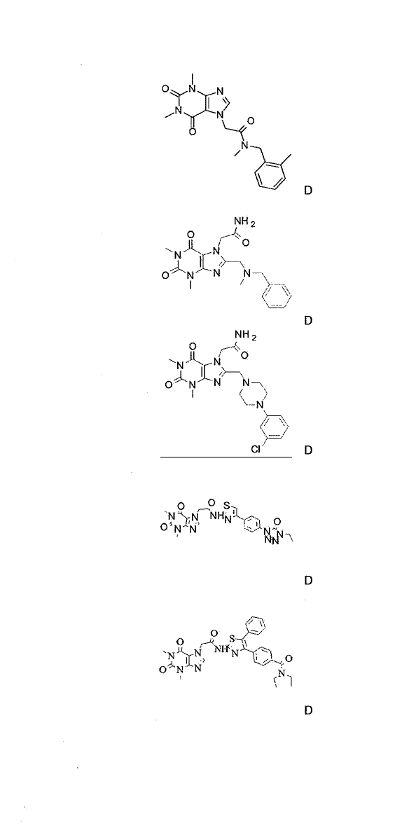

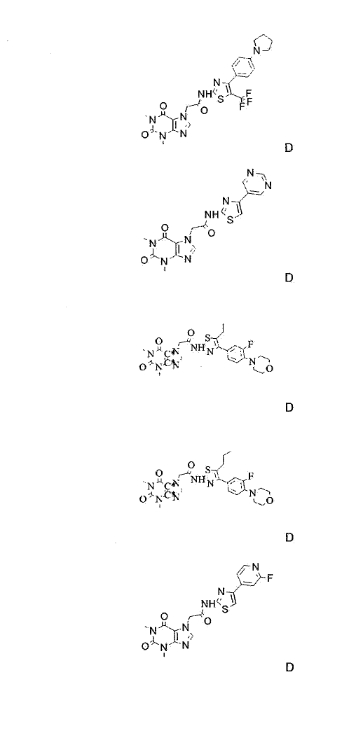

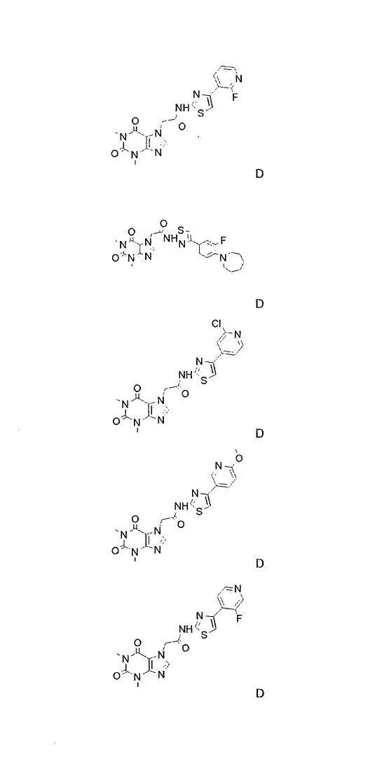

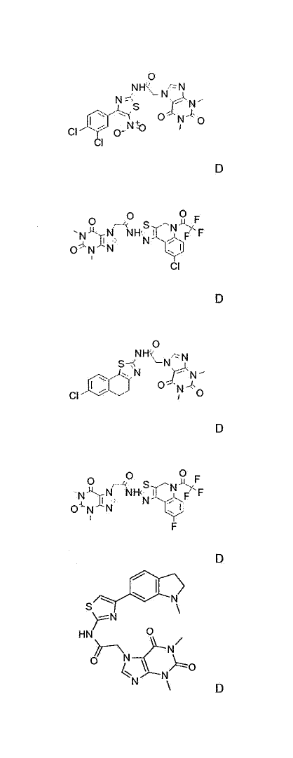

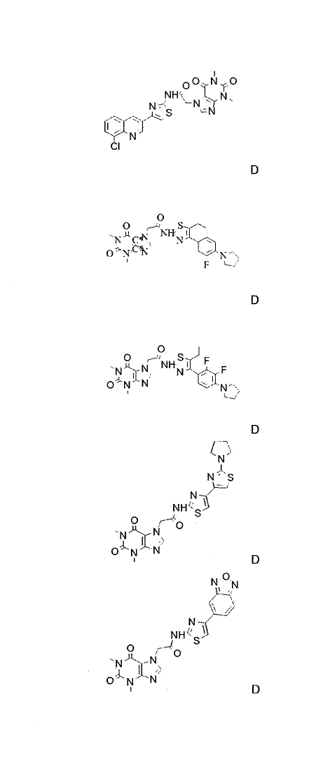

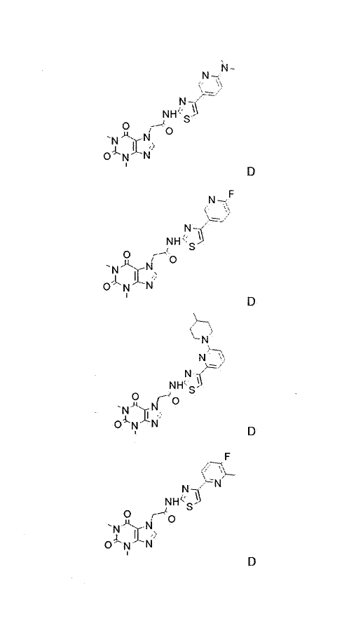

Additional exemplary compounds are provided in Table 4, FIG. 4.

One aspect of the present invention provides a pharmaceutical preparation suitable for use in a human patient, or for veterinary use, comprising an effective amount of any of the compounds shown above (e.g., a compound described hereing or a salt thereof, or a solvate, hydrate, oxidative metabolite or prodrug of the compound or its salt), and one or more pharmaceutically acceptable excipients. In certain embodiments, the pharmaceutical preparations may be for use in treating or preventing a condition involving activation of TRPA1 or for which reduced TRPA1 activity can reduce the severity. In certain embodiments, the pharmaceutical preparations have a low enough pyrogen activity to be suitable for use in a human patient, or for veterinary use. In certain embodiments, the pharmaceutical preparation comprises an effective amount of any of the compounds shown above, wherein the compound inhibits TRPA1 with an IC₅₀ of 10 micromolar or less. In certain embodiments, the pharmaceutical preparation comprises a compound which inhibits TRPA1 with an IC₅₀ of 1 micromolar or less, or with an IC₅₀ of 500 nM or less, 250 nM or less, 200 nM or less, or 100 nM or less.

In certain embodiments, the TRPA1 inhibitor for use in methods or pharmaceutical preparations of the present invention is selected from a compound depicted in Tables 1, 2, 3, or 4. In certain embodiments, the present invention contemplates the use of any compound as depicted in optionally substituted in any of the methods or pharmaceutical preparations of the present invention.

One aspect of the current invention provides use of a TRPA1 inhibitor in the manufacture of a medicament for treating or preventing a condition involving activation of TRPA1 or for which reduced TRPA1 activity can reduce the severity, wherein the TRPA1 inhibitor is represented by any of the compounds shown above (e.g., a compound described herein or a salt thereof, or a solvate, hydrate, oxidative metabolite or prodrug of the compound or its salt). In certain embodiments, the compound inhibits a TRPA1 mediated current with an IC₅₀ of less than 10 micromolar.

In certain embodiments of the above formula, substituted substituents may be substituted with one or more of: alkyl, alkenyl, alkynyl, cycloalkyl, heterocyclyl, aryl, heteroaryl, cycloalkylalkyl, heterocyclylalkyl, aralkyl, or heteroaralkyl, any of which may itself be further substituted, or halogen, hydroxyl, carbonyl (e.g., ester, carboxyl, or formyl), thiocarbonyl (e.g., thioester, thiocarboxylate, or thioformate), ketone, aldehyde, amino, acylamino, amido, amidino, cyano, nitro, azido, sulfonyl, sulfoxido, sulfate, sulfonate, sulfamoyl, sulfonamido, and phosphoryl.

In certain embodiments, the invention contemplates that any of the particular compounds depicted in Tables 1, 2, 3, or 4 can be administered to treat any of the diseases or conditions disclosed herein. In some embodiments, the compound is formulated as a pharmaceutical preparation prior to administration. In certain embodiments, the TRPA1 inhibitor for use in methods or pharmaceutical preparations of the present invention is selected from a compound depicted in Tables 1, 2, 3, or 4. In certain embodiments, the present invention contemplates the use of any compound as depicted in Tables 1, 2, 3, or 4 in any of the methods or pharmaceutical preparations of the present invention.

The particular compounds and structural formulas disclosed herein are merely exemplary. The use of small molecule TRPA1 inhibitors having one or more of the functional or structural characteristics described herein are similarly contemplated,

Compounds of any of the above structures may be used in the manufacture of medicaments for the treatment of any diseases disclosed herein.

Compounds of any of the above structures may be used to inhibit a function of a TRPA1 channel in vitro or in vivo.

In certain embodiments, compounds that include all or a functional portion of any of the foregoing structures may be used in the manufacture of medicaments for the treatment of any of the diseases disclosed herein. Additionally or alternatively, such compounds may be used in in vitro or in vivo methods of inhibiting TRPA1 function, such as a TRPA1-mediated current.

In particular embodiments, a small molecule TRPA1 antagonist is chosen for use because it is more selective for one TRP isoform than others, e.g., 10-fold, and more preferably at least 20, 40, 50, 60, 70, 80, or at least 100- or 1000-fold more selective for TRPA1 over one or more of TRPC6, TRPV5, TRPV6, TRPM8, TRPV1, TRPV2, TRPV4, and/or TRPV3. In other embodiments, the differential is smaller, e.g., it more strongly inhibits TRPA1 than TRPM8, TRPV1, TRPV2, TRPV3, and/or TRPV4, preferably at least twice, three times, five times, or ten times more strongly. Such comparisons may be made, for example, by comparing IC₅₀ values.

In particular embodiments, a small molecule TRPA1 antagonist is chosen for use because it is more selective for one TRPA1 than for other non-TRP ion channels, e.g., 10-fold, and more preferably at least 20, 40, 50, 60, 70, 80, or at least 100- or 1000-fold more selective for TRPA1 over one or more of NaV1.2, Cav1.2, Cav3.1, HERG, and/or mitochondrial uniporter. In other embodiments, the differential is smaller, e.g., it more strongly inhibits TRPA1 than NaV1.2, Cav1.2, Cav3.1, HERG, and/or mitochondrial uniporter, preferably at least twice, three times, five times, or ten times more strongly. Such comparisons may be made, for example, by comparing IC₅₀ values.

In certain embodiments, a compound which is an antagonist of TRPA1 is chosen to selectively antagonize TRPA1 over other ion channels, e.g., the compound modulates the activity of TRPA1 at least an order of magnitude more strongly than it modulates the activity of one or more of NaV1.2, Cav1.2, Cav3.1, HERG, and/or mitochondrial uniporter, preferably at least two orders of magnitude more strongly, more preferably at least three orders of magnitude more strongly. In certain embodiments, the compound modulates the activity of TRPA1 at least 1.5 orders of magnitude more strongly than the activity of one or more of NaV1.2, Cav1.2, Cav3.1, HERG, or mitochondrial uniporter. Such comparisons may be made, for example, by comparing IC₅₀ values.

Similarly, in particular embodiments, a small molecule is chosen for use because it lacks significant activity against one or more targets other than TRPA1. For example, the compound may have an IC₅₀ above 500 nM, above 1 μM, or above 10 μM or 100 μM for inhibiting one or more of TRPC6, TRPV5, TRPV6, Cav1.2, Cav3.1, NaV1.2, HERG, and the mitochondrial uniporter.

In particular embodiments, the small molecule is chosen for use because it is more selective for one TRP isoform than others, e.g., 10-fold, and more preferably at least 100- or 1000-fold more selective for TRPA1 over one or more of TRPC6, TRPV5, TRPV6, TRPM8, TRPV1, HERG, NaV1.2, mitochondrial uniporter, TRPV3 and/or TRPV4. In other embodiments, the differential is smaller, e.g., it more strongly inhibits TRPA1 than TRPM8, TRPV1 and/or TRPV4, preferably at least twice, three times, five times, or ten times more strongly. Such comparisons may be made, for example, by comparing IC₅₀ values.

In certain embodiment, a small molecule is chosen because it antagonizes the function of both TRPA1 and TRPM8, TRPV1 and/or TRPV3. Although such compounds selectively antagonize the function of both ion channels, the IC₅₀ values need not be identical.

In certain embodiments of any of the foregoing, the small molecule may be chosen because it inhibits a TRPA1 function with an IC₅₀ less than or equal to 1 uM, or less than or equal to 700, 600, 500, 400, 300, 250, 200, or 100 nM. In other embodiments, the small molecule is chosen because it inhibits a TRPA1 function with an IC₅₀ less than or equal to 75 nM, less than or equal to 50 nM, or less than or equal to 25, 10, 5, or 1 nM. In certain other embodiments of any of the foregoing, the small molecule inhibits TRPA1 function with an IC₅₀ less than or equal to 10 micromolar or less than or equal to 5 micromolar or less than or equal to 2.5 micromolar or less than or equal to 1.5 micromolar.

In certain embodiments of any of the foregoing, the compound may be chosen based on the rate of inhibition of a TRPA1 function. In one embodiment, the compound inhibits a TRPA1 function in less than 5 minutes, preferably less than 4, 3, or 2 minutes. In another embodiment, the compound inhibits a TRPA1 function in less than about 1 minute. In yet another embodiment, the compound inhibits a TRPA1 function in less than about 30 seconds.

In any of the foregoing embodiments, the small molecule antagonist of TRPA1 function may inhibit the outward current, the inward current, or any combination of one or more of these currents. Compounds that inhibit more than one of the foregoing currents may do so with the same or with differing IC₅₀ values. In any of the foregoing, the ability of a compound to inhibit a particular current can be assessed either in vitro or in vivo. Compounds that inhibit any of the foregoing currents in an in vitro or in vivo assay are characterized as compounds that inhibit a function of TRPA1. Stated another way, an exemplary function of TRPA1 that may be inhibited by the present compounds is a TRPA1-mediated current. Additionally or alternatively, a further exemplary function of TRPA1 that may be inhibited by the present compounds is ion flux mediated by TRPA1.

In any of the foregoing or following embodiments, the small molecule is characterized by some level of activity versus other ion channels (e.g., certain compounds are selective for inhibiting TRPA1 and other compounds exhibit a level of cross reactivity against one or more other ion channel). When a small molecule is characterized by its activity against another ion channel, inhibition of a function or activity of the other ion channel is defined analogously to the way in which a function of a TRPA1 channel is defined. Thus, inhibiting the function of another ion channel means, for example, inhibiting ion flux mediated by that other ion channel or inhibiting the current mediated by that other ion channel.

In certain embodiments of any of the foregoing, inhibition of a TRPA1 function means that a function, for example a TRPA1 mediated current, is decreased by greater than 50% in the presence of an effective amount of a compound in comparison to in the absence of the compound or in comparison to an ineffective amount of a compound. In certain other embodiments, the inhibition of a TRPA1 function means that a function, for example a TRPA1 mediated current or TRPA1 mediated ion flux, is decreased by at least 50%, 60%, 70%, 75%, 80%, 85%, or 90% in the presence of an effective amount of a compound in comparison to in the absence of the compound. In still other embodiments, the inhibition of a TRPA1 function means that a function, for example a TRPA1 mediated current, is decreased by at least 92%, 95%, 97%, 98%, 99%, or 100% in the presence of an effective amount of a compound in comparison to in the absence of the compound.

In any of the foregoing embodiments, IC₅₀ values are measured in vitro using, for example, patch clamp analysis or standard measurements of calcium flux. Exemplary in vitro methods for calcium flux-based IC₅₀ estimation are described in Example 1. Methods used to obtain more definitive IC₅₀ measurements are described in Example 2. Alternatively, estimates of % inhibition of current or ion flux can also be calculated and used to assess efficacy of a compound as an inhibitor.

Without being bound by theory, a compound may inhibit a function of TRPA1 by binding covalently or non-covalently to a portion of TRPA1. Alternatively, a compound may inhibit a function of TRPA1 indirectly, for example, by associating with a protein or non-protein cofactor necessary for a function of TRPA1. One of skill in the art will readily appreciate that an inhibitory compound may associate reversibly or irreversibly with TRPA1 or a cofactor thereof. Compounds that reversibly associate with TRPA1 or a cofactor thereof may continue to inhibit a function of TRPA1 after dissociation.

In certain embodiments of any of the foregoing, the compound that inhibits a function of TRPA1 is a small organic molecule or a small inorganic molecule. Exemplary small molecules include, but are not limited to, small molecules that bind to a TRPA1 channel and inhibit one or more function of a TRPA1 channel.

The compounds described herein are useful for the treatment or prevention of respiratory conditions. Such conditions affect the lung, pleural cavity, bronchial tubes, trachea, upper respiratory tract as well as the nerves and muscles involved in breathing. Respiratory diseases that may be treated with the compounds described herein include obstructive diseases such as chronic obstructive pulmonary disease (COPD), emphysema, chronic bronchitis, asthma (including asthma caused by industrial irritants), cystic fibrosis, bronchiectasis, bronchiolitis, allergic bronchopulmonary aspergillosis, and tuberculosis; restrictive lung disease including asbestosis, radiation fibrosis, hypersensitivity pneumonitis, acute respiratory distress syndrome, infant respiratory distress syndrome (ARDS), idiopathic pulmonary fibrosis, idiopathic pulmonary fibrosis, idiopathic interstial pneumonia, sarcoidosis, eosinophilic pneumonia, lymphangioleiomyomatosis, pulmonary Langerhan's cell histiocytosis, and pulmonary alveolar proteinosis; respiratory tract infections including upper respiratory tract infections (e.g., common cold, sinusitis, tonsillitis, pharyngitis and laryngitis) and lower respiratory tract infections (e.g., pneumonia); respiratory tumors whether malignant (e.g., small cell lung cancer, non-small cell lung cancer, adenocarcinoma, squamous cell carcinoma, large cell undifferentiated carcinoma, carcinoid, mesothelioma, metastatic cancer of the lung, metastatic germ cell cancer, metastatic renal cell carcinoma) or benign (e.g., pulmonary hamartoma, congenital malformations such as pulmonary sequestration and congenital cystic adenomatoid malformation (CCAM)); pleural cavity diseases (e.g., empyema and mesothelioma); and pulmonary vascular diseases (e.g, pulmonary embolism such as thromboembolism, and air embolism (iatrogenic), pulmonary arterial hypertension, pulmonary edema, pulmonary hemorrhage, inflammation and damage to capillaries in the lung resulting in blood leaking into the alveoli. Other conditions that may be treated include disorders that affect breathing mechanics (e.g., obstructive sleep apnea, central sleep apnea, amyotrophic lateral sclerosis, Guillan-Barre syndrome, and myasthenia gravis). In one embodiment, the respiratory condition is not asthma, COPD, or chronic cough. The present compounds can also be useful for treating, reducing, or preventing one or more symptoms associated with respiratory conditions including, for example, shortness of breath or dyspnea, cough (with or without the production of sputum), coughing blood (haemoptysis), chest pain including pleuritic chest pain, noisy breathing, wheezing, and cyanosis.

Individuals that may be treated with the present compounds are identified using any standard techniques known in the art for diagnosing respiratory conditions including, for example, X-rays, pulmonary function test, computed tomography scan, culture of microorganisms from bodily fluids (e.g., blood, sputum, urine, tears, and saliva), bronchoscopy, biopsy (e.g., of the lung or pleura), ventilation test or perfusion scan, breathing devices such as a peak flow meter or by spirometry, and ultrasound scan.

The subject TRPA1 inhibitors can be used alone or in combination with other pharmaceutically active agents. Examples of such other pharmaceutically active agents include, but are not limited to, anti-inflammatory agents (e.g., NSAIDS, bradykinin receptor antagonists, hormones and autacoids such as corticosteroids), anti-proliferative agents (e.g., anti-eczema agents, anti-cancer), anti-fungal agents, anti-viral agents, anti-septic agents (e.g., antibacterials), and local anaesthetics. Certain active agents belong to more than one category.

For any of the foregoing, a TRPA1 inhibitor can be formulated for administration by a route appropriate for the disease or injury being treated. For example, the TRPA1 inhibitor can be formulated, for example, for oral, transdermal, topical, intraperitoneal, intravenous, intravascular, intrathecal, intrapericardial, intramyocardial, subcutaneous, rectal, vaginal, or urethral delivery. Furthermore, the TRPA1 inhibitor can be formulated for delivery via a device. Exemplary devices include, but are not limited to, a catheter, wire, stent, or other intraluminal device. Further exemplary delivery devices also include a patch, bandage, mouthguard, or dental apparatus.

The invention contemplates pharmaceutical compositions of any of the foregoing TRPA1 inhibitors. Exemplary pharmaceutical compositions are formulated in a pharmaceutically acceptable carrier.

The subject TRPA1 inhibitors can be used alone or as part of a therapeutic regimen combined with other treatments, therapies, or interventions appropriate for the particular disease, condition, injury or disorder being treated. When used as part of a therapeutic regimen, the invention contemplates use of TRPA1 inhibitors in combination with one or more of the following treatment modalities: administration of non-TRPA1 inhibitor pharmaceuticals, chemotherapy, radiotherapy, homeopathic therapy, diet, stress management, and surgery.

When administered alone or as part of a therapeutic regimen, in certain embodiments, the invention contemplates administration of TRPA1 inhibitors to treat a particular primary disease, injury, disorder, or condition, for example a respiratory condition described herein. In still other embodiments, the invention contemplates administration of TRPA1 inhibitors to treat symptoms secondary to the primary disease, injury, disorder, or conditions.

The invention contemplates pharmaceutical preparations and uses of TRPA1 antagonists having any combination of the foregoing or following characteristics, as well as any combination of the structural or functional characteristics of the TRPA1 antagonists described herein. Any such antagonists or preparations can be used in the treatment of any of the diseases or conditions described herein. Additionally, the invention contemplates the use of any such antagonists or preparations for inhibiting a TRPA1 mediated current in vitro. Combinations of any of the foregoing or following aspects and embodiments of the invention are also contemplated. For example, the invention contemplates that TRPA1 antagonists having any of the particular potencies and specificities outlined herein can be formulated for the appropriate route of administration and can be used in treating any of the conditions or diseases detailed herein.

In certain embodiments of any of the foregoing, TRPA1 antagonist compounds for use in the methods of the present invention have one or more of any of the foregoing properties (e.g., IC₅₀, specificity, selectivity, activity, formulation, etc.). Compounds and uses of antagonist compounds having any combination of the foregoing properties are specifically contemplated.

The terms “antagonist” and “inhibitor” are used interchangeably to refer to an agent that decreases or suppresses a biological activity, such as to repress an activity of an ion channel, such as TRPA1. TRPA1 inhibitors include inhibitors having any combination of the structural and/or functional properties disclosed herein.

An “effective amount” of, e.g., a TRPA1 antagonist, with respect to the subject methods of inhibition or treatment, refers to an amount of the antagonist in a preparation which, when applied as part of a desired dosage regimen brings about a desired clinical or functional result. Without being bound by theory, an effective amount of a TRPA1 antagonist for use in the methods of the present invention, includes an amount of a TRPA1 antagonist effective to decrease one or more in vitro or in vivo function of a TRPA1 channel. Exemplary functions include, but are not limited to, membrane polarization (e.g., an antagonist may promote hyperpolarization of a cell), ion flux, ion concentration in a cell, outward current, and inward current. Compounds that antagonize TRPA1 function include compounds that antagonize an in vitro or in vivo functional activity of TRPA1. When a particular functional activity is only readily observable in an in vitro assay, the ability of a compound to inhibit TRPA1 function in that in vitro assay serves as a reasonable proxy for the activity of that compound. In certain embodiments, an effective amount is an amount sufficient to inhibit a TRPA1-mediated current and/or the amount sufficient to inhibit TRPA1 mediated ion flux.

The TRPA1 inhibitors for use in the methods of the present invention may be characterized according to their activity, or lack of activity, against one or more other ion channels. When other ion channels are referred to, inhibition of a function of such other ion channels is defined similarly. For example, inhibition of an ion channel or an activity of an ion channel means the antagonist inhibits one or more functional activities of the other ion channel. Such functions include the current mediated by the particular ion channel, ion flux, or membrane polarization.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “polypeptide”, and the terms “protein” and “peptide” which are used interchangeably herein, refers to a polymer of amino acids. Exemplary polypeptides include gene products, naturally-occurring proteins, homologs, orthologs, paralogs, fragments, and other equivalents, variants and analogs of the foregoing.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The term “small molecule” refers to a compound having a molecular weight less than about 2500 amu, preferably less than about 2000 amu, more preferably less than about 1500 amu, still more preferably less than about 1000 amu, or most preferably less than about 750 amu.

The terms “stringent conditions” or “stringent hybridization conditions” refer to conditions which promote specific hybridization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of the polynucleotide sequence hybridizes to a perfectly matched complementary strand. In certain cases it may be desirable to increase the stringency of the hybridization conditions to be about equal to the Tm for a particular duplex. In certain embodiments, stringent hybridization conditions include a wash step of 0.2×SSC at 65° C.

The terms “TRPA1”, “TRPA1 protein”, and “TRPA1 channel” are used interchangeably throughout the application. These terms refer to an ion channel (e.g., a polypeptide) comprising the amino acid sequence set forth in SEQ ID NO: 1, SEQ ID NO:3, or SEQ ID NO: 5, or an equivalent polypeptide, or a functional bioactive fragment thereof. In certain embodiments, the term refers to a polypeptide comprising, consisting of, or consisting essentially of, the amino acid sequence set forth in SEQ ID NO: 1, SEQ ID NO:3, or SEQ ID NO: 5. TRPA1 includes polypeptides that retain a function of TRPA1 and comprise (i) all or a portion of the amino acid sequence set forth in SEQ ID NO: 1, SEQ ID NO:3 or SEQ ID NO: 5; (ii) the amino acid sequence set forth in SEQ ID NO: 1, SEQ ID NO:3 or SEQ ID NO: 5 with 1 to about 2, 3, 5, 7, 10, 15, 20, 30, 50, 75 or more conservative amino acid substitutions; (iii) an amino acid sequence that is at least 70%, 75%, 80%, 90%, 95%, 96%, 97%, 98%, or 99% identical to SEQ ID NO: 1, SEQ ID NO:3 or SEQ ID NO: 5; and (iv) functional fragments thereof. Polypeptides of the invention also include homologs, e.g., orthologs and paralogs, of SEQ ID NO: 1, SEQ ID NO: 3 or SEQ ID NO: 5.

The term “TRPA1” further refers to a nucleic acid encoding a polypeptide of the invention, e.g., a nucleic acid comprising a sequence consisting of, or consisting essentially of, the polynucleotide sequence set forth in SEQ ID NO: 2, SEQ ID NO: 4 or SEQ ID NO: 6. A nucleic acid of the invention may comprise all, or a portion of: the nucleotide sequence of SEQ ID NO: 2, SEQ ID NO: 4 or SEQ ID NO: 6; a nucleotide sequence at least 70%, 75%, 80%, 90%, 95%, 96%, 97%, 98% or 99% identical to SEQ ID NO: 2, SEQ ID NO: 4 or SEQ ID NO: 6; a nucleotide sequence that hybridizes under stringent conditions to SEQ ID NO: 2, SEQ ID NO: 4 or SEQ ID NO: 6; nucleotide sequences encoding polypeptides that are functionally equivalent to polypeptides of the invention; nucleotide sequences encoding polypeptides at least about 70%, 75%, 80%, 85%, 90%, 95%, 98%, 99% homologous or identical with an amino acid sequence of SEQ ID NO: 1, SEQ ID NO:3 or SEQ ID NO: 5; nucleotide sequences encoding polypeptides having an activity of a polypeptide of the invention and having at least about 70%, 75%, 80%, 85%, 90%, 95%, 98%, 99% or more homology or identity with SEQ ID NO: 1, SEQ ID NO:3 or SEQ ID NO: 5; nucleotide sequences that differ by 1 to about 2, 3, 5, 7, 10, 15, 20, 30, 50, 75 or more nucleotide substitutions, additions or deletions, such as allelic variants, of SEQ ID NO: 2, SEQ ID NO: 4 or SEQ ID NO: 6; nucleic acids derived from and evolutionarily related to SEQ ID NO: 2, SEQ ID NO: 4 or SEQ ID NO: 6; and complements of, and nucleotide sequences resulting from the degeneracy of the genetic code, for all of the foregoing and other nucleic acids of the invention. Nucleic acids of the invention also include homologs, e.g., orthologs and paralogs, of SEQ ID NO: 2, SEQ ID NO: 4 or SEQ ID NO: 6 and also variants of SEQ ID NO: 2, SEQ ID NO: 4 or SEQ ID NO: 6 which have been codon optimized for expression in a particular organism (e.g., host cell). Where not explicitly stated, one of skill in the art can readily assess whether TRPA1 refers to a nucleic acid or a protein.

The term “oxidative metabolite” is intended to encompass compounds that are produced by metabolism of the parent compound under normal physiological conditions. Specifically, an oxidative metabolite is formed by oxidation of the parent compound during metabolism. For example, a thioether group may be oxidized to the corresponding sulfoxide or sulfone.

The term “solvate” as used herein, refers to a compound formed by solvation (e.g., a compound formed by the combination of solvent molecules with molecules or ions of the solute).

The term “hydrate” as used herein, refers to a compound formed by the union of water with the parent compound.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The terms “compound” and “agent” are used interchangeably to refer to the inhibitors/antagonists of the invention. In certain embodiments, the compounds are small organic or inorganic molecules, e.g., with molecular weights less than 7500 amu, preferably less than 5000 amu, and more preferably less than 2000, 1500, 1000, or 500 amu. One class of small organic or inorganic molecules are non-peptidyl, e.g., containing 2, 1, or no peptide and/or saccharide linkages. In certain other embodiments, the compounds are peptidyl agents such as polypeptides or antibodies. In certain other embodiments, the compounds are proteins, for example, antibodies or aptamers. Such compounds can bind to and inhibit a function of TRPA1. In certain other embodiments, the compounds are nucleic acids, for example, TRPA1 antisense oligonucleotides or TRPA1 RNAi constructs. Such compounds can inhibit the expression of TRPA1, thereby inhibiting the activity of TRPA1. Other exemplary compounds that may act as inhibitors include ribozymes and peptide fragments.

The term “acyl” is art-recognized and refers to a group represented by the general formula hydrocarbylC(O)—, preferably alkylC(O)—.

The term “acylamino” is art-recognized and refers to a moiety that can be represented by the general formula:

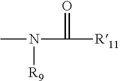

wherein R₉ is as defined above, and R′11 represents a hydrogen, an alkyl, an alkenyl or —(CH2)m-R8, where m and R8 are as defined above.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The terms “alkoxyl” or “alkoxy” as used herein refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH2)m-R8, where m and R8 are described above.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C1-C30 for straight chains, C3-C30 for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethylthio, and the like.

The terms “amine” and “amino” are art-recognized and refer to both unsubstituted and substituted amines, e.g., a moiety that can be represented by the general formula:

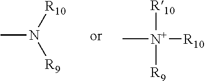

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In certain such embodiments, neither R₉ and R₁₀ is attached to N by a carbonyl, e.g., the amine is not an amide or imide, and the amine is preferably basic, e.g., its conjugate acid has a pKₐ above 7. In more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” is art-recognized as an amino-substituted carbonyl and includes a moiety that can be represented by the general formula:

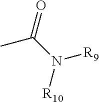

wherein R₉, R₁₀ are as defined above. Preferred embodiments of the amide will not include imides that may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle or cyclyl”, as used herein, refers to an aromatic or non-aromatic ring in which each atom of the ring is carbon.

The term “carbonyl” is art-recognized and includes such moieties as can be represented by the general formula:

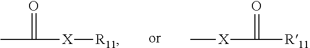

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “electron withdrawing group” refers to chemical groups which withdraw electron density from the atom or group of atoms to which electron withdrawing group is attached. The withdrawal of electron density includes withdrawal both by inductive and by delocalization/resonance effects. Examples of electron withdrawing groups attached to aromatic rings include perhaloalkyl groups, such as trifluoromethyl, halogens, azides, carbonyl containing groups such as acyl groups, cyano groups, and imine containing groups.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “nitro” means —NO2; the term “halogen” designates —F, —Cl, —Br or —I; the term “sulfhydryl” means —SH; the term “hydroxyl” means —OH; and the term “sulfonyl” means —SO2-.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

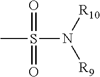

in which R9 and R10 are as defined above.

The term “sulfate” is art recognized and includes a moiety that can be represented by the general formula:

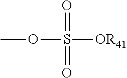

in which R41 is as defined above.

The term “sulfonamide” is art recognized and includes a moiety that can be represented by the general formula:

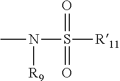

in which R9 and R′11 are as defined above.

The term “sulfonate” is art-recognized and includes a moiety that can be represented by the general formula:

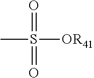

in which R41 is an electron pair, hydrogen, alkyl, cycloalkyl, or aryl.

The terms “sulfoxido” or “sulfinyl”, as used herein, refers to a moiety that can be represented by the general formula:

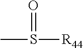

in which R44 is selected from the group consisting of hydrogen, alkyl, alkenyl, alkynyl, cycloalkyl, heterocyclyl, aralkyl, or aryl.

The term “thioester”, as used herein, refers to a group —C(O)SR⁹ or —SC(O)R⁹ wherein R⁹ represents a hydrocarbyl.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, Acc. Chem. Res. 23: 128 (1990).

Contemplated equivalents of the compounds described above include compounds which otherwise correspond thereto, and which have the same general properties thereof (e.g., the ability to inhibit TRPA1 activity), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The symbol [symbol] , whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, and the like. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs form the various salt forms in certain physical properties, such as solubility in polar solvents, but otherwise the salts are equivalent to the parent form of the compound for the purposes of the present invention.

The term “low enough pyrogen activity”, with reference to a pharmaceutical preparation, refers to a preparation that does not contain a pyrogen in an amount that would lead to an adverse effect (e.g., irritation, fever, inflammation, diarrhea, respiratory distress, endotoxic shock, etc.) in a subject to which the preparation has been administered. For example, the term is meant to encompass preparations that are free of, or substantially free of, an endotoxin such as, for example, a lipopolysaccharide (LPS).

### Diseases, Disorders, or Conditions Related to TRPA1 Function

In certain embodiments, the invention provides methods and compositions for inhibiting a function of a TRPA1 channel in vitro or in vivo. Exemplary functions include, but are not limited to, TRPA1-mediated current. In certain embodiments, the invention provides methods for preventing or treating a disease or disorder or condition by administering an agent that modulates the level and/or activity of a TRPA1 protein. In other embodiments, the compound selectively inhibits the expression level and/or activity of a TRPA1 protein. In other words, in certain embodiment, the compound inhibits the activity of a TRPA1 protein preferentially in comparison to the activity of one or more other ion channels.

The compounds described herein are useful for the treatment or prevention of respiratory conditions. Such conditions affect the lung, pleural cavity, bronchial tubes, trachea, upper respiratory tract as well as the nerves and muscles involved in breathing. Respiratory diseases that may be treated with the compounds described herein include obstructive diseases such as chronic obstructive pulmonary disease (COPD), emphysema, chronic bronchitis, asthma (including asthma caused by industrial irritants), cystic fibrosis, bronchiectasis, bronchiolitis, allergic bronchopulmonary aspergillosis, and tuberculosis; restrictive lung disease including asbestosis, radiation fibrosis, hypersensitivity pneumonitis, infant respiratory distress syndrome, idiopathic pulmonary fibrosis, idiopathic pulmonary fibrosis, idiopathic interstial pneumonia sarcoidosis, eosinophilic pneumonia, lymphangioleiomyomatosis, pulmonary Langerhan's cell histiocytosis, and pulmonary alveolar proteinosis; respiratory tract infections including upper respiratory tract infections (e.g., common cold, sinusitis, tonsillitis, pharyngitis and laryngitis) and lower respiratory tract infections (e.g., pneumonia); respiratory tumors whether malignant (e.g., small cell lung cancer, non-small cell lung cancer, adenocarcinoma, squamous cell carcinoma, large cell undifferentiated carcinoma, carcinoid, mesothelioma, metastatic cancer of the lung, metastatic germ cell cancer, metastatic renal cell carcinoma) or benign (e.g., pulmonary hamartoma, congenital malformations such as pulmonary sequestration and congenital cystic adenomatoid malformation (CCAM)); pleural cavity diseases (e.g., empyema and mesothelioma); and pulmonary vascular diseases (e.g, pulmonary embolism such as thromboembolism, and air embolism (iatrogenic), pulmonary arterial hypertension, pulmonary edema, pulmonary hemorrhage, inflammation and damage to capillaries in the lung resulting in blood leaking into the alveoli. Other conditions that may be treated include disorders that affect breathing mechanics (e.g., obstructive sleep apnea, central sleep apnea, amyotrophic lateral sclerosis, Guillan-Barre syndrome, and myasthenia gravis). In one embodiment, the respiratory condition is not asthma, COPD, or chronic cough. The present compounds can also be useful for treating, reducing, or preventing one or more symptoms associated with respiratory conditions including, for example, shortness of breath or dyspnea, cough (with or without the production of sputum), coughing blood (haemoptysis), chest pain including pleuritic chest pain, noisy breathing, wheezing, and cyanosis.

Individuals that may be treated with the present compounds are identified using any standard techniques known in the art for diagnosing respiratory conditions including, for example, X-rays, pulmonary function test, computed tomography scan, culture of microorganisms from bodily fluids (e.g., blood, sputum, urine, tears, and saliva), bronchoscopy, biopsy (e.g., of the lung or pleura), ventilation test or perfusion scan, breathing devices such as a peak flow meter or by spirometry, and ultrasound scan.

TRPA1 inhibitors described herein can be used in the treatment of any of the foregoing or following diseases or conditions. When used in a method of treatment, an inhibitor can be selected and formulated based on the intended route of administration. Inhibitors can be used to treat the underlying disease or condition, or to relieve a symptom of the disease or condition. Exemplary symptoms include pain associated with a disease or condition.

### Disease and Injury Models

Compounds that antagonize TRPA1 function may be useful in the prophylaxis and treatment of any of the foregoing injuries, diseases, disorders, or conditions. In addition to in vitro assays of the activity of these compounds, their efficacy can be readily tested in one or more animal models. By way of example, numerous well known animal models exist. One or more suitable animal models (e.g., suitable in light of the particular indication) can be selected.

For testing the efficacy of TRPA1 antagonists for the treatment of cough, experiments using the conscious guinea pig model of cough can be readily conducted. Tanaka and Maruyama (2003) Journal Pharmacol Sci 93: 465-470; McLeod et al. (2001) Br J Pharmacol 132: 1175-1178. Briefly, guinea pigs serve as a useful animal model for cough because, unlike other rodents such as mice and rats, guinea pigs actually cough. Furthermore, guinea pig coughing appears to mimic human coughing in terms of the posture, behavior, and appearance of the coughing animal.

To induce cough, conscious guinea pigs are exposed to an inducing agent such as citric acid or capsaicin. The response of the animal is measured by counting the number of coughs. The effectiveness of a cough suppressing agent, for example a compound that inhibits TRPA1, can be measured by administering the agent and assessing the ability of the agent to decrease the number of coughs elicited by exposure to citric acid, capsaicin, or other similar cough-inducing agent. In this way, TRPA1 inhibitors for use in the treatment of cough can be readily evaluated and identified.

Additional models of cough include the unconscious guinea pig model. Rouget et al. (2004) Br J Pharmacol 141: 1077-1083. Either of the foregoing models can be adapted for use with other animals capable of coughing. Exemplary additional animals capable of coughing include cats and dogs.

### Combination Therapy

Another aspect of the invention provides a conjoint therapy wherein one or more other therapeutic agents are administered with the TRPA1 modulators. Such conjoint treatment may be achieved by way of the simultaneous, sequential, or separate dosing of the individual components of the treatment.

In certain embodiments, a compound of the invention is conjointly administered with an analgesic. Suitable analgesics include, but are not limited to, opioids, glucocorticosteroids, non-steroidal anti-inflammatories, naphthylalkanones, oxicams, para-aminophenol derivatives, propionic acids, propionic acid derivatives, salicylates, fenamates, fenamate derivatives, pyrozoles, and pyrozole derivatives. Examples of such analgesic compounds include, but are not limited to, codeine, hydrocodone, hydromorphone, levorpharnol, morphine, oxycodone, oxymorphone, butorphanol, dezocine, nalbuphine, pentazocine, etodolac, indomethacin, sulindac, tolmetin, nabumetone, piroxicam, acetaminophen, fenoprofen, flurbiprofen, ibuprofen, ketoprofen, naproxen, diclofenac, oxaprozin, aspirin, diflunisal, meclofenamic acid, mefanamic acid, prednisolone, and dexamethasone. Preferred analgesics are non-steroidal anti-inflammatories and opioids (preferably morphine).

In certain embodiments, a compound of the invention is conjointly administered with a non-steroidal anti-inflammatory. Suitable non-steroidal anti-inflammatory compounds include, but are not limited to, piroxicam, diclofenac, etodolac, indomethacin, ketoralac, oxaprozin, tolmetin, naproxen, flubiprofen, fenoprofen, ketoprofen, ibuprofen, mefenamic acid, sulindac, apazone, phenylbutazone, aspirin, celecoxib and rofecoxib.

In certain embodiments, a compound of the invention is conjointly administered with an antiviral agent. Suitable antiviral agents include, but are not limited to, amantadine, acyclovir, cidofovir, desciclovir, deoxyacyclovir, famciclovir, foscamet, ganciclovir, penciclovir, azidouridine, anasmycin, amantadine, bromovinyldeoxusidine, chlorovinyldeoxusidine, cytarbine, didanosine, deoxynojirimycin, dideoxycitidine, dideoxyinosine, dideoxynucleoside, edoxuidine, enviroxime, fiacitabine, foscamet, fialuridine, fluorothymidine, floxuridine, hypericin, interferon, interleukin, isethionate, nevirapine, pentamidine, ribavirin, rimantadine, stavirdine, sargramostin, suramin, trichosanthin, tribromothymidine, trichlorothymidine, vidarabine, zidoviridine, zalcitabine 3-azido-3-deoxythymidine, 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-fluorocytosine (F-ddC), trisodium phosphomonoformate, trifluorothymidine, 3′ azido-3′ thymidine (AZT), dideoxyinosine (ddI), and idoxuridine.

In certain embodiments, a compound of the invention is conjointly administered with an antibacterial agent. Suitable antibacterial agents include, but are not limited to, amanfadine hydrochloride, amanfadine sulfate, amikacin, amikacin sulfate, amoglycosides, amoxicillin, ampicillin, amsamycins, bacitracin, beta-lactams, candicidin, capreomycin, carbenicillin, cephalexin, cephaloridine, cephalothin, cefazolin, cephapirin, cephradine, cephaloglycin, chilomphenicols, chlorhexidine, chloshexidine gluconate, chlorhexidine hydrochloride, chloroxine, chlorquiraldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erhmycin stearate, farnesol, floxacillin, gentamicin, gentamicin sulfate, gramicidin, giseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenine, methenamine hippurate, methenamine mandelate, methicillin, metonidazole, miconazole, miconazole hydrochloride, minocycline, minocycline hydrochloride, mupirocin, nafcillin, neomycin, neomycin sulfate, netimicin, netilmicin sulfate, nitrofurazone, norfloxacin, nystatin, octopirox, oleandomycin, orcephalosporins, oxacillin, oxyteacline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, struptomycin, sulfonamide, tetracyclines, tetracycline, tobramycin, tobramycin sulfate, triclocarbon, triclosan, trimethoprim-sulfamethoxazole, tylosin, vancomycin, and yrothricin.

In certain embodiments, a compound of the invention is conjointly administered with a cough suppressant, decongestant, or expectorant.

The subject TRPA1 inhibitors can also be administered with vitamins and derivatives thereof including Vitamin A, ascorbic acid (Vitamin C), alpha-tocopherol (Vitamin E), 7-dehydrocholesterol (Vitamin D), Vitamin K, alpha-lipoic acid, lipid soluble anti-oxidants, and the like.

In certain embodiments, two or more compounds of the invention are conjointly administered. When two or more compounds of the invention are conjointly administered, the two or more compounds may have a similar selectivity profile and functional activity, or the two or more compounds may have a different selectivity profile and functional activity. By way of example, the two or more compounds may both be approximately 10, 100, or 1000 fold selective for antagonizing a function of TRPA1 over TRPV1, TRPV5, and TRPV6 (e.g., the two or more compounds have a similar selectivity profile), and further may inhibit a function of TRPA1 with a similar IC50 (e.g., a similar functional activity). Alternatively, the one of the two or more compounds may selectively inhibit TRPA1 while the other of the two or more compounds inhibits both TRPA1 and TRPV1 (e.g., the two or more compounds have differing selectivity profiles). Administration of combinations of two or more compounds of the invention having similar or differing properties are contemplated.

In certain embodiments, a compound of the invention is conjointly administered with one or more additional compounds that antagonize the function of a different channel. By way of example, a compound of the invention may be conjointly administered with one or more compounds that antagonize TRPV1, TRPM8, and/or TRPV3. The compound(s) that antagonize TRPV1, TPRM8, or TRPV3 may be selective for TRPV1, TRPM8 or TRPV3 (e.g., inhibit TRPV1 or TRPV3 10, 100, or 1000 fold more strongly than TRPA1). Alternatively, the compound(s) that antagonize TRPV1 or TRPV3 may cross react with other TRP channels.

In certain other embodiments, a compound of the invention is conjointly administered with one or more additional agents or therapeutic regimens appropriate for the particular injury, disease, condition, or disorder being treated.

### Pharmaceutical Compositions

While it is possible for a compound of the present invention to be administered alone, it is preferable to administer the compound as a pharmaceutical formulation (composition). The compounds according to the invention may be formulated for administration in any convenient way for use in human or veterinary medicine. In certain embodiments, the compound included in the pharmaceutical preparation may be active itself, or may be a prodrug, e.g., capable of being converted to an active compound in a physiological setting.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) for inhalation. However, in certain embodiments the subject compounds may be simply dissolved or suspended in sterile water. In certain embodiments, the pharmaceutical preparation is non-pyrogenic, i.e., does not elevate the body temperature of a patient.

The phrase “therapeutically effective amount” as used herein means that amount of a compound, material, or composition comprising a compound of the present invention which is effective for producing some desired therapeutic effect by inhibiting TRPA1 function in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

It is known that sterols, such as cholesterol, will form complexes with cyclodextrins. Thus, in preferred embodiments, where the inhibitor is a steroidal alkaloid, it may be formulated with cyclodextrins, such as α-, β- and γ-cyclodextrin, dimethyl-β cyclodextrin and 2-hydroxypropyl-β-cyclodextrin.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable and/or sterile carriers and can also be administered in conjunction with other antimicrobial agents such as penicillins, cephalosporins, aminoglycosides and glycopeptides. Conjunctive therapy thus includes sequential, simultaneous and separate administration of the active compound in a way that the therapeutic effects of the first administered one are still detectable when the subsequent therapy is administered.

The present invention contemplates formulation of the subject compounds in any of the aforementioned pharmaceutical compositions and preparations. Furthermore, the present invention contemplates administration via any of the foregoing routes of administration. One of skill in the art can select the appropriate formulation and route of administration based on the condition being treated and the overall health, age, and size of the patient being treated.

### EXAMPLES

### Example 1

### High Thoughput Screening Assay

The assay depended on detection of the rise in intracellular Ca²⁺ concentration ([Ca²⁺]ᵢ) following channel activation in cells inducibly expressing the TRPA1 channel. Ca²⁺ rise was quantified with the use of fluorescent Ca²⁺ indicators that were loaded into cells and thereafter indicated the [Ca²⁺]ᵢ. Ca²⁺ influx followed activation of the TRPA1 channel. Compounds inhibiting the [Ca²⁺]ᵢ rise were considered hits for further investigation.

The commercially available HEK293/TREx line (Invitrogen) was stably transfected with a TRPA1 construct (specifically a construct encoding a TRPA1 protein with an amino acid sequence depicted in SEQ ID NO: 1) and screened by conventional calcium imaging to find clones with TRPA1 expression following stimulation with 1 μg/ml tetracycline. These cells were maintained in the growth medium recommended by the manufacturer supplemented with 100 μg/ml hygromycin to promote retention of the TRPA1 construct. After growing to near confluency, cells were plated at a density of ˜25,000 cells/well in 384 well CellBind plates (Corning) in the presence of 1 μg/ml tetracycline, and allowed to grow for 20-30 hrs. A nearly confluent monolayer resulted. Cells were then loaded with Ca²⁺ dye: Fura-2/AM or Fluo4/AM was added to the wells to a final concentration of 2 μM or 1 μM, respectively, and incubated for 80 min or 60 min, respectively, at room temperature. Supernatant was then removed from the cells by inverting plates with a sharp flick, and 40 μl Hank's Balanced Salt Solution (HBSS; 0.185 g/l D-glucose, 0.9767 g/l MgSO₄ (anhydrous), 0.4 g/l KCl, 0.06 g/l KH₂PO₄ (anhydrous), 0.35 g/l NaHCO₃, 8.0 g/l NaCl, and 0.04788 g/l Na₂HPO₄ (anhydrous); pH 7.4) was then added to each well. Following ˜1 hour for recovery from loading, cells were assayed using the Hamamatsu FDSS 6000 system, which permitted illumination alternately at 340 nM and 380 nM for Fura-2 experiments, or at 485 nM for Fluo4 experiments. Frames were acquired at a rate of 0.2 Hz. During the assay, the plates were continuously vortexed, with pipette mixing of wells following addition of each reagent. For the screening assay, 13 μl of a diluted stock (at 50 μM) was added to each well for 2 minutes following the collection of a short (4 frame) baseline. 13 μl 37.5 μM AITC (allylisothiocyanate) was then added to each well, achieving a final concentration of 10 μM each compound and 7.5 μM AITC. Data was collected for ˜3 minutes following addition of AITC, where the fluorescent intensity (for Fluo4) and the F340/F380 ratio (for Fura-2) were proportional to the [Ca²⁺]ᵢ. Negative controls consisted of HEK293/TREx TRPA1 cells exposed to AITC, but no compound. Positive control cells were usually HEK293/TREx (“parental”) cells exposed to AITC but no compound, but sometimes normal HEK/293 TREx TRPA1 cells were also used, but not exposed to AITC or compound. These controls defined a screening window, and “hits” were defined as those compounds inhibiting the fluorescence response by at least 40%. IC₅₀ values were determined for compounds defined as “hits.” The Fluo4 cell-based fluorescence assay was used to determine the intracellular Ca²⁺ concentration in the presence of varying drug concentration. Concentrations tested were 40 μM, 20 μM, 10 μM, 5 μM, 2.5 μM, 1.25 μM, and 0.625 μM. Compounds were tested in triplicate at all concentrations. Standard software was used to fit IC₅₀ curves.

Additionally or alternatively, efficacy can be represented as % inhibition in the presence (of a given concentration of compound) versus the absence of compound or in comparison to a control compound. For example, efficacy can be represented as % inhibition of ion flux in the presence versus the absence of compound.

### Example 2

### Patch Clamp Experiments

Patch clamp experiments permit the detection of currents through the TRPA1 channel in the cell line described above. To permit recording of current at a stable level and prevent the “rundown” observed by other labs, it is necessary to use the perforated patch technique, which prevents dialysis of the cytoplasm with the pipette solution. In normal whole-cell patch clamp recordings, a glass electrode is brought into contact with a single cell and a high-resistance (gigaohm) seal is established with the cell membrane. The membrane is then ruptured to achieve the whole-cell configuration, permitting control of the voltage of the cell membrane and measurement of currents flowing across the membrane using the amplifier attached to the electrode and resulting in the replacement of cytoplasm with the pipette solution. In contrast, in the perforated patch mode, an antibiotic, amphotericin, is present in the pipette solution and diffuses into contact with the cell after the seal is achieved, over the course of several minutes. The amphotericin forms ion-permeable pores in the membrane under the pipette, permitting passage of some ions but maintaining most native cytosolic components. A perfusion system permits control of the extracellular solution, including the addition of blockers and activators of the current. The current can be activated by addition of 5 μM AITC to the solution.

TRPA1 cells were induced 20-48 hours, removed from growth plates, and replated at low density (to attain good single-cell physical separation) on glass coverslips for measurement. In some cases, cells were grown in low density overnight on glass coverslips. Patch clamp recordings were made in the whole-cell mode with a holding potential of −40 mV. Every 5 seconds, a voltage ramp was applied from −120 to +100 mV, 400 ms in duration. Currents elicited were quantified at −80 mV and +80 mV. The internal solution consisted of 140 mM cesium aspartate, 10 mM EGTA, 2.2 mM CaCl₂, 2.08 mM MgCl₂ and 10 mM HEPES, pH 7.2, with 50 nM calculated free Ca²⁺ and 60 mg/ml amphotericin added immediately prior to experiments. The external solution consisted of 150 mM NaCl, 4.5 mM KCl, 3 mM MgCl₂, 10 mM HEPES, 10 mM glutamine, 1 mM EGTA, pH 7.4. Upon addition of AITC, TRPA1 current was induced only in TRPA1-expressing cells and not in parental HEK293 TREx cells. Removal of the AITC stimulus causes most of the current to go away. Potential blockers were tested for ability to block both inward and outward currents in the continued presence of AITC.

IC₅₀ of compounds was estimated by testing each compound at 5 μM and 500 nM. When 5 μM compound showed no block, IC₅₀ was estimated as >10 μM. When 5 μM compound showed 50% or less block, a rough estimate of IC₅₀ in the range of 5-10 μM could be made. IC₅₀ for compounds between 500 nM and 5 μM was similarly estimated. Compounds blocking 50% or more at 500 nM are retested at multiple concentrations, and the % block at each is fitted by standard equations to determine IC₅₀ accurately using a 5-6 point concentration/response experiment. Except where indicated, the IC₅₀ values presented in Tables 1, 2, 3, and 4 were obtained from patch clamp experiments.

### Example 3

### Other Screening Assays

Although the exemplary TRPA1 inhibitors provided herein were identified using the assays described in Examples 1 and 2, other cell-based assays can be used to identify and/or characterize TRPA1 inhibitors. One such assay is described in U.S. application Ser. No. 11/078,188, filed Mar. 11, 2005, the contents of which are hereby incorporated by reference in their entirety. TRPA1 protein can be expressed in the prokaryotic cell system described in application Ser. No. 11/078,188, and this system can be used to screen for compounds that modulate an activity of the TRPA1 protein. Alternatively, an ion channel other than TRPA1 can be expressed in the prokaryotic cell system, and the system can be used to evaluate the activity profile of an identified TRPA1 inhibitors with respect to other ion channels.

Any assays performed to identify and/or characterize compounds that inhibit an activity of TRPA1 can be performed in a high-throughput fashion, or can be performed on a smaller scale examining individual compounds or small numbers of compounds. Additionally, any of these assays can be performed (i) as a primary assay to identify compounds that inhibit a function of TRPA1; (ii) as a secondary assay to assess the specificity of a compound with respect to its activity against other ion channels; (iii) as an assay used in a medicinal chemistry program to optimize subject compounds.

### Example 4

### Synthetic Methods

### General Procedure A for the Preparation of Amides by Coupling Using EDCI

To a mixture of theophylline-7-acetic acid (2 mmol), DMAP (2 mmol), substituted phenethylamine (2 mmol) and DIPEA (4 mmol) in DMF (20 mL) is added EDCI (2 mmol). The reaction mixture is heated to 40° C. and stirred over night. The solution is concentrated in vacuo and the residue is dissolved in EtOAc (100 mL), washed with H₂O, citric acid (10%), NaHCO₃ (sat.) and brine, dried over Na₂SO₄ and concentrated in vacuo. The crude product is purified by flash chromatography on silica gel eluting with MeOH/EtOAc (1-8%).

### General Procedure B for the Preparation of Amides Via Acid Chloride

A suspension of theophylline-7-acetic acid (2 mmol) in CHCl₃ (15 mL) and MeCN (15 mL) is cooled in an ice-water bath. Oxalyl chloride (2.2 mmol) is then added dropwise. Catalytic DMF (˜25 μL) is then added. The mixture is stirred at room temperature over night. The solution is then cooled in an ice-water bath, and DMAP (2.5 mmol) is added in one portion. The substituted phenethylamine is added dropwise and the reaction mixture is stirred at room temperature over night. After diluting with CHCl₃ (50 mL), the mixture is washed with H₂O, citric acid (10% in H₂O), NaHCO₃ (sat.), dried over Na₂SO₄ and concentrated in vacuo. The crude product is purified by flash chromatography on silica gel eluting with MeOH/EtOAc (1-8%).

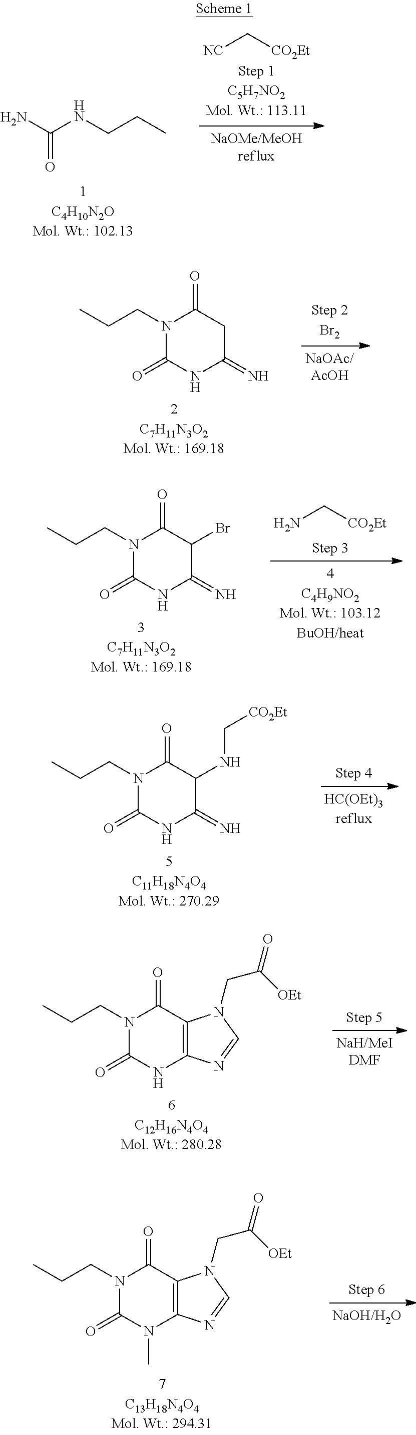

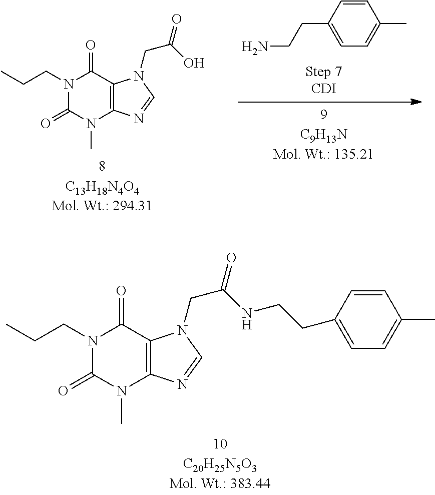

Dihydropyrimidine-dione 2 can be prepared by reacting 1-propylurea (1) and ethyl 2-cyanoacetate, which can be subsequently treated with bromine, ethyl 2-aminoacetate, and triethoxymethane to yield compound 6, ethyl 2-(2,6-dioxo-1-propyl-2,3-dihydro-1H-purin-7(6H)-yl)acetate. The obtained dihydropurine 6 can be transformed to compound 10 through methylation, hydrolysis, and a coupling reaction under CDI.

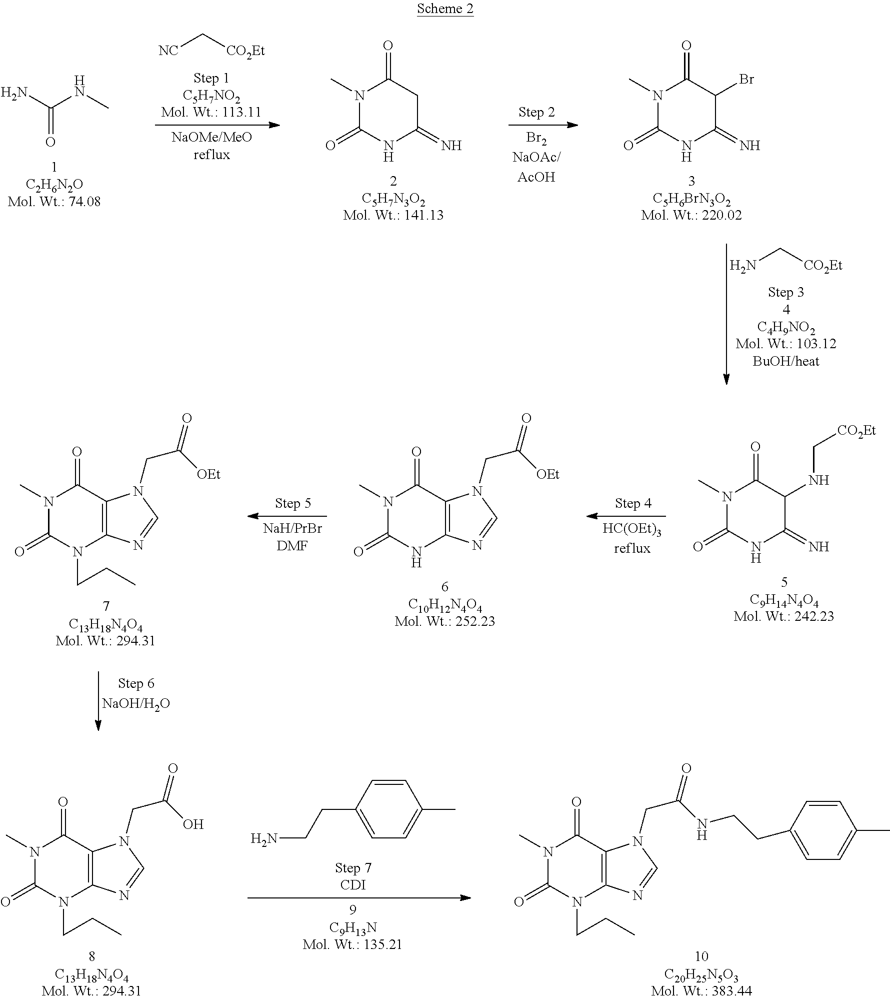

Dihydropyrimidine-dione 2 can be prepared by reacting 1-propylurea (1) and ethyl 2-cyanoacetate, which can be subsequently treated with bromine, ethyl 2-aminoacetate, and triethoxymethane to yield compound 6, ethyl 2-(1-methyl-2,6-dioxo-2,3-dihydro-1H-purin-7(6H)-yl)acetate. The obtained dihydropurine 6 can be converted to compound 10 through alkylation reaction, hydrolysis, and a coupling reaction catalyzed by CDI.

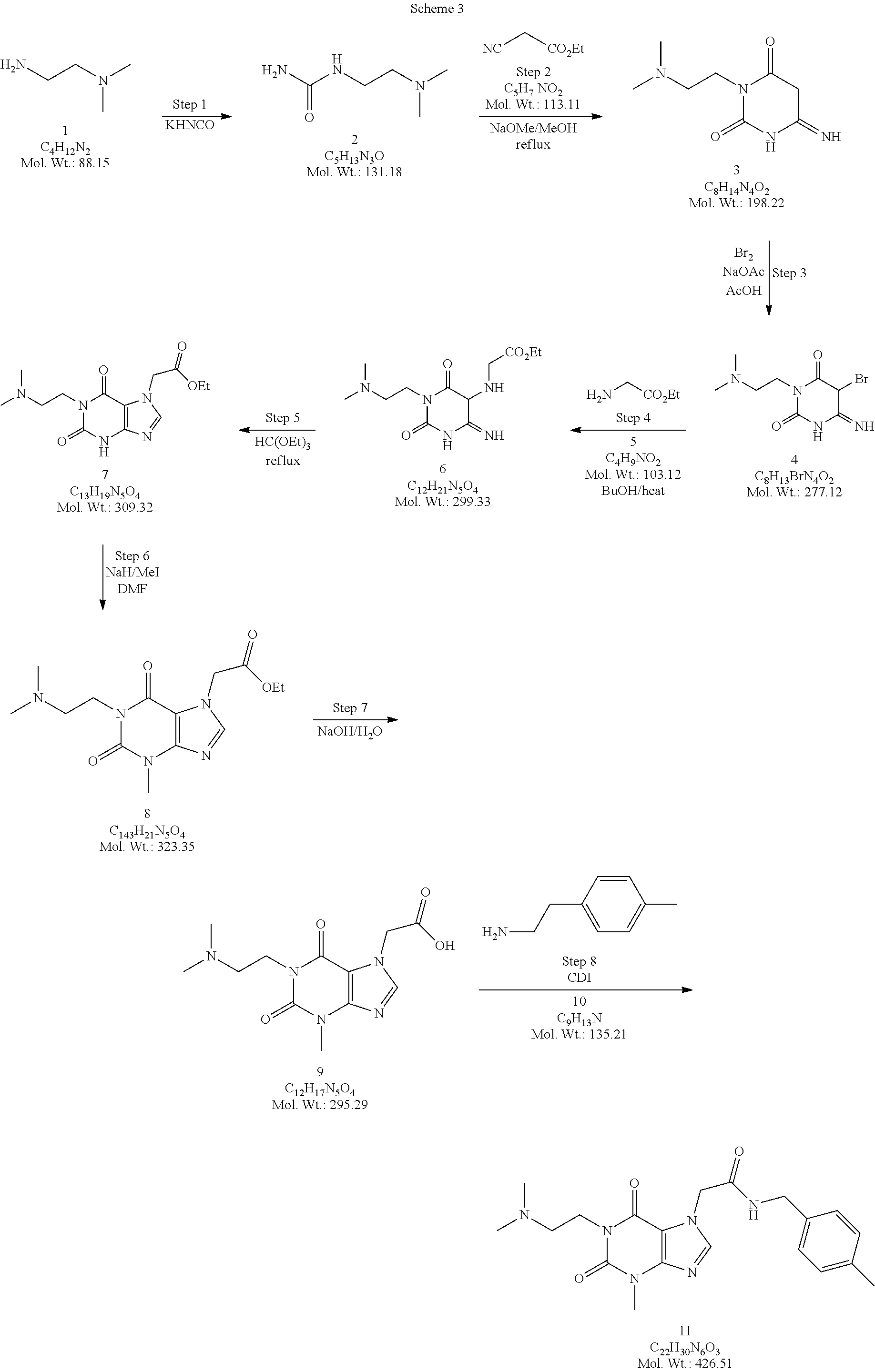

N,N-Dimethylethane-1,2-diamine can be converted to urea 2, which can then react with ethyl 2-cyanoacetate to give dihydropyrimidine-dione 3. Compound 3 can be subsequently treated with bromine, ethyl 2-aminoacetate, and triethoxymethane to yield compound 7, ethyl 24142-(dimethylamino)ethyl)-2,6-dioxo-2,3-dihydro-1H-purin-7(6H)-yl)acetate. The obtained dihydropurine 7 can be transformed to compound 11 through alkylation reaction, hydrolysis, and a coupling reaction catalyzed by CDI.

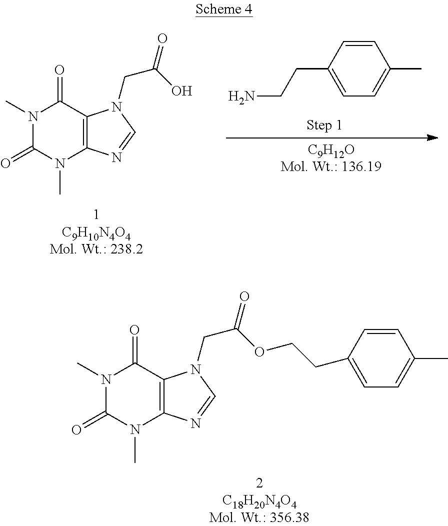

Compound 2 can be prepared by coupling dihydropurine 1 with 2-p-tolylethanol.

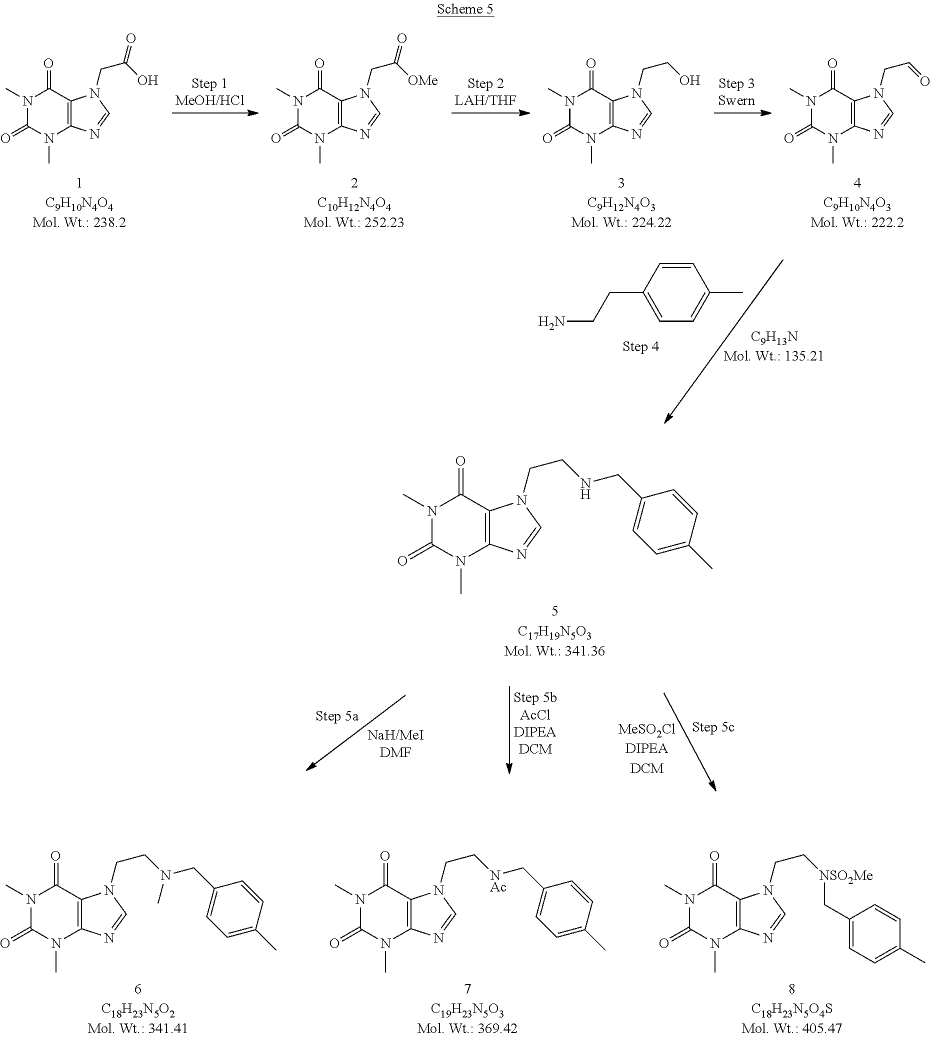

Esterification of dihydropurine 1, followed by reduction with LAH, Swern oxidation and coupling reaction can yield compound 5, which subsequently can be converted to compound 6, compound 7, and compound 8 through methylation, acylation, or sulphonylation.

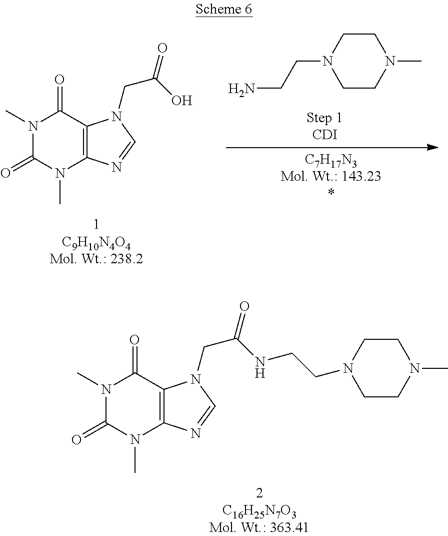

Dihydropurine 1 can be coupled with 2-(4-methylpiperazin-1-yl)ethanamine by CDI to give compound 2.

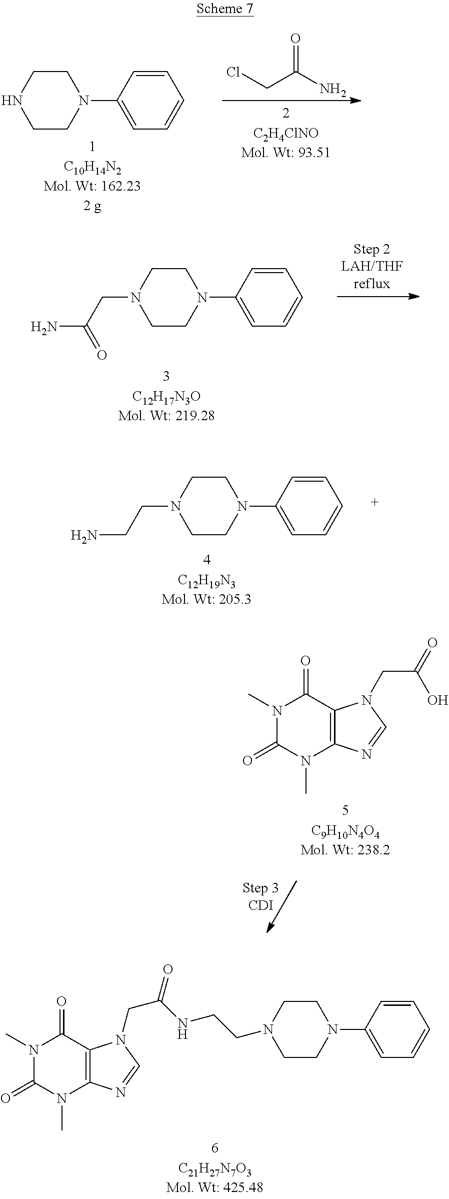

2-(4-Phenylpiperazin-1-yl)ethanamine 4 can be prepared by reacting 1-phenylpiperazine with 2-chloroacetamide, followed by a reduction reaction with LAH. Amine 4 then can be coupled with dihydropurine 5 to yield compound 6.

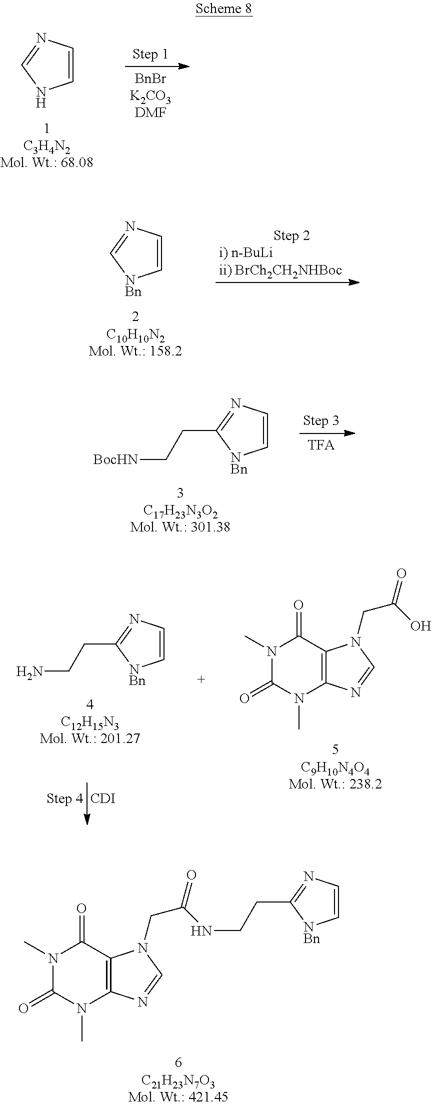

2-(1-Benzyl-1H-imidazol-2-yl)ethanamine 4 can be prepared by protection of imidazole, followed by alkylation and a deprotection reaction with TFA. Amine 4 then can be coupled with dihydropurine 5 to afford compound 6.

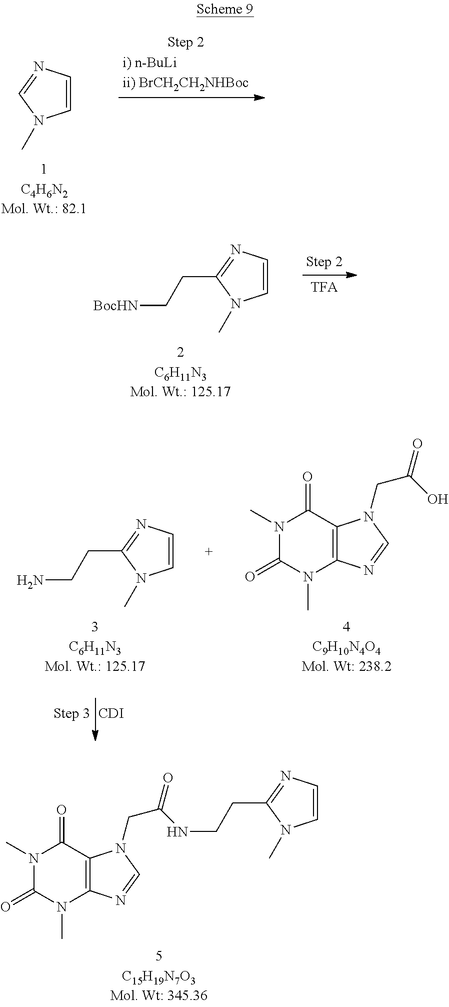

Treatment of imidazole 1 with n-BuLi, followed by an alkylation reaction, and a deprotection reaction with TFA affords 2-(1-methyl-1H-imidazol-2-yl)ethanamine. Amine 3 can be coupled with dihydropurine 4 to give compound 5.

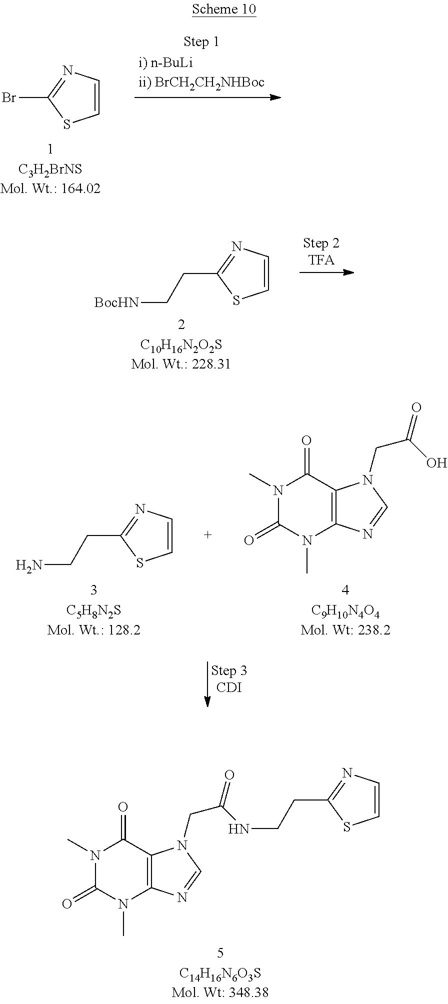

2-(Thiazol-2-yl)ethanamine 3 can be prepared by treatment of thiazole 1 with n-BuLi, followed by addition of Boc-protected 2-bromoethanamine and a deprotection reaction with TFA. The obtained amine 3 then can be coupled with carboxylic acid 4 to afford compound 5.

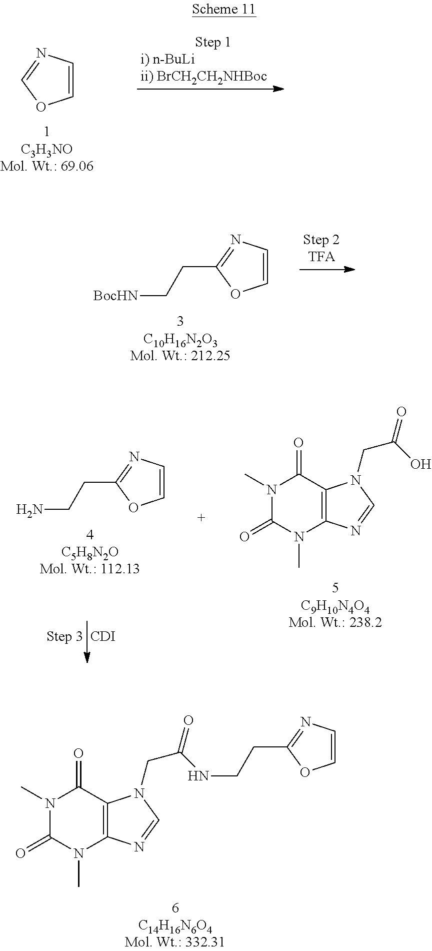

Treatment of oxazole 1 with n-BuLi, followed by an alkylation reaction, and a deprotection reaction with TFA affords 2-(oxazol-2-yl)ethanamine 3. Amine 3 can be coupled with dihydropurine 4 to give compound 6.

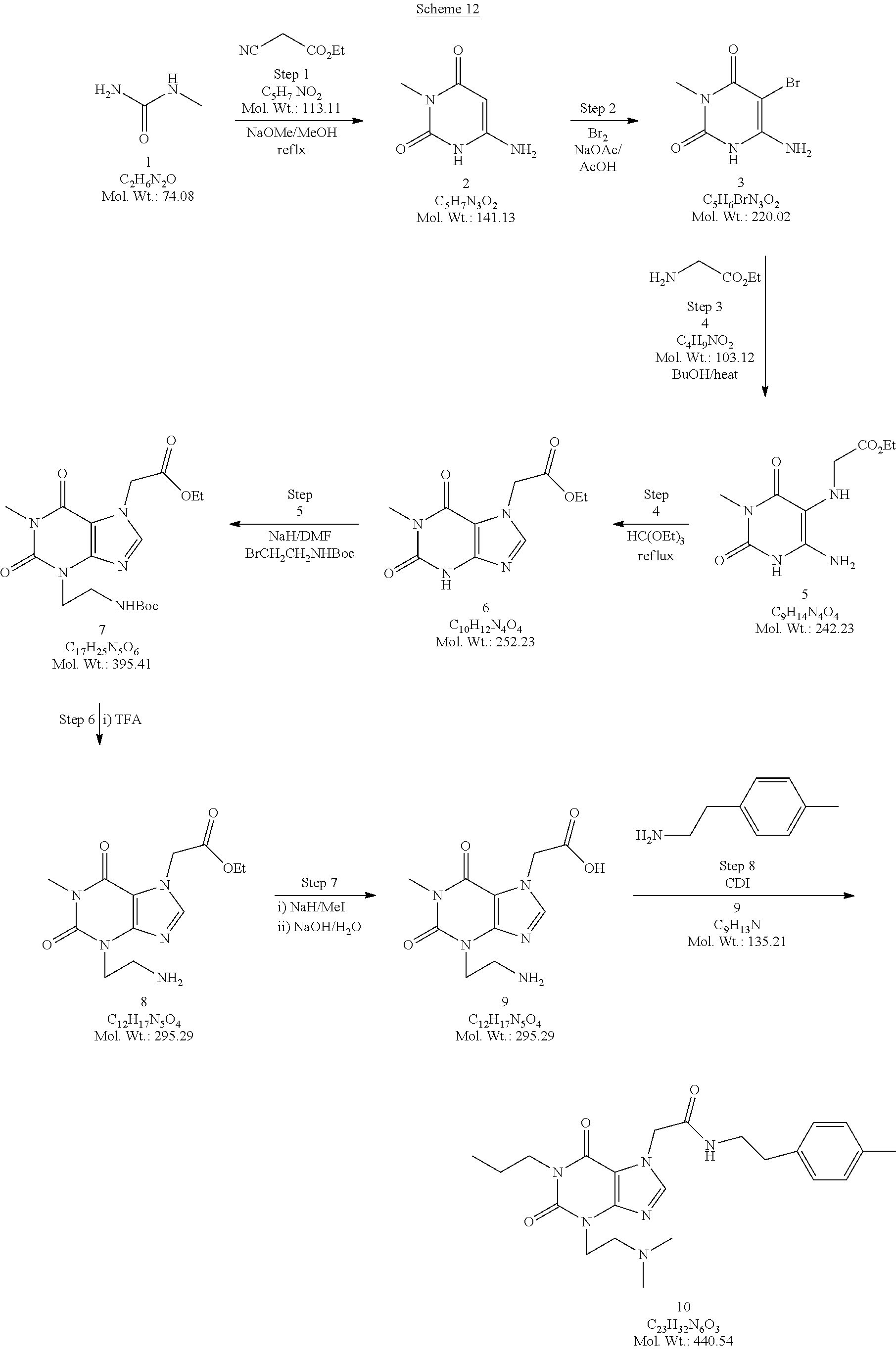

Compound 10 (Scheme 12) can be prepared according to similar reaction procedures shown in Scheme 1.

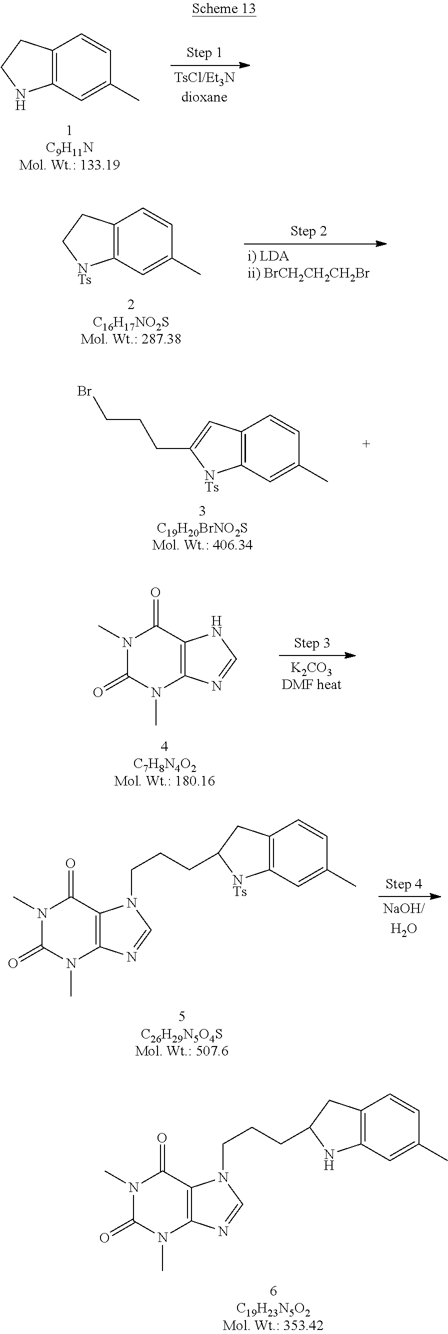

Treatment of Protected indoline 2 with LDA and dibromopropane gives compound 3, which subsequently can react with purine-dione 4, followed by hydrolysis reaction to yield compound 6.

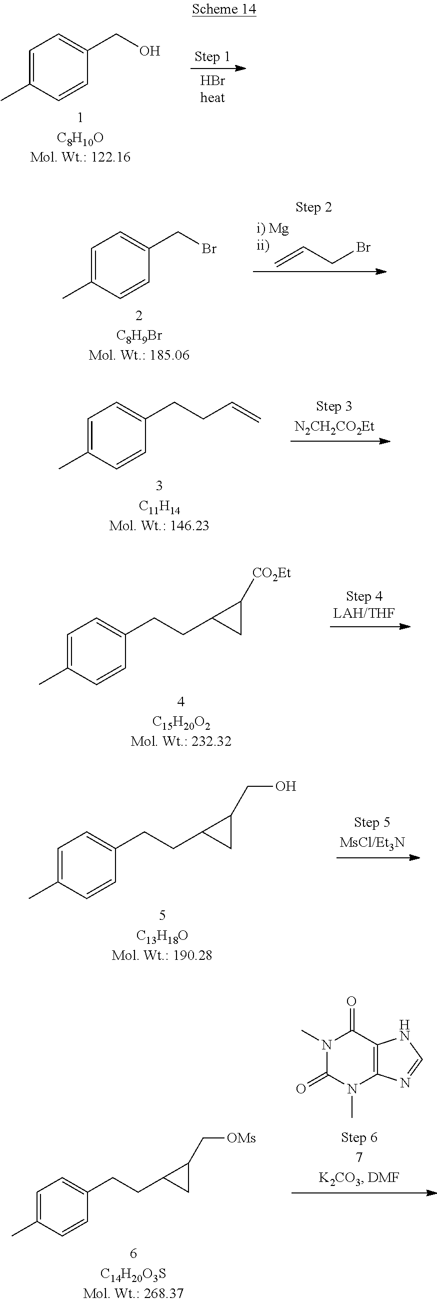

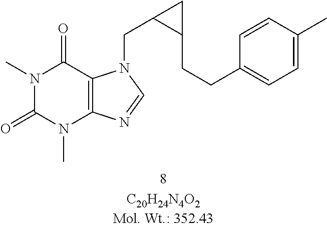

p-Tolylmethanol (1) can be converted to 1-(bromomethyl)-4-methylbenzene 2, which can be treated with Mg and allyl bromide to give compound 3. Treatment of alkene 3 with N₂CH₂CO₂Et, followed by a reduction reaction affords (2-(4-methylphenethyl)cyclopropyl)methanol (5). Cyclopropylmethanol 5 can react with MsCl, and the resulting compound 6 can be coupled with purine-dione 7 to afford compound 8.

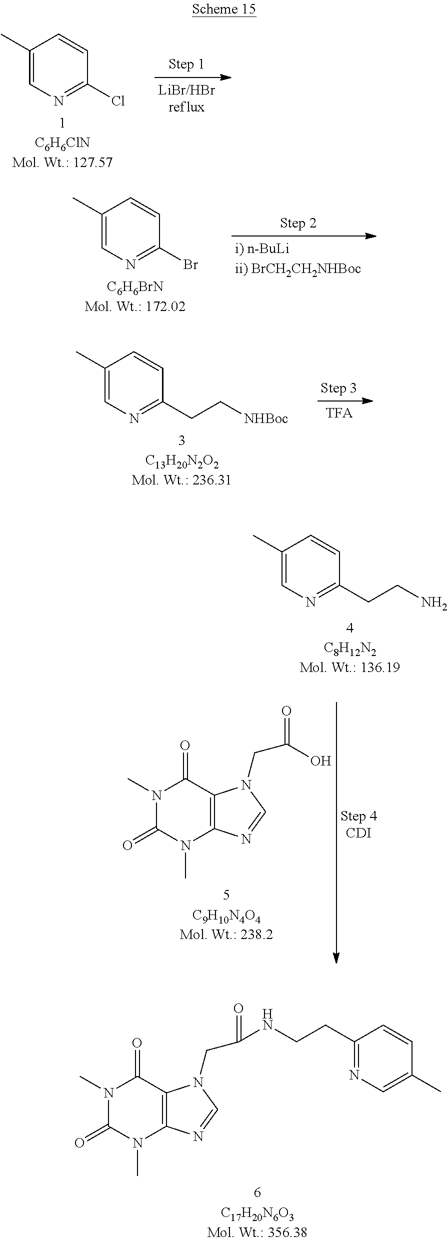

2-(5-Methylpyridin-2-yl)ethanamine 4 can be prepared by converting 2-chloro-5-methylpyridine (1) to 2-bromo-5-methylpyridine, followed by reacting with Boc-protected 2-bromoethanamine and removal of the protecting group with TFA. The obtained amine 4 then can be coupled with carboxylic acid 5 to afford compound 6.

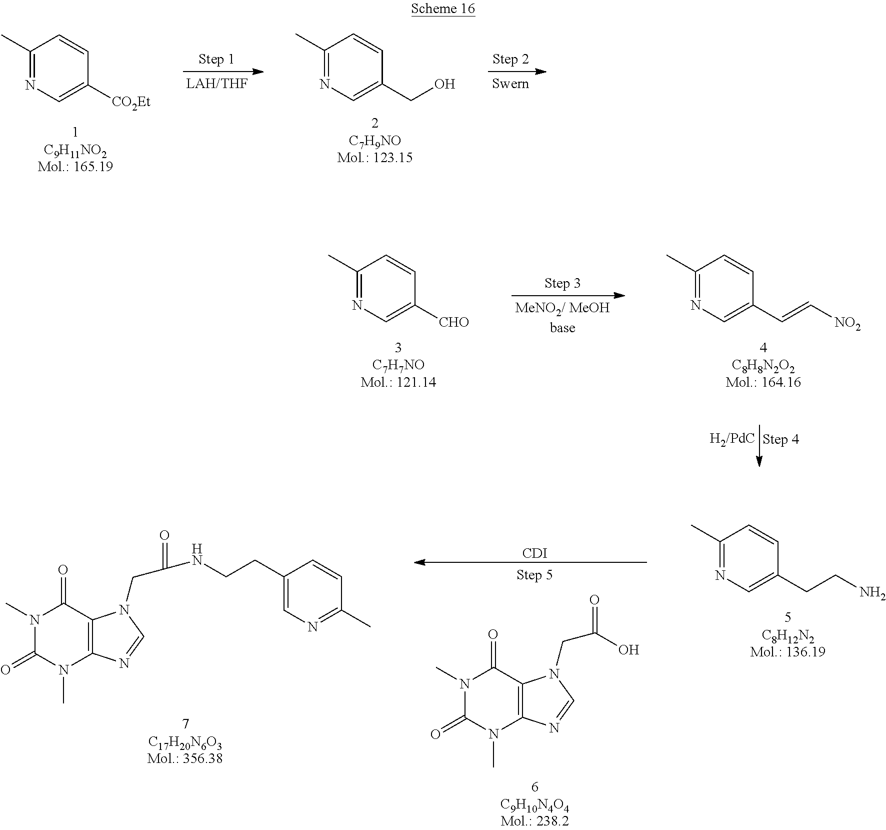

LAH can reduce ethyl 6-methylnicotinate to give alcohol 2, which can be oxidized and subsequently treated with MeNO₂ to yield compound 4. Compound 4 can be reduced to amine 4, which can be coupled with carboxylic acid 6 to give compound 7.

### Example 7

### Synthesis of N-(4-(4-(diethylamino)phenyl)thiazol-2-yl)-2-(1,3-dimethyl-2,6-dioxo-3,4,5,6-tetrahydro-1H-purin-7(2H)-yl)acetamide

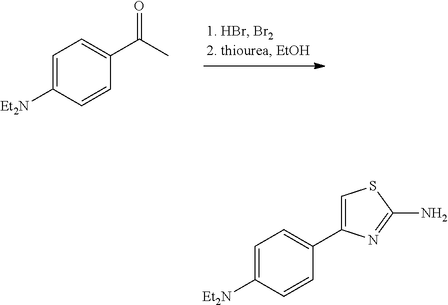

To a solution of 4′-diethylamionoacetophenone (20.80 g, 0.109 mol) in 45 mL HBr (48% in water), a solution of bromine (5.50 mL, 0.109 mol) in 35 mL HBr was added slowly via addition funnel over 20 min. Reaction mixture was stirred at room temperature overnight, diluted with 300 mL water and poured onto NaHCO₃/ice mixture. The mixture was extracted with CHCl₃ (2×400 mL), and the combined organic phase was washed with brine, and then dried over Na₂SO₄. After evaporation, the green oil was dissolved in 120 mL EtOH followed by addition of thiourea (8.30 g, 0.109 mol) and the solution was refluxed for 2 hrs. After evaporation of about 50 mL EtOH on rotovap, lot of solid precipitated in the flask. The mixture was filtered, washed with EtOH (100 mL), and then dried under vacuum to get brownish solid 20.86 g (77%). Ref: J. Org. Chem. 2003, 68, 839-853.

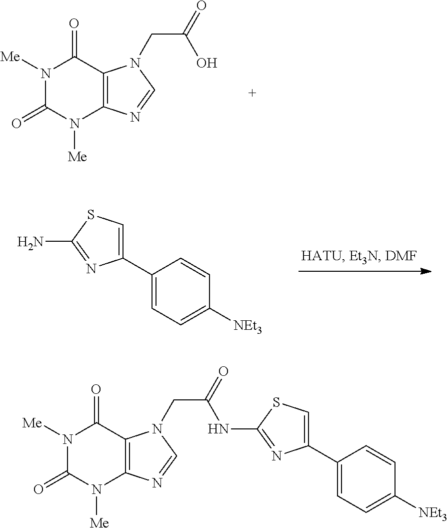

In an oven-dried round-bottom flask, theophylline-7-acetic acid (3.18 g, 13.3 mmol) and triethylamine (2.5 mL, 18.2 mmol) was dissolved in 60 mL DMF, and then 4-(diethylamino-phenyl)-thiazol-2-ylamine (3.00 g, 12.1 mmol) was added. After the amine totally dissolved, the solution was cooled in an ice-water bath for 20 min, and then HATU was added in one portion. The reaction mixture was warmed up to room temperature gradually and stirred at this temperature for 90 min. Mass and TLC showed consumption of amine. The solution was poured into 500 mL brine at 0° C., and the cloudy suspension was stirred for 30 min at this temperature. The suspension was filtered and washed with water and ether. Solid was dried in oven (50° C.) for 2 h, the off-white solid was then suspended in 500 mL EtOAc/10% MeOH and refluxed for 2 hrs. Hot filtration was performed, and the solid was washed with ether and dried on vacuum to give white solid 4.00 g (71%). mp: 305-307° C. R_{f}=0.31 (EtOAc).

### INCORPORATION BY REFERENCE

All publications and patents mentioned herein, are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating a respiratory condition in a subject, the method comprising administering a compound of formula (VIII), 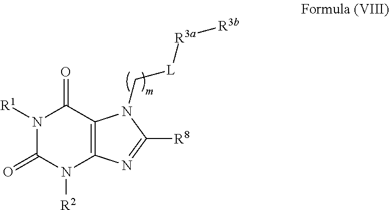
wherein,
R¹ and R² are each independently C₁-C₆ alkyl, C₂-C₆ alkenyl, or C₂-C₆ alkynyl, each of which is optionally substituted with 1-4 R⁵;
L is NR⁶SO₂, SO₂NR⁶, OC(O)NR⁶, NR⁶C(O)O, NR⁶C(O)NR⁶, NR⁶C(O), C(O)NR⁶, O, C(O), S, S(O), S(O)₂, NR₆, or CH₂,
each of R^{3a} and R^{3b} is independently cyclyl, heterocyclyl, aryl, heteroaryl, each of which is optionally substituted with 1-4 R⁷;
each R⁵ is independently halo, hydroxyl, alkoxy, amino, alkylamino, dialkylamino, cyano, nitro, amido(e.g., where the nitrogen of the amide is substituted by an alkyl, or where the nitrogen of the amide together with two carbons to which it is attached, forms a ring), alkylamido, dialkylamido, thioyl, sulfonyl, cyclyl, heterocyclyl, aryl, or heteroaryl;
each R⁶ is independently H, C₁-C₆ alkyl, C₁-C₆ alkenyl, hydroxyC₁-C₆ alkyl, alkoxyC₁-C₆ alkyl, cyanoalkyl, haloalkyl, arylalkyl, S(O)alkyl, acyl, amino, amidyl, or S(O)₂H, aryl, alkoxyaryl;
each R⁷ is independently C₁-C₆ alkyl, C₂-C₆ alkenyl, or C₂-C₆ alkynyl, halo, hydroxyl, alkoxy, oxo, aryl, heteroaryl, cyclyl, heterocyclyl, arylalkyl, heteroarylalkyl, cyclylalkyl, heterocyclylalkyl, aryloxy, arylalkoxy, amino, alkylamino, dialkylamino, thioyl, alkylthioyl, sulfonyl, sulfonamidyl, amido (e.g., where the nitrogen of the amide is substituted by an alkyl, or where the nitrogen of the amide together with two carbons to which it is attached, forms a ring), hydroxyl alkoxyl, alkoxy —C(O)OH, —C(O)Oalkyl, urea, sulfonylurea, acyl, nitro, cyano, each of which is optionally substituted with 1-3 R⁸;
each R⁸ is independently C₁-C₆ alkyl, C₂-C₆ alkenyl, or C₂-C₆ alkynyl, aryl, heteroaryl, cyclyl, halo, hydroxyl, alkoxy, oxo, aryloxy, amino, alkylamino, dialkylamino, C(O)OH, —C(O)Oalkyl, thioyl, sulfonyl, sulfonamidyl, amido (e.g., where the nitrogen of the amide is substituted by an alkyl, or where the nitrogen of the amide together with two carbons to which it is attached, forms a ring), urea, sulfonylurea, acyl, nitro, cyano, cyclyl, heterocyclyl, aryl, or heteroaryl;
R⁹ is H, C₁-C₆ alkyl, C₂-C₆ alkenyl, or C₂-C₆ alkynyl, halo, C₁-C₆ haloalkyl, hydroxyl, alkoxy, aryloxy, arylalkoxy, amino, alkylamino, dialkylamino, thioyl, alkylthioyl, sulfonyl, sulfonamidyl, amido, urea, sulfonylurea, acyl, nitro, cyano, each of which is optionally substituted with 1-3 R⁸;
m is 1, 2, 3, 4, 5, or 6.

2. The method of claim 1, wherein m is at least 2 when L is connected to the methylene carbon via a heteroatom.

3. The method of claim 1, wherein when L is CH₂, S, C(O)NR⁶ or NR⁶C(O), R^{3a} is not a 5-membered heterocyclyl, 5-membered heteroaryl, or piperazine.

4. The method of claim 1, wherein when L is C(O)NH, R^{3a} and R^{3b} are not both phenyl.

5. The method of claim 1, m is at least 2 when L is connected to the methylene carbon via a heteroatom.

6. The method of claim 1, wherein R¹ is C₁-C₆ alkyl.

7. The method of claim 1, wherein R² is C₁-C₆ alkyl.

8. The method of claim 1, wherein R^{3a} is monocyclic.

9. The method of claim 1, wherein R^{3a} is aryl, for example, phenyl.

10. The method of claim 1, wherein R^{3a} is

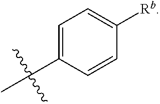

11. The method of claim 1, wherein R^{3a} is heterocyclyl.

12. The method of claim 1, wherein R^{3a} is heteroaryl.

13. The method of claim 12, wherein R^{3a} is a nitrogen containing heteroaryl.

14. The method of claim 13, wherein R^{3a} is

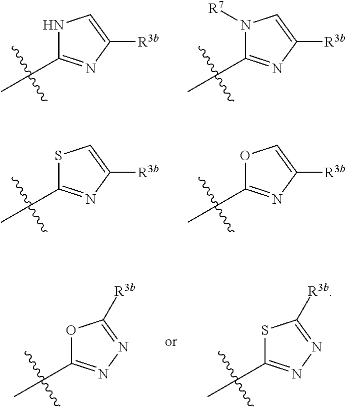

15. The method of claim 14, wherein R^{3a} and R^{3b} together form 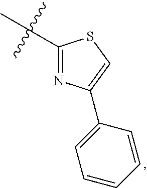
wherein R^{3a} and/or R^{3b} is optionally further substituted by 1-4 R⁷.

16. The method of claim 1, wherein the compound is formula (VIII′) A method of treating a TRPA1 mediated disorder in a subject, the method comprising administering a compound of (VIII′)

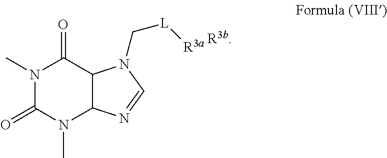

17. The method of claim 16, wherein L is C(O)NR⁶.

18. The method of claim 1, wherein the compound is a compound of Formula (VIII″) 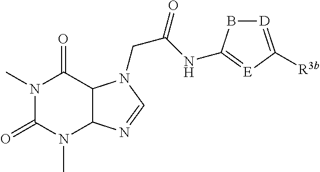
Formula (VIII″), wherein B is O, S, or NR⁶; D and E are independently CH, CR⁷ or N.

19. The method of claim 1, wherein the compound is a compound of Formula (VIII′″) 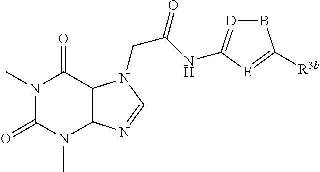
Formula (VIII′″), wherein B is O, S, or NR⁶; D and E are independently CH, CR⁷ or N.

20. A method of treating a respiratory condition in a subject, the method comprising administering a compound of formula (VIIIa) 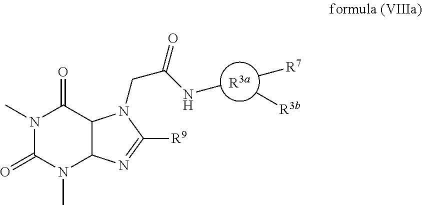
wherein
R^{3a} cyclyl, heterocyclyl, aryl, heteroaryl,
R^{3b} is cyclyl, heterocyclyl, aryl, heteroaryl; optionally substituted with 1-3 R⁷;
each R⁷ is independently C₁-C₆ alkyl, C₂-C₆ alkenyl, or C₂-C₆ alkynyl, halo, oxo, hydroxyl, alkoxy, aryl, heteroaryl, cyclyl, heterocyclyl, aryloxy, arylalkoxy, amino, alkylamino, dialkylamino, thioyl, alkylthioyl, sulfonyl, sulfonamidyl (e.g., where the nitrogen of the sulfonamide is substituted by an alkyl, or where the nitrogen of the sulfonamide together with two carbons to which it is attached, forms a ring), amido(e.g., where the nitrogen of the amide is substituted by an alkyl, or where the nitrogen of the amide together with two carbons to which it is attached, forms a ring), hydroxyl alkoxyl, alkoxy alkoxyl, urea, sulfonylurea, acyl, nitro, cyano, each of which is optionally substituted with 1-3 R⁸;
each R⁸ is independently C₁-C₆ alkyl, C₂-C₆ alkenyl, or C₂-C₆ alkynyl, aryl, heteroaryl, cyclyl, halo, hydroxyl, alkoxy, aryloxy, amino, alkylamino, dialkylamino, thioyl, sulfonyl, sulfonamidyl, amido, urea, sulfonylurea acyl, nitro, cyano, cyclyl, heterocyclyl, aryl, or heteroaryl; and
R⁹ is H or halo.

21. A method of treating a respiratory condition in a subject, the method comprising administering a compound of formula (VIIIb) 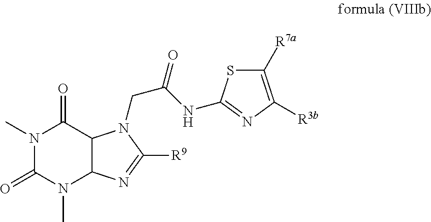
wherein R^{3b} is aryl or heteroaryl; optionally substituted with 1-3 R⁷;
each R⁷ is independently C₁-C₆ alkyl, C₂-C₆ alkenyl, or C₂-C₆ alkynyl, halo, hydroxyl, alkoxy, aryl, heteroaryl, cyclyl, heterocyclyl, aryloxy, arylalkoxy, amino, alkylamino, dialkylamino, thioyl, alkylthioyl, sulfonyl, sulfonamidyl, amido, hydroxyl alkoxyl, alkoxy alkoxyl, urea, sulfonylurea, acyl, nitro, cyano, each of which is optionally substituted with 1-3 R⁸;
R^{7a} is H, C₁-C₆ alkyl, C₂-C₆ alkenyl, or C₂-C₆ alkynyl, halo, hydroxyl, alkoxy, aryl, heteroaryl, cyclyl, heterocyclyl, aryloxy, arylalkoxy, amino, alkylamino, dialkylamino, thioyl, alkylthioyl, sulfonyl, sulfonamidyl, amido, hydroxyl alkoxyl, alkoxy alkoxyl, urea, sulfonylurea, acyl, nitro, cyano, each of which is optionally substituted with 1-3 R⁸;
each R⁸ is independently C₁-C₆ alkyl, C₂-C₆ alkenyl, or C₂-C₆ alkynyl, aryl, heteroaryl, cyclyl, halo, hydroxyl, alkoxy, aryloxy, amino, alkylamino, dialkylamino, thioyl, sulfonyl, sulfonamidyl, amido, urea, sulfonylurea acyl, nitro, cyano, cyclyl, heterocyclyl, aryl, or heteroaryl; and
R⁹ is H or halo.

22. The method of claim 1, 20, or 21, wherein said respiratory condition is not asthma, COPD, or chronic cough.
